SEQUENCE LISTING (4 sequences)

SEQ ID NO: 1 - DNA, 19 bp, synthetic construct
  source 1..19: mol_type = other DNA
agatgtgtataagagacag

SEQ ID NO: 2 - DNA, 16 bp, synthetic construct
  source 1..16: mol_type = other DNA
ctgtctcttatacaca

SEQ ID NO: 3 - DNA, 83 bp, synthetic construct
  source 1..83: mol_type = other DNA
  modified_base 50: mod_base = OTHER; note = deoxyuridine
tcttctacagtnnnnnnagatcggaagagcacacgtctgaactccagtctacactctttccctacacgacgctcttccgatct

SEQ ID NO: 4 - DNA, 27 bp, synthetic construct
  source 1..27: mol_type = other DNA
gtagggaaagagtgtagactggagttc

(19) United States
(12) Patent Application Publication
(10) Pub. No.: US 2025/0002972 A1
(43) Pub. Date: Jan. 2, 2025

(54) METHOD FOR MEASURING SOMATIC DNA MUTATION AND DNA DAMAGE PROFILES AND A DIAGNOSTIC KIT SUITABLE THEREFORE

(71) Applicant: Albert Einstein College of Medicine, Bronx, NY (US)

(72) Inventors: Alexander Y. Maslov, Stamford, CT (US); Jan Vijg, New York, NY (US)

(21) Appl. No.: 18/709,334

(22) PCT Filed: Nov. 10, 2022
(86) PCT No.: PCT/US2022/049548

§ 371 Date: May 10, 2024

Related U.S. Application Data
(60) Provisional application No. 63/277,955, filed on Nov. 10, 2021.

(51) Int. Cl. C12Q 1/6806; C12N 15/10; C12N 15/113; C12Q 1/6827; C12Q 1/6883; G16B 20/20

(52) U.S. Cl. CPC C12Q 1/6806; C12N 15/1093; C12N 15/113; C12Q 1/6827; C12Q 1/6883; G16B 20/20; C12N 2310/122; C12Q 2600/156

(57) ABSTRACT

Disclosed are compositions and methods related to detecting rare mutations (e.g., somatic mutations) or genome structure variants using rolling circle-based linear amplification and next generation sequencing.

### CROSS-REFERENCE TO RELATED APPLICATIONS

This application claims the benefit of U.S. Provisional Application No. 63/277,955, filed on Nov. 10, 2021, the entire contents of which are incorporated herein in their entirety by this reference.

### GOVERNMENT SUPPORT

This invention was made with government support under grant numbers P01 AG017242, U01 ES029519, and U01 HL145560 awarded by the National Institutes for Health. The government has certain rights in the invention.

### BACKGROUND

Mutations in the genome of somatic cells of multicellular organisms are the inevitable consequence of errors during DNA repair or replication. Somatic mutations cause cancer and have been implicated in other pathologies. Attempts have been made in the past to develop assays for the quantitative analysis of various types of mutations in cells and tissues. In view of the dramatic progress of DNA sequencing one would think that somatic mutations should be easy to detect quantitatively in human or animal cells and tissues. Indeed, in a very short time an enormous amount of information has become available about somatic mutations in human tumors. However, tumors are clonal lineages with many mutations shared between the individual cells of the tumor. Mutations in normal tissues, however, are mostly unique for each cell and their detection by sequencing remains a challenge because somatic mutations occur at low abundance and are spread through the reads, indistinguishable from sequencing errors. One way to overcome this problem is utilizing a single cell-based approach. However, while the single cell approach is currently the only method allowing comprehensive genome-wide assessment of somatic mutational loads, this method is resource- and time-consuming with a high price tag, which limits its broad application. An alternative approach, Duplex-Seq, is based on a comparative analysis of the complementary DNA strands and allows accurate quantitative identification of ultra-rare somatic single-nucleotide variants (SNVs) in bulk DNA. While less demanding technically than single cell sequencing, Duplex-Seq's capacity to suppress errors is limited to the square of the probability of errors on one strand. Moreover, it also suffers from low effective coverage due to the need for redundant PCR amplification, which restricts its practical application to the analysis of small targets, such as mitochondrial DNA, plasmids, or individual genes.

Accordingly, there is a great need in the art for compositions and methods for the accurate cost-effective assessment of somatic single nucleotide variants (SNVs) in bulk DNA extracted from normal cells and tissues.

### SUMMARY

Provided herein are compositions and methods for Single Molecule Mutation Sequencing (SMM-Seq) for the accurate and cost-effective assessment of somatic single nucleotide variants (SNVs) in bulk DNA extracted from normal cells and tissues.

### BRIEF DESCRIPTION OF THE DRAWINGS

FIG. 1A-FIG. 1B show the outline of SMM-Seq workflow and variant calling algorithm. (FIG. 1A) Both ends of end-repaired and A-tailed DNA fragments are ligated with a hairpin-like adapter. The adapter contains a 6-nt long unique molecular identifier (UMI) in its stem part allowing identification of sequencing reads from the same original DNA fragment (UMI-family) as well as identification of strand families. The hairpin-like adapter contains uracil in its loop part, allowing Uracil-DNA Glycosylase (UDG)-mediated breakage and PCR amplification when a conventional sequencing library is needed. The resulting dumbbell-like constructs, with intact uracils, serve as templates for the subsequent pulse-RCA reaction. Single stranded DNA contigs are then PCR-amplified to obtain multiple independent replicates of the original DNA fragments. Sequencing reads are aligned to the corresponding reference genome, UMI families identified and somatic variants are identified according to the computational algorithm shown (FIG. 1B).

FIG. 2A-FIG. 2C show quantitative detection of induced somatic SNVs. (FIG. 2A) Relative mutation frequency as a function of strand family size. (FIG. 2B) Frequency of somatic SNVs in IMR90 cells 72 hours after treatment with different doses ENU. (FIG. 2C) Spectra of somatic SNVs in control cells and cells treated with ENU. All data points represent three biological replicates. Data shown as average±SD; asterisk (*) designates a statistically significant difference with its control (**P<0.01; ***P<0.001).

FIG. 3A-FIG. 3D show quantitative detection of somatic SNVs in normal human liver. (FIG. 3A) Frequency of somatic SNVs in normal human liver of different ages. (FIG. 3B) Spectra of somatic SNVs in normal human liver of different ages. (FIG. 3C)—two mutational signatures de novo identified among variants detected by SMM-Seq in two different age groups. (FIG. 3D) Contributions of signatures S1 and S2 to somatic SNVs found in hepatocytes of young and aged groups. All data points represent three biological replicates. Data shown as average±SD.

FIG. 4 depicts a computing node according to an embodiment of the present disclosure.

FIG. 5 shows spectra of somatic SNVs in control IMR90 cells and cells treated with ENU.

FIG. 6 shows quantitative detection of somatic SNVs in normal human liver using SMM-Seq and single cell sequencing-based approaches. For the single cell study the bars indicate the median mutation frequencies between 3 individual cells±SD.

FIG. 7 shows spectra of somatic SNVs in normal human liver of young and old individuals.

FIG. 8 shows contributions of signatures S1 and S2 to somatic SNVs found in hepatocytes of young and aged individuals.

FIG. 9A-FIG. 9C show genomic structural variation (SV). (FIG. 9A)—formation of artificial chimeric sequences. (FIG. 9B)—SMM-SV design. (FIG. 9C)—somatic SV frequency in human mammary epithelial cells upon exposure to different doses of bleomycin. Data are shown as average±SD; n=3 for all data points; asterisk (*) designates a statistically significant difference with its control (***P<0.001).

### DETAILED DESCRIPTION

Postzygotic somatic mutations have been found associated with human disease, including cancer and diseases other than cancer. Most information on somatic mutations has come from studying clonally amplified mutant cells, based on a growth advantage or genetic drift. However, almost all somatic mutations are unique for each cell and the quantitative analysis of such low-abundance mutations in normal tissues remains a major challenge in biology.

Provided herein are compositions and methods for Single Molecule Mutation Sequencing (SMM-Seq) for quantitative identification of point mutations in normal cells and tissues.

Provided also herein are compositions and methods for modified SMM-Seq for detecting genome structural variants (SVs).

### SMM-Seq for Quantitative Identification of Point Mutations

This invention relates to a method for measuring genetic and epigenetic DNA mutational profiles as well as DNA damage profiles in primary normal cells and tissues. The method of the present disclosure uses double stranded DNA fragments to create multiple independent copies of both DNA strands of each DNA fragments. These copies then sequenced and analyzed to reconstruct the sequence of the original DNA fragment determined as a consensus sequence of all copies derived from this fragment. Genetic and epigenetic mutations are determined as changes in DNA sequence observed on copies from both DNA strands. DNA damage events are determined as changes in DNA sequence observed on copies of only one DNA strand.

There are various approaches utilizing analysis of DNA strands for the identification of rare mutations, i.e., the original Duplex-Seq, BotSeqS, and NanoSeq. The error rate of these approaches is determined by the probability of two complementary errors in both strands and can be defined as P(E)², where P(E) is the probability of error on any of two strands.

The method of the present disclosure is not limited to two strands only since it utilizes sequencing data from multiple independent copies of each strand for variant calling. Conversely, SMM-Seq's error rate can be calculated as P(E)^{N}, where N is the number of independent copies produced in the linear amplification step. Thus, unlike existing assays accuracy of the method of the present disclosure in base calling is virtually unlimited.

### Modified SMM-Seq for Detection of Genome Structural Variations (SVs)

SMM-seq and all other single-molecule mutation assays, e.g., Duplex-seq, Nano-seq, can detect base substitutions and small insertions or deletions. These are called point mutations and are important in causing cancer and other diseases. However, none of these assays can detect a larger type of mutation, called genome structural variation or SV for short. SVs include deletions, inversions, insertions, duplications, and translocations that can affect large stretches of genomic DNA, from about 50 basepairs to thousands and millions of basepairs. It is generally known that such large mutations are much more impactful than point mutations. Hence, their inclusion in assays such as our SMM-seq assay would significantly extend the application range of SMM-seq. Indeed, SVs are causally related to human diseases, such as cancer, much more often than point mutations (Spielmann et al. (2018) Nat Rev Genet 19 (7): 453-467). However, they are impossible to detect as somatic mutations, either by single-cell or single-molecule assays. Below we describe how we developed a modification of SMM-seq that allows to detect such mutations.

### Exemplary Utilities

There is a great need for an accurate, genome-wide measure for DNA mutation loads. For example, for cancer patients exposed to chemotherapeutic agents, workers who might have been exposed to radioactive or chemical agents, victims of a terrorist attack with a dirty bomb or simply as a diagnostic measure for genetic disease, such as cancer, or aging rate. Additional utilities for the compositions and methods of the present disclosure include the following:
Testing mutagenicity of new or existing chemical compounds. This is now being done by the Ames test, which essentially tests for mutation induction in Salmonella. It is well known that the predictivity of this assay is very low, the main problem being the many compounds that result in false positives. This means that industry, including pharmaceutical industry, discards many more compounds than necessary.
Biohazards exposure diagnostic. Thus far it has been impossible to assess individuals at risk for cancer or other genetic diseases because of exposure to mutagenic agents. Examples are industrial accidents, nuclear disasters like Chernobyl and dirty bombs as part of terrorist attacks. To have an assay that could quickly and accurately report on the level of exposure would be instrumental in taking further action.
Individual risk assessment. It is conceivable that somatic mutation loads are a general marker for individual aging rate. Routine application of the assay would allow the identification of individuals at risk who could then use more extensive prevention measures than they would otherwise do, varying from more frequent colonoscopies to sunscreen.

### Definitions

As used herein, the singular forms “a,” “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

All numerical ranges provided herein are understood to be shorthand for all of the decimal and fractional values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9 and all intervening fractional values between the aforementioned integers such as, for example, ½, ⅓, ¼, ⅕, ⅙, ⅛, and 1/9, and all multiples of the aforementioned values. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “comprise” is generally used in the sense of include, that is to say permitting the presence of one or more features or components. Wherever embodiments, are described herein with the language “comprising,” otherwise analogous embodiments described in terms of “consisting of,” and/or “consisting essentially of” are also provided.

As used herein, two nucleic acid sequences “complement” one another or are “complementary” to one another if they base pair one another at each position.

The terms “polynucleotide” and “nucleic acid” are used herein interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

The term “somatic mutation” refers to an alteration in DNA that occurs after conception. Somatic mutations can occur in any of the cells of the body except the germ cells (sperm and egg) and therefore are not passed on to children.

The term “indel” refers to an insertion or deletion of bases in the genome of an organism.

### Unique Molecular Identifier (UMI)

Unique molecular identifiers (UMIs) are a type of molecular barcoding that provides error correction and increased accuracy during sequencing. These molecular barcodes are short sequences used to uniquely tag each molecule in a sample library.

Thus, UMIs are complex indices added to sequencing libraries before any PCR amplification steps, enabling the accurate bioinformatic identification of PCR duplicates. UMIs are also known as “Molecular Barcodes” or “Random Barcodes”.

UMIs are valuable tools for both quantitative sequencing applications (e.g. RNA-Seq, ChIP-Seq) and also for genomic variant detection, especially the detection of rare mutations. UMI sequence information in conjunction with alignment coordinates enables grouping of sequencing data into read families representing individual sample DNA or RNA fragments.

### The Problems UMIs are Addressing:

Quantitative analysis: Many sequencing library preparation protocols enable high-throughput sequencing (HTS) from low amounts of starting material. Their preparation requires PCR amplification of the libraries. While the PCR polymerases and reagents have been improved greatly in recent years enabling a mostly unbiased amplification of sequencing libraries, some biases still remain against sequences with extreme GC contents and against long fragments. When starting from ultra-low input samples, stochastic effects in the first rounds of the PCR add to the problems. These issues can potentially cause erroneous quantitation data. Removal of PCR duplicates using alignment coordinate information is especially inefficient such for low input situations but also for deep sequencing data. In the latter case alignment coordinate-based de-duplification will remove large numbers of biological duplicate reads from the data, especially for the most abundant transcripts.

UMIs alleviate the PCR duplicate problem by adding unique molecular tags to the sequencing library molecules before amplification.

Rare variant analysis: Sequencing provides data with low error rates (˜0.1 to 0.5%) for most applications. These low error rates nevertheless interfere with the confident identification of low abundance variants. Data without UMI cannot distinguish between these and sequencing errors. UMIs in combination with deep sequencing yielding multiple reads for each of the sample DNA fragments solves this problem and increases the accuracy of the sequencing data significantly.

A UMI may comprise at least or about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 nucleotides. In some embodiments, A UMI may comprise less than about 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 nucleotides. In some embodiments, a UMI may comprise at least or about 5 nucleotides, but less than about 100 nucleotides In some embodiments, a UMI may comprise at least one spacer. In some embodiments, the at least one spacer may comprise at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

### Tagmentation

Tagmentation is the initial step in library prep where a hyperactive transposase is used to simultaneously fragment target DNA and append universal adapter sequences.

The first step in tagmentation is the formation of the transposome complexes, composed of a hyperactive variant of the Tn5 transposase homodimer complexed with sequences that contain the 19-bp double-stranded Mosaic End (ME) sequence recognized by the enzyme. In a traditional transposition reaction, Tn5 would be loaded with a single, continuous stretch of double-stranded transposon DNA flanked by ME sequences; whereas in tagmentation, the transposon DNA is discontinuous, with two, unlinked adapter sequences. The adapter itself is composed of the ME sequence with an additional 5′ overhang of single-stranded DNA on the transfer strand (i.e., the strand that becomes covalently bound to the target DNA) that is a mix of either forward or reverse adapter sequences to be used as PCR handles in subsequent processing steps. The single-stranded component is to prevent the action of the enzyme on the actual adapter complexes themselves. Tn5 has a high propensity to insert into free double-stranded DNA, and making the only double-stranded portion the ME, which is protected by the Tn5 enzyme, prevents this “self-tagmentation” from happening. On a related note, the in vitro assembly of transposome complexes should be performed in the absence of Mg2+, which is required for the tagmentation reaction to occur, in order to prevent tagmentation within the 19-bp double-stranded ME region of adapters that has not yet formed a complex. The other major aspects of adapter design include the use of a 5′ phosphorylated ME reverse complement. This bottom strand can also be reduced in length from the full 19-bp segment, with 16-bp versions (trimmed from 3′ end) providing comparable efficiency (Adey and Shendure (2012) Genome Res 22:1139-1143, which is incorporated herein by reference). The 19-bp segment of ME contains the sequence of 5′-AGATGTGTATAAGAGACAG-3′. The 16-bp version is not ME but is a complement to ME, and contains the sequence of 5′-CTGTCTCTTATACACA-3′. In standard tagmentation assays, transposome assembly is composed of mixing a 1:1:2 ratio of the forward and reverse adapters and purified Tn5 monomer. The Tn5 protein can be produced using published methods (Picelli et al. (2014) Genome Res 24:2033-2040; Kia et al. (2017) BMC Biotechnol 17:6, each of which are incorporated herein by reference). One important note is that what may appear to be a poor quality Tn5 preparation, may in fact be driven by the use of poor-quality oligonucleotides. As such, it is critical to always use HPLC-purified oligonucleotides and perform activity-based quantification using standard adapters and benchmarking against commercially-available options. Other modes of failure include protein that has not properly folded or inaccurate quantification of active enzyme, the latter of which can be addressed by performing activity-based quantification by titrating across several possible concentrations and benchmarking against commercially-available options.

Purified DNA is then exposed to these transposome complexes within a buffer that contains Mg2+, which is required for the transposition reaction to occur. The complexes act on the target DNA by binding tightly and completing cleavage and strand transfer at two positions that are 9 bp apart. The result is a break in the target DNA at both strands with a 9-bp space in between. At each of these nicks, the transfer strand oligonucleotide containing the ME sequence and either a forward or reverse adapter is covalently attached. From a single tagmentation event, adapters are incorporated in an outward-facing manner; thus, in order to form a viable sequencing library molecule, a second tagmentation event needs to be completed successfully nearby (i.e., within a length suitable for PCR and sequencing, typically <1000 bp). The tagmentation enables the production of libraries from as little as 10 μg of starting material in its initial description, approaching the single-cell range of input.

After the transposition reaction itself, a process referred to here as end repair must be performed before denaturation of the template DNA for subsequent PCR amplification. This process first involves the removal of the Tn5 protein, which remains tightly bound to the target DNA in order to free up the DNA present at the site of tagmentation. For sequencing applications, Tn5 removal is facilitated by a cleanup procedure or treatment with a detergent (SDS). Skipping the Tn5 removal step is possible, although it results in a much lower efficiency of end repair, which may be acceptable for applications in which efficiency is of less value than a rapid workflow.

The removal of Tn5 effectively releases the two end fragments from one another that were generated during the reaction, each receiving one of the adapters from the transposome complex and retaining one strand of the 9-bp region in between the two cut sites. Extension using a DNA polymerase from 3′ end of the strand that was not subjected to strand transfer then copies the 9-bp overlap region and the ME sequence, terminating at the end of the adapter. The 9-bp region is effectively copied and is the sequence present at the outermost ends of sequencing library molecules, where two adjacent library molecules each overlap at the same 9-bp segment.

After end repair, templates are denatured and carried through PCR with primer sequences corresponding to the forward and reverse adapters that contain an overhang with an optional index sequence and terminate in the sequences used for cluster generation on a sequencer flowcell. Libraries are then sequenced using primers that correspond to the full forward or reverse adapters to provide reads of the intervening genomic DNA.

### Sequencing

Any of a variety of sequencing reactions known in the art can be used to directly sequence a biomarker gene and detect mutations. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

In certain embodiments, detection of a mutation can be accomplished using methods including, but not limited to, sequencing by hybridization (SBH), sequencing by ligation (SBL), quantitative incremental fluorescent nucleotide addition sequencing (QIFNAS), pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al. (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), and the like. High-throughput sequencing methods, e.g., on cyclic array sequencing using platforms such as Roche 454, Illumina Solexa or MiSeq or HiSeq, AB-SOLID, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenom. 1:95-100; and Shi (2001) Clin. Chem. 47:164-172) (see, for example, U.S. Pat. Publ. Nos. 2013/0274117, 2013/0137587, and 2011/0039304).

Next-generation sequencing (NGS) is a technology for determining the sequence of DNA or RNA to study genetic variation associated with diseases or other biological phenomena. Introduced for commercial use in 2005, this method was initially called “massively-parallel sequencing”, because it enabled the sequencing of many DNA strands at the same time, instead of one at a time as with traditional Sanger sequencing by capillary electrophoresis (CE).

Because of the speed, throughput, and accuracy of NGS, NGS enables the interrogation of hundreds to thousands of genes at one time in multiple samples, as well as discovery and analysis of different types of genomic features in a single sequencing run, from single nucleotide variants (SNVs), to copy number and structural variants, and even RNA fusions. NGS provides the ideal throughput per run, and studies can be performed quickly and cost-effectively. Additional advantages of NGS include lower sample input requirements, higher accuracy, and ability to detect variants at lower allele frequencies than with Sanger sequencing.

Analyzing the whole genome using next-generation sequencing (NGS) delivers a base-by-base view of all genomic alterations, including single nucleotide variants (SNV), insertions and deletions, copy number changes, and structural variations. Paired-end whole-genome sequencing involves sequencing both ends of a DNA fragment, which increases the likelihood of alignment to the reference and facilitates detection of genomic rearrangements, repetitive sequences, and gene fusions.

In some embodiments, the Illumina “Phased Sequencing” platform, which employs a combination of long and short pair-ends, can be used. In other embodiments, the third-generation single-molecule sequencing technologies (e.g., ONT and PacBio) can produce much longer reads of DNA sequences.

In preferred embodiments, the “Deep Sequencing” or high-coverage version of Illumina NGS can be used to explore microheterogeneity in DNA sequences. Deep Sequencing refers to sequencing a genomic region multiple times, sometimes hundreds or even thousands of times. The Deep Sequencing allows detection of rare clonal types, cells, or microbes comprising as little as 1% of the original sample. Illumina's NovaSeq performs such whole-genome sequencing efficiently and cost-effectively, and its scalable output generates up to 6 Tb and 20 billion reads in dual flow cell mode with simple streamlined automated workflows.

### Genome Structure Variants (SVs) and Related Diseases

### Types of Structural Variants

Structural variation is an important type of human genetic variation that contributes to phenotypic diversity. There are microscopic and submicroscopic structural variants which include deletions, duplications (e.g., tandem duplications, dispersed duplications), and large copy number variants, as well as insertions (including mobile element insertions), inversions, and translocations. These are several different types of structural variants in the human genome and they are quite distinctive from each other. A translocation is a chromosomal rearrangement, at the inter- or intra-chromosomal level, where a section of a chromosome changes position but with no change in the whole DNA content. A Section of DNA that is larger than 1 kb and occurs in two or more copies per haploid genome, in which the different copies share greater than 90% of the same sequence, are considered to be segmental duplications or low-copy repeats.

An inversion is a section of DNA on a chromosome that is reversed in its orientation in comparison to the reference genome. There have been many studies identifying inversions because they have been found to have a big role in many diseases. A study found that forty percent of haemophilia A patients had a factor 8 gene inversion of a certain region that was four hundred kb in size. The inversion breakpoint was found to be around a segmental duplication which is observed in many other inversion events.

### Implications in Diseases or Conditions

### Charcot-Marie Tooth (CMT) Disease

There are several structural variants in the human genome that have been observed but have not led to any obvious phenotypic effects. There are some, however, that play a role in gene dosage which could lead to genetic diseases or distinct phenotypes. Structural variants can directly affect gene expression, such as with copy-number variants, or indirectly through position effects. These effects can have significant implications in susceptibility to disease. The first gene dosage effect that was observed, and considered to be an autosomal dominant disease from an inherited DNA rearrangement, was Charcot-Marie Tooth (CMT) disease. Most of the associations found with CMT were with a 1.5 Mb tandem duplication in 17p11.2-p12 at the PMP22 gene. When an individual has three copies of the normal gene, it results in the disease phenotype. If the individual had only one copy of the PMP22 gene, on the other hand, the result was a clinically different hereditary neuropathy with liability to pressure palsies. The differences in gene dosage created vastly different disease phenotypes which revealed the significant role that structural variation has on phenotype and susceptibility to disease.

### HIV Susceptibility

A study on the influence of the CCL3L1 gene on HIV-1/AIDS susceptibility tested if the copy number of the CCL3L1 gene had any effect on an individual's susceptibility to HIV-1/AIDS. The study sampled several different individuals and populations for their CCL3L1 copy number and compared it to their HIV acquirement risk. They found that there is an association between higher amounts in the copy number of CCL3L1 and susceptibility to HIV and AIDS since individuals who were more prone to HIV had a low copy number of CCL3L1. This difference in copy number was shown to play a possibly significant role in HIV susceptibility due to this association.

### Obesity

Another study that focused on the pathogenesis of human obesity tested if structural variation of the NPY4R gene was significant in obesity. Studies had previously shown that 10q11.22 copy number variations (CNV) had an association with obesity and that several copy number variants were associated with obesity. Their CNV analysis revealed that the NPY4R gene had a much higher frequency of 10q11.22 CNV loss in the patient population. The control population, on the other hand, had more CNV gain in the same region. This led the researchers to conclude that the NPY4R gene played an important role in the pathogenesis of obesity due to its copy number variation.

### Schizophrenia

It had been previously shown that variation at an MHC locus was associated with the development of schizophrenia. This study found that the association is caused partly by the complement component 4 (C4) genes and therefore implying that allele variants of the C4 genes contribute to the development of schizophrenia. Linkage disequilibrium helped researchers identify which C4 structural variant an individual had by looking at the SNP haplotypes. The SNP haplotypes and the C4 alleles were linked which was why they were in linkage disequilibrium, meaning that they segregated together. A single structural C4 variant was associated with many different SNP haplotypes, but different SNP haplotypes where associated with only one C4 structural variant. This was due to the linkage disequilibrium which allowed the researchers to determine the C4 structural variant easily by looking at the SNP haplotype. Their data suggested this because the results showed that the structural variants of C4 express the C4A protein at different levels and this difference in higher C4A protein expressions were associated with higher rates of schizophrenia development. The different structural variant alleles of the same gene were shown to have different phenotypes and susceptibility to disease. These studies exhibit the breadth of the involvement and significance of structural variation on the human genome. Its importance is demonstrated with its contribution to phenotypic diversity and disease susceptibility.

### Other Diseases

As indicated above, various diseases and conditions are characterized by different SVs. Additional examples include insertions (Tay-Sachs disease), deletions (Williams syndrome, Duchenne muscular dystrophy, Smith-Magenis syndrome, Carney Complex), interspersed duplications (APP in Alzheimer's disease, PotockiLupski syndrome, Prader-Willi syndrome, Angelman syndrome), translocations (Down syndrome, XX male syndrome (SRY), schizophrenia (chr 11), Burkitt's Lymphoma), inversions (Hemophilia A, Hunter Syndrome, EmeryDreifuss muscular dystrophy), tandem duplications (FMR1 in Fragile-X, Huntington's disease, Spinocerebellar ataxia), and duplications (Charcot-Marie Tooth disease). It is further known that various SVs are associated with cancers.

### Somatic Mutations in the Brain

Somatic SNVs accumulate during human brain development, with an estimated 200-400 somatic SNVs already present per cell at mid-gestation. Mutations acquired during development may be functionally silent, while serving to identify cells descended from the same progenitor for lineage tracing. If such mutations alter cellular physiology, they can alter tissue structure and function and result in developmental neurological disorders. For example, pathogenic somatic mutations in mTOR pathway genes in certain brain progenitors result in hemimegalencephaly, and similar mutations in a more limited distribution produce focal cortical dysplasia. Somatic mutations may also directly affect the electrical physiology of neurons, as the expression of the Braf V600E variant in mouse neuronal progenitors contributes to epileptogenicity. Somatic mutations have also enabled studies tracing the origin of cancers—for example, providing evidence that glioblastoma tumors share somatic mutations with subventricular zone progenitor cells, their potential cellular origin.

### Somatic Mutations and Signatures in Aging

Somatic mutations have been identified as increasing in neurons during the course of human aging. In neurons, somatic SNV levels rise with age at a rate of approximately 20 new mutations per year, a concept known as genosenium that reveals novel insights about the aging process. Analysis of the specific DNA base changes and their trinucleotide contexts can identify signatures that reflect the origin of those somatic mutations.

Cancer genome analyses have identified a number of mutational signatures. Notably, Catalogue of Somatic Mutations in Cancer (COSMIC) signatures 1 and 5 (analogous to the singlebase signatures SBS1 and SBS5 in the most recent version, COSMIC v3.2; Available at World Wide Web cancer.sanger.ac.uk/cosmic/signatures) were identified in tumor genomes as increasing with age in a clock-like manner, such that the abundance of these signatures corresponds to the age of an individual. Signature 1 contains predominantly C>T mutations, while signature 5 contains primarily C>T and T>C mutations. Single cell whole genome sequencing of 161 neurons derived from healthy and prematurely aging brains revealed a mutational signature, named signature A, that resembled signature 5 and correlated with age. A subsequent study using bulk exome sequencing also found an abundance of signature 5 in aged brain samples. While such study was not able to detect the full extent of mutations that can be found with single-cell experiments, it is noteworthy that the likely clonal somatic mutations detectable in bulk exome sequencing also showed aging-associated mutational signature 5 in the brain. Indeed, the aging-associated mutational signatures observed in the brain are similar to those seen in other tissues (Table 1).

TABLE 1
Studies of somatic single-nucleotide variant signatures in the brain in aging
and neurodegeneration, along with selected other human tissues (adapted from
Miller et al. (2021) Annual Review of Genomics and Human Genetics. 22: 239-
56, which is incorporated herein in its entirety by this reference).

| | | | | |
|---|---|---|---|---|
| | | | Biological | Mutational |
| Study | Tissue/cell | Method | context | signature(s) |

Brain

| | | | | |
|---|---|---|---|---|
| Hoang et al. | Bulk brain | Dilution | Aging | None |
| (51) | (frontal cortex), | followed by | | identified |
| | colon, kidney | bulk whole- | | |
| | | genome | | |
| | | sequencing | | |
| | | (BotSeqS) | | |
| Park et al. | Bulk brain | Bulk whole- | Alzheimer's | COSMIC |
| (93) | (hippocampus) | exome | disease | signature |
| | | sequencing | | SBS18 |
| Lodato et | Neurons | Single-cell | Aging | COSMIC |
| al. (73) | (prefrontal | whole- | | signature 5 |
| | cortex) | genome | DNA repair | COSMIC |
| | | sequencing | deficiency | signature 8 |
| | | | neurodegeneration | |

Other tissues

| | | | | |
|---|---|---|---|---|
| Blokzijt et | Adult stem cells | Whole- | Aging | COSMIC |
| al. (12) | of small intestine, | genome | | signature 5 |
| | colon, liver | sequencing of | | |
| | | clonal | | |
| | | organoid | | |
| | | cultures | | |
| | | derived from | | |
| | | primary | | |
| | | multipotent | | |
| | | cells | | |
| Osorio et al. | Hematopoietic | Whole- | Aging | COSMIC |
| (900 | stem cells | genome | | signature 5 |
| | | sequencing of | | |
| | | clonal cultures | | |
| Franco et | Skeletal muscle | Whole- | Aging | COSMIC |
| al. (37) | resident | genome | | signatures 1, |
| | progenitor/stem | sequencing of | | 5, and 8 |
| | (satellite) cells | in vitro | | |
| | | clonally | | |
| | | expanded | | |
| | | single cells | | |
| Zhang et al. | B lymphocytes | Single-cell | Aging | COSMIC |
| (132) | | whole- | | signatures 1 |
| | | genome | | and 5 |
| | | sequencing | | |
| Lee-Six et | Colon (crypts) | Whole- | Aging | COSMIC |
| al. (70) | | genome | | signatures |
| | | sequencing of | | SBS5 and |
| | | colorectal | | SBS1 |
| | | cyrpts, to | | |
| | | represent | | |
| | | clonse from | | |
| | | colorectal | | |
| | | stem cells | | |
| Franco et | Kidney tubules, | Whole- | Aging | COSMIC |
| al. (36) | epidermis, | genome | | signatures |
| | subcutaneous | sequencing of | | SBS1, SBS3/8, |
| | adipose tissue, | in vitro | | SBS5, and |
| | visceral adipose | clonally | | SBS40 |
| | tissue | expanded | | |
| | | single cells | | |

### Somatic Mutations as a Potential Cause of Alzheimer's Disease (AD)

While germline mutations in the genes APP, PSEN1, and PSEN2 are known to cause early-onset familial AD, these mutations account for only a small fraction of cases, as the majority of individuals with AD develop the disease without a fully penetrant genetic cause. Such nonfamilial AD (also referred to as sporadic or non-Mendelian AD) often arises later in life than familial AD and thus significantly overlaps with late-onset AD. Therefore, it has been hypothesized that somatic mutations in familial AD genes may cause late-onset AD, with the lower cell fraction or limited spatial distribution of mosaic mutations serving to explain the later onset of disease. In such a case, misfolded proteins first generated from a sparse somatic mutation might spread to other areas of the brain by means of templated protein misfolding, in a similar manner as occurs during the spread and misfolding of prions. Indeed, both Aβ and tau have shown such templated misfolding in various systems, implicating the somatic mutation in late-onset AD pathogenesis.

### Somatic Mutations in Cancer Genes and Implications for Neurodegeneration

While much of the attention on somatic mutations in AD and other neurodegenerative diseases has focused on a somatic version of familial disease genetics, clues from other disorders suggest that multiple somatic mechanisms may produce neurodegeneration. For example, certain neurodegenerative phenotypes can occur in patients with the somatic mutation-driven neoplasm Langerhans cell histiocytosis, which results from the proliferation of myeloid cell precursors, often driven by BRAF V600E and other MAPK pathway variants. In such individuals, lesions occur in the cerebellum and basal ganglia, with corresponding clinical neurological symptoms.

To further investigate the possible role of somatic mutations and histiocytosis in neurodegeneration, Mass et al. developed mice expressing Braf V600E in specific yolk sac erythro-myeloid progenitors that populate the brain in early development and generate microglia, the brain tissue-resident macrophages. These mice showed clonal expansion of tissue-resident macrophages and severe late-onset neurodegenerative disease, bolstering the link between somatic mutation-driven proliferation and neurodegeneration. Indeed, a diverse group of somatic variants can cause histiocytosis diseases, providing a variety of potential genes that could lead to neuronal dysfunction in a similar manner as in Langerhans cell histiocytosis. Whereas limited studies have so far not revealed BRAF V600E mutations in AD brain, small numbers of cases show mutations in DNMT3A or TET2, which are cancer-associated genes that are also mutated in clonal hematopoiesis, or in the PI3K, MAPK, or AMPK pathways.

### Genome-Wide Somatic Mutations

Beyond the effect of variants in a single gene, the full aggregate of somatic mutations in the genome carries the potential to significantly impact cellular function and health. While sequencing technology has developed dramatically in recent years, the majority of studies are performed on bulk tissue and are thus best suited to detecting variants present in multiple clonal cells, as discussed above. Bulk approaches are generally unable to detect private mutations in individual cells, which limits the inferences that can be made from negative results, and indeed current studies generate conflicting conclusions. Some bulk sequencing studies have suggested that there are somatic mutations that are uniquely present in AD brains and absent in controls in targeted sequencing data.

Single-cell methods are able to detect mutations that are present only in individual cells, which indeed may make up the majority of a neuron's somatic mutation burden. These single-cell mutations appear to be present in the hundreds at birth but then, remarkably, increase at a rate of approximately 20 SNVs per year, leaving neurons with thousands of such somatic SNVs in old age. In individuals with a neurodegenerative phenotype linked to deficient nucleotide excision repair (NER), manifesting as Cockayne syndrome or xeroderma pigmentosum, single cell whole genome sequencing on neurons revealed a significant increase in somatic SNVs compared with normal neurons. This observation suggests that a genome-wide increase in neuronal somatic mutations may also occur in other neurodegenerative diseases. The somatic SNVs in NER-deficient neurons do not fall in a single gene or genomic area, but instead are broadly distributed across the genome, in a similar manner as somatic SNVs acquired during the aging process. Furthermore, the somatic mutations in NER-deficient neurons showed a distinct composition of mutational signature patterns compared with controls.

### Mechanisms of Somatic Mutation in Neurodegeneration

Mutational signature analysis of single cell whole genome sequencing data from NER-deficient neurons showed an abundance of signature C above the levels seen in control neurons. Signature C and the overall mutational profile in NER-deficient neurons point to specific mutagens and cellular processes that influence somatic mutation in these cells, and may act more broadly in neurodegeneration. Signature C contains C>A mutations, which are associated with oxidative damage to DNA in the form of 8-oxo-guanine and other altered bases, a result of reactive oxygen species produced during cellular metabolism. Indeed, oxidative damage has been previously identified in AD brain tissue. Interestingly, exome sequencing of the hippocampus in AD also identified an oxidative mutational signature, more than half of which consisted of C>A mutations, whose detection by bulk sequencing indicates that they may potentially arise in a different manner than the predominantly private mutations identified in single cells. Increased oxidative DNA damage and reduced histone deacetylase HDAC1 activity were observed in transgenic mice expressing five germline AD-linked mutations, and this increase in oxidative damage is also observed in HDAC1-deficient mice, suggesting a link between chromatin structure and DNA damage, which may in turn lead to increased somatic mutations.

The observation of signature C mutations in human neurons that are genetically deficient in NER indicates the involvement of NER in repairing lesions that lead to signature C somatic mutations. Therefore, somatic mutations may result from increased oxidative damage that accumulates beyond the capacity for NER and other DNA damage repair pathways to correct the DNA lesions. Furthermore, there is evidence linking AD-associated misfolding of tau and Aβ to DNA damage, potentially involving a toxic feed-forward loop between these mechanisms.

### Potential Effects of Abundant Genomic Somatic Mutations in Neurodegeneration

The DNA damage theory of aging postulates that DNA damage contributes to genomic instability and the overall process of aging. Somatic mutations indeed accumulate in neurons during typical aging, and more so in neurodegeneration from NER deficiency. How might these mutations lead to dysfunction in cells? These neurons show more nonsynonymous mutations, which change the encoded amino acid, and stop-gain mutations, which create a new stop codon that truncates protein translation. These changes can impair the function of processes that rely on full dosage of particular genes. Also, as mutations accumulate, this accumulation produces exponential increases in the proportion of cells that have biallelic inactivation, with modeling showing such an increase of so-called knockout neurons. The increase in nonsynonymous mutations also leads to a projected increase in neoantigen peptides that are produced in the cell and then presented by major histocompatibility complex (MHC) class I molecules to CD8+ T lymphocytes for immune surveillance. Whether from gain or loss of function, somatic mutation accumulation stands to affect individual genes and the broader genome, which can play a role in cellular dysfunction and potentially cell death.

More Diseases Other than Cancer Caused by Somatic Mutation

Rare disorders that have a clear basis in somatic variations include those of the hematopoietic system, in which stems cells can mutate and expand to produce disease phenotypes. These include paroxysmal nocturnal hemoglobinuria 1 (PNH1) caused by PIG-A mutations and X-linked alpha-thalassemia mental retardation caused by mutations in ATRX. PNH1 is an acquired hemolytic anemia that presents with hemoglobinuria, abdominal pain, smooth muscle dystonias, fatigue, and thrombosis. It is caused by expansion of hematopoietic stem cells with a mutation in the PIG-A gene—a change that is acquired somatically. X-linked alpha-thalassemia mental retardation is sometimes associated with myelodysplastic syndrome, with cases often associated with somatic mutations. Interestingly, in the case of ATRX mutations, somatic variants appear to confer more severe myelodysplastic syndrome disease than do germline mutations. Clearly, the ability to clonally expand hematopoietic stem cells can provide a mechanism by which somatic mutation can confer disease risk.

Neurofibromatosis 1 (NF1), a disorder that maps to a segment of chromosome 17q, presents with cafe-au-lait spots, Lisch nodules in the eye, and fibromatous tumors of the skin. Several studies have shown that a large minority of NF1 cases are due to somatic mutations, often deletions or microdeletions in this chromosomal region (up to 40% of cases). Other cases are caused by somatic mitochondrial DNA (mtDNA) mutations. In either case, it is clear that somatic changes are often causative of NF1. Similarly, NF2 has been shown to often be caused by somatic mutation as well (25-30% of cases).

Diseases of other tissues can be shown to be somatic in origin by careful characterization of resected tissue. Examples include diseases of the heart and kidney. For example, mutations in connexin 40, a cardiac myocyte-expressed protein encoded by GJA5, have been shown to affect electrical communication and associate with a large minority of atrial fibrillation cases. Most of the GJA5 mutations found in cardiac myocytes of patients were not present in blood, indicating a somatic origin. A similar situation has been found in some Alport syndrome cases. Alport syndrome is an X-linked dominant disorder characterized by kidney disease, hearing loss, and eye abnormalities. It is caused by mutations in collagen IV components, mostly COL4A5. Although most Alport syndrome cases are inherited through the germline, it has been reported that males with a less severe phenotype have COL4A5 somatic mutations. As with many X-linked diseases that would otherwise be extremely severe in presentation or lethal in males, somatic mutations can present with milder forms of disease.

Somatic mutation has also played a role in some neurological diseases, including epilepsy, autism spectrum disorders (e.g., Rett syndrome), and intellectual disability, although comparisons of monozygotic twins for multiple sclerosis (MS) have been essentially negative. The latter example is based on whole genomic data of discordant monozygotic twins, but the data were derived from lymphoctyes-clearly not the ideal tissue for MS. Neurological disease may be particularly sensitive to somatic mutation because even less than 10% of cells carrying a mutation can affect phenotypes based on the distribution of these cells in the brain. For example, hemimegalencephaly (HMG), which presents with an enlargement and malformation of an entire hemisphere, is associated with somatic mutations of AKT3 and other mutations in the PI3K-AKT3-mTOR pathway, even when as few as 8% (and generally fewer than 35%) of cells carry the somatic mutation. However, because of the broad distribution of the mutation-carrying cells, individuals can still present with HMG. The effects of even rare somatic mutations may be due to the unique development pattern of the brain and its complex clonal migration patterns, such that clonality is not limited to adjacent or nearby cells.

Lissencephaly, or smooth brain, can be caused by mutations in two genes: Doublecortin X (DCX) or Lissencaphaly 1 (LIS1). Mutations in LIS1, which maps to 17p1, are usually lethal in males, but milder forms have been associated with somatic mosaics in two patients with predominantly posterior subcortical band heterotopia. In these patients, 18-24% of blood cells and 21-34% of hair roots were mutated. Somatic mutations of DCX1 have also been shown to associate with similar disease phenotypes. As with the neurological diseases above, not all neuronal cells carry the mutations, but they do exist in leukocytes, suggesting early somatic mutation.

Mutations in the X-linked pyruvate dehydrogenase A1 (PDHA1) can present with metabolic or neurological traits. Metabolic disease usually leads to death in infancy from lactic acidosis, but the neurological form presents with symptoms including epilepsy, mental retardation, and spasticity. A continuum exits between these two presentations. A high proportion of heterozygous females present with severe disease, but a report showed that a female with mild disease had evidence of preferential X-inactivation and somatic mutation. Similarly, a male with a mild form of disease had an exon skipping mutation in both skin and muscle tissue, but not lymphocytes. Although limited to single clinical cases, both of these examples show that somatic mutations in a single gene can affect disease risk. And of note, both cases caused by somatic variation presented with milder forms of disease.

Lastly, autoimmune diseases can be caused by somatic mutations. A recent study of autoimmune lymphoproliferative syndrome (ALPS), a disease of benign lymphoproliferation, elevated immunoglobulins, plasma IL-10 and FAS-L, and accumulation of double-negative T cells, showed that in several cases this was due to somatic mutation. Inherited heterozygosity of TNFRSF6 precedes this disease, followed by a genetic events in the second allele. In this study, seven patients fit this profile; three had somatic mutations in their second allele, and four had evidence of loss of heterozygosity. Two different types of somatic events were therefore shown to cause this disease in individuals with susceptible (heterozygote) genotypes.

### Somatic Mutations in Psychiatric Disorders

Recent progress has identified candidate risk genes for a variety of psychiatric disorder. For example, a large-scale genome-wide association study (GWAS) identified 108 genomic loci associated with schizophrenia using single-nucleotide polymorphism (SNP) microarray technology. Additional studies have identified several copy-number variations (CNVs) associated with either schizophrenia or autism spectrum disorder (ASD).

Although the remaining liability to psychiatric disorders has classically been attributed to environmental factors, recent psychiatric research has focused on the role of de novo mutations, which represent a type of non-inherited genetic factor. De novo mutations occur prior to fertilization, before or during spermatogenesis/oocytogenesis. Some de novo mutations occurring before spermatogenesis/oocytogenesis are derived from genomic chimerism in either parent, which can be detected in a part of the somatic tissues of the parent. In contrast, de novo mutations occurring during spermatogenesis/oocytogenesis cannot be detected in the tissues of the parents, except for in a limited number of germ cells. Trio analyses have revealed that de novo mutations in SETD1A, CHD8, and other critical variants are associated with an increased risk of multiple psychiatric disorders. Large case-control studies have validated these findings regarding SETD1A and CHD8 in patients with schizophrenia and ASD, respectively.

In addition to germline de novo mutations, somatic or postzygotic mutations may occur following fertilization. Following such mutations, the genome in each somatic cell is not completely identical in one individual. Somatic mutations have also been well characterized as a pathological mechanism associated with cancer, and as an adaptive physiological mechanism associated with somatic rearrangement of immunoglobulin genes. Cancers are caused by somatic mutations in key-driver genes in a specific tissue, and numerous additional somatic mutations may accrue with advancement. In addition to cancerous tissues, recent genomic studies have systematically identified somatic mutations at the genome-scale in non-cancerous human tissues. Furthermore, some mutations originally labeled as germline de novo mutations have subsequently been identified as somatic mutations that occurred after fertilization in the children, or prior to spermatogenesis/oocytogenesis in the parents. Several human diseases are known to result from somatic mutations, and accumulating evidence indicates that somatic mutations may explain in part the liability to psychiatric disorders. Such mutations can be observed in various tissues during the early developmental period, including peripheral tissues (e.g., blood cells) as well as brain cells. In contrast, somatic mutations that occur following differentiation exist within a limited region of a single tissue type (e.g., brain), and thus can be detected only in that tissue. Somatic mutations occur due to environmental insults, including inflammation and oxidative stress, as well as stochastic changes during development.

While polymorphisms and the variants transmitted from ancestries are inherited genetic factors, the other three mutation types of de novo and somatic mutations are non-inherited genetic factors. Nonetheless, these all four types of germline and somatic variants (mutations) likely have an additive effect on the individual phenotype. For example, research has indicated that germline de novo mutations and inherited variants additively contribute to the risk for ASD. In principle, mutations resulting in embryonic lethality or severe congenital diseases cannot exist in the germline genome, although they may exist as somatic mutations, possibly resulting in relatively less severe physiological consequences. Previous studies regarding epileptic encephalopathy have revealed that single somatic mutations of PCDH19 result in less severe pathology than de novo mutations of the same gene.

The estimated rate of de novo mutations is 1-1.5×10-8 per nucleotide per generation. Somatic mutations may be more common than de novo mutations. Assuming a conservative estimate of 2.8 substitution mutations per cell per cell division and symmetrical divisions in development, 86 billion neurons would have gone through at least 36 divisions, thus resulting in a minimum of 100 single-nucleotide variants (SNVs) in one neuron. In fact, neurons likely undergo many more cell divisions, and mutation within neural tissues occurs via mechanisms other than replication errors during cell division. In addition, other types of mutations (e.g., structural variants) may occur, increasing the number of mutational events beyond this minimum estimation.

TABLE 2
Somatic Mutations in Patients with Neuropsychiatric diseases (adapted from Nishioka et al. (2019)
Molecular Psychiatry 24: 839-856, which is incorporated herein in its entirety by this reference).

| Disease/disorder | Implicated gene | Mutation type | Sample |
|---|---|---|---|
| Rett syndrome | MECP2 | Small deletion | Peripheral blood |
| | | | lymphocytes |
| Autism spectrum | KMT2C, NCKAP1, MYH10, and | SNV | Blood |
| disorder | others | | |
| | MFRP, MYO9B, PTK7, TANC2, | SNV | Blood |
| | MEGF11, and others | | |
| | KLF16, MSANTD2, SCN2A, | SNV | Blood |
| | HNRNPU, SMARCA4, and others | | |
| | CHD2, CTNNB1, KMT2C, | SNV | Blood |
| | SYNGAP1, RELN, and others | | |
| Monozytotic twin | | | |
| Darier disease | ATP2A2 | Small deletion | Blood (MZ) |
| Van der Woude syndrome | IRF6 | SNV | Blood (MZ) |
| Dravet syndrome | SCN1A | SNV | Lymphocytes (MZ) |
| | | | Hair-follicle cells (MZ) |
| | | | Cheek cells (MZ) |
| | | | Fibroblasts (MZ) |
| | | | Olfactory neuroepithelium |
| | | | (MZ) |
| Neurofibromatosis type 1 | NF1 | SNV | Blood (MZ) |
| | | | Buccal swab (MZ) |
| | | | Urine (MZ) |
| Parkinson-related diseases | 31 loci | CNV | Blood (MZ) |
| Fragile X syndrome (severity) | FMR1 | CGG repeat | Blood (MZ) |
| Gender dysphoria | FBXO38, SMOC2, TDRP | SNV | Blood lymphocytes (MZ) |
| Delusional disorder | ABCC9 | SNV | Blood (MZ) |
| Postmortem brain | | | |
| Hemimegalencephaly | PIK3CA, AKT3, MTOR | SNV | The affected brain region |
| | 1q trisomy/tetrasomy | CNV | The affected brain region |
| Cortical dysplasia type II | MTOR | SNV | The affected brain region |
| Sturge-Weber syndrome | GNAQ | SNV | Brain |
| Huntington's disease | HD | CAG repeat | Striatum |
| | HD | CAG repeat | Frontal cortex |
| | | | Cerebellum |
| Rett syndrome | LINE1 copy number | MEI | iPS-derived NPC |
| Ataxia telangiectasia | LINE1 copy number | MEI | Hippocampal neuronal |
| | | | nuclei |
| Cockayne syndrome | Global SNV increase | SNV | Prefrontal cortex neuron |
| Xeroderma pigmentosum | Global SNV increase | SNV | Prefrontal cortex neuron |
| Alzheimer's disease | PSEN1 | SNV | Cerebral cortex |
| | MAPT, PSEN2 | SNV | Entorhinal cortex |
| | Many (not validated) | SNV | Hippocampus |
| | APP copy-number increase | CNV | Prefrontal cortex |
| | | | Cerebellum |
| | No different aneuploidy | Aneuploidy | Frontal cortex |
| | Chromosome 21 loss/gain | Aneuploidy | Cerebral cortex |
| | Excess aneuploidy | Aneuploidy | Entorhinal cortex |
| | | | Occipital cortex |
| Autism spectrum disorder | CACNA1C, SCN1A, SETD2 | SNV | Prefrontal cortex |
| | | | Cerebellum |
| Schizophrenia | LINE-1 copy number | MEI | Cortex neuronal nuclei |
| | | | Cortex non-neuronal |
| | | | nuclei |
| | | | iPS-derived neurons |
| | LINE-1 copy number | MEI | Dorsolateral prefrontal |
| | | | cortex |
| | Deletions in PRKRA and others | CNV | Prefrontal cortex |
| | | | Cerebellum |
| | Ip36.21, Ip13.3 | CNV | Striatum |
| | Chromosome 1 loss/gain | Aneuploidy | Cortex (Brodmann area |
| | | | 10) |
| Peripheral tissues | | | |
| Hemimegalencephaly | PIK3CA | SNV | Blood |
| | | | Saliva |
| | | | Buccal swab |
| Megalencephaly | PIK3CA | SNV | Blood |
| Double cortex syndrome | DCX, LIS1 | SNV | Blood |
| Periventricular nodular | FLNA | SNV | Blood |
| heterotopia | | | |
| Pachygyria | TUBB2B | SNV | Blood |

### Cancer

As described above, cancers are caused by somatic mutations in key-driver genes in a specific tissue, and numerous additional somatic mutations may accrue with advancement.

Cancer, tumor, or hyperproliferative disease refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, (e.g., multiple myeloma, Diffuse large B-cell lymphoma (DLBCL), Follicular lymphoma, Chronic lymphocytic leukemia (CLL), small lymphocytic lymphoma (SLL), Mantle cell lymphoma (MCL), Marginal zone lymphomas, Burkitt lymphoma, Waldenström's macroglobulinemia, Hairy cell leukemia, Primary central nervous system (CNS) lymphoma, Primary intraocular lymphoma, the heavy chain diseases, such as, for example, alpha chain disease, gamma chain disease, and mu chain disease, benign monoclonal gammopathy, and immunocytic amyloidosis), T cell cancer (e.g., T-lymphoblastic lymphoma/leukemia, non-Hodgkin lymphomas, Peripheral T-cell lymphomas, Cutaneous T-cell lymphomas (e.g., mycosis fungoides, Sezary syndrome), Adult T-cell leukemia/lymphoma, Angioimmunoblastic T-cell lymphoma, Extranodal natural killer/T-cell lymphoma, Enteropathy-associated intestinal T-cell lymphoma (EATL), Anaplastic large cell lymphoma (ALCL), Hodgkin lymphoma), melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

### Mutagenic Agents/Potentially Mutagenic Cancer Therapies

Examples of mutagenic agents or potentially mutagenic cancer therapies include chemotherapy and radiation therapy.

Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, elastogens, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; antimitotic agents: halichondrin, colchicine, and rhizoxin; and elastogens: bleomycin actinomycin D, camptothecin, and methotrexate, as well as non-therapeutic elastogens such as acridine yellow, benzene, ethylene oxide, arsenic, phosphine, mimosine, methyl acrylate, resorcinol, 5-fluorodeoxyuridine, and 1,2-dimethylhydrazine (a known colon carcinogen). Compositions comprising one or more chemotherapeutic agents (e.g., FLAG, CHOP) are often used in the clinic. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6 (5H)-phenanthridinone (Trevigen); benzamide (U.S. Pat. No. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly (ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

### Types of Somatic Mutations

The types of somatic mutations that are associated with various diseases include, but are not limited to, changes in ploidy number, aneuploidy, copy number variation, loss of heterozygosity, retrotransposons, indels, insertion of one or more nucleotides, deletion of one or more nucleotides, duplication of one or more nucleotides, substitution of one or more nucleotides, and single nucleotide variation.

Somatic mutations may occur in chromosomal DNA as well as mitochondrial DNA. It has long been known that as people age they can accumulate mtDNA mutations that increase their levels of heteroplasmy. This has been especially well studied in muscles. In addition, some of the somatic mtDNA mutations that accumulate with age have been associated with disease. For example, T414G was reported to be present as a somatic mutation in the brain tissue of Alzheimer's patients but not controls. T414G also accumulates with age in fibroblasts and skeletal muscle.

Several other somatic heteroplasmy changes have been reported. T408A mutation has been reported as an age-related somatic mutation in muscle, as has A189G mutation. A recent study of mtDNA heteroplasmy variation among tissues of the same individuals has confirmed some of these patterns and extended them in an unexpected way. Using massively parallel sequences of ten common tissues taken at autopsy from two cancer-free individuals, the patterns of mtDNA heteroplasmy was assessed across tissues and subjects. Of 20 observable mtDNA heteroplasmies, 10 were recurrent. That is, they were observed in both subjects in the heteroplasmic state, but importantly only in the same tissues: kidney, liver, or skeletal muscle. These heteroplasmic sites included previously identified ones, such as A189G and T408A described above, as well as ones described in another study that sequenced mtDNA from multiple autopsy tissues. Importantly, the two studies showed that the tissue-specific pattern of mtDNA heteroplasmic sites was consistent, lending support to the hypothesis that certain heteroplasmies develop preferentially in very specific tissues only. Since the recurrent heteroplasmies were observable only in the highest copy number tissues and in proximity to or in DNA replication control regions, it was hypothesized that these mutations affected DNA replication. Considering their totality, the data clearly indicate that mtDNA mutations accumulate somatically in the heteroplasmic state with age, occur in a tissue-specific fashion, and may affect disease.

### Control

A control refers to any suitable reference standard, such as a normal patient, cultured primary cells/tissues isolated from a subject such as a normal subject, adjacent normal cells/tissues obtained from the same organ or body location of the patient, a tissue or cell sample isolated from a normal subject, or a primary cells/tissues obtained from a depository. In other embodiments, the control may comprise at least one mutation detected by the methods and/or compositions of the present disclosure. In some embodiments, the at least one mutation is from a subject or a cell that has not been exposed to a mutagenic chemical or radiation compound. In some embodiments, the at least one mutation is from a subject or a cell that has not been exposed to a biohazard material (e.g., carcinogens, chemotherapeutic agents, environmental toxins).

Such a control sample may comprise any suitable sample, including but not limited to a sample from a control diseased patient (can be stored sample or previous sample measurement) with a known outcome; normal tissue or cells isolated from a subject, such as a normal patient or the diseased patient, cultured primary cells/tissues isolated from a subject such as a normal subject or the diseased patient, adjacent normal cells/tissues obtained from the same organ or body location of the diseased patient, a tissue or cell sample isolated from a normal subject, or a primary cells/tissues obtained from a depository. In some embodiments, the control may comprise a reference standard product (e.g., known sequence, e.g., polymorphism of the sequence in normal patients or diseased patients) from any suitable source, including but not limited to at least one mutation from normal tissue (or other previously analyzed control sample), a previously determined sequences within a test sample from a group of patients, or a set of patients with a certain outcome (e.g., susceptibility to a disease) or receiving a certain treatment (e.g., standard of care cancer therapy). It will be understood by those of skill in the art that such control samples and reference standard product levels (e.g., number and/or types of mutations) can be used in combination as controls in the methods of the present invention.

### Diagnostic Methods

The present invention provides, in part, methods, systems, and code for accurately classifying whether a biological sample comprises a number and/or type of mutations that confer certain conditions (e.g., early stage of a disease, disease risk). As described herein, the present invention is useful in accurately identifying mutations (e.g., somatic mutations) that are low in abundance. Such mutations may be indicative of a risk (e.g., disease risk), aging, or the degree of exposure to a mutagen (e.g., chemotherapy, environmental toxin). Accordingly, in some embodiments, the present invention is useful for classifying a sample (e.g., from a subject) as associated with or at risk for a disease (e.g., cancer, autism, etc.) using a statistical algorithm and/or empirical data.

In certain embodiments, the number of mutations in the test sample as compared with the control is indicative of a disease risk or the degree of exposure to a biohazard material (e.g., chemical or radioactive compound). In some embodiments, the number of mutations in the test sample is increased by at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, or 1000% relative to the control (e.g., a number of mutations in a healthy subject).

In certain embodiments, the type of mutations in the test sample as compared with the control is indicative of a disease risk or the degree of exposure to a biohazard material (e.g., chemical or radioactive compound). As described herein, certain disease risk comprises a set of mutations (e.g., SNV, copy number variation, etc.) that are infrequent in normal tissues. In addition, an ordinarily skilled artisan would understand that certain chemicals induce certain types of mutations.

In certain embodiments, the presence of a single mutation (e.g., somatic mutation) identifies the subject as having a disease risk or having been exposed to a biohazard material (e.g., chemical or radioactive compound).

In other embodiments, the presence of more than one mutation identifies the subject as having a disease risk or having been exposed to a biohazard material (e.g., chemical or radioactive compound).

In yet other embodiments, the a profile of multiple mutations (i.e., mutation signature) (e.g., at least, about, or no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mutations identifies the subject as having a disease risk or having been exposed to a biohazard material (e.g., chemical or radioactive compound).

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician (a non-specialist, e.g., primary care physician; and/or a specialist, e.g., a histopathologist or an oncologist).

In some embodiments, the method of the present disclosure further provides a diagnosis in the form of a probability that the individual has a disease (e.g., cancer, autism, neurological disease, etc.). For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having the cancer. In some instances, the method of classifying a sample as a cancer sample may be further based on the symptoms (e.g., clinical factors) of the individual from which the sample is obtained. The symptoms or group of symptoms can be, for example, lymphocyte count, white cell count, erythrocyte sedimentation rate, diarrhea, abdominal pain, bloating, pelvic pain, lower back pain, cramping, fever, anemia, weight loss, anxiety, depression, and combinations thereof. In some embodiments, the diagnosis of an individual as having a disease (e.g., cancer) is followed by administering to the individual a therapeutically effective amount of a therapy (e.g., cancer therapy).

### Sample

Biological samples can be collected from a variety of sources from a subject including a body fluid sample, cell sample, or a tissue sample. In some embodiments, the subject and/or control sample is selected from the group consisting of cells, cell lines, whole blood, serum, plasma, buccal scrape, saliva, cerebrospinal fluid, and bone marrow. In some embodiments, samples can contain live cells/tissue, fresh frozen cells, fresh tissue, biopsies, fixed cells/tissue, cells/tissue embedded in a medium.

The samples can be collected from individuals repeatedly over a longitudinal period of time (e.g., once or more on the order of days, weeks, months, annually, biannually, etc.).

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of nuclease inhibitors, addition of denaturants, desalting of samples, concentration of sample proteins, extraction and purification of nucleic acid (e.g., genomic DNA).

### Kit

The present invention also encompasses kits for detecting the presence or the level of at least one mutation in a biological sample. For example, the kit can comprise a labeled compound or agent useful in detecting a mutation in a biological sample (e.g., agents for preparing the genomic DNA library, a single-stranded nucleic acid molecule comprising a hairpin structure (“adapter”), restriction enzymes, ligase, buffers, DNA polymerase for repairing the ends (e.g., Klenow or T4 DNA polymerase), enzymes for dA-tailing the genomic DNA fragments, high fidelity polymerase for RCA and/or PCR, etc.). The compound or agent can be packaged in a suitable container.

A kit can include additional components to facilitate the particular application for which the kit is designed. For example, kits can be provided which contain agents/apparatus (e.g., columns) for purifying DNA.

A kit can include reagents necessary for controls (e.g., cells, genomic DNA, DNA comprising a certain gene of interest).

A kit may additionally include buffers and other reagents recognized for use in a method of the disclosed invention. A kit of the present invention can also include instructional materials disclosing or describing the use of the kit.

### Exemplary Embodiments

1. A single-stranded nucleic acid molecule comprising a hairpin structure, wherein the hairpin comprises:
(a) a blunt end or an overhang;
(b) a unique molecular identifier (UMI) in the stem of the hairpin; and
(c) at least one priming site for polymerase chain reaction (PCR) and/or rolling circle-based linear amplification (RCA), preferably in the hairpin loop.
   2. The single-stranded nucleic acid molecule of 1, wherein the single-stranded nucleic acid molecule comprises two PCR priming sites and an RCA priming site.
   3. The single-stranded nucleic acid molecule of 1 or 2, wherein the hairpin loop comprises at least 1, 2, or 3 uracils, optionally wherein the at least 1, 2, or 3 uracils are not present in one or more PCR priming sites.
   4. The single-stranded nucleic acid molecule of 2 or 3, wherein the two PCR priming sites do not overlap.
   5. The single-stranded nucleic acid molecule of any one of 2-4, wherein (a) the RCA priming site overlaps with at least one PCR priming site; or (b) the RCA priming site overlaps with two PCR priming sites.
   6. The single-stranded nucleic acid molecule of any one of 1-5, wherein the overhang is a 3′ overhang.
   7. The single-stranded nucleic acid molecule of any one of 1-6, wherein the overhang comprises at least one thymidine or at least one uracil.
   8. The single-stranded nucleic acid molecule of any one of 1-7, wherein
(a) the overhang comprises at least 1, 2, 3, 4, 5, 6, 7, 8, 9, 10, 11, 12, 13, 14, 15, 16, 17, 18, 19, or 20 nucleotides;
(b) the overhang consists of one thymidine; or
(c) the overhang comprises at least 1, 2, or 3 uracils.
   9. The single-stranded nucleic acid molecule of any one of 1-8, wherein the UMI comprises at least 4, 5, 6, 7, 8, 9, 10, 11, 12, 13, 14, 15, 16, 17, 18, 19, or 20 nucleotides, preferably at least 6 nucleotides.
   10. The single-stranded nucleic acid molecule of any one of 1-9, wherein the hairpin loop is:
(a) at least 3-nucleotide-long; and/or
(b) no more than 3000-nucleotide-long.
   11. The single-stranded nucleic acid molecule of any one of 1-10, wherein the hairpin stem is:
(a) at least 3-nucleotide-long; and/or
(b) no more than 3000-nucleotide-long.
   12. The single-stranded nucleic acid molecule of any one of 1-11, wherein the single-stranded nucleic acid molecule comprises the sequence of TCTTC TACAGT NNNNNN AGATCG GAAGAG CACACG TCTGAA CTCCAG TC/at least one deoxyuridine (deoxyU)/ACACTC TTTCCC TACACG ACGCTC TTCCGA TCT, wherein N is any nucleotide, optionally wherein the at least one deoxyU comprises at least one Int deoxyuridine (ideoxyU).
   13. The single-stranded nucleic acid molecule of any one of 1-12, further comprising at least one genomic DNA fragment.
   14. A method of preparing a genomic DNA library (e.g., for Single Molecule Mutation Sequencing (SMM-Seq)), the method comprising:
(a) preparing genomic DNA fragments that are ligated at both ends to the hairpin structure formed by the single-stranded nucleic acid molecule of any one of 1-12;
(b) preparing single-stranded DNA (ssDNA) concatemers by performing a pulse-RCA on the genomic DNA fragments generated in step (a), wherein the pulse-RCA comprises at least one cycle of denaturation-annealing-extension by a DNA polymerase; and

(c) preparing double-stranded DNA comprising the genomic DNA fragments by performing a PCR reaction on the ssDNA concatemers.

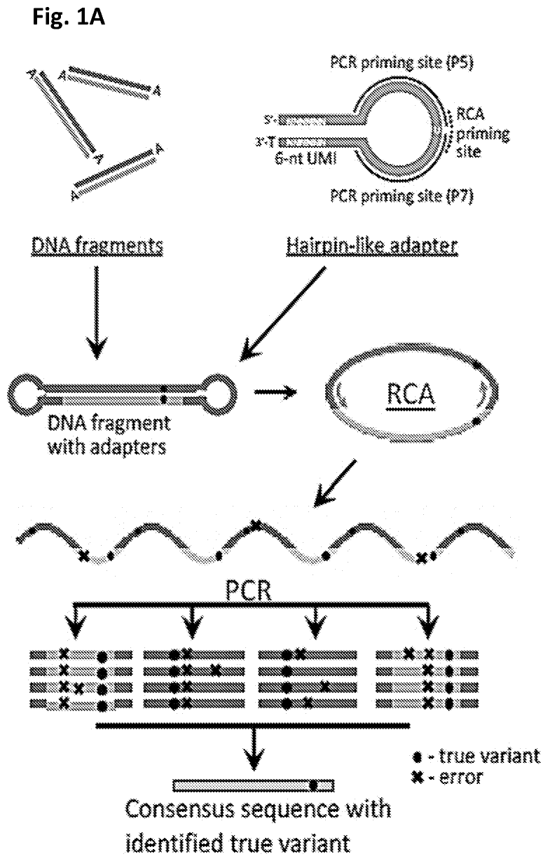

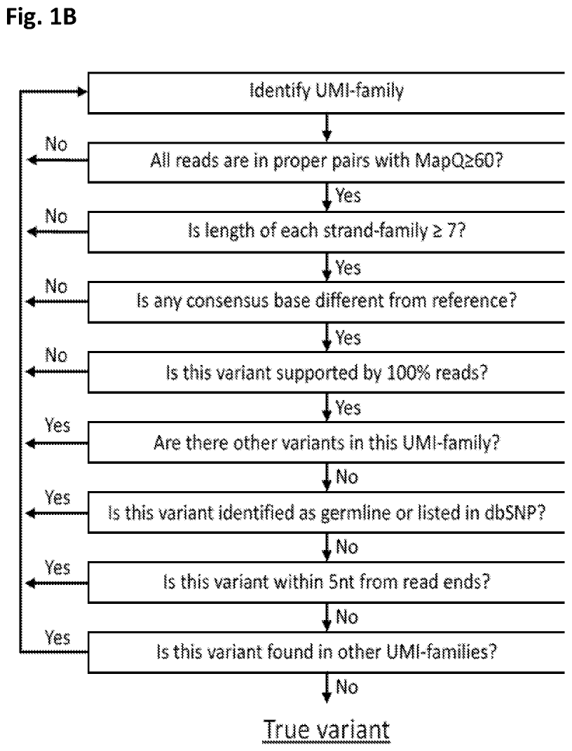

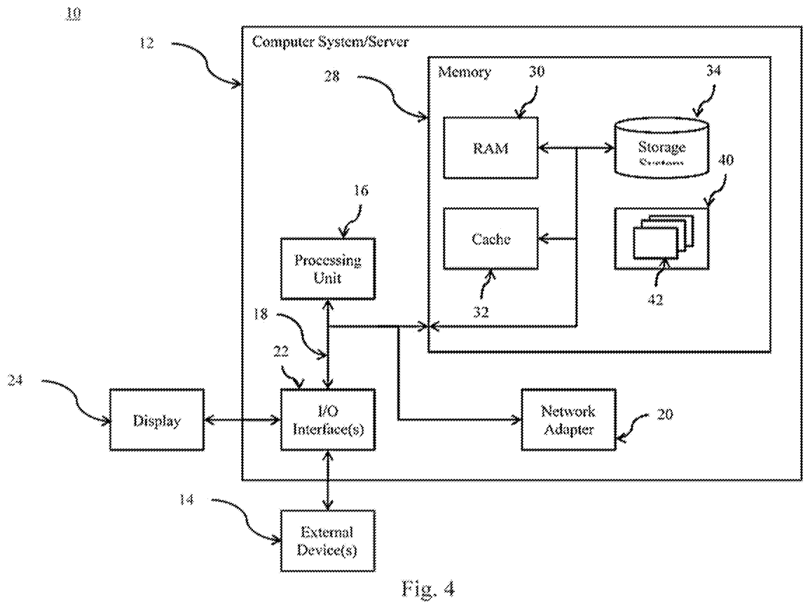

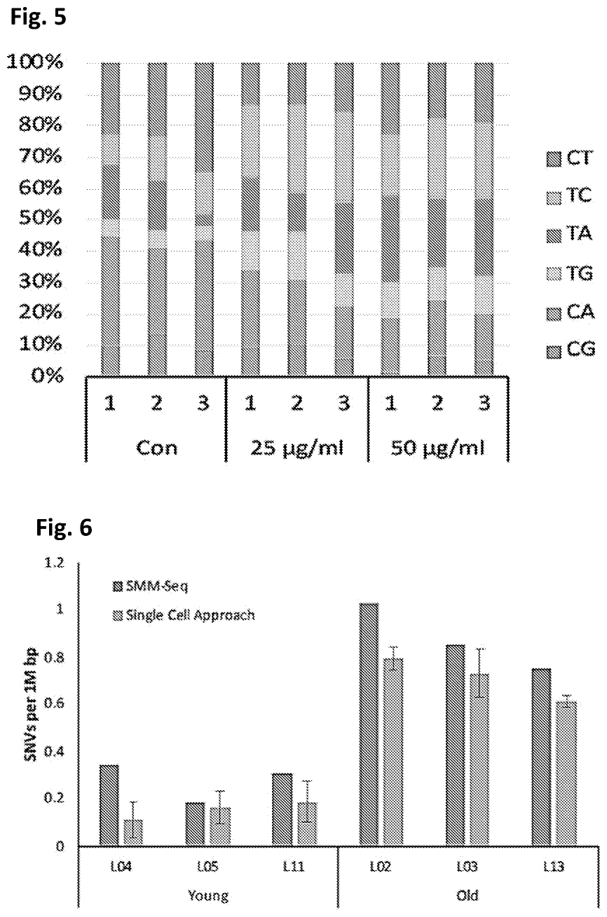

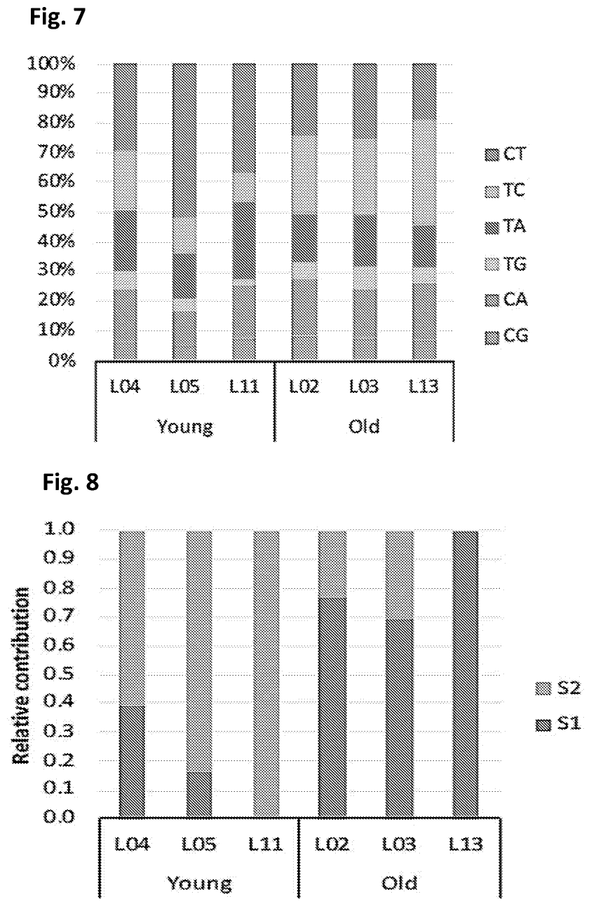

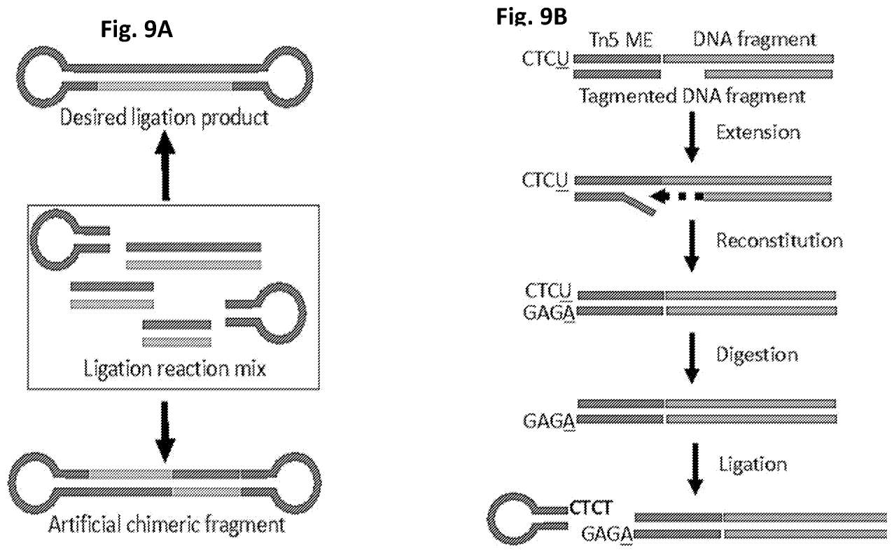

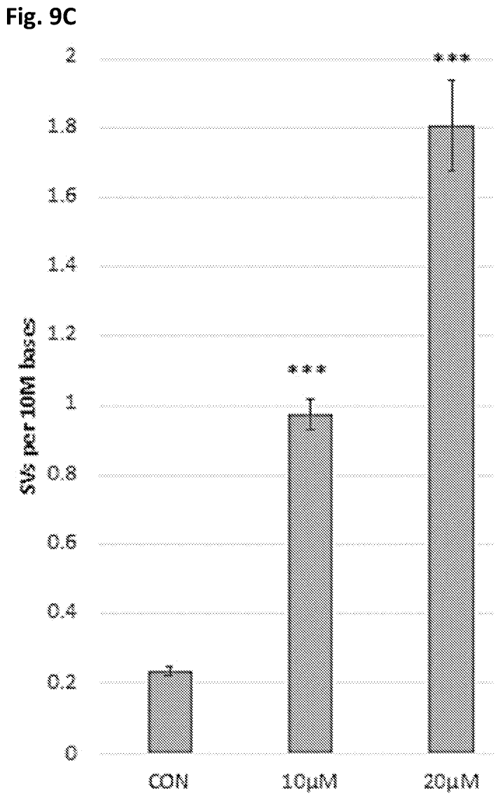

15. The method of 14, wherein the preparation of genomic DNA fragments of step (a) comprises:
(a) creating the genomic DNA fragments by digestion with at least one endonuclease or by sonication;
(b) repairing the ends of the genomic DNA fragments, optionally wherein the repairing comprises making blunt ends (e.g., via micrococcal nuclease, Klenow fragment, or T4 DNA polymerase), phosphorylating 5′ end, and/or dA tailing; and/or
(c) ligating the genomic DNA fragments to the single-stranded nucleic acid molecule of any one of 1-12.
   16. The method of 15, wherein the at least one endonuclease comprises an endonuclease that creates a blunt end (e.g., AluI) and/or an endonuclease that creates an overhang (e.g., MluCI).
   17. A method of preparing a genomic DNA library for detecting a genome Structural Variant (SV), the method comprising:
(a) tagmenting genomic DNA using Tn5-mediated transposition reaction with transposon comprising a uracil residue 5′ to the Tn5 Mosaic End (ME);
(b) extending to fill a 9-nucleotide gap created by Tn5 and reconstituting the same strand (see e.g., FIG. 9B);
(c) digesting using Uracil-DNA Glycosylase (UDG) to release the uracil residue and expose a 3′ overhang;
(d) ligating the genomic DNA fragments generated by steps (a)-(c) to the single-stranded nucleic acid molecule of any one of 1-12;
(e) preparing single-stranded DNA (ssDNA) concatemers by performing a pulse-RCA on the genomic DNA fragments generated in step (a), wherein the pulse-RCA comprises at least one cycle of denaturation-annealing-extension by a DNA polymerase; and
(f) preparing double-stranded DNA comprising the genomic DNA fragments by performing a PCR reaction on the ssDNA concatemers.
   18. The method of any one of 14-17, wherein the DNA polymerase for the pulse-RCA and/or PCR reaction is strong strand displacement or a high-fidelity DNA polymerase (e.g., SD polymerase, strand displacement polymerase HS, Phusion® High-Fidelity DNA Polymerase).
   19. The method of any one of 14-18, wherein the pulse-RCA comprises at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 cycles of the denaturation-annealing-extension by a DNA polymerase.
   20. The method of any one of 14-19, wherein the PCR reaction comprises at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 cycles of the PCR reaction.
   21. The method of any one of 14-20, wherein
(a) the pulse-RCA comprises at least 1 cycle of denaturation-annealing-extension by a DNA polymerase; and/or
(b) the PCR reaction comprises at least or about 6 cycles.
   22. A genomic DNA library prepared by the method according to any one of 14-21.
   23. A method of detecting at least one mutation or at least one structural variant (SV) in a cell or a plurality of cells, the method comprising:
(a) obtaining the cell or the plurality of cells;
(b) preparing a library comprising the genomic DNA fragments of the cell or the plurality of cells according to the method of any one of 14-21; and
(c) sequencing the library.
   24. The method of 23, further comprising aligning the UMIs.
   25. The method of 23 or 24, wherein the sequences are analyzed according to the computational algorithm shown in FIG. 1B and/or using the computing node shown in FIG. 4 (see also claims 62-69).
   26. The method of any one of 23-25, wherein the cell is a primary cell of a subject or a cell from an immortalized cell line.
   27. The method of any one of 23-26, wherein the library is sequenced by Next-Generation Sequencing (NGS), optionally wherein the NGS is Deep Sequencing (e.g., Illumina NovaSeq).
   28. The method of any one of 23-27, wherein the at least one mutation comprises a single nucleotide variant (SNV), a deletion of one or more nucleotides, a insertion of one or more nucleotides, a duplication of one or more nucleotides, a substitution of one or more nucleotides, a point mutation, a translocation, a copy number variation, a loss of heterozygosity, a retrotransposon, or any combination thereof; optionally wherein the mutation is an SNV.
   29. The method of any one of 23-27, wherein the at least one SV comprises a deletion, inversion, insertion, duplication, translocation, or any combination thereof.
   30. The method of 29, wherein the SV comprises at least or about 40, 41, 42, 43, 44, 45, 46, 47, 48, 49, 50, 51, 52, 53, 54, 55, 56, 57, 58, 59, or 60 basepairs of a genome, optionally 50 basepairs of a genome.
   31. The method of any one of 23-30, wherein the at least one mutation or at least one SV comprises at least, about, or no more than 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 mutations or SVs.
   32. The method of any one of 23-31, wherein the method detects a Catalogue of Somatic Mutations in Cancer (COSMIC) signature.
   33. The method of any one of 23-32, wherein the at least one mutation is a somatic mutation or a germline mutation, optionally wherein the mutation is a somatic mutation.
   34. The method of any one of 23-33, wherein the at least one mutation or at least one SV is induced by a chemical agent (e.g., N-ethyl-N-nitrosourea (ENU), bleomycin, chemotherapy) and/or a radioactive agent (radiation therapy).
   35. The method of any one of 23-34, wherein the at least one mutation or at least one SV is related to aging.
   36. The method of any one of 23-35, wherein the method is performed in vivo, in vitro, or ex vivo.
   37. The method of any one of 23-36, wherein the subject is healthy or diseased (e.g., afflicted with a cancer).
   38. The method of any one of 23-37, wherein the subject has been exposed to a chemical agent (e.g., N-ethyl-N-nitrosourea (ENU), bleomycin, chemotherapy) and/or a radioactive agent (radiation therapy).
   39. The method of any one of 23-38, wherein the subject is old (e.g., over 55 years of age) or young (e.g., under 20 years of age).
   40. The method of any one of 23-39, wherein the method detects the DNA damage profile (e.g., a change in DNA sequence observed on copies of only one DNA strand) of a subject afflicted with a cancer, wherein the subject received a chemotherapy or a radiation therapy.
   41. A method of diagnosing a disease risk (e.g., susceptibility to a disease) and/or a disease (e.g., an early stage) in a subject, the method comprising:
(a) detecting at least one mutation (e.g., somatic mutation) or at least one SV in the subject according to the method of any one of 23-40; and
(b) diagnosing the subject as having a disease risk or a disease, if the at least one mutation or at least one SV identified in (a) is associated with said disease.
   42. The method of 41, wherein the disease is a cancer or a disease other than a cancer.
   43. The method of 42, wherein the cancer is selected from: sarcomas, carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, heavy chain disease, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, lung cancer (e.g., non-small-cell-lung cancer, small cell lung cancer), skin cancer, nonpapillary renal cell carcinoma, cervical carcinoma, and ovarian carcinoma (e.g., serous ovarian carcinoma).
   44. The method of 42, wherein the disease other than the cancer is a neurological disease, a hematological disease, or autoimmune disease.
   45. The method of 42 or 44, wherein the disease other than the cancer is selected from Alzheimer's disease (e.g., late onset; age-related), a neurodegenerative disease, a psychiatric disorder, schizophrenia, myelodysplastic syndrome, Neurofibromatosis 1, Cockayne syndrome, xeroderma pigmentosum, Alport syndrome, epilepsy, an autism spectrum disorder, Rett syndrome, intellectual disability, hemimegalencephaly, Lissencephaly, mental retardation, spasticity, and autoimmune lymphoproliferative syndrome.
   46. The method of any one of 41-45, wherein the at least one mutation comprises a COSMIC signature, optionally wherein the COSMIC signature is selected from COSMIC version 3.2 (e.g., DBS GRCh37, DBS GRCh38, ID GRCh37, SBS GRCh37, SBS GRCh38).
   47. The method of any one of 41-46, wherein the disease and/or the at least one mutation is selected from those listed in Table 1.
   48. A method of testing mutagenicity of an agent (e.g., a chemical or a radioactive compound; radiation; a mutagenic agent), the method comprising:
(a) exposing (e.g., contacting, irradiation) a cell to the agent;
(b) detecting at least one mutation or at least one SV in the cell exposed to the agent using the method according any one of 23-40; and
(c) comparing the number and/or types (e.g., SNV, deletion, insertion, short indel, SV, etc.) of mutations identified in (b) cells to a control,
wherein the number and/or types of mutations in (b) cells relative to the control indicates the mutagenicity of the chemical or radioactive compound.
   49. The method of 48, wherein the cell is a primary cell of a subject or a cell from an immortalized cell line.
   50. The method of 48 or 49, wherein the control is the number and/or type of mutations identified in a cell that is not exposed to the agent, preferably wherein the control cell is of the same cell type as the cell that is exposed to the agent.
   51. A method of testing in vivo mutagenicity of an agent, the method comprising:
(a) exposing (e.g., contacting, injecting, inhalation, irradiation) an animal to the agent;
(b) obtaining a cell from the animal exposed to the agent;
(c) detecting at least one mutation or at least one SV in the cell from (b), using the method according any one of 23-40;
(d) comparing the number and/or types (e.g., SNV, deletion, insertion, short indel, etc.) of the at least one mutation or the at least one SV identified in (c) and a control,
wherein the number and/or types of the at least one mutation or the at least one SV in (c) relative to the control indicate the mutagenicity of the agent.
   52. The method of 51, wherein the animal is a mouse, rat, guinea pig, dog, chicken, monkey, or cat.
   53. The method of 51 or 52, wherein the control is the number and/or type of a mutation identified in the cell of an animal that is not exposed to the agent.
   54. The method of 53, wherein (a) the control is from an animal of the same species as the animal that is exposed to the agent; and/or (b) the control is the same cell type as the cell of the animal exposed to the agent.
   55. A method of determining a subject's exposure to a biohazard material (e.g., an environmental toxin, a mutagenic chemical or radioactive compound), the method comprising:
(a) obtaining a cell from the subject exposed to the biohazard material (e.g., an environmental toxin, a mutagenic chemical or radioactive compound);
(b) detecting at least one mutation or at least one SV in the cell from (a), using the method according any one of 23-40,
(c) comparing the number and/or type (e.g., SNV, deletion, insertion, short indel, etc.) of the at least one mutation or the at least one SV identified in (b) and a control,
wherein the number and/or type of the at least one mutation or the at least one SV in (b) relative to the control indicates the subject's exposure to the biohazard material.
   56. The method of 55, wherein the control is the number and/or type of at least one mutation or at least one SV identified in a cell of a subject who is not exposed to the biohazard material.
   57. The method of 56, wherein (a) the control is from subject of the same species as the subject that is exposed to the biohazard material; and/or (b) the control is the same cell type as the cell of the subject exposed to the agent.
   58. The method of any one of 26-57, wherein the subject is a mammal, optionally wherein the mammal is a mouse, rat, guinea pig, dog, cat, monkey, or human.
   59. The method of 58, wherein the mammal is a human.
   60. The method of any one of 23-59, wherein the cell is a mammalian cell, optionally wherein the mammalian cell is a human cell.
   61. A kit comprising the single-stranded nucleic acid molecule of any one of 1-13 and/or the genomic library of 22.
   62. A method for identifying one or more single nucleotide mutations, the method comprising:
receiving a plurality of sequencing reads of a DNA fragment, wherein the plurality of sequencing reads of the DNA fragment comprise first and second strand families, each strand family including reads uniquely associated with the respective strand;
receiving a unique molecular identifier (UMI), the UMI corresponding to the sequencing reads of the DNA fragment, wherein the plurality of sequencing reads of the DNA fragment correspond to a UMI family;
identifying the one or more single nucleotide mutations in the plurality of sequencing reads when:
   each sequencing read corresponds to a paired read with a mapping quality score greater than or equal to a predetermined score;
   a length of each strand family is greater than or equal to a predetermined length;
   one or more variants are determined from the plurality of sequencing reads relative to a reference genome, wherein a predetermined amount of the plurality of sequencing reads correspond to the one or more variants;
   the one or more variants are not known variants;
   the one or more variants are located within a predetermined number of nucleotides from an end of the plurality of sequencing reads; and
   the one or more variants are not found in other UMI families.
      63. The method of 62, wherein the predetermined score is 60.
      64. The method of 62, wherein the predetermined length is 7.
      65. The method of 62, wherein the predetermined amount is 100%.
      66. The method of 62, wherein the predetermined number of nucleotides is 5.
      67. The method of 62, wherein known variants comprise germline variants and variants from a known variant database.
      68. The method of 67, wherein the known variant database comprises dbSNP.
      69. A computer program product for distributed order processing, the computer program product comprising a computer readable storage medium having program instructions embodied therewith, the program instructions executable by a processor to cause the processor to perform a method comprising:
receiving a plurality of sequencing reads of a DNA fragment, wherein the plurality of sequencing reads of the DNA fragment comprise first and second strand families, each strand family including reads uniquely associated with the respective strand;
receiving a unique molecular identifier (UMI), the UMI corresponding to the sequencing reads of the DNA fragment, wherein the plurality of sequencing reads of the DNA fragment correspond to a UMI family;
identifying the one or more single nucleotide mutations in the plurality of sequencing reads when:
   each sequencing read corresponds to a paired read with a mapping quality score greater than or equal to a predetermined score;
   a length of each strand family is greater than or equal to a predetermined length;
   one or more variants are determined from the plurality of sequencing reads relative to a reference genome, wherein a predetermined amount of the plurality of sequencing reads correspond to the one or more variants;
   the one or more variants are not known variants;
   the one or more variants are located within a predetermined number of nucleotides from an end of the plurality of sequencing reads; and
   the one or more variants are not found in other UMI families.

### EXAMPLES

The invention now being generally described, it will be more readily understood by reference to the following examples that are included merely for purposes of illustration of certain aspects and embodiments of the present invention, and are not intended to limit the invention.

### Example 1. Materials and Methods

### Cell Culture and Treatment

Human normal lung IMR90 fibroblasts were maintained in 10% CO₂ and 3% O₂ atmosphere at 37° C. in DMEM (GIBCO, Grand Island, NY, USA) supplemented with 10% FBS (GIBCO). Twenty-four hours after cell seeding the culturing media was changed for media containing different doses of ENU. Cells were harvested 72 hours after ENU was applied. Complete media supplemented with ENU (SIGMA, San Louis, MO, USA) was prepared immediately before application from stock solution (100 mg/ml in 100% ethyl alcohol). Control cells were cultured in the presence of the vehicle only.

### Human Specimens

Frozen human hepatocyte samples were purchased from Lonza Walkersville Inc. All 6 selected hepatocyte donors were healthy participants of various age and gender without any liver cancer or other liver pathology history (Table 3).

TABLE 3
Human liver donor information list

| | | | | | | | Tobacco/ | Serologies | |
|---|---|---|---|---|---|---|---|---|---|
| | Donor | | | | | Liver | alcohol/ | (HBV/HCV/ | Tissue/ |
| # | ID | Age | | Gender | Diagnosis | status | drug use | HIV/EBV/CMV) | method |
| 1 | L05 | 5 | mos. | Male | Anoxia | Normal | N/N/N | Negative | Perfused |
| | | | | | | | | | liver |
| 2 | L11 | 16 | mos. | Female | Anoxia/ | Normal | N/N/N | Negative | Perfused |
| | | | | | Pland | | | | liver |
| | | | | | Syndrome | | | | |
| 3 | L04 | 18 | yrs. | Female | Anoxia, | Normal | N/N/N | Negative | Perfused |
| | | | | | cerebral | | | | liver |
| | | | | | edema | | | | |
| 9 | L02 | 56 | yrs. | Male | Cardiac | Normal | Y/N/Y | Negative | Perfused |
| | | | | | arrest | | | | liver |
| 10 | L13 | 61 | yrs. | Female | Anoxia | Normal | N/N/N | Negative | Perfused |
| | | | | | | | | | liver |
| 12 | L03 | 77 | yrs. | Male | Anoxia, | Normal | N/N/N | Negative | Perfused |
| | | | | | cardiac | | | | liver |
| | | | | | arrest | | | | |

### DNA Isolation

DNA from fibroblasts and hepatocytes was isolated using Quick-gDNA™ Blood MiniPrep (Zymo Research Corporation, Irvine, CA, USA) according to the manufacturer instructions and quantified using QUBIT kit (ThermoFisher Scientific, USA).

### SMM Library Preparation and Sequencing

Genomic DNA was first fragmented by double digestion with restriction endonucleases AluI and MluCI (NEB, USA), overnight at 37° C. After purification using 1.5×AMPure XP beads (Beckman Coulter, USA) the fragmented DNA was further processed using NEBNext Ultra II DNA Library Prep Kit for Illumina (NEB). The adapter provided with the kit was replaced with custom adapter P5_HP6N. After double sided size-selection using AMPure XP beads (Beckman Coulter), resulting dumbbell-like product was quantified with QUBIT kit (ThermoFisher) and analyzed on 2100 Bioanalyzer instrument with High Sensitivity DNA kit (Agilent, USA). To prepare P5_HP6N adapter 86 μl of 10 μM solution of oligonucleotide 5′-TCTTC TACAGT NNNNNN AGATCG GAAGAG CACACG TCTGAA CTCCAG TC/ideoxyU/ACACTC TTTCCC TACACG ACGCTC TTCCGA TCT-3′ (IDT, USA) in 0.1×TE buffer was first exposed to 95° C. for 5′ followed by 37° C. for 5′ to form a hairpin. Next, to fill-in the extending 5′-end the self-annealed oligonucleotide was supplemented with 10 μl of Cutsmart buffer (NEB), 2 μl of 10 mM dNTPs mix (NEB) and 10 U of Klenow Fragment (3′→5′ exo-) (NEB) and incubated at 37° C. for 30′. After purification with QIAquick Nucleotide Removal Kit (QIAGEN, USA), the hairpins were digested with 10U of HpyCH4III (NEB) for 1 hour at 37° C., then purified again with QIAquick Nucleotide Removal Kit (QIAGEN) and eluted with 100 μl of EB to obtain ready to use adapter solution.

Next, for SMM sequencing library preparation samples were diluted based on assessed molar concentration. Assuming 150PE sequencing mode and 30 Gb of data per sample, the dilution coefficient (D) was calculated using the formula D=M*NA/1025, where M—sample concentration (pM); NA—Avogadro constant. Next, 1 μl of diluted sample was used as a template in pulse-RCA reaction. The pulse-RCA was performed in 20 μl reaction containing 1 μl of diluted sample, 1 μl of P5-RCA oligo (5′-GTAGGGAAAGAGTGTAGACTGGAGTTC-3′), 25U (0.5 μl) of SD polymerase HS (BIORON Diagnostics GmbH, Germany), 2 μl of SD polymerase buffer, 1 μl of 10 mM dNTPs mix (NEB), 0.6 μl of 100 mM MgCl2, and 13.9 μl of water. The pulse-RCA program was set as follows: 92° C. for 2′ (1); 92° C. for 30″ (2); 60° C. for 30″ (3); 65° C. for 150″ (4); go to (3) 9 times; hold at 4° C. Product of amplification reaction was purified with 1.5×229 AMPure XP beads and resuspended in 23 μl TE buffer. The entire volume of RC amplification was PCR amplified in 50 μl reaction volume containing 23 μl of RCA product, 25 μl of NEBNext Ultra II Q5 Master Mix and 1 μl of P5 and P7 dual index oligos. The PCR program was set as follows: 98° C. for 30″ (1); 98° C. for 10″ (2); 65° C. for 75″ (3); go to (2) 8 times (4); 65° C. for 5′ (5); 4° C. forever. The PCR product was purified with 0.7×AMPure XP beads and resuspended in 30 μl of TE buffer. After quantification with Qubit, samples were pooled and sequenced on Illumina NovaSeq instrument using 150 paired-end mode.

Conventional sequencing library was prepared by PCR amplification of adapter ligated samples in 30 μl reaction volume containing 11 μl of undiluted ligated sample, 2U of USER enzyme (NEB) 15 μl of NEBNext Ultra II Q5 Master Mix and 1 μl of P5 and P7 dual index oligos. The PCR program was set as follows: 37° C. for 15′ (1); 98° C. for 30″ (2); 98° C. for 10″ (3); 65° C. for 75″ (4); go to (3) 4 times (4); 65° C. for 5′ (5); 4° C. forever. The PCR product was purified with 0.7×AMPure XP beads and resuspended in 30 μl of TE buffer. After quantification with Qubit, samples were pooled and sequenced on Illumina NovaSeq instrument using 150 paired-end mode.

### Data Processing and Variant Calling

Raw sequence reads were adapter and quality trimmed, aligned to human reference genome, realigned and recalibrated based on known indels as we described previously except that deduplication step was omitted.

For variant calling, we developed a set of filters which were applied to each position in SMM sequencing data. Only reads in proper pairs, with mapping quality not less than 60 and without secondary alignments were taken in consideration. Positions in SMM sequencing data was considered as qualified for variant calling if it is covered by UMI-family containing not less than 7 reads from each strand and this position is covered at least 20× in regular sequencing data. The qualified position was considered as a potential variant if all the reads within a given UMI family reported the same base at this position and this base was different from corresponding reference genome. Next, to filter out germline variants we checked if a found potential variant is in a list of single nucleotide polymorphisms (SNPs) of this DNA sample as well as in dbSNP. List of sample specific germline SNPs was prepared by analysis of conventional sequencing data with GATK haplotype caller. Finally, a variant was rejected if one or more reads of different UMI family in SMM data or in conventional data contained the same variant. SNVs frequency was calculated as a ratio of the number of identified variants to the total number of qualified positions.

TABLE 4
Summary of SMM-Seq analysis, mutation calling and
mutation spectra in IMR90 cells treated with ENU.

| | Sample | | Unique | Qualified | sSNVs | | | | | | | |
|---|---|---|---|---|---|---|---|---|---|---|---|---|
| # | ID | Cond | positions | positions | count | sSNVs frq | CG | CA | TG | TA | TC | CT |
| 1 | CON_1 | CON | 408878671 | 191359799 | 40 | 0.20903032 | 4 | 14 | 2 | 7 | 4 | 9 |
| 2 | CON_2 | CON | 564027867 | 252704169 | 51 | 0.20181701 | 7 | 14 | 3 | 8 | 7 | 12 |
| 3 | CON_3 | CON | 219566377 | 98900754 | 23 | 0.23255637 | 2 | 8 | 1 | 1 | 3 | 8 |
| 4 | E25_1 | E25 | 400139643 | 184905288 | 76 | 0.41102124 | 7 | 19 | 9 | 13 | 18 | 10 |
| 5 | E25_2 | E25 | 498176098 | 241793364 | 78 | 0.3225895 | 8 | 16 | 12 | 10 | 22 | 10 |
| 6 | E25_3 | E25 | 384505582 | 194702349 | 70 | 0.35952314 | 4 | 12 | 7 | 16 | 20 | 11 |
| 7 | E50_1 | E50 | 538283986 | 246936658 | 141 | 0.57099663 | 2 | 24 | 17 | 38 | 28 | 32 |
| 8 | E50_2 | E50 | 346288300 | 166489495 | 90 | 0.54057465 | 6 | 16 | 9 | 20 | 23 | 16 |
| 9 | E50_3 | E30 | 447787723 | 217064453 | \|110 | 0.50676192 | 6 | 16 | 13 | 28 | 26 | 21 |

TABLE 5
Summary of SMM-Seq analysis, mutation calling and mutation
spectra in human liver of young and old subjects.

| | Sample | Age | Unique | Qualified | sSNVs | | | | | | | |
|---|---|---|---|---|---|---|---|---|---|---|---|---|
| # | ID | group | positions | positions | count | sSNVs frq | CG | CA | TG | TA | TC | CT |
| 1 | L04 | Young | 749221420 | 751,040,680 | 301 | 0.40077723 | 21 | 50 | 16 | 65 | 62 | 87 |
| 2 | L05 | Young | 756113304 | 850,990,459 | 207 | 0.24324597 | 13 | 28 | 12 | 29 | 35 | 90 |
| 3 | L11 | Young | 709594619 | 705,204,674 | 271 | 0.3842856 | 18 | 51 | 8 | 73 | 30 | 91 |
| 4 | L02 | Old | 761086732 | 794,515,157 | 901 | 1.13402494 | 72 | 172 | 59 | 156 | 235 | 207 |
| 5 | L03 | Old | 674305216 | 670,894,859 | 630 | 0.93904431 | 48 | 107 | 49 | 110 | 166 | 150 |
| 6 | L13 | Old | 722700676 | 849,192,135 | 690 | 0.81253696 | 50 | 136 | 36 | 101 | 240 | 127 |

### Statistical Analysis

Statistic tests were performed using Microsoft Office Excel (2013). All the experiments were performed in three biological replicates and results are expressed as mean and standard deviation. Statistical significance of differences between experimental groups was determined using 2-tailed 264 t-test.

### Example 2. SMM-Seq Library Preparation

The key feature of SMM-Seq is a novel two-step library preparation protocol (FIG. 1). First, Rolling Circle-based linear amplification (RCA) is utilized to produce single-stranded DNA (ssDNA) molecules composed of multiple concatemerized copies of equally represented DNA strands of each particular DNA fragment. The amplification is carried out using a novel artificial thermostable polymerase possessing a strong strand displacement activity (SD polymerase). This allows multiple cycles of denaturation-annealing-extension to ensure efficient and less biased amplification in a reaction we termed pulse-RCA. Since all these copies are independent replicas of the original DNA fragment, potential errors of amplification remain unique for each copy and do not propagate further. Copies of opposite strands are in an end-to-end orientation and separated by common spacers used as PCR priming sites during the second step of the process when concatemerized copies are individually amplified and converted into a sequencing library (FIG. 1A). Thus, the resulting sequencing library is composed of PCR-duplicates of multiple independent copies of an original DNA fragment assembled in RC-amplicons.

### Example 3. SMM-Seq Data Analysis and Variant Calling

Sequencing reads originating from the same fragment are recognized based on unique molecular identifiers (UMIs) introduced as part of hairpin-like adapters during library preparation. UMI families composed of reads originating from both strands of the original fragments are then used to identify the consensus sequence of each fragment. Consensus calls different from the corresponding positions on the reference genome are compared with a list of single nucleotide polymorphisms (SNPs) of this particular DNA sample as well as with dbSNP. This allows to filter out germline variants and identify potential de novo somatic mutations. A list of germline SNPs is obtained by analysis of conventional sequencing data of the same DNA sample performed in parallel with SMM-Seq. The resulting list of potential somatic SNVs is further filtered to exclude low confidence candidates and then saved for further analysis (FIG. 1B).

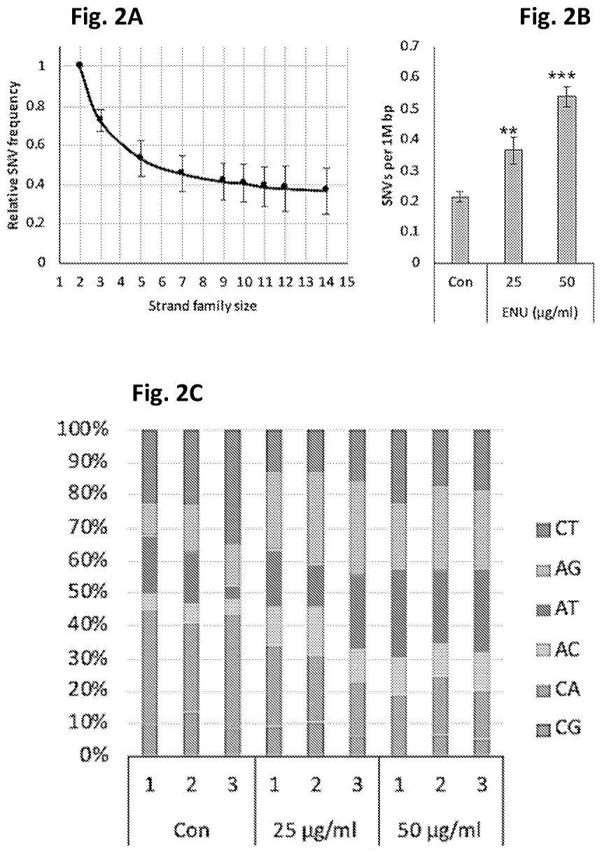

To determine the optimal analysis parameters, we assessed the frequency of somatic SNVs detected by the SMM variant-calling pipeline as a function of strand family size, i.e., the number of reads representing each strand in a UMI-family. We reasoned that each variant detected by SMM-Seq is falling into one of the following categories-true positive (TP) or false positive (FP). Then, mutation frequency is a sum of frequencies of TP and FP, i.e., (TP+FP)/number of analyzed bases. The SMM library contains PCR replicates of multiple independent RC copies of each strand of the original fragments. The size of the UMI strand families (shown in green and red in FIG. 1) determines the chance of multiple PCR duplicates of the same RCA error. Thus, greater UMI strand families are less prone to FP calls. Hence, the frequency of FP mutations should decline with increasing family size. To test this, we determined the mutation frequency at different family size and normalized the results to the mutation frequency at a strand family size of two, as has been used in NanoSeq, a modified version of Duplex-Seq. We found that increasing the minimum required strand family size from two to an arbitrary seven resulted in a more than two-fold decrease in observed mutation frequency (54% change) while further increase of strand family size did not lead to any significant decline in detected mutation frequency (less than 10% change) (FIG. 2A). Thus, we used a cutoff level of 7 reads per strand family as a qualifying criterion for variant calling.

### Example 4. Detection of Induced SNVs by SMM-Seq

As a proof of principle, we first performed SMM-Seq analysis of DNA extracted from normal human IMR90 fibroblasts subjected in vitro to a single treatment with two different doses of N-ethyl-N-nitrosourea (ENU), a potent point mutagen. Here, we used sub-lethal doses of ENU which do not cause any noticeable cell death on IMR90 cells. Analysis of SMM-Seq data revealed that there are ˜200M suitable for variant calling positions per sample on average. The regular sequencing library from IMR90 DNA was prepared, sequenced and analyzed in parallel with SMM-Seq to obtain a list of IMR90-specific germline SNPs. We found that our SMM-Seq assay allows detection of mutagenic effects of ENU in all tested conditions (FIG. 2B and Table 4). The lowest dose of ENU (25 μg/ml) increased the mutation frequency in IMR90 cells from 0.21±0.02 to 0.36=0.04 SNV/1 Mb (p=0.005), while ENU at 50 μg/ml led to a more than 2-fold increase of mutation frequency (0.54±0.03 SNV/1 Mb; p=9.7*10-5). We also tested mutation spectra of somatic SNVs in control, non-treated cells and cells subjected to ENU treatment. We observed a distinct shift of mutational spectra upon ENU treatment, with the relative representations of AT/TA and AT/CG mutations (specific for ENU (13))>2-times larger than in the untreated control cells (FIG. 2C). Thus, SMM-Seq is capable of detecting somatic SNVs induced by low doses of mutagen.

### Example 5. Detection of Aging-Associated SNVs by SMM-Seq

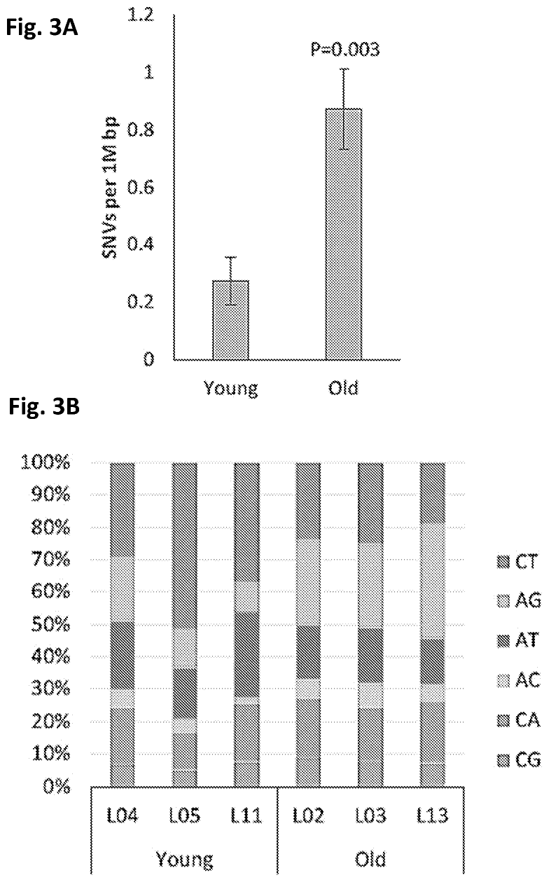

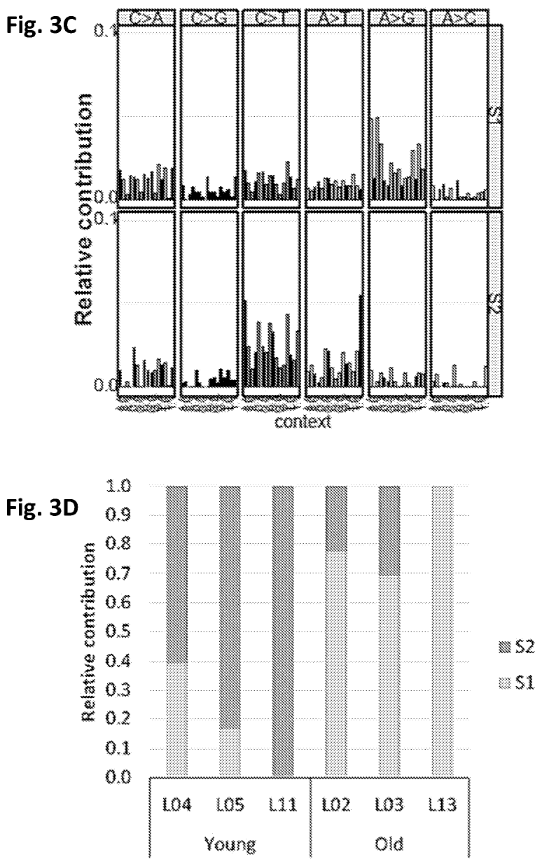

Next, we tested if SMM-Seq is capable of detecting physiological mutation burdens in human tissues accumulated during aging. We took advantage of our recently published study on the age-related mutational load in human liver that was performed using the gold standard single cell-based approach and re-analyzed the same samples using SMM-Seq assay. We used the whole genome sequences of bulk DNAs from each subject from that same study for subtracting germline SNPs. SMM-Seq libraries were prepared from DNA samples extracted from liver tissue of three young (5 months, 16 months, and 18 years old) and three aged people (56, 61, and 77 years old). Analysis of SMM-Seq data in this experiment revealed that there are ˜770M positions qualified for variant calling per sample on average. As shown in FIG. 3A, SMM-seq confirmed the age-related elevation in the somatic mutation frequency observed by the single-cell approach (Table 5). Mutation frequencies assayed by SMM-Seq were 0.34±0.09 and 0.96±0.16 somatic SNVs/1 Mb in the young and aged group, respectively (p=0.003). Analysis of somatic SNV spectra revealed an almost two-fold increase in the relative representation of AT to GC mutations in the liver DNA of aged people (16.2% in young vs. 29.1% in aged) (FIG. 3B), similar to what has been observed by the single-cell approach.

### Example 6. Assessment of Mutation Signature Using SMM-Seq Data

To get further insight into the mutation spectra in the aged human liver, we performed non-negative matrix factorization and extracted two de novo mutation signatures, S1 and S2 (FIG. 3C), from the mutation spectra of 6 analyzed samples. Signature S1 was found to be substantially increased in the aged group (p=0.0134) and associated with aging signature SBS5 (cosine similarity: 0.904). Signature S2 was dominant in the young group, with an abundance of CG to TA transitions and AT to TA transversions, but the source of signature S2 is not clear, and we did not find any significant similarity between signature S2 and known COSMIC signatures. These signature analysis results as well as our results on age-related increase of mutational load are in good agreement with our previous findings. Thus, SMM-Seq is capable of detecting somatic SNVs accumulated in normal human tissues under physiological conditions.

The various approaches utilizing duplex consensus sequencing for the identification of rare mutations, i.e., the original Duplex-Seq, BotSeqS, and NanoSeq, are all based on analysis of the two opposite DNA strands to eliminate potential errors. The error rate of these approaches is determined by the probability of two complementary errors in both strands and can be defined as P(E)², where P(E) is the probability of error on any of two strands. SMM-seq is not limited to two strands only since it utilizes sequencing data from multiple independent copies of each strand for variant calling. Conversely, SMM-Seq's error rate can be calculated as P(E)^{N}, where N is the number of independent copies produced in the linear amplification step. Naturally, copies of the same strand cannot be distinguished from the sequencing data, but our results on variant calling using strand families of different sizes clearly demonstrated that the detected mutation frequency is plateauing at a family size of 7 and further (FIG. 2A). This indicates, that at this size each strand family contains descendants of more than one copy of the original DNA fragment and no further improvement of accuracy is possible. Of note, despite virtually unlimited accuracy in base calling on each strand, it is still necessary to have representatives of both to filter out possible artifacts produced by DNA damage, which are expected to be present on one strand only (FIG. 1B).

As demonstrated herein, SMM-Seq is capable of detecting both induced and naturally occurring somatic SNVs in normal human cells and tissues. The SMM-Seq results are in line with results obtained using the single cell-based approach, currently the gold standard in the field. However, usage of SMM-Seq is significantly less resource demanding. Most importantly, SMM-Seq is more accurate than Duplex-Seq-based approaches due to the presence of multiple independent copies of the original DNA fragment. Thus, SMM-Seq is a practical approach which, together with our previously developed SVS assay for detecting somatic structural variants, is well suited for the comprehensive assessment of genome integrity in large scale human studies.

Referring now to FIG. 4, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Sequencing data may be accessed at dbGaP—phs001956.v1.p1 (World Wide Web at ncbi.nlm.hin.gov/projects/gap/cgi-bin/study.cgi?study_id=phs001956.v1.p1) and World Wide Web at dataview.ncbi.nlm.nih.gov/object/PRJNA758911. Analysis scripts may be accessed at World Wide Web at zenodo.org/record/5804750#. Ycnt8S-B2-Y and World Wide Web at github.com/msd-ru/SMM.

### Example 7. Detection of Structural Variants (SVs) in SMM-Seq

As explained, SMM is capable of identifying SNVs and small indels, but not SVs. This is due to the overwhelming amount of chimeric DNA fragments created during library construction. For SMM-seq and other single-molecule assays the process of linking DNA fragments to the sequencing adapters also results in random ligation of DNA fragments to each other. The resulting chimeric sequences are not distinguishable from true SVs since both strands are identical and represent the same SV (FIG. 9A). To address this problem, we used transposase-mediated tagmentation, i.e., the initial step in library preparation where high molecular weight DNA is cleaved and tagged for analysis. While commonly used in library preparation, we used it here in a different, unexpected way. Indeed, as commonly used tagmentation generates priming sites for subsequent PCR which in turn is prone to produce artificial chimeras, giving a similar problem as other approaches. It occurred to us that instead of using the transposome approach in the regular way, we could utilize transposase-mediated tagmentation to create single strand overhangs of extended length which would allow sticky-end ligation of DNA fragments to sequencing adapters completely prohibiting blunt-end ligation. This would prevent artificial chimeric sequences and allow utilization of SMM for detection of SVs. One preferred protocol includes (FIG. 9B):
1. Simultaneous fragmentation and tagmentation of DNA using Tn5-mediated transposition reaction with transposon containing uracil residue 5′ to the Tn5 Mosaic End (ME)
2. Polymerase extension to fill 9 nt gap created by Tn5 and reconstitute bottom strand
3. Digestion using Uracil-DNA Glycosylase (UDG) which recognizes and releases uracil from uracil containing DNA. This step creates 3′-end identical overhangs on both ends of all DNA fragments.
4. Ligation of SMM hairpin adapters with protruding 3′-end complementary to the engineered DNA fragments 3′-end overhangs
   Thus, forming of artificial chimeric sequences is precluded and resulting dumbbell-like structures can be used in SMM analysis for identification of SVs. To validate SMM-SV we evaluated somatic SV frequency in human primary mammary cells treated with two different doses of bleomycin, a potent elastogen known to induce somatic SVs. We found that SMM-SV allows detection of induced SVs in a dose-dependent manner (FIG. 9C). This modification of SMM allows detection of large deletion, translocations, duplications and inversions.

TABLE 6
COSMIC DATA (COSMIC_v3.2_DBS_GRCh37)

| Type | DBS1 | DBS2 | DBS3 | DBS4 | DBS5 | DBS6 |
|---|---|---|---|---|---|---|
| AC > CA | 4.97E−05 | 0.000374079 | 0.006347323 | 0.004141857 | 0.002139822 | 0.002660571 |
| AC > CG | 2.64E−05 | 3.17E−05 | 4.65E−05 | 4.96E−05 | 0.000461962 | 0.000146031 |
| AC > CT | 4.29E−03 | 1.26E−04 | 0.003018727 | 8.32E−18 | 0.002019832 | 0.000240052 |
| AC > GA | 0.000127032 | 0.00023705 | 0.005847534 | 0.004401974 | 0.000557954 | 0.008581841 |
| AC > GG | 5.71E−05 | 4.17E−18 | 0.00044981 | 7.01E−18 | 1.11E−17 | 0.000104022 |
| AC > GT | 0.002960753 | 0.000280059 | 0.010095742 | 1.12E−08 | 0.000821932 | 0.000132028 |
| AC > TA | 0.001200305 | 0.001480313 | 0.001009574 | 0.00127057 | 0.001479877 | 0.010102168 |
| AC > TG | 1.72E−04 | 6.36E−06 | 1.11E−17 | 5.31E−05 | 0.001119907 | 0.000449096 |
| AC > TT | 0.026506737 | 0.002920618 | 1.78E−06 | 0.000161072 | 0.007469379 | 0.003180682 |
| AT > CA | 0.000482123 | 3.78E−05 | 0.007037032 | 0.000916411 | 0.000107991 | 0.000114024 |
| AT > CC | 0.000297076 | 4.17E−18 | 0.009076172 | 7.00E−18 | 2.13E−05 | 6.39E−05 |
| AT > CG | 3.98E−06 | 4.17E−18 | 0.014593845 | 9.46E−18 | 1.05E−17 | 1.82E−06 |
| AT > GA | 2.12E−03 | 1.68E−04 | 0.001809237 | 3.24E−04 | 0.001109908 | 0.001620348 |
| AT > GC | 1.74E−04 | 5.85E−06 | 0.005247787 | 1.76E−05 | 1.05E−17 | 1.72E−05 |
| AT > TA | 2.34E−03 | 4.58E−04 | 0.002349009 | 4.53E−03 | 0.000792934 | 0.002200472 |
| CC > AA | 1.06E−05 | 0.724153238 | 0.009086168 | 7.00E−18 | 1.05E−17 | 8.30E−18 |
| CC > AG | 1.23E−05 | 0.072615366 | 1.11E−17 | 5.43E−03 | 0.024297979 | 0.06161322 |
| CC > AT | 0.018704754 | 0.100021165 | 0.005887517 | 0.015707044 | 0.002429798 | 0.000438094 |
| CC > GA | 0.000134034 | 0.017703746 | 0.000634732 | 0.003551593 | 6.76E−05 | 0.035507619 |
| CC > GG | 9.82E−05 | 4.50E−04 | 7.25E−05 | 7.30E−05 | 1.19E−05 | 0.018103885 |
| CC > GT | 0.006921759 | 0.001100233 | 1.14E−17 | 7.99E−05 | 2.48E−05 | 0.003440738 |
| CC > TA | 0.010402644 | 0.016003386 | 0.020791232 | 0.000170076 | 0.001269894 | 0.002220476 |
| CC > TG | 0.002550648 | 0.00023705 | 2.90E−05 | 8.30E−06 | 0.0048096 | 0.006871474 |
| CC > TT | 0.858218125 | 4.17E−18 | 1.11E−17 | 7.00E−18 | 1.05E−17 | 8.30E−18 |
| CG > AT | 6.03E−07 | 0.001150243 | 0.00028388 | 0.00029013 | 0.001639864 | 0.007541618 |
| CG > GC | 5.42E−06 | 0.000369078 | 1.11E−17 | 5.10E−06 | 9.92E−05 | 0.000590127 |
| CG > GT | 1.70E−06 | 0.001280271 | 1.11E−17 | 7.00E−18 | 0.006479461 | 0.003220691 |
| CG > TA | 0.000356091 | 4.17E−18 | 0.097858731 | 7.00E−18 | 1.05E−17 | 8.30E−18 |
| CG > TC | 1.26E−05 | 0.002150455 | 0.000721696 | 7.00E−18 | 2.98E−05 | 0.008911912 |
| CG > TT | 1.24E−04 | 5.56E−03 | 0.007166978 | 0.000361162 | 0.002219815 | 0.018704013 |
| CT > AA | 2.77E−05 | 3.21E−03 | 0.000102957 | 7.00E−18 | 0.465961237 | 0.004480961 |
| CT > AC | 0.000419107 | 4.17E−18 | 0.029087733 | 7.00E−18 | 0.208982615 | 0.001950419 |
| CT > AG | 2.85E−18 | 0.000587124 | 1.14E−01 | 0.000363163 | 0.026697779 | 0.039508477 |
| CT > GA | 8.92E−05 | 1.81E−05 | 1.72E−05 | 0.000673302 | 0.016098661 | 7.34E−03 |
| CT > GC | 2.52E−04 | 1.34E−04 | 2.69E−05 | 7.22E−05 | 0.004849597 | 1.79E−03 |
| CT > GG | 1.08E−05 | 8.98E−05 | 5.99E−05 | 0.000926415 | 0.000569953 | 0.006811462 |
| CT > TA | 7.81E−03 | 3.38E−03 | 0.000183922 | 7.00E−18 | 0.003339722 | 8.84E−05 |
| CT > TC | 0.010502669 | 3.84E−05 | 0.006337327 | 7.00E−18 | 0.00023698 | 8.30E−18 |
| CT > TG | 2.24E−03 | 0.000390083 | 0.052177995 | 3.08E−05 | 0.000873927 | 0.011202404 |
| GC > AA | 1.58E−03 | 5.08E−03 | 2.16E−05 | 0.328147163 | 0.000132989 | 8.30E−18 |
| GC > AG | 1.07E−04 | 3.25E−04 | 5.22E−04 | 0.01130507 | 0.002659779 | 0.003660785 |
| GC > AT | 4.87E−05 | 0.000953202 | 3.99E−03 | 0.009244146 | 0.001569869 | 1.88E−03 |
| GC > CA | 5.76E−05 | 1.06E−03 | 0.0002369 | 0.011905339 | 0.004579619 | 0.008691865 |
| GC > CG | 5.15E−06 | 6.69E−05 | 1.11E−17 | 9.83E−07 | 0.000322973 | 9.04E−18 |
| GC > TA | 8.27E−07 | 0.000736156 | 4.23E−03 | 0.017707941 | 0.000221982 | 0.000829178 |
| TA > AT | 5.05E−04 | 0.002750582 | 1.45E−03 | 0.030413639 | 0.001869844 | 0.008571839 |
| TA > CG | 7.55E−06 | 1.89E−05 | 3.34E−03 | 0.000389175 | 7.89E−06 | 2.59E−04 |
| TA > CT | 7.02E−04 | 2.80E−04 | 3.44E−04 | 0.008723912 | 0.002269811 | 2.61E−02 |
| TA > GC | 2.01E−05 | 8.22E−05 | 0.001989161 | 7.02E−05 | 5.22E−05 | 0.000264057 |
| TA > GG | 7.03E−05 | 7.89E−05 | 0.005457698 | 0.002220996 | 0.001629864 | 0.000218047 |
| TA > GT | 0.001150292 | 0.001300275 | 0.000361847 | 0.008063616 | 0.001909841 | 6.25E−05 |
| TC > AA | 1.09E−05 | 0.001450307 | 0.005487686 | 0.379170045 | 0.040396639 | 0.045809829 |
| TC > AG | 3.09E−06 | 0.000200042 | 6.14E−06 | 1.87E−03 | 1.49E−02 | 0.008281777 |
| TC > AT | 0.002460625 | 4.17E−18 | 1.12E−03 | 0.008413773 | 0.000747938 | 0.000594127 |
| TC > CA | 4.42E−06 | 6.82E−04 | 6.55E−02 | 8.46E−02 | 0.001429881 | 0.011802532 |
| TC > CG | 3.30E−05 | 3.89E−06 | 0.000115951 | 0.000400179 | 1.06E−17 | 0.000862185 |
| TC > CT | 1.90E−03 | 0.003350709 | 1.94E−02 | 8.28E−06 | 0.001049913 | 0.009392015 |
| TC > GA | 6.42E−05 | 8.85E−04 | 7.78E−02 | 3.64E−02 | 2.46E−05 | 0.007341575 |
| TC > GG | 4.11E−04 | 9.87E−06 | 1.77E−04 | 1.04E−05 | 3.48E−05 | 0.001510324 |
| TC > GT | 0.005101297 | 8.57E−05 | 4.34E−02 | 7.00E−18 | 3.86E−05 | 8.30E−18 |
| TG > AA | 1.37E−03 | 2.96E−03 | 6.68E−05 | 0.007633423 | 0.017998503 | 0.025505473 |
| TG > AC | 0.00023706 | 0.000220047 | 8.04E−05 | 0.000118053 | 0.001459879 | 0.010102168 |
| TG > AT | 2.41E−03 | 4.29E−03 | 9.58E−04 | 7.00E−18 | 0.020398303 | 0.22104743 |
| TG > CA | 0.000279071 | 0.00014403 | 1.59E−02 | 8.04E−04 | 4.76E−04 | 0.011402447 |
| TG > CC | 0.000103026 | 5.94E−05 | 4.84E−04 | 0.000120054 | 5.06E−04 | 7.89E−03 |
| TG > CT | 0.00185047 | 0.00307065 | 6.07E−03 | 8.21E−05 | 0.000327973 | 0.27505902 |
| TG > GA | 4.07E−04 | 1.47E−03 | 5.23E−04 | 7.85E−03 | 0.003269728 | 0.007441597 |
| TG > GC | 8.89E−05 | 0.000272058 | 0.00049779 | 0.000604271 | 0.000291976 | 0.005601202 |
| TG > GT | 0.001900483 | 6.40E−03 | 1.03E−02 | 3.23E−05 | 0.082693121 | 0.027405881 |
| TT > AA | 0.005491396 | 4.31E−03 | 3.22E−03 | 1.80E−05 | 0.004919591 | 1.93E−04 |
| TT > AC | 0.000720183 | 3.45E−04 | 2.43E−03 | 6.60E−07 | 0.00096592 | 1.28E−04 |
| TT > AG | 0.000418106 | 0.000215046 | 2.29E−02 | 7.00E−18 | 0.000892926 | 2.12E−03 |
| TT > CA | 0.000725184 | 4.17E−18 | 0.000693707 | 7.00E−18 | 1.05E−17 | 8.30E−18 |
| TT > CC | 5.42E−03 | 2.98E−06 | 0.00815656 | 7.00E−18 | 0.000772936 | 8.30E−18 |
| TT > CG | 8.38E−05 | 1.52E−05 | 0.031786595 | 7.25E−18 | 1.05E−17 | 1.12E−05 |
| TT > GA | 1.86E−03 | 4.79E−05 | 1.23E−02 | 7.00E−18 | 2.99E−05 | 8.30E−18 |
| TT > GC | 0.001060269 | 1.54E−06 | 0.066471967 | 7.00E−18 | 5.66E−05 | 4.09E−07 |
| TT > GG | 0.001610409 | 4.17E−18 | 0.170927916 | 7.00E−18 | 1.05E−17 | 8.30E−18 |

| Type | DBS7 | DBS8 | DBS9 | DBS10 | DBS11 |
|---|---|---|---|---|---|
| AC > CA | 0.045880132 | 0.214923552 | 0.024591818 | 0.009630417 | 0.001369907 |
| AC > CG | 0.006247295 | 0.000972654 | 0.002069312 | 1.77E−05 | 0.000193987 |
| AC > CT | 0.013594113 | 10.317886928 | 0.028490521 | 0.013500585 | 0.000755948 |
| AC > GA | 0.009725788 | 0.001159588 | 0.025291585 | 0.005350232 | 4.42E−04 |
| AC > GG | 0.002538901 | 0.000809712 | 0.007127629 | 0.000590026 | 2.25E−03 |
| AC > GT | 0.004498052 | 0.000150946 | 1.21E−02 | 2.87E−02 | 0.005709611 |
| AC > TA | 0.005417654 | 0.012395591 | 0.041186297 | 0.000105005 | 0.000263982 |
| AC > TG | 0.000997568 | 0.002569086 | 0.017894047 | 7.66E−06 | 0.000238984 |
| AC > TT | 0.001179489 | 0.000267905 | 0.044385233 | 0.000710031 | 0.002819808 |
| AT > CA | 0.011095195 | 2.78E−05 | 0.012695776 | 0.003530153 | 0.009479354 |
| AT > CC | 0.00625729 | 0.000142949 | 0.001779408 | 0.00116005 | 0.00189987 |
| AT > CG | 1.11E−03 | 0.000265905 | 9.22E−18 | 2.59E−03 | 1.74E−05 |
| AT > GA | 0.014393767 | 0.000161942 | 6.37E−03 | 8.06E−05 | 0.011199236 |
| AT > GC | 9.29E−03 | 0.00022592 | 1.81E−03 | 0.004080177 | 0.004179715 |
| AT > TA | 3.40E−02 | 0.000314888 | 0.00919694 | 0.002930127 | 0.003839738 |
| CC > AA | 9.04E−18 | 0.038086453 | 9.02E−18 | 0.013700594 | 7.35E−18 |
| CC > AG | 8.30E−05 | 0.015494489 | 2.64E−02 | 7.13E−05 | 0.000971934 |
| CC > AT | 0.000851631 | 0.028789759 | 1.95E−02 | 0.010500455 | 0.034497648 |
| CC > GA | 3.02E−04 | 0.000808712 | 0.010996342 | 0.003390147 | 0.000952935 |
| CC > GG | 0.002049113 | 5.60E−05 | 0.003058982 | 7.41E−18 | 0.003449765 |
| CC > GT | 0.000162929 | 0.000228919 | 0.017894047 | 0.001080047 | 0.069495262 |
| CC > TA | 9.04E−18 | 0.000715745 | 0.000686772 | 0.023901036 | 0.058795991 |
| CC > TG | 0.000235898 | 0.004208503 | 0.015494845 | 7.41E−18 | 0.032297798 |
| CC > TT | 9.04E−18 | 0.004378443 | 9.02E−18 | 2.06E−02 | 0.331977366 |
| CG > AT | 6.21E−05 | 0.000321886 | 0.000218927 | 7.41E−18 | 1.82E−03 |
| CG > GC | 0.000186919 | 1.13E−17 | 1.20E−05 | 1.90E−05 | 0.000165989 |
| CG > GT | 2.07E−05 | 0.00120957 | 9.94E−05 | 1.02E−05 | 0.00117992 |
| CG > TA | 9.04E−18 | 5.06E−04 | 9.02E−18 | 0.358015516 | 7.38E−18 |
| CG > TC | 4.87E−05 | 0.000904678 | 1.33E−04 | 2.65E−03 | 6.85E−04 |
| CG > TT | 3.13E−05 | 0.000328883 | 8.29E−05 | 7.77E−03 | 0.000698952 |
| CT > AA | 9.04E−18 | 0.020592675 | 9.02E−18 | 0.00393017 | 0.0043997 |
| CT > AC | 1.01E−17 | 0.013995022 | 9.02E−18 | 0.013000563 | 0.00234984 |
| CT > AG | 0.001259455 | 0.020392746 | 0.000581806 | 0.026501149 | 0.000495966 |
| CT > GA | 0.001869191 | 0.005568019 | 0.002839055 | 7.41E−18 | 0.011599209 |
| CT > GC | 0.015593248 | 2.37E−04 | 3.11E−03 | 7.41E−18 | 0.006109583 |
| CT > GG | 0.003998269 | 3.06E−04 | 1.04E−02 | 7.41E−18 | 0.004019726 |
| CT > TA | 9.04E−18 | 0.010896124 | 9.02E−18 | 0.004030175 | 4.17E−02 |
| CT > TC | 0.063172644 | 0.017793671 | 3.00E−05 | 0.002750119 | 0.002989796 |
| CT > TG | 9.04E−18 | 0.000965657 | 9.46E−18 | 4.57E−02 | 1.48E−03 |
| GC > AA | 9.04E−18 | 1.29E−02 | 9.02E−18 | 0.004080177 | 0.000999932 |
| GC > AG | 7.30E−05 | 8.52E−03 | 1.24E−01 | 7.41E−18 | 0.003289776 |
| GC > AT | 0.000349849 | 5.51E−03 | 1.26E−01 | 0.007750336 | 0.027198146 |
| GC > CA | 6.70E−04 | 0.004828283 | 0.027390887 | 0.000105005 | 0.002109856 |
| GC > CG | 0.002648853 | 0.000241914 | 0.002499169 | 7.41E−18 | 0.004039725 |
| GC > TA | 6.24E−06 | 0.008556956 | 0.013995344 | 0.002810122 | 2.43E−04 |
| TA > AT | 0.055775847 | 9.70E−03 | 1.55E−02 | 0.010400451 | 2.23E−03 |
| TA > CG | 0.00304868 | 2.09E−05 | 1.35E−03 | 0.004110178 | 5.84E−04 |
| TA > CT | 2.91E−02 | 4.10E−03 | 1.42E−02 | 0.00162007 | 3.38E−03 |
| TA > GC | 1.51E−03 | 0.000788719 | 0.000578807 | 7.41E−18 | 0.00101993 |
| TA > GG | 0.004308134 | 0.000216923 | 0.003248919 | 0.008590372 | 3.75E−04 |
| TA > GT | 2.63E−02 | 0.000152946 | 0.007777412 | 0.003610156 | 0.002669818 |
| TC > AA | 0.019591516 | 0.038786204 | 0.003688773 | 0.007560328 | 0.000216985 |
| TC > AG | 4.00E−06 | 1.16E−02 | 4.67E−02 | 2.71E−04 | 0.047396768 |
| TC > AT | 0.002289009 | 0.018493422 | 5.18E−02 | 3.09E−03 | 0.084594232 |
| TC > CA | 0.002708827 | 0.017593742 | 0.04878377 | 0.025201092 | 2.82E−04 |
| TC > CG | 1.63E−03 | 0.010896124 | 0.007787409 | 0.000842036 | 0.003239779 |
| TC > CT | 9.19E−03 | 0.008756885 | 0.051382905 | 0.026201136 | 0.02009863 |
| TC > GA | 9.04E−18 | 9.61E−03 | 1.73E−02 | 0.043801898 | 2.35E−04 |
| TC > GG | 0.000705694 | 0.001909321 | 3.73E−03 | 7.41E−18 | 7.33E−03 |
| TC > GT | 9.04E−18 | 2.76E−04 | 9.15E−18 | 0.045901989 | 1.52E−02 |
| TG > AA | 0.022590218 | 0.008646924 | 0.015794745 | 0.001420062 | 0.007829466 |
| TG > AC | 2.62E−03 | 0.003848631 | 0.007267582 | 0.000207009 | 0.000865941 |
| TG > AT | 0.001289442 | 8.22E−03 | 7.46E−04 | 0.002500108 | 1.05E−02 |
| TG > CA | 0.023889655 | 5.06E−03 | 2.28E−02 | 0.029201266 | 5.61E−02 |
| TG > CC | 0.001049546 | 0.001249556 | 1.18E−02 | 0.000190008 | 3.13E−03 |
| TG > CT | 9.04E−18 | 0.019493066 | 9.68E−04 | 0.004330188 | 5.58E−03 |
| TG > GA | 0.008246429 | 0.000270904 | 0.006177945 | 2.02E−05 | 0.007309502 |
| TG > GC | 0.003878321 | 0.003538741 | 0.0039087 | 7.41E−18 | 0.006119583 |
| TG > GT | 8.71E−03 | 0.007857205 | 0.003338889 | 0.002170094 | 0.005239643 |
| TT > AA | 2.35E−01 | 3.58E−03 | 3.41E−04 | 0.014500628 | 4.77E−03 |
| TT > AC | 4.00E−02 | 1.13E−17 | 6.66E−03 | 7.41E−18 | 1.04E−03 |
| TT > AG | 5.39E−02 | 0.014594809 | 9.18E−04 | 0.006960302 | 1.27E−03 |
| TT > CA | 0.09765771 | 0.006287763 | 9.03E−18 | 0.001290056 | 0.002019862 |
| TT > CC | 0.035384677 | 0.000293895 | 10.005158284 | 0.002880125 | 0.003999727 |
| TT > CG | 0.002668844 | 0.000115959 | 1.98E−05 | 0.008580372 | 9.96E−18 |
| TT > GA | 0.043581128 | 0.003628709 | 9.17E−18 | 0.008690377 | 7.07E−04 |
| TT > GC | 0.003668411 | 0.000285898 | 3.56E−05 | 0.025801118 | 5.78E−06 |
| TT > GG | 9.04E−18 | 0.000537809 | 9.02E−18 | 8.47E−02 | 7.34E−18 |

TABLE 7
COSMIC DATA (COSMIC_v3.2_DBS_GRCh38)

| Type | DBS1 | DBS2 | DBS3 | DBS4 | DBS5 | DBS6 |
|---|---|---|---|---|---|---|
| AC > CA | 5.02E−05 | 0.000377632 | 0.006337519 | 0.004146665 | 0.002141988 | 0.002665185 |
| AC > CG | 2.66E−05 | 3.20E−05 | 4.64E−05 | 4.97E−05 | 0.000462429 | 0.000146285 |
| AC > CT | 0.004330586 | 0.000127224 | 0.003014064 | 8.33E−18 | 0.002021876 | 0.000240468 |
| AC > GA | 0.000128202 | 0.000239302 | 0.005838502 | 0.004407083 | 0.000558518 | 0.008596725 |
| AC > GG | 5.76E−05 | 4.21E−18 | 0.000449116 | 7.02E−18 | 1.11E−17 | 0.000104203 |
| AC > GT | 0.002988003 | 0.000282719 | 0.010080149 | 1.12E−08 | 0.000822764 | 0.000132257 |
| AC > TA | 0.001211353 | 0.001494372 | 0.001008015 | 0.001272044 | 0.001481375 | 0.010119688 |
| AC > TG | 0.000173627 | 6.42E−06 | 1.11E−17 | 5.32E−05 | 0.00112104 | 0.000449875 |
| AC > TT | 0.026750707 | 0.002948356 | 1.78E−06 | 0.000161259 | 0.007476939 | 0.003186199 |
| AT > CA | 0.000484053 | 3.80E−05 | 0.006989959 | 0.000912747 | 0.000107543 | 0.000113634 |
| AT > CC | 0.000298265 | 4.19E−18 | 0.009015459 | 6.98E−18 | 2.12E−05 | 6.37E−05 |
| AT > CG | 4.00E−06 | 4.19E−18 | 0.014496222 | 9.43E−18 | 1.05E−17 | 1.81E−06 |
| AT > GA | 0.002129029 | 0.000168757 | 0.001797134 | 0.000322849 | 0.001105306 | 0.001614794 |
| AT > GC | 0.000174741 | 5.88E−06 | 0.005212683 | 1.75E−05 | 1.05E−17 | 1.71E−05 |
| AT > TA | 0.002349966 | 0.000460065 | 0.002333296 | 0.004513913 | 0.000789647 | 0.002192931 |
| CC > AA | 1.06E−05 | 0.723624795 | 0.008980225 | 6.94E−18 | 1.04E−17 | 8.23E−18 |
| CC > AG | 1.23E−05 | 0.072562376 | 1.10E−17 | 0.005383642 | 0.024076164 | 0.061094801 |
| CC > AT | 0.018685674 | 0.099948176 | 0.00581887 | 0.015565963 | 0.002407616 | 0.000434408 |
| CC > GA | 0.000133897 | 0.017690827 | 0.000627331 | 0.003519692 | 6.70E−05 | 0.035208854 |
| CC > GG | 9.81E−05 | 0.000449767 | 7.16E−05 | 7.24E−05 | 1.18E−05 | 0.017951557 |
| CC > GT | 0.006914699 | 0.00109943 | 1.13E−17 | 7.92E−05 | 2.46E−05 | 0.003411788 |
| CC > TA | 0.010392033 | 0.015991708 | 0.02054881 | 0.000168549 | 0.001258302 | 0.002201793 |
| CC > TG | 0.002548046 | 0.000236877 | 2.86E−05 | 8.23E−06 | 0.004765693 | 0.006813657 |
| CC > TT | 0.857342694 | 4.17E−18 | 1.10E−17 | 6.94E−18 | 1.04E−17 | 8.23E−18 |
| CG > AT | 6.17E−07 | 0.001176331 | 0.000287143 | 0.00029426 | 0.00166296 | 0.007653355 |
| CG > GC | 5.54E−06 | 0.000377449 | 1.12E−17 | 5.17E−06 | 0.000100589 | 0.00059887 |
| CG > GT | 1.74E−06 | 0.001309308 | 1.12E−17 | 7.10E−18 | 0.00657072 | 0.003268409 |
| CG > TA | 0.000364061 | 4.27E−18 | 0.098983541 | 7.10E−18 | 1.06E−17 | 8.42E−18 |
| CG > TC | 1.29E−05 | 0.002199228 | 0.000729991 | 7.10E−18 | 3.02E−05 | 0.009043951 |
| CG > TT | 0.000126808 | 0.005687306 | 0.007249356 | 0.000366303 | 0.00225108 | 0.018981132 |
| CT > AA | 2.79E−05 | 0.003237827 | 0.000102691 | 7.00E−18 | 0.465951396 | 0.004484099 |
| CT > AC | 0.000422527 | 4.21E−18 | 0.029012824 | 7.00E−18 | 0.208978201 | 0.001951784 |
| CT > AG | 2.87E−18 | 0.000592089 | 0.113658487 | 0.000363209 | 0.026697215 | 0.039536145 |
| CT > GA | 9.00E−05 | 1.83E−05 | 1.71E−05 | 0.000673388 | 0.016098321 | 0.007346717 |
| CT > GC | 0.000254121 | 0.000135162 | 2.68E−05 | 7.22E−05 | 0.004849494 | 0.001791638 |
| CT > GG | 1.09E−05 | 9.06E−05 | 5.97E−05 | 0.000926533 | 0.000569941 | 0.006816232 |
| CT > TA | 0.007875749 | 0.0034093 | 0.000183449 | 7.00E−18 | 0.003339652 | 8.85E−05 |
| CT > TC | 0.010588395 | 3.87E−05 | 0.006321007 | 7.00E−18 | 0.000236975 | 8.31E−18 |
| CT > TG | 0.002258858 | 0.000393381 | 0.052043623 | 3.08E−05 | 0.000873909 | 0.011210249 |
| GC > AA | 0.001584499 | 0.00509573 | 2.14E−05 | 0.326375815 | 0.000132251 | 8.26E−18 |
| GC > AG | 0.000107305 | 0.000326006 | 0.000517561 | 0.011244045 | 0.002645029 | 0.003643111 |
| GC > AT | 4.88E−05 | 0.000955951 | 0.003956071 | 0.009194246 | 0.001561164 | 0.001871325 |
| GC > CA | 5.78E−05 | 0.001063282 | 0.000234985 | 0.011841074 | 0.004554223 | 0.0086499 |
| GC > CG | 5.16E−06 | 6.71E−05 | 1.10E−17 | 9.78E−07 | 0.000321182 | 9.00E−18 |
| GC > TA | 8.29E−07 | 0.000738279 | 0.00419403 | 0.017612353 | 0.000220751 | 0.000825175 |
| TA > AT | 0.000504971 | 0.002750525 | 0.001433506 | 0.030161852 | 0.00185409 | 0.008505747 |
| TA > CG | 7.55E−06 | 1.89E−05 | 0.003302006 | 0.000385953 | 7.82E−06 | 0.000257058 |
| TA > CT | 0.00070196 | 0.000280053 | 0.000340087 | 0.008651689 | 0.002250686 | 0.025904317 |
| TA > GC | 2.01E−05 | 8.22E−05 | 0.001967363 | 6.97E−05 | 5.18E−05 | 0.000262021 |
| TA > GG | 7.03E−05 | 7.89E−05 | 0.005397891 | 0.002202609 | 0.001616132 | 0.000216366 |
| TA > GT | 0.001149934 | 0.001300248 | 0.000357882 | 0.00799686 | 0.001893749 | 6.20E−05 |
| TC > AA | 1.10E−05 | 0.001470066 | 0.005501607 | 0.381161856 | 0.040602827 | 0.046076863 |
| TC > AG | 3.13E−06 | 0.000202768 | 6.15E−06 | 0.001880667 | 0.014974805 | 0.008330053 |
| TC > AT | 0.002493424 | 4.23E−18 | 0.001122368 | 0.008457972 | 0.000751755 | 0.000597591 |
| TC > CA | 4.48E−06 | 0.000691438 | 0.065638482 | 0.085082567 | 0.001437179 | 0.011871332 |
| TC > CG | 3.34E−05 | 3.94E−06 | 0.000116245 | 0.000402282 | 1.07E−17 | 0.000867211 |
| TC > CT | 0.001925815 | 0.003396359 | 0.019441016 | 8.33E−06 | 0.001055271 | 0.009446763 |
| TC > GA | 6.51E−05 | 0.000897247 | 0.077964487 | 0.036607629 | 2.47E−05 | 0.007384371 |
| TC > GG | 0.000416584 | 1.00E−05 | 0.000177374 | 1.05E−05 | 3.50E−05 | 0.001519128 |
| TC > GT | 0.005169294 | 8.69E−05 | 0.043491757 | 7.04E−18 | 3.88E−05 | 8.35E−18 |
| TG > AA | 0.001381174 | 0.002984883 | 6.66E−05 | 0.007632407 | 0.01799344 | 0.025516693 |
| TG > AC | 0.000238933 | 0.000221849 | 8.01E−05 | 0.000118037 | 0.001459468 | 0.010106612 |
| TG > AT | 0.002429656 | 0.004326063 | 0.000954882 | 7.00E−18 | 0.020392565 | 0.221144672 |
| TG > CA | 0.000281276 | 0.000145211 | 0.015848243 | 0.000804254 | 0.000475827 | 0.011407463 |
| TG > CC | 0.00010384 | 5.99E−05 | 0.000482425 | 0.000120038 | 0.000505816 | 0.007895165 |
| TG > CT | 0.001865089 | 0.003095807 | 0.006050241 | 8.21E−05 | 0.00032788 | 0.275180021 |
| TG > GA | 0.00041032 | 0.001482357 | 0.000521298 | 0.007852477 | 0.003268808 | 0.00744487 |
| TG > GC | 8.96E−05 | 0.000274287 | 0.000496379 | 0.000604191 | 0.000291894 | 0.005603666 |
| TG > GT | 0.001915497 | 0.0064538 | 0.010266472 | 3.23E−05 | 0.082669859 | 0.027417937 |
| TT > AA | 0.005547203 | 0.004355988 | 0.003216724 | 1.80E−05 | 0.004929248 | 0.00019356 |
| TT > AC | 0.000727502 | 0.000348681 | 0.002427528 | 6.62E−07 | 0.000967816 | 0.000128371 |
| TT > AG | 0.000422355 | 0.000217294 | 0.022876701 | 7.02E−18 | 0.000894679 | 0.00212615 |
| TT > CA | 0.000732554 | 4.21E−18 | 0.000693294 | 7.02E−18 | 1.05E−17 | 8.32E−18 |
| TT > CC | 0.005476474 | 3.01E−06 | 0.008151698 | 7.02E−18 | 0.000774453 | 8.32E−18 |
| TT > CG | 8.47E−05 | 1.54E−05 | 0.031767646 | 7.27E−18 | 1.05E−17 | 1.12E−05 |
| TT > GA | 0.00187938 | 4.84E−05 | 0.012287486 | 7.02E−18 | 3.00E−05 | 8.32E−18 |
| TT > GC | 0.001071045 | 1.56E−06 | 0.066432342 | 7.02E−18 | 5.67E−05 | 4.10E−07 |
| TT > GG | 0.001626775 | 4.21E−18 | 0.170826022 | 7.02E−18 | 1.05E−17 | 8.32E−18 |

| Type | DBS7 | DBS8 | DBS9 | DBS10 | DBS11 |
|---|---|---|---|---|---|
| AC > CA | 0.045932011 | 0.215131968 | 0.024655689 | 0.009589644 | 0.001377212 |
| AC > CG | 0.006254359 | 0.000973597 | 0.002074686 | 1.76E−05 | 0.000195021 |
| AC > CT | 0.013609485 | 0.31819519 | 0.028564518 | 0.013443426 | 0.00075998 |
| AC > GA | 0.009736786 | 0.001160712 | 0.025357273 | 0.00532758 | 0.000444327 |
| AC > GG | 0.002541771 | 0.000810497 | 0.007146141 | 0.000587527 | 0.002261844 |
| AC > GT | 0.004503138 | 0.000151093 | 0.012127392 | 0.028579727 | 0.005740058 |
| AC > TA | 0.00542378 | 0.012407611 | 0.041293268 | 0.00010456 | 0.00026539 |
| AC > TG | 0.000998696 | 0.002571577 | 0.017940522 | 7.63E−06 | 0.000240258 |
| AC > TT | 0.001180823 | 0.000268164 | 0.044500512 | 0.000707025 | 0.002834845 |
| AT > CA | 0.011050506 | 2.77E−05 | 0.012663162 | 0.003497094 | 0.009480799 |
| AT > CC | 0.006232087 | 0.00014235 | 0.001774837 | 0.001149187 | 0.00190016 |
| AT > CG | 0.001105051 | 0.000264792 | 9.19E−18 | 0.002565857 | 1.74E−05 |
| AT > GA | 0.014335792 | 0.000161264 | 0.006351523 | 7.98E−05 | 0.011200944 |
| AT > GC | 0.009248577 | 0.000224973 | 0.00180475 | 0.004041967 | 0.004180352 |
| AT > TA | 0.033848398 | 0.000313569 | 0.009173314 | 0.002902687 | 0.003840324 |
| CC > AA | 8.95E−18 | 0.037737162 | 8.95E−18 | 0.013504376 | 7.31E−18 |
| CC > AG | 8.22E−05 | 0.015352389 | 0.026191703 | 7.03E−05 | 0.000967218 |
| CC > AT | 0.000843957 | 0.028525729 | 0.019346144 | 0.010350069 | 0.034330257 |
| CC > GA | 0.000299149 | 0.000801296 | 0.01091321 | 0.003341594 | 0.000948311 |
| CC > GG | 0.002030647 | 5.55E−05 | 0.003035856 | 7.30E−18 | 0.003433026 |
| CC > GT | 0.000161461 | 0.000226819 | 0.017758768 | 0.001064579 | 0.069158053 |
| CC > TA | 8.95E−18 | 0.000709181 | 0.00068158 | 0.023558729 | 0.058510698 |
| CC > TG | 0.000233772 | 0.004169907 | 0.015377704 | 7.30E−18 | 0.032141081 |
| CC > TT | 8.95E−18 | 0.004338288 | 8.95E−18 | 0.02030585 | 0.330366527 |
| CG > AT | 6.30E−05 | 0.000326405 | 0.000222362 | 7.48E−18 | 0.001853473 |
| CG > GC | 0.000189574 | 1.15E−17 | 1.22E−05 | 1.92E−05 | 0.000169053 |
| CG > GT | 2.10E−05 | 0.001226554 | 0.000100926 | 1.03E−05 | 0.001201702 |
| CG > TA | 9.16E−18 | 0.000512922 | 9.16E−18 | 0.361155258 | 7.52E−18 |
| CG > TC | 4.94E−05 | 0.000917381 | 0.000135042 | 0.002673356 | 0.000697598 |
| CG > TT | 3.17E−05 | 0.000333501 | 8.42E−05 | 0.007838481 | 0.000711856 |
| CT > AA | 9.04E−18 | 0.020591366 | 9.03E−18 | 0.003909491 | 0.004418596 |
| CT > AC | 1.01E−17 | 0.013994132 | 9.03E−18 | 0.012932158 | 0.002359932 |
| CT > AG | 0.001259577 | 0.02039145 | 0.000582715 | 0.026361706 | 0.000498096 |
| CT > GA | 0.001869372 | 0.005567666 | 0.002843491 | 7.37E−18 | 0.011649026 |
| CT > GC | 0.015594765 | 0.000236901 | 0.003113823 | 7.37E−18 | 0.006135823 |
| CT > GG | 0.003998658 | 0.000305872 | 0.010412783 | 7.37E−18 | 0.00403699 |
| CT > TA | 9.04E−18 | 0.010895432 | 9.03E−18 | 0.004008969 | 0.041876241 |
| CT > TC | 0.063178791 | 0.01779254 | 3.00E−05 | 0.002735649 | 0.003002637 |
| CT > TG | 9.04E−18 | 0.000965595 | 9.47E−18 | 0.045461508 | 0.001486255 |
| GC > AA | 8.99E−18 | 0.012823358 | 8.98E−18 | 0.004036286 | 0.000998679 |
| GC > AG | 7.26E−05 | 0.008469381 | 0.123466542 | 7.33E−18 | 0.003285653 |
| GC > AT | 0.00034795 | 0.005477264 | 0.125457938 | 0.007666964 | 0.027164058 |
| GC > CA | 0.000666075 | 0.004801304 | 0.027282123 | 0.000103875 | 0.002107212 |
| GC > CG | 0.002634476 | 0.000240562 | 0.002489245 | 7.33E−18 | 0.004034662 |
| GC > TA | 6.20E−06 | 0.008509143 | 0.013939771 | 0.002779893 | 0.000242679 |
| TA > AT | 0.055312443 | 0.009614442 | 0.015388617 | 0.010258772 | 0.002220603 |
| TA > CG | 0.00302335 | 2.07E−05 | 0.001340299 | 0.004054188 | 0.000581539 |
| TA > CT | 0.028845737 | 0.004063836 | 0.014097959 | 0.001598001 | 0.003365757 |
| TA > GC | 0.001496806 | 0.000782041 | 0.000574839 | 7.31E−18 | 0.001015702 |
| TA > GG | 0.004272341 | 0.000215086 | 0.003226646 | 0.008473351 | 0.00037342 |
| TA > GT | 0.026070202 | 0.00015165 | 0.007724093 | 0.003560978 | 0.002658749 |
| TC > AA | 0.019693845 | 0.038982517 | 0.003713471 | 0.007559092 | 0.000219034 |
| TC > AG | 4.02E−06 | 0.011654567 | 0.046997048 | 0.000270967 | 0.047844298 |
| TC > AT | 0.002300965 | 0.018587025 | 0.052129488 | 0.003089629 | 0.085392987 |
| TC > CA | 0.002722975 | 0.017682791 | 0.049110405 | 0.025196974 | 0.000284643 |
| TC > CG | 0.001637804 | 0.010951274 | 0.00783955 | 0.000841899 | 0.00327037 |
| TC > CT | 0.009234002 | 0.008801207 | 0.051726943 | 0.026196854 | 0.020288405 |
| TC > GA | 9.08E−18 | 0.009655206 | 0.017410041 | 0.04379474 | 0.000237203 |
| TC > GG | 0.00070938 | 0.001918985 | 0.003753726 | 7.41E−18 | 0.007398707 |
| TC > GT | 9.08E−18 | 0.000277298 | 9.21E−18 | 0.045894488 | 0.015342475 |
| TG > AA | 0.022586537 | 0.008644125 | 0.015815305 | 0.001412222 | 0.007861047 |
| TG > AC | 0.002618439 | 0.003847385 | 0.007277042 | 0.000205866 | 0.000869434 |
| TG > AT | 0.001289232 | 0.008214417 | 0.000746723 | 0.002486306 | 0.010541634 |
| TG > CA | 0.023885763 | 0.005056563 | 0.022822085 | 0.029040058 | 0.056322442 |
| TG > CC | 0.001049375 | 0.001249151 | 0.01181143 | 0.000188959 | 0.003142411 |
| TG > CT | 9.03E−18 | 0.019486756 | 0.000968938 | 0.004306283 | 0.005602125 |
| TG > GA | 0.008245086 | 0.000270816 | 0.006185986 | 2.01E−05 | 0.007338985 |
| TG > GC | 0.003877689 | 0.003537596 | 0.003913787 | 7.37E−18 | 0.006144266 |
| TG > GT | 0.008704812 | 0.007854662 | 0.003343235 | 0.002158114 | 0.005260777 |
| TT > AA | 0.235387282 | 0.0035856 | 0.000342097 | 0.014452951 | 0.004799665 |
| TT > AC | 0.04006592 | 1.13E−17 | 0.006681418 | 7.39E−18 | 0.001046468 |
| TT > AG | 0.053988828 | 0.014622839 | 0.000920952 | 0.006937417 | 0.001277898 |
| TT > CA | 0.09786101 | 0.00629984 | 9.06E−18 | 0.001285814 | 0.002032562 |
| TT > CC | 0.03545834 | 0.00029446 | 0.005176594 | 0.002870655 | 0.004024876 |
| TT > CG | 0.0026744 | 0.000116181 | 1.99E−05 | 0.00855216 | 1.00E−17 |
| TT > GA | 0.043671853 | 0.003635678 | 9.20E−18 | 0.008661803 | 0.000711397 |
| TT > GC | 0.003676048 | 0.000286447 | 3.57E−05 | 0.025716286 | 5.82E−06 |
| TT > GG | 9.05E−18 | 0.000538842 | 9.05E−18 | 0.084425171 | 7.39E−18 |

TABLE 8
COSMIC DATA (COSMIC_v3.2_ID_GRCh37)

| Type | ID1 | ID2 | ID3 | ID4 | ID5 | ID6 | ID7 | ID8 |
|---|---|---|---|---|---|---|---|---|
| 1:Del:C:0 | 0.000159889 | 0.004824116 | 0.124727109 | 0.007249717 | 0.022202108 | 0.030506799 | 0.000466561 | 0.039827558 |
| 1:Del:C:1 | 0.000773523 | 2.21E−05 | 0.20887617 | 0.002734869 | 0.028547215 | 0.005491903 | 0 | 0.023915938 |
| 1:Del:C:2 | 3.31E−18 | 3.11E−06 | 0.176324218 | 0.002041063 | 0.026596927 | 0.008033994 | 0.008561945 | 0.003330554 |
| 1:Del:C:3 | 0.001907613 | 0.002472076 | 0.064042764 | 0.001112283 | 0.014159122 | 0.001102379 | 0.042360147 | 0.005403153 |
| 1:Del:C:4 | 0.00070599 | 0.003856976 | 0.043989808 | 0.001075684 | 0.002873422 | 0.00247817 | 0.114891908 | 0.001275709 |
| 1:Del:C:5 | 0.003370115 | 0.014615424 | 0.022417486 | 0.002246178 | 0.002602449 | 0.001322073 | 0.25176314 | 0.000933729 |
| 1:Del:T:0 | 0.001311605 | 0.007905966 | 0.03759923 | 0.006635052 | 0.101481148 | 0.004284162 | 0.00266749 | 0.034243687 |
| 1:Del:T:1 | 3.31E−18 | 3.95E−18 | 0.013309824 | 0.001622466 | 0.10609961 | 0.000677688 | 0.007732725 | 0.010452076 |
| 1:Del:T:2 | 0.000936453 | 0.007036418 | 0.016501851 | 0.002633405 | 0.114128463 | 0.011659665 | 0.00025476 | 0.00899203 |
| 1:Del:T:3 | 0.002295855 | 0.020234743 | 0.020141477 | 0.000306038 | 0.088962207 | 0.022242391 | 0.028972742 | 0.009825045 |
| 1:Del:T:4 | 0.004291422 | 0.047119925 | 0.015814364 | 0.001023202 | 0.052259532 | 0.017697141 | 0.158850552 | 0.00127378 |
| 1:Del:T:5 | 3.31E−18 | 0.814697227 | 0.008066314 | 0.000154442 | 0.030610523 | 0.007954345 | 1.92E−07 | 0.010620342 |
| 1:Ins:C:0 | 3.31E−18 | 3.95E−18 | 0.002083092 | 6.62E−18 | 0.000542252 | 0.002086887 | 0.000672367 | 0.002764271 |
| 1:Ins:C:1 | 3.31E−18 | 0.000227026 | 0.001578887 | 0.000272399 | 0.001242898 | 0.001773433 | 2.62E−05 | 0.002169119 |
| 1:Ins:C:2 | 3.31E−18 | 2.62E−05 | 0.001747946 | 6.88E−05 | 0.003151667 | 0.000246169 | 2.58E−05 | 0.001527 |
| 1:Ins:C:3 | 0.000845986 | 0.00043005 | 0.006177647 | 0.000512481 | 0.000602848 | 0.000232169 | 0.002407735 | 0.000116524 |
| 1:Ins:C:4 | 0.001785525 | 0.002911846 | 0.007104447 | 6.62E−18 | 0.000197582 | 0.000376592 | 0.015485431 | 0.000554177 |
| 1:Ins:C:5 | 0.024296536 | 0.009017536 | 0.007351101 | 0.000249143 | 0.000433544 | 0.000247997 | 0.060343229 | 0.000489489 |
| 1:Ins:T:0 | 0.002283824 | 0.001328556 | 0.018722365 | 0.000333665 | 0.006361965 | 0.006396251 | 1.36E−06 | 0.004507979 |
| 1:Ins:T:1 | 4.03E−05 | 3.95E−18 | 0.043119029 | 0.001010044 | 0.013462697 | 0.001041187 | 0.000373648 | 0.004868696 |
| 1:Ins:T:2 | 0.000192865 | 0.000647033 | 0.04015704 | 1.06E−05 | 0.029721144 | 0.000738848 | 0.018382706 | 0.001429636 |
| 1:Ins:T:3 | 0.004243267 | 0.001082879 | 0.025125434 | 0.000173935 | 0.01884025 | 0.000930638 | 0.008891635 | 0.001087286 |
| 1:Ins:T:4 | 0.016649127 | 0.003487461 | 0.006563305 | 1.60E−05 | 0.011268752 | 0.001349852 | 0.012787969 | 0.000822618 |
| 1:Ins:T:5 | 0.890906468 | 0.003132585 | 0.026181239 | 0.000832926 | 0.001439215 | 0.014390041 | 0.010789849 | 0.020161248 |
| 2:Del:R:0 | 0.000686046 | 0.000163809 | 0.006897553 | 0.071724214 | 0.028054407 | 0.001783647 | 2.33E−05 | 0.008769219 |
| 2:Del:R:1 | 0.001219969 | 0.003568885 | 0.008474654 | 0.230512879 | 0.024562729 | 0.002454347 | 0.008182302 | 0.010307145 |
| 2:Del:R:2 | 0.000472749 | 0.001849574 | 0.011800641 | 0.055059396 | 0.004562618 | 0.0020886 | 0.008991541 | 0.000532026 |
| 2:Del:R:3 | 0.00042672 | 0.000739044 | 0.006671125 | 0.012811006 | 0.000395919 | 0.000525746 | 0.072132138 | 2.71E−05 |
| 2:Del:R:4 | 5.46E−05 | 0.000881932 | 0.002822525 | 0.003219218 | 0.000867563 | 0.000232527 | 0.052250842 | 0.000274027 |
| 2:Del:R:5 | 3.31E−18 | 4.67E−18 | 0.000101239 | 0.002382721 | 0.02142678 | 0.000215685 | 0.057046331 | 0.000833079 |
| 3:Del:R:0 | 0.000128045 | 1.25E−05 | 0.000589124 | 0.005068954 | 0.01713637 | 0.001051047 | 0.000625412 | 0.010964101 |
| 3:Del:R:1 | 0.005011085 | 0.005003258 | 0.000372386 | 0.097114466 | 0.005909794 | 0.005413653 | 0.011689003 | 0.001348867 |
| 3:Del:R:2 | 0.000769383 | 0.002289891 | 9.63E−05 | 0.021484282 | 0.00084271 | 0.000536054 | 0.005474849 | 0.000376571 |
| 3:Del:R:3 | 0.000371256 | 0.000402587 | 2.45E−05 | 0.003837627 | 0.000621155 | 4.70E−05 | 0.003456748 | 0.000253509 |
| 3:Del:R:4 | 3.31E−18 | 3.97E−18 | 3.82E−06 | 0.000786295 | 0.005867075 | 7.31E−05 | 0.000492537 | 2.68E−06 |
| 3:Del:R:5 | 3.31E−18 | 3.95E−18 | 3.83E−07 | 0.001138609 | 0.008378911 | 0.000150616 | 0.000436589 | 9.10E−05 |
| 4:Del:R:0 | 9.97E−05 | 1.21E−05 | 0.000177645 | 0.007252429 | 0.010791265 | 0.002315098 | 0.000130877 | 0.006691309 |
| 4:Del:R:1 | 0.000900503 | 0.002115787 | 0.000634573 | 0.06352373 | 0.002843569 | 0.002806729 | 0.003396804 | 0.001622955 |
| 4:Del:R:2 | 3.23E−06 | 0.000543292 | 0.000116799 | 0.014548922 | 0.000164644 | 0.00047449 | 0.002777387 | 0.000272623 |
| 4:Del:R:3 | 3.31E−18 | 7.27E−06 | 4.12E−18 | 0.004554752 | 0.003877814 | 0.000291345 | 0.000439586 | 2.88E−05 |
| 4:Del:R:4 | 3.31E−18 | 3.95E−18 | 4.12E−18 | 0.000849679 | 0.004557254 | 0.000110576 | 0.00020181 | 9.73E−05 |
| 4:Del:R:5 | 2.89E−05 | 0.000210413 | 2.83E−06 | 0.00061078 | 0.001584436 | 5.68E−05 | 0.000250764 | 6.34E−06 |
| 5:Del:R:0 | 8.35E−06 | 3.85E−05 | 0.001776785 | 0.004132826 | 0.018447949 | 0.019333291 | 8.55E−05 | 0.237978862 |
| 5:Del:R:1 | 0.000190676 | 0.002977512 | 7.41E−05 | 0.015718533 | 0.000410335 | 0.007501825 | 0.003476729 | 0.000572257 |
| 5:Del:R:2 | 0.000126355 | 0.000397525 | 2.78E−06 | 0.002343845 | 0.000470925 | 0.000370524 | 0.0011689 | 0.000516838 |
| 5:Del:R:3 | 1.89E−05 | 0.000158151 | 4.12E−18 | 0.00118248 | 0.000257167 | 0.000168175 | 0.000406617 | 8.16E−06 |
| 5:Del:R:4 | 3.11E−06 | 8.47E−05 | 4.37E−05 | 0.000146923 | 9.73E−05 | 5.32E−05 | 0.000125882 | 0.000112898 |
| 5:Del:R:5 | 3.13E−05 | 2.12E−05 | 3.82E−06 | 0.000106174 | 0.000585018 | 1.15E−05 | 2.08E−05 | 4.43E−05 |
| 2:Ins:R:0 | 0.000402036 | 0.000331853 | 0.00263595 | 0.000492922 | 0.006349454 | 0.001998387 | 3.04E−05 | 0.00191671 |
| 2:Ins:R:1 | 7.88E−06 | 0.000271665 | 0.002242683 | 0.000553302 | 0.018387642 | 0.000208533 | 0.000397626 | 0.001661816 |
| 2:Ins:R:2 | 0.000221347 | 0.001035341 | 0.000955604 | 0.000266335 | 0.007207952 | 0.000790414 | 7.01E−05 | 0.000304586 |
| 2:Ins:R:3 | 0.000514268 | 0.000381326 | 0.000897238 | 0.000137794 | 0.001172292 | 0.000499074 | 0.003147039 | 0.000177583 |
| 2:Ins:R:4 | 0.0029131 | 0.001977423 | 0.000253085 | 7.19E−05 | 6.88E−05 | 0.000189469 | 0.00182828 | 1.66E−05 |
| 2:Ins:R:5 | 0.018121584 | 0.013633376 | 6.67E−06 | 0.000103689 | 0.000994122 | 0.000748642 | 0.00024477 | 0.001764384 |
| 3:Ins:R:0 | 3.06E−07 | 5.78E−05 | 9.60E−05 | 2.63E−05 | 0.003792478 | 0.001219185 | 0 | 0.000667863 |
| 3:Ins:R:1 | 0.000254695 | 0.00122705 | 0.000286144 | 0.000450983 | 0.006122761 | 0.000288615 | 5.51E−06 | 0.000671723 |
| 3:Ins:R:2 | 4.41E−05 | 0.000210075 | 9.75E−05 | 0.000600335 | 0.001143468 | 0.000746533 | 5.47E−05 | 0.000135768 |
| 3:Ins:R:3 | 0.000166702 | 3.02E−06 | 1.36E−05 | 7.42E−05 | 1.02E−05 | 0.000249737 | 0.00017783 | 3.79E−05 |
| 3:Ins:R:4 | 8.89E−05 | 0.000141807 | 1.56E−06 | 7.40E−05 | 1.18E−05 | 0.000155952 | 0.00041560 | 0.000122041 |
| 3:Ins:R:5 | 0.001248696 | 0.001864707 | 4.12E−18 | 3.96E−05 | 0.000102574 | 1.98E−05 | 0.00076927 | 3.66E−05 |
| 4:Ins:R:0 | 3.31E−18 | 1.68E−05 | 4.12E−18 | 0.000166645 | 0.002439186 | 0.001467917 | 0 | 0.000777758 |
| 4:Ins:R:1 | 0.000744381 | 0.001203728 | 0.000175982 | 0.001266435 | 0.003612298 | 0.000476283 | 2.61E−05 | 0.001103901 |
| 4:Ins:R:2 | 7.55E−05 | 1.22E−05 | 2.74E−05 | 9.84E−05 | 0.000376459 | 0.000528663 | 0.000194817 | 7.99E−05 |
| 4:Ins:R:3 | 0.000157544 | 0.000134169 | 4.77E−05 | 5.25E−05 | 1.87E−06 | 0.000334824 | 0.000273742 | 8.58E−06 |
| 4:Ins:R:4 | 0.000319653 | 0.000249385 | 4.18E−06 | 0.000146911 | 6.41E−06 | 5.60E−05 | 0.000258757 | 0.000205515 |
| 4:Ins:R:5 | 0.00100132 | 0.002193545 | 3.29E−05 | 0.000135205 | 0.000635977 | 0.000106861 | 8.74E−06 | 0.000885225 |
| 5:Ins:R:0 | 3.31E−18 | 3.95E−18 | 8.83E−05 | 8.58E−05 | 0.004750071 | 0.008716781 | 0 | 0.000318329 |
| 5:Ins:R:1 | 0.001443126 | 0.000135658 | 0.00065622 | 0.002135852 | 0.004793431 | 0.005405643 | 0 | 0.009761114 |
| 5:Ins:R:2 | 0.000254832 | 5.53E−06 | 5.51E−06 | 0.000222027 | 0.000298796 | 0.001321843 | 8.84E−06 | 0.000101153 |
| 5:Ins:R:3 | 0.000237087 | 9.77E−05 | 3.32E−05 | 7.59E−06 | 0.000165546 | 2.88E−05 | 4.46E−06 | 7.09E−05 |
| 5:Ins:R:4 | 0.000261865 | 2.15E−06 | 7.87E−06 | 2.53E−05 | 0.000126707 | 4.83E−05 | 1.06E−05 | 6.74E−06 |
| 5:Ins:R:5 | 0.000119834 | 0.000115629 | 3.48E−06 | 0.000160229 | 0.000626342 | 6.17E−05 | 4.28E−06 | 0.000190551 |
| 2:Del:M:1 | 0.001197815 | 0.000175553 | 0.006930764 | 0.188997489 | 0.028687946 | 0.000632866 | 0.003127058 | 0.004913672 |
| 3:Del:M:1 | 8.19E−05 | 5.46E−07 | 0.000344612 | 0.007034156 | 0.013157147 | 0.000990097 | 0.00025476 | 0.009290337 |
| 3:Del:M:2 | 0.001479593 | 0.002944063 | 0.00030285 | 0.049416601 | 0.00900421 | 0.00443363 | 0.004635639 | 0.004302693 |
| 4:Del:M:1 | 8.38E−05 | 0.000147005 | 7.35E−05 | 0.010981719 | 0.008732202 | 0.002745395 | 0.000183827 | 0.005809551 |
| 4:Del:M:2 | 0.000145479 | 0.000550614 | 0.000340216 | 0.029483877 | 0.006047187 | 0.00868271 | 0.001019041 | 0.001591587 |
| 4:Del:M:3 | 0.000160684 | 0.000693566 | 0.000130463 | 0.04886664 | 0.003442442 | 0.004773094 | 0.000717325 | 0.001098342 |
| 5:Del:M:1 | 18.16E−07 | 0.000624963 | 0.001369634 | 0.000453121 | 0.008617073 | 0.071066579 | 2.36E−05 | 0.28072426 |
| 5:Del:M:2 | 2.38E−10 | 0.000117708 | 0.001751272 | 0.000136232 | 0.007851021 | 0.253108348 | 0.000245769 | 0.123610163 |
| 5:Del:M:3 | 2.33E−12 | 5.67E−05 | 0.000774958 | 0.001112236 | 0.007157975 | 0.216806033 | 6.16E−05 | 0.046130408 |
| 5:Del:M:4 | 3.88E−05 | 0.000462842 | 0.000640555 | 0.003652946 | 0.002492549 | 0.120483943 | 6.82E−05 | 0.016367256 |
| 5:Del:M:5 | 0.000665116 | 0.002367319 | 0.00036297 | 4.56E−05 | 0.001112367 | 0.099282999 | 0.001738365 | 0.012814882 |

| Type | ID9 | ID10 | ID11 | ID12 | ID13 |
|---|---|---|---|---|---|
| 1:Del:C:0 | 0.33493988 | 0.025105414 | 0.001576565 | 0.052787741 | 0.065548371 |
| 1:Del:C:1 | 0.099994226 | 0.02930632 | 0.000550318 | 0.014674665 | 0.017536463 |
| 1:Del:C:2 | 0.002690655 | 0.018804056 | 0.000357338 | 0.000561932 | 0.005120219 |
| 1:Del:C:3 | 0.010625416 | 0.01650356 | 9.08E−18 | 0.000995577 | 0.005350516 |
| 1:Del:C:4 | 0.012496902 | 0.002930632 | 9.08E−18 | 0.00224005 | 0.004346005 |
| 1:Del:C:5 | 4.48E−05 | 8.42E−18 | 0.000156465 | 0.029410808 | 0.000843056 |
| 1:Del:T:0 | 0.105105373 | 0.028306105 | 0.000944206 | 0.045135118 | 0.053698762 |
| 1:Del:T:1 | 0.044293641 | 8.42E−18 | 0.000258505 | 1.42E−17 | 0.498224165 |
| 1:Del:T:2 | 0.085861956 | 0.001510326 | 0.000516519 | 0.010969204 | 0.023777805 |
| 1:Del:T:3 | 0.113250663 | 0.001420306 | 0.00042568 | 0.022109366 | 0.062780952 |
| 1:Del:T:4 | 0.094412737 | 0.00241052 | 0.001932451 | 0.044077964 | 0.023004382 |
| 1:Del:T:5 | 0.000929835 | 1.27E−17 | 0.006539395 | 0.000348512 | 0.006079575 |
| 1:Ins:C:0 | 5.41E−18 | 8.42E−18 | 0.000186238 | 1.42E−17 | 0.002850428 |
| 1:Ins:C:1 | 1.93E−06 | 0.000345074 | 0.163482884 | 0.000167697 | 0.001652256 |
| 1:Ins:C:2 | 0.000352623 | 0.000272059 | 0.093738843 | 0.001596586 | 0.00082125 |
| 1:Ins:C:3 | 5.41E−18 | 0.002580556 | 0.03617564 | 0.000450319 | 0.000375555 |
| 1:Ins:C:4 | 2.16E−06 | 0.000411089 | 0.011189789 | 0.00439393 | 0.000148578 |
| 1:Ins:C:5 | 7.37E−05 | 0.000851184 | 0.003412242 | 0.01705149 | 0.000706981 |
| 1:Ins:T:0 | 0.012614484 | 0.017003667 | 0.000406945 | 0.004265301 | 0.041137013 |
| 1:Ins:T:1 | 0.010385274 | 0.043809449 | 0.314277276 | 0.002496518 | 0.004748853 |
| 1:Ins:T:2 | 0.002314663 | 0.003650787 | 0.161807823 | 1.42E−17 | 0.003054171 |
| 1:Ins:T:3 | 0.001600885 | 0.003750809 | 0.107725327 | 0.000105674 | 0.009271857 |
| 1:Ins:T:4 | 0.001585355 | 9.66E−06 | 0.04903068 | 0.00033648 | 0.012098641 |
| 1:Ins:T:5 | 0.00577248 | 1.11E−08 | 0.023629043 | 0.00016655 | 0.011099794 |
| 2:Del:R:0 | 0.013174747 | 0.000839181 | 9.08E−18 | 0.006268105 | 0.022184399 |
| 2:Del:R:1 | 0.003198541 | 0.009472042 | 0.001363099 | 0.000278425 | 0.004333215 |
| 2:Del:R:2 | 0.001189697 | 0.006541411 | 0.000150729 | 0.001398167 | 0.003108924 |
| 2:Del:R:3 | 9.59E−05 | 0.00018504 | 9.08E−18 | 0.00067331 | 0.000105566 |
| 2:Del:R:4 | 5.41E−18 | 8.42E−18 | 9.13E−06 | 0.011980739 | 6.13E−06 |
| 2:Del:R:5 | 5.41E−18 | 8.42E−18 | 0.004527713 | 0.251519844 | 6.63E−05 |
| 3:Del:R:0 | 0.002858095 | 0.001200259 | 0.000189442 | 0.007599859 | 0.003039667 |
| 3:Del:R:1 | 0.00430191 | 0.042709211 | 0.000390863 | 0.000569429 | 0.001186963 |
| 3:Del:R:2 | 0.001275717 | 0.005061091 | 0.000170422 | 0.002680339 | 0.000384 |
| 3:Del:R:3 | 0.00030211 | 0.002730589 | 0.000127576 | 0.005920051 | 6.90E−05 |
| 3:Del:R:4 | 5.41E−18 | 8.42E−18 | 0.001202108 | 0.068622143 | 3.09E−06 |
| 3:Del:R:5 | 5.41E−18 | 8.42E−18 | 0.001742363 | 0.097480944 | 9.96E−05 |
| 4:Del:R:0 | 0.001432774 | 0.001470317 | 9.08E−18 | 0.001734516 | 0.002155349 |
| 4:Del:R:1 | 0.004594217 | 0.000521112 | 0.000355504 | 0.002302595 | 2.30E−05 |
| 4:Del:R:2 | 0.000896024 | 7.50E−05 | 9.08E−18 | 0.000611679 | 7.92E−05 |
| 4:Del:R:3 | 2.38E−05 | 0.000132028 | 0.000312137 | 0.039259058 | 3.92E−05 |
| 4:Del:R:4 | 5.41E−18 | 8.42E−18 | 0.000687366 | 0.053287873 | 9.47E−06 |
| 4:Del:R:5 | 1.37E−05 | 0.000209045 | 0.000157603 | 0.018644449 | 6.30E−05 |
| 5:Del:R:0 | 0.000772052 | 0.0013903 | 0.001281454 | 0.003382885 | 0.020097536 |
| 5:Del:R:1 | 0.000479551 | 0.026205652 | 0.000474634 | 0.002797035 | 0.002980534 |
| 5:Del:R:2 | 0.000264969 | 0.001770382 | 9.08E−18 | 0.005103842 | 6.99E−05 |
| 5:Del:R:3 | 6.12E−06 | 0.00013703 | 9.08E−18 | 0.002200952 | 6.40E−06 |
| 5:Del:R:4 | 7.25E−06 | 0.000125027 | 9.08E−18 | 0.001256186 | 1.46E−06 |
| 5:Del:R:5 | 2.99E−05 | 0.001120242 | 8.18E−08 | 0.00618126 | 2.35E−06 |
| 2:Ins:R:0 | 0.001162339 | 0.025305458 | 0.000190467 | 0.007440331 | 0.010618411 |
| 2:Ins:R:1 | 0.002100192 | 0.038908392 | 9.08E−18 | 0.001062301 | 0.001801368 |
| 2:Ins:R:2 | 0.000760941 | 0.026205652 | 0.000247885 | 0.000550238 | 0.000688524 |
| 2:Ins:R:3 | 0.000351635 | 0.00028006 | 0.000372356 | 0.000165525 | 0.000743544 |
| 2:Ins:R:4 | 3.07E−05 | 8.42E−18 | 0.000442384 | 0.001018206 | 0.000213354 |
| 2:Ins:R:5 | 5.41E−18 | 8.42E−18 | 0.000288801 | 0.038005197 | 0.000846575 |
| 3:Ins:R:0 | 0.000254573 | 0.018103905 | 9.08E−18 | 0.01515014 | 8.21E−05 |
| 3:Ins:R:1 | 0.000823355 | 0.055211908 | 0.000207083 | 0.000869573 | 0.003688328 |
| 3:Ins:R:2 | 0.000118261 | 0.009432034 | 0.000221526 | 0.000183891 | 0.000202511 |
| 3:Ins:R:3 | 5.18E−06 | 0.000461099 | 2.18E−05 | 8.70E−05 | 2.37E−05 |
| 3:Ins:R:4 | 1.61E−05 | 0.00018704 | 9.08E−18 | 0.000340788 | 3.49E−06 |
| 3:Ins:R:5 | 5.41E−18 | 0.00018504 | 9.01E−05 | 0.002840312 | 3.54E−05 |
| 4:Ins:R:0 | 0.000164387 | 0.016003452 | 0.000159621 | 0.005701178 | 0.001118797 |
| 4:Ins:R:1 | 0.000715565 | 0.074616092 | 0.000377636 | 0.001908886 | 0.002691635 |
| 4:Ins:R:2 | 4.24E−05 | 0.004090882 | 9.08E−18 | 0.00021849 | 6.93E−05 |
| 4:Ins:R:3 | 2.15E−05 | 0.000239052 | 9.08E−18 | 0.000176569 | 6.39E−07 |
| 4:Ins:R:4 | 5.41E−18 | 0.002280492 | 9.08E−18 | 0.000636053 | 1.21E−05 |
| 4:Ins:R:5 | 2.18E−06 | 0.013802977 | 9.08E−18 | 0.011670537 | 3.14E−07 |
| 5:Ins:R:0 | 0.000165846 | 0.049210613 | 0.00066606 | 0.013790645 | 0.002590231 |
| 5:Ins:R:1 | 0.001253996 | 0.278059972 | 0.00115191 | 0.003834265 | 0.013501952 |
| 5:Ins:R:2 | 0.000102849 | 0.009221989 | 0.000366007 | 0.002130016 | 4.59E−05 |
| 5:Ins:R:3 | 8.02E−05 | 0.003660789 | 8.34E−07 | 0.002660489 | 4.61E−05 |
| 5:Ins:R:4 | 5.05E−05 | 0.00334072 | 2.25E−06 | 0.001592407 | 2.43E−06 |
| 5:Ins:R:5 | 9.89E−06 | 0.002740591 | 1.84E−06 | 0.00613607 | 4.33E−05 |
| 2:Del:M:1 | 0.003900972 | 0.000638138 | 0.000382064 | 0.002653503 | 0.008872408 |
| 3:Del:M:1 | 0.003011328 | 0.003960854 | 0.000200443 | 0.010328352 | 0.001140083 |
| 3:Del:M:2 | 0.001543302 | 0.019304164 | 0.000301744 | 0.006061158 | 0.000450306 |
| 4:Del:M:1 | 0.001927721 | 0.000179039 | 9.08E−18 | 0.003771718 | 0.001270798 |
| 4:Del:M:2 | 0.003297448 | 0.000387083 | 9.08E−18 | 0.004573153 | 0.000159723 |
| 4:Del:M:3 | 0.002544079 | 8.22E−05 | 0.000789868 | 0.000525263 | 0.000270578 |
| 5:Del:M:1 | 3.54E−05 | 0.001330287 | 0.000282794 | 0.000563378 | 0.011740777 |
| 5:Del:M:2 | 0.000221236 | 0.000124027 | 0.000372771 | 1.42E−17 | 0.007055421 |
| 5:Del:M:3 | 0.000570907 | 5.65E−05 | 0.000750127 | 0.000323568 | 0.002165984 |
| 5:Del:M:4 | 0.000448308 | 0.000104022 | 0.000130278 | 0.000277663 | 0.000281736 |
| 5:Del:M:5 | 5.41E−18 | 0.041408931 | 0.000518955 | 0.006657973 | 0.013131907 |

| Type | ID14 | ID15 | ID16 | ID17 | ID18 |
|---|---|---|---|---|---|
| 1:Del:C:0 | 0.012016009 | 0.026527369 | 0.000952769 | 0.006117599 | 0.006012048 |
| 1:Del:C:1 | 0.001946232 | 0.121009787 | 2.64E−18 | 0.003667555 | 0.002004016 |
| 1:Del:C:2 | 0.000683696 | 0.147828864 | 8.15E−05 | 0.001220327 | 0.002004016 |
| 1:Del:C:3 | 0.000487468 | 0.136006058 | 0.001624914 | 2.26E−08 | 0.004008032 |
| 1:Del:C:4 | 0.002071921 | 0.084197696 | 0.003378643 | 1.94E−08 | 0 |
| 1:Del:C:5 | 0.002370276 | 0.018458293 | 2.65E−18 | 0.001220933 | 0.002004016 |
| 1:Del:T:0 | 0.018309803 | 0.023951637 | 0.00062865 | 0.006088347 | 0.08617269 |
| 1:Del:T:1 | 0.000852559 | 0.07996011 | 0.00015704 | 0.004903196 | 0.17234538 |
| 1:Del:T:2 | 0.010856423 | 0.120410466 | 2.66E−18 | 0.001225482 | 0.118236947 |
| 1:Del:T:3 | 0.01718395 | 0.113431215 | 2.66E−18 | 0.00122381 | 0.23847791 |
| 1:Del:T:4 | 0.013347903 | 0.079178832 | 0.001001651 | 1.34E−09 | 0.090180723 |
| 1:Del:T:5 | 0.038616999 | 0.046903215 | 2.79E−18 | 4.63E−08 | 0.070140562 |
| 1:Ins:C:0 | 0.454386958 | 6.58E−18 | 0.060271337 | 0.004878118 | 0 |
| 1:Ins:C:1 | 0.012777431 | 6.58E−18 | 0.136484095 | 0.014767199 | 0.002004016 |
| 1:Ins:C:2 | 0.00065885 | 6.58E−18 | 0.124683558 | 0.029260405 | 0 |
| 1:Ins:C:3 | 0.001564403 | 6.58E−18 | 0.098883121 | 0.049254522 | 0 |
| 1:Ins:C:4 | 0.001539502 | 6.58E−18 | 0.062094379 | 0.024538408 | 0 |
| 1:Ins:C:5 | 0.002292999 | 6.58E−18 | 0.031034603 | 0.009790503 | 0 |
| 1:Ins:T:0 | 0.032612007 | 6.58E−18 | 0.030194287 | 0.004892794 | 0 |
| 1:Ins:T:1 | 0.003165883 | 6.58E−18 | 0.103160227 | 0.038893641 | 0.002004016 |
| 1:Ins:T:2 | 0.001774606 | 6.58E−18 | 0.108738483 | 0.043924608 | 0 |
| 1:Ins:T:3 | 0.001173836 | 6.58E−18 | 0.100939445 | 0.004872091 | 0.002000008 |
| 1:Ins:T:4 | 0.004784999 | 6.58E−18 | 0.051962376 | 0.004903486 | 0.008016064 |
| 1:Ins:T:5 | 0.232747555 | 6.58E−18 | 0.035007686 | 0.00487383 | 0.004008032 |
| 2:Del:R:0 | 0.000661996 | 6.58E−18 | 0.001922942 | 0.014682054 | 0 |
| 2:Del:R:1 | 0.006600036 | 6.58E−18 | 0.002014646 | 0.008535951 | 0.036072289 |
| 2:Del:R:2 | 0.00270193 | 6.58E−18 | 0.001116794 | 1.46E−08 | 0.002004016 |
| 2:Del:R:3 | 0.000392799 | 6.58E−18 | 0.000472257 | 3.50E−08 | 0 |
| 2:Del:R:4 | 1.41E−06 | 6.58E−18 | 3.88E−05 | 4.34E−08 | 0.002004016 |
| 2:Del:R:5 | 4.37E−05 | 6.58E−18 | 2.64E−18 | 3.89E−08 | 0 |
| 3:Del:R:0 | 0.001146659 | 6.58E−18 | 0.001060141 | 0.008558573 | 0.004008032 |
| 3:Del:R:1 | 0.004840165 | 6.58E−18 | 0.002325923 | 0.002445446 | 0.006012048 |
| 3:Del:R:2 | 0.001053481 | 9.35E−05 | 0.001150375 | 3.41E−08 | 0 |
| 3:Del:R:3 | 0.000249605 | 6.58E−18 | 0.000120553 | 1.99E−08 | 0 |
| 3:Del:R:4 | 9.19E−18 | 6.58E−18 | 1.61E−05 | 2.13E−08 | 0 |
| 3:Del:R:5 | 9.19E−18 | 6.58E−18 | 9.58E−06 | 4.51E−08 | 0 |
| 4:Del:R:0 | 0.000193687 | 6.58E−18 | 0.000698912 | 0.006109585 | 0.004008032 |
| 4:Del:R:1 | 0.000617337 | 6.58E−18 | 0.000426296 | 1.55E−08 | 0.002004016 |
| 4:Del:R:2 | 0.000524295 | 6.58E−18 | 7.83E−05 | 3.83E−08 | 0 |
| 4:Del:R:3 | 5.03E−05 | 1.39E−06 | 2.64E−18 | 1.46E−08 | 0 |
| 4:Del:R:4 | 7.73E−06 | 6.58E−18 | 2.63E−06 | 3.19E−09 | 0 |
| 4:Del:R:5 | 9.19E−18 | 1.76E−06 | 2.64E−18 | 4.44E−09 | 0 |
| 5:Del:R:0 | 0.006086814 | 0.000284018 | 0.00556879 | 0.023396376 | 0.024048192 |
| 5:Del:R:1 | 0.000314462 | 6.58E−18 | 0.000110241 | 1.92E−08 | 0 |
| 5:Del:R:2 | 2.13E−05 | 6.58E−18 | 1.54E−05 | 4.86E−08 | 0 |
| 5:Del:R:3 | 2.85E−05 | 1.25E−06 | 6.73E−06 | 3.43E−08 | 0 |
| 5:Del:R:4 | 1.50E−05 | 6.58E−18 | 2.67E−07 | 9.21E−09 | 0 |
| 5:Del:R:5 | 9.08E−05 | 1.74E−06 | 2.63E−18 | 3.34E−08 | 0 |
| 2:Ins:R:0 | 0.023942256 | 6.58E−18 | 0.002191793 | 0.034379414 | 0 |
| 2:Ins:R:1 | 0.000123773 | 6.58E−18 | 0.001087848 | 0.058508649 | 0 |
| 2:Ins:R:2 | 0.001114199 | 6.58E−18 | 0.000729932 | 0.063694193 | 0 |
| 2:Ins:R:3 | 0.000871483 | 6.58E−18 | 9.48E−05 | 0.009762713 | 0.002004016 |
| 2:Ins:R:4 | 0.002973978 | 6.58E−18 | 0.000131664 | 3.84E−09 | 0 |
| 2:Ins:R:5 | 0.000544275 | 6.58E−18 | 1.18E−06 | 4.64E−08 | 0.002004016 |
| 3:Ins:R:0 | 0.006623497 | 6.58E−18 | 0.000570589 | 0.019583137 | 0.004008032 |
| 3:Ins:R:1 | 0.0025232 | 6.58E−18 | 0.001092184 | 0.112273274 | 0 |
| 3:Ins:R:2 | 0.000204501 | 6.58E−18 | 0.000281271 | 0.038874479 | 0 |
| 3:Ins:R:3 | 0.000327493 | 6.58E−18 | 3.18E−05 | 1.39E−08 | 0 |
| 3:Ins:R:4 | 0.00060186 | 6.58E−18 | 7.56E−05 | 2.84E−08 | 0 |
| 3:Ins:R:5 | 0.002205997 | 6.58E−18 | 2.29E−05 | 2.78E−08 | 0 |
| 4:Ins:R:0 | 0.00594693 | 6.58E−18 | 0.00032756 | 0.014692452 | 0 |
| 4:Ins:R:1 | 0.001326926 | 6.58E−18 | 0.001506978 | 0.210245111 | 0.002004016 |
| 4:Ins:R:2 | 8.45E−05 | 6.58E−18 | 4.50E−05 | 0.009749097 | 0 |
| 4:Ins:R:3 | 0.000713852 | 1.36E−06 | 3.02E−07 | 1.86E−08 | 0 |
| 4:Ins:R:4 | 0.00166501 | 1.45E−06 | 9.40E−06 | 9.68E−09 | 0 |
| 4:Ins:R:5 | 0.000274449 | 6.58E−18 | 2.66E−18 | 1.17E−08 | 0 |
| 5:Ins:R:0 | 0.002712141 | 6.58E−18 | 0.001172587 | 0.014673882 | 0.012024096 |
| 5:Ins:R:1 | 0.026455525 | 6.58E−18 | 0.001101192 | 0.004919532 | 0.002004016 |
| 5:Ins:R:2 | 0.000488313 | 1.18E−06 | 8.28E−07 | 4.64E−08 | 0 |
| 5:Ins:R:3 | 0.000103831 | 1.62E−06 | 2.66E−18 | 4.76E−08 | 0 |
| 5:Ins:R:4 | 0.000157593 | 6.58E−18 | 2.66E−18 | 3.96E−08 | 0 |
| 5:Ins:R:5 | 2.15E−05 | 6.58E−18 | 2.65E−18 | 4.48E−08 | 0 |
| 2:Del:M:1 | 0.002640925 | 6.58E−18 | 0.001374201 | 0.003668966 | 0.018036144 |
| 3:Del:M:1 | 0.001575173 | 6.58E−18 | 0.00076447 | 0.004887304 | 0 |
| 3:Del:M:2 | 0.003236935 | 6.58E−18 | 0.000980364 | 0.003681076 | 0 |
| 4:Del:M:1 | 8.20E−06 | 6.58E−18 | 0.000504718 | 0.010979731 | 0.006012048 |
| 4:Del:M:2 | 0.000950493 | 6.58E−18 | 0.0004098 | 0.004890442 | 0 |
| 4:Del:M:3 | 0.002230434 | 6.58E−18 | 0.000402368 | 0.003659617 | 0 |
| 5:Del:M:1 | 0.006551706 | 0.000660877 | 0.005362049 | 0.020771328 | 0.030060241 |
| 5:Del:M:2 | 0.004498634 | 0.000432409 | 0.005021715 | 0.012290058 | 0.022044176 |
| 5:Del:M:3 | 0.001404487 | 0.000266126 | 0.00333884 | 0.008596258 | 0.006012048 |
| 5:Del:M:4 | 0.000100143 | 0.000247928 | 0.001329897 | 0.004897913 | 0.004008032 |
| 5:Del:M:5 | 0.000931512 | 0.000139829 | 0.001601629 | 0.006075636 | 0 |

TABLE 9
COSMIC DATA (COSMIC_v3.2_SBS_GRCh37)

| Type | SBS1 | SBS2 | SBS3 | SBS4 | SBS5 | SBS6 | SBS7a | SBS7b | SBS7c | SBS7d |
|---|---|---|---|---|---|---|---|---|---|---|
| A[C > A]A | 0.000886157 | 5.80E−07 | 0.020808323 | 0.042196498 | 0.0119976 | 0.000425233 | 6.70E−05 | 0.002329386 | 0.004830406 | 4.04E−05 |
| A[C > A]C | 0.002280405 | 0.000148004 | 0.016506603 | 0.033297236 | 0.009438112 | 0.000524287 | 0.000179116 | 0.000460879 | 0.001150097 | 0.000764883 |
| A[C > A]G | 0.000177031 | 5.23E−05 | 0.0017507 | 0.015598705 | 0.00184963 | 5.20E−05 | 7.12E−05 | 0.000185951 | 0.000377032 | 0.000249962 |
| A[C > A]T | 0.001280227 | 9.78E−05 | 0.012204882 | 0.029497552 | 0.006608678 | 0.000180099 | 0.000248161 | 0.000709813 | 0.001960165 | 0.004049378 |
| A[C > G]A | 0.00186033 | 2.23E−16 | 0.019707883 | 0.006889428 | 0.01009798 | 0.000471258 | 6.49E−05 | 8.56E−06 | 0.001120094 | 0.001179819 |
| A[C > G]C | 0.001220217 | 0.000133004 | 0.011704682 | 0.002839764 | 0.00569886 | 0.000289158 | 2.16E−05 | 0.00018895 | 0.000319027 | 0.002549609 |
| A[C > G]G | 0.00011502 | 1.52E−05 | 0.000253101 | 0.001279894 | 0.001719656 | 2.22E−16 | 5.08E−05 | 0.000504867 | 2.22E−16 | 0.000905861 |
| A[C > G]T | 0.001140202 | 9.12E−05 | 0.017406963 | 0.003549705 | 0.01009798 | 0.000254139 | 9.57E−05 | 0.000284925 | 0.001930162 | 0.002909553 |
| A[C > T]A | 0.025004437 | 6.11E−05 | 0.014205682 | 0.008699278 | 0.032593481 | 0.064135143 | 0.000122079 | 0.001409629 | 0.001330112 | 0.003329489 |
| A[C > T]C | 0.006321122 | 0.00138004 | 0.012404962 | 0.004179653 | 0.017896421 | 0.022912555 | 0.017711494 | 0.038089962 | 0.007680645 | 0.001399785 |
| A[C > T]G | 0.365064773 | 3.27E−05 | 0.002571028 | 0.000782935 | 0.006178764 | 0.12006579 | 2.23E−16 | 0.000542857 | 0.000927078 | 0.001209814 |
| A[C > T]T | 0.0095817 | 0.001860054 | 0.012104842 | 0.004249647 | 0.021995601 | 0.021311678 | 0.007424818 | 0.010797155 | 0.007480629 | 0.032894951 |
| A[T > A]A | 0.000800142 | 9.59E−05 | 0.005492197 | 0.009419218 | 0.007378524 | 9.83E−05 | 0.00194126 | 0.002489344 | 0.012401042 | 8.89E−05 |
| A[T > A]C | 0.002230396 | 0.000878025 | 0.007212885 | 0.003529707 | 0.006928614 | 0.000546299 | 0.000670435 | 0.000466877 | 0.003420287 | 0.001079834 |
| A[T > A]G | 0.001140202 | 2.55E−05 | 0.009643858 | 0.012398971 | 0.009118176 | 2.22E−16 | 0.000587381 | 0.001559589 | 0.000211018 | 0.003699432 |
| A[T > A]T | 0.000183032 | 2.23E−16 | 0.006112445 | 0.004209651 | 0.00619876 | 0.00166091 | 0.003712409 | 0.008287816 | 0.050704261 | 0.041093692 |
| A[T > C]A | 0.001090193 | 6.17E−05 | 0.016506603 | 0.00794934 | 0.046190762 | 0.002211212 | 0.000490318 | 0.001019731 | 0.000288024 | 0.004359331 |
| A[T > C]C | 0.003040539 | 2.15E−05 | 0.007763105 | 0.000714941 | 0.013397321 | 0.00113062 | 0.000227147 | 0.00045588 | 0.000533045 | 0.003929397 |
| A[T > C]G | 0.000106019 | 1.32E−05 | 0.012304922 | 0.004999585 | 0.038192362 | 0.004242325 | 0.000187121 | 0.001499605 | 0.00107009 | 0.00918859 |
| A[T > C]T | 0.005741019 | 0.000155004 | 0.017306923 | 0.001559871 | 0.038292342 | 0.000502275 | 0.000615399 | 0.002149434 | 0.002650223 | 0.050992173 |
| A[T > G]A | 0.000172031 | 0.000238007 | 0.003951581 | 0.001049913 | 0.003859228 | 2.57E−05 | 0.000473307 | 8.44E−05 | 0.011901 | 0.003699432 |
| A[T > G]C | 0.000207037 | 7.46E−05 | 0.00260104 | 0.000158987 | 0.00259948 | 0.000204112 | 0.00024716 | 0.000931754 | 0.001050088 | 0.002659592 |
| A[T > G]G | 0.000268048 | 2.05E−06 | 0.006302521 | 0.001419882 | 0.007938412 | 0.000107059 | 0.000276179 | 0.001469613 | 0.000604051 | 0.010598373 |
| A[T > G]T | 0.00011202 | 3.76E−06 | 0.003971589 | 0.000215982 | 0.004829034 | 0.000386212 | 0.000309201 | 0.000732807 | 2.22E−16 | 0.014297805 |
| C[C > A]A | 0.000312055 | 0.000208006 | 0.022509004 | 0.080693302 | 0.007428514 | 0.001820998 | 0.000455295 | 0.0011397 | 0.000109009 | 0.014497775 |
| C[C > A]C | 0.001790318 | 9.53E−05 | 0.025310124 | 0.079693385 | 0.006138772 | 0.004092242 | 0.000240156 | 0.001549592 | 0.00309026 | 0.003219506 |
| C[C > A]G | 9.32E−05 | 2.23E−16 | 0.002511004 | 0.024497967 | 0.003459308 | 0.001650905 | 2.23E−16 | 0.000407893 | 0.001910161 | 8.51E−05 |
| C[C > A]T | 2.23E−16 | 0.000421012 | 0.015406162 | 0.068994273 | 0.006488702 | 0.009605263 | 0.000372242 | 0.001489607 | 0.013501135 | 0.000116982 |
| C[C > G]A | 2.41E−05 | 2.23E−16 | 0.019307723 | 0.00782935 | 0.006958608 | 4.11E−05 | 2.23E−16 | 0.000457879 | 0.000206017 | 0.001319797 |
| C[C > G]C | 7.68E−05 | 0.00035101 | 0.014105642 | 0.00686943 | 0.009068186 | 3.72E−05 | 0.000131085 | 0.000959747 | 0.00226019 | 0.000285956 |
| C[C > G]G | 0.000352062 | 4.87E−05 | 0.0015006 | 0.003399718 | 0.002489502 | 8.43E−05 | 7.76E−05 | 0.000790792 | 0.000262022 | 0.001319797 |
| C[C > G]T | 2.23E−16 | 6.36E−05 | 0.022308924 | 0.006819434 | 0.009528094 | 1.44E−05 | 9.57E−05 | 0.000889766 | 0.004240356 | 2.22E−16 |
| C[C > T]A | 0.002000355 | 0.004320125 | 0.016106443 | 0.012698946 | 0.019396121 | 0.013607456 | 0.050032468 | 0.106971809 | 0.002710228 | 0.035594536 |
| C[C > T]C | 0.000270048 | 0.000170005 | 0.020208083 | 0.011499046 | 0.018996201 | 0.014808114 | 0.055335909 | 0.181952048 | 0.005970502 | 0.002599601 |
| C[C > T]G | 0.196034782 | 0.00277008 | 0.002240896 | 0.003309725 | 0.017496501 | 0.122066887 | 0.006714357 | 0.022094177 | 0.008940751 | 0.002639595 |
| C[C > T]T | 0.000196035 | 0.001450042 | 0.023009204 | 0.015998672 | 0.023095381 | 0.013807566 | 0.056636753 | 0.125966803 | 0.020601731 | 0.000137979 |
| C[T > A]A | 4.30E−05 | 0.000243007 | 0.007943177 | 0.014398805 | 0.003539292 | 6.66E−05 | 0.000177115 | 0.001119705 | 0.003470292 | 0.001609753 |
| C[T > A]C | 0.00039307 | 0.000244007 | 0.017807123 | 0.012198987 | 0.005188962 | 0.000551302 | 0.000261169 | 0.000957748 | 0.013401126 | 8.13E−05 |
| C[T > A]G | 0.000324057 | 0.000149004 | 0.013205282 | 0.032497303 | 0.005008998 | 0.000115063 | 0.000112073 | 0.000882767 | 0.000961081 | 0.001199816 |
| C[T > A]T | 0.000260046 | 2.23E−16 | 0.012705082 | 0.011799021 | 0.00429914 | 0.000179098 | 0.000371241 | 0.00011197 | 0.048704093 | 0.001149824 |
| C[T > C]A | 2.23E−16 | 0.000302009 | 0.008833533 | 0.00517957 | 0.013297341 | 0.00166091 | 0.000451293 | 0.001529597 | 0.007570636 | 0.010198435 |
| C[T > C]C | 0.002500444 | 2.85E−05 | 0.014505802 | 0.002359804 | 0.01039792 | 0.001030565 | 0.000259168 | 0.000839779 | 0.003580301 | 8.89E−05 |
| C[T > C]G | 0.000360064 | 2.23E−16 | 0.010104042 | 0.005499544 | 0.020795841 | 0.005322917 | 0.000104068 | 0.000729808 | 0.002170182 | 0.000934856 |
| C[T > C]T | 4.26E−05 | 0.000186005 | 0.015806323 | 0.002559788 | 0.014897021 | 0.001270696 | 0.001821182 | 0.00572849 | 0.043103622 | 0.039693907 |
| C[T > G]A | 3.55E−05 | 0.000153004 | 0.004371749 | 0.000869928 | 0.002589482 | 4.95E−05 | 8.43E−05 | 0.000358905 | 0.003280276 | 0.001479773 |
| C[T > G]C | 0.000212038 | 0.000155004 | 0.007042817 | 0.000440963 | 0.004609078 | 0.00078443 | 0.000172112 | 0.000791791 | 0.005580469 | 4.98E−05 |
| C[T > G]G | 0.000128023 | 0.000121003 | 0.010704282 | 0.003789685 | 0.006128774 | 0.000924507 | 2.23E−16 | 0.00128966 | 0.0011901 | 2.58E−05 |
| C[T > G]T | 0.00017103 | 0.000203006 | 0.007032813 | 0.0011999 | 0.00729854 | 0.001300713 | 2.65E−05 | 3.68E−06 | 0.003400286 | 0.000155976 |
| G[C > A]A | 0.00158028 | 8.62E−05 | 0.007132853 | 0.031597377 | 0.01019796 | 0.000427234 | 4.06E−05 | 0.000745803 | 0.001050088 | 0.000167974 |
| G[C > A]C | 0.00033906 | 2.23E−16 | 0.010904362 | 0.034797112 | 0.00764847 | 0.000913501 | 0.00013809 | 0.000214943 | 0.000742062 | 2.22E−16 |
| G[C > A]G | 0.000587104 | 1.39E−05 | 0.001430572 | 0.01529873 | 0.002339532 | 0.000357196 | 4.25E−05 | 7.29E−05 | 2.22E−16 | 1.82E−05 |
| G[C > A]T | 2.23E−16 | 5.12E−05 | 0.010004002 | 0.021198241 | 0.006818636 | 0.001230674 | 2.23E−16 | 0.000146961 | 0.000363031 | 7.40E−05 |
| G[C > G]A | 9.59E−06 | 6.54E−05 | 0.012705082 | 0.005289561 | 0.004679064 | 1.94E−05 | 1.96E−05 | 5.11E−05 | 0.000182015 | 0.00019597 |
| G[C > G]C | 0.000164029 | 0.000261008 | 0.008633453 | 0.005749523 | 0.005078984 | 0.000685376 | 3.32E−05 | 0.000451881 | 0.00047304 | 2.22E−16 |
| G[C > G]G | 0.000166029 | 2.23E−16 | 0.002420968 | 0.003299726 | 0.001509698 | 2.22E−16 | 4.51E−05 | 0.000336911 | 0.000180015 | 0.000544916 |
| G[C > G]T | 2.23E−16 | 0.000134004 | 0.014705882 | 0.003039748 | 0.006718656 | 0.000321176 | 4.20E−05 | 0.000276927 | 0.001280108 | 0.000881865 |
| G[C > T]A | 0.004440788 | 2.23E−16 | 0.016406563 | 0.004089661 | 0.019396121 | 0.093651316 | 8.50E−05 | 0.000854775 | 0.000157013 | 0.000364944 |
| G[C > T]C | 9.28E−05 | 1.29E−07 | 0.013405362 | 0.00445963 | 0.021595681 | 0.111060856 | 0.011707597 | 0.026293071 | 9.51E−06 | 0.000180972 |
| G[C > T]G | 0.218038687 | 2.23E−16 | 0.000528211 | 0.00048196 | 0.01229754 | 0.178097589 | 5.52E−06 | 0.000175954 | 3.43E−05 | 0.000156976 |
| G[C > T]T | 3.84E−05 | 5.04E−05 | 0.011304522 | 0.003809684 | 0.018596281 | 0.07444079 | 0.016110455 | 0.00538858 | 0.000758064 | 0.042093539 |
| G[T > A]A | 8.12E−05 | 2.23E−16 | 0.007062825 | 0.009219235 | 0.003669266 | 5.10E−05 | 0.00040126 | 0.000525861 | 0.001730145 | 2.37E−05 |
| G[T > A]C | 0.000129023 | 0.000131004 | 0.007482993 | 0.003599701 | 0.002909418 | 0.000489268 | 0.000224145 | 0.000255933 | 0.002270191 | 0.000111983 |
| G[T > A]G | 0.000246044 | 6.38E−05 | 0.011604642 | 0.013098913 | 0.004109178 | 0.000195107 | 0.000290188 | 0.000655827 | 2.22E−16 | 0.001349793 |
| G[T > A]T | 0.000258046 | 6.28E−05 | 0.012304922 | 0.005519542 | 0.003219356 | 0.000248136 | 0.000306199 | 5.59E−05 | 0.020301706 | 0.008758656 |
| G[T > C]A | 0.001050186 | 2.23E−16 | 0.009983994 | 0.002899759 | 0.014097181 | 0.002571409 | 0.000101066 | 0.00026593 | 2.22E−16 | 0.000799877 |
| G[T > C]C | 0.001900337 | 2.74E−05 | 0.005642257 | 0.001179902 | 0.007938412 | 0.001110609 | 7.41E−05 | 2.22E−16 | 0.000423036 | 0.002319644 |
| G[T > C]G | 0.001170208 | 0.000107003 | 0.010904362 | 0.002369803 | 0.014997001 | 0.002451343 | 7.25E−05 | 0.000528861 | 0.000232019 | 0.002569606 |
| G[T > C]T | 7.13E−05 | 1.25E−05 | 0.010204082 | 0.000754937 | 0.013697261 | 0.000906497 | 0.000811527 | 0.006848195 | 0.007070594 | 0.317951194 |
| G[T > G]A | 2.23E−16 | 0.000209006 | 0.004421769 | 0.00059795 | 0.002519496 | 4.35E−05 | 9.07E−05 | 0.000322915 | 0.002480208 | 7.48E−05 |
| G[T > G]C | 2.23E−16 | 0.000133004 | 0.002340936 | 0.000252979 | 0.001709658 | 0.000213117 | 0.000121079 | 0.000320915 | 0.00166014 | 2.23E−05 |
| G[T > G]G | 0.000348062 | 3.71E−05 | 0.010904362 | 0.002429798 | 0.00529894 | 0.00027415 | 0.000325211 | 0.002409365 | 0.006030507 | 0.003799417 |
| G[T > G]T | 1.46E−05 | 2.23E−16 | 0.005832333 | 0.000251979 | 0.00234953 | 0.000787431 | 0.000835542 | 0.001829518 | 0.006750567 | 0.00683895 |
| T[C > A]A | 6.58E−05 | 0.000639018 | 0.008413365 | 0.039796697 | 0.007858428 | 0.000356195 | 0.000390253 | 0.002899236 | 0.002520212 | 0.01139825 |
| T[C > A]C | 0.002530449 | 0.000170005 | 0.016806723 | 0.046896108 | 0.009168166 | 0.000498273 | 0.000636413 | 0.003519073 | 0.014201193 | 0.007758809 |
| T[C > A]G | 2.23E−16 | 8.53E−05 | 0.001410564 | 0.010899095 | 0.00229954 | 0.00020011 | 3.44E−05 | 0.000299921 | 2.22E−16 | 2.22E−16 |
| T[C > A]T | 5.88E−06 | 0.000440013 | 0.00997399 | 0.041296572 | 0.012697461 | 0.00074741 | 0.000613398 | 0.001409629 | 0.003730313 | 0.000190971 |
| T[C > G]A | 2.23E−16 | 2.23E−16 | 0.013305322 | 0.003579703 | 0.007808438 | 4.97E−05 | 2.23E−16 | 0.000253933 | 0.001940163 | 0.003739426 |
| T[C > G]C | 0.000203036 | 9.87E−05 | 0.020308123 | 0.005949506 | 0.00759848 | 0.000113062 | 5.09E−05 | 0.001619573 | 0.004610387 | 0.00058791 |
| T[C > G]G | 6.94E−05 | 4.12E−05 | 0.000520208 | 0.001829848 | 0.001709658 | 1.95E−06 | 6.35E−06 | 0.000363904 | 8.75E−06 | 0.000872866 |
| T[C > G]T | 0.001560277 | 0.001180034 | 0.023509404 | 0.003379719 | 0.0124975 | 0.00012807 | 0.000281182 | 0.000713812 | 0.012301034 | 8.53E−05 |
| T[C > T]A | 0.001110197 | 0.536015481 | 0.005762305 | 0.00373969 | 0.020295941 | 0.012006579 | 0.238154546 | 0.006608258 | 0.017301454 | 0.000206968 |
| T[C > T]C | 3.73E−05 | 0.09730281 | 0.013305322 | 0.00698942 | 0.022995401 | 0.010505757 | 0.331214935 | 0.210944408 | 0.013701151 | 0.005649133 |
| T[C > T]G | 0.110019521 | 0.044201277 | 0.000989396 | 0.000280977 | 0.015896821 | 0.052028509 | 0.074148117 | 0.057384877 | 4.64E−05 | 0.001749731 |
| T[C > T]T | 2.23E−16 | 0.300008665 | 0.008173269 | 0.007219401 | 0.020995801 | 0.006993832 | 0.107069481 | 0.101973126 | 0.002320195 | 0.049992326 |
| T[T > A]A | 0.006721193 | 0.000422012 | 0.006512605 | 0.008879263 | 0.006308738 | 0.000454249 | 0.000356231 | 0.003239146 | 0.015401294 | 0.016697437 |
| T[T > A]C | 2.23E−16 | 0.000192006 | 0.010504202 | 0.004309642 | 0.004139172 | 0.000114063 | 0.000389253 | 0.00223941 | 0.034202874 | 0.001229811 |
| T[T > A]G | 2.80E−05 | 5.01E−05 | 0.006712685 | 0.009539208 | 0.004789042 | 2.95E−05 | 2.23E−16 | 0.001699552 | 0.012201025 | 0.005599141 |
| T[T > A]T | 0.002250399 | 0.000205006 | 0.013005202 | 0.005619534 | 0.00874825 | 0.000426234 | 0.000798518 | 2.22E−16 | 0.279023449 | 0.02729581 |
| T[T > C]A | 0.000255045 | 2.23E−16 | 0.015206082 | 0.0024098 | 0.017996401 | 0.001480811 | 0.002151396 | 0.005998419 | 0.030202538 | 0.081187538 |
| T[T > C]C | 0.003390602 | 4.35E−05 | 0.008993597 | 0.000502958 | 0.009708058 | 0.001090598 | 0.000596387 | 0.001729544 | 0.020901757 | 0.014297805 |
| T[T > C]G | 0.000416074 | 0.000117003 | 0.006932773 | 0.001739856 | 0.012697461 | 0.002131168 | 0.000319207 | 0.003808996 | 0.012901084 | 0.022796501 |
| T[T > C]T | 0.004330768 | 3.58E−05 | 0.013905562 | 0.000635947 | 0.018496301 | 0.001740954 | 0.000812527 | 0.002969217 | 0.088107405 | 0.043093385 |
| T[T > G]A | 2.23E−16 | 1.67E−05 | 0.007252901 | 0.000376969 | 0.005218956 | 0.000105058 | 0.000128083 | 0.000954748 | 0.019301622 | 0.000210968 |
| T[T > G]C | 5.51E−05 | 7.04E−05 | 0.006282513 | 0.000173986 | 0.006558688 | 0.000287157 | 0.000116075 | 0.001549592 | 0.017401462 | 0.000114982 |
| T[T > G]G | 0.000583103 | 9.54E−05 | 0.008053221 | 0.002319807 | 0.006938612 | 0.000324178 | 2.23E−16 | 0.001349644 | 0.007640642 | 0.000124981 |
| T[T > G]T | 2.23E−16 | 2.23E−16 | 0.010504202 | 0.000567953 | 0.013497301 | 0.001010554 | 8.30E−05 | 0.001769534 | 0.021701824 | 0.000165975 |
| Type | SBS8 | SBS9 | SBS10a | SBS10b | SBS10c | SBS10d | SBS11 | SBS12 | SBS13 | SBS14 |
| A[C > A]A | 0.044098218 | 0.000557954 | 0.00219017 | 0.000181997 | 0.004331453 | 0.01011397 | 0.000146208 | 0.004520592 | 0.00181992 | 0.001120492 |
| A[C > A]C | 0.047798069 | 0.004089661 | 0.001770137 | 0.006539908 | 0.014830219 | 0.01844606 | 0.000552786 | 0.001130148 | 0.000720968 | 0.01310575 |
| A[C > A]G | 0.004619813 | 0.000425965 | 0.000150012 | 5.35E−05 | 0.000657116 | 0.000726535 | 9.42E−05 | 0.000540071 | 0.000263988 | 0.000413181 |
| A[C > A]T | 0.046998101 | 0.003049747 | 0.01700132 | 1.63E−05 | 0.013127762 | 0.014196653 | 0.000266379 | 0.00122016 | 0.000347985 | 0.082636256 |
| A[C > G]A | 0.004329825 | 0.004839598 | 2.23E−16 | 0.000304996 | 0.000347634 | 0.000128897 | 0.000170242 | 0.00122016 | 0.00386983 | 2.22E−16 |
| A[C > G]C | 0.002949881 | 0.001959837 | 2.43E−05 | 0.000133998 | 0.000325131 | 0.000292096 | 0.000290413 | 0.000642084 | 0.000950958 | 5.78E−06 |
| A[C > G]G | 0.000285988 | 0.001359887 | 2.23E−16 | 2.22E−16 | 4.24E−05 | 2.69E−05 | 5.97E−05 | 7.51E−05 | 0.000263988 | 3.91E−05 |
| A[C > G]T | 0.005709769 | 0.006409468 | 2.23E−16 | 2.22E−16 | 0.000516306 | 0.000106581 | 0.000535762 | 0.001440189 | 0.003999824 | 0.00020409 |
| A[C > T]A | 0.006809725 | 0.008099328 | 0.001040081 | 0.000548992 | 0.004867067 | 0.001367902 | 0.025836745 | 0.005510722 | 0.007179684 | 0.012405443 |
| A[C > T]C | 0.002819886 | 0.00541955 | 0.000834065 | 0.005249927 | 0.005249436 | 0.001963422 | 0.147209359 | 0.002120278 | 2.22E−16 | 0.010104433 |
| A[C > T]G | 0.00322987 | 0.004379636 | 0.005570433 | 0.000413994 | 0.013019985 | 0.00070293 | 0.007590796 | 0.000118015 | 0.000302987 | 0.026511632 |
| A[C > T]T | 0.012499495 | 0.006489461 | 0.002990232 | 0.007929889 | 0.006156213 | 0.002507721 | 0.109155239 | 0.002380312 | 0.000317986 | 0.016107067 |
| A[T > A]A | 0.019499212 | 0.012698946 | 5.07E−05 | 0.001819975 | 0.004020058 | 0.001903975 | 0.000417594 | 0.005170677 | 2.22E−16 | 0.000613269 |
| A[T > A]C | 0.02129914 | 0.002819766 | 0.000194015 | 0.000336995 | 0.013642365 | 0.003186628 | 0.000441628 | 0.002710355 | 6.52E−05 | 0.000372163 |
| A[T > A]G | 0.022799079 | 0.002759771 | 2.23E−16 | 2.34E−05 | 0.001230091 | 0.00066045 | 3.94E−05 | 0.003120409 | 0.000589974 | 0.000354155 |
| A[T > A]T | 0.02499899 | 0.018898431 | 0.006660517 | 0.00284996 | 0.071776727 | 0.022163794 | 3.40E−06 | 0.002820369 | 0.000605973 | 0.001850812 |
| A[T > C]A | 0.009769605 | 0.020298315 | 0.000745058 | 0.009349869 | 0.018222538 | 0.005214793 | 0.001141624 | 0.073609643 | 0.001909916 | 0.012605531 |
| A[T > C]C | 0.005009798 | 0.010499129 | 0.003620281 | 0.000509993 | 0.009079555 | 0.003227955 | 0.000939336 | 0.04030528 | 0.000141994 | 0.006302765 |
| A[T > C]G | 0.006979718 | 0.014298813 | 0.002140166 | 0.000530993 | 0.016943034 | 0.008555875 | 0.000320456 | 0.070509237 | 0.000427981 | 0.012405443 |
| A[T > C]T | 0.014899398 | 0.028997593 | 0.003360261 | 0.000149998 | 0.012778988 | 0.00568142 | 0.001191695 | 0.063808359 | 0.000958958 | 0.00597262 |
| A[T > G]A | 0.001639934 | 0.045096257 | 0.001920149 | 3.47E−05 | 0.002519236 | 0.006932235 | 0.00021831 | 0.002010263 | 6.14E−05 | 0.000212093 |
| A[T > G]C | 0.000667973 | 0.006929425 | 0.001240096 | 5.33E−05 | 0.000978868 | 0.00061364 | 4.68E−05 | 0.000664087 | 2.22E−16 | 0.002971304 |
| A[T > G]G | 0.004769807 | 0.005329558 | 0.000320025 | 0.000522993 | 0.001038768 | 0.000939707 | 0.000179255 | 0.000808106 | 8.25E−05 | 0.000709311 |
| A[T > G]T | 0.00073997 | 0.023898016 | 0.009220716 | 2.22E−16 | 0.013055858 | 0.004855706 | 0.000285406 | 0.001280168 | 0.000319986 | 0.011104872 |
| C[C > A]A | 0.04009838 | 0.004799602 | 0.003180247 | 0.000520993 | 0.017432934 | 0.010003683 | 0.00061888 | 0.00542071 | 0.001399938 | 0.016307155 |
| C[C > A]C | 0.038798433 | 0.001919841 | 0.000202016 | 0.003179955 | 0.009670422 | 0.014848678 | 0.000987404 | 0.004280561 | 0.000967957 | 0.019708647 |
| C[C > A]G | 0.003409862 | 0.001369886 | 4.64E−05 | 8.38E−05 | 0.002404606 | 0.001019607 | 9.21E−06 | 0.001170153 | 4.33E−06 | 0.001380606 |
| C[C > A]T | 0.032998667 | 0.003059746 | 0.017301343 | 0.008329883 | 0.044887394 | 0.018549972 | 0.000150214 | 0.001100144 | 2.22E−16 | 0.260114122 |
| C[C > G]A | 0.004999798 | 0.000892926 | 4.57E−05 | 2.99E−05 | 0.000110202 | 8.17E−05 | 0.000261372 | 0.001340176 | 0.005609753 | 8.55E−05 |
| C[C > G]C | 0.005459779 | 0.001539872 | 2.23E−16 | 2.22E−16 | 0.000207583 | 0.000124853 | 0.000177252 | 0.000891117 | 0.000647971 | 7.86E−05 |
| C[C > G]G | 1.05E−05 | 0.000436964 | 2.01E−05 | 2.22E−16 | 0.000180223 | 2.90E−05 | 1.12E−05 | 0.000173023 | 0.000487979 | 1.91E−05 |
| C[C > G]T | 0.005489778 | 0.003029749 | 3.01E−05 | 2.22E−16 | 0.000238268 | 0.000230805 | 9.65E−06 | 0.001560204 | 0.006399718 | 0.000178078 |
| C[C > T]A | 0.002669892 | 0.007239399 | 3.68E−05 | 0.000150998 | 0.003761173 | 0.000907688 | 0.004075797 | 0.006590863 | 1.93E−05 | 0.002731198 |
| C[C > T]C | 0.004019838 | 0.008759273 | 2.23E−16 | 8.44E−05 | 0.006643822 | 0.0022028 | 0.126179451 | 0.000254033 | 0.001349941 | 0.001380606 |
| C[C > T]G | 0.00148994 | 0.00650946 | 2.23E−16 | 0.003699948 | 0.009471593 | 0.000591946 | 0.00092131 | 4.09E−05 | 0.001989912 | 0.011605092 |
| C[C > T]T | 0.014499414 | 0.011099079 | 0.000103008 | 0.000594992 | 0.00983726 | 0.002541076 | 0.065192716 | 0.003380443 | 0.001449936 | 0.001360597 |
| C[T > A]A | 0.018999232 | 0.004789602 | 2.23E−16 | 7.09E−05 | 0.000673176 | 0.001216124 | 0.000184262 | 0.000546072 | 0.000208991 | 0.000155068 |
| C[T > A]C | 0.02919882 | 0.00312974 | 2.23E−16 | 0.000105999 | 0.002791598 | 0.000614976 | 0.000212302 | 0.005600734 | 0.000341985 | 0.002951295 |
| C[T > A]G | 0.027598885 | 0.005309559 | 2.23E−16 | 2.22E−16 | 0.000900188 | 0.001003822 | 0.000305434 | 0.001410185 | 0.00022599 | 0.000317139 |
| C[T > A]T | 0.041798311 | 0.004439632 | 0.000376029 | 0.000233997 | 0.003353168 | 0.002109104 | 0.000162231 | 0.001500197 | 0.000522977 | 0.001380606 |
| C[T > C]A | 0.002389903 | 0.013698863 | 0.000122009 | 0.001319982 | 0.011971489 | 0.002599258 | 0.000328467 | 0.045405948 | 4.74E−05 | 0.003161387 |
| C[T > C]C | 0.006999717 | 0.01529873 | 4.12E−05 | 0.000123998 | 0.009783289 | 0.002265801 | 0.000714015 | 0.049706512 | 0.000121995 | 0.005342344 |
| C[T > C]G | 0.001259949 | 0.015398722 | 0.001050082 | 2.53E−05 | 0.014627787 | 0.003317489 | 0.000131187 | 0.0374049 | 2.22E−16 | 0.004471962 |
| C[T > C]T | 0.006459739 | 0.03289727 | 2.44E−06 | 0.000814989 | 0.019248987 | 0.004746927 | 8.46E−05 | 0.045906014 | 0.000111995 | 0.002060904 |
| C[T > G]A | 0.001219951 | 0.025997842 | 0.000155012 | 0.000226997 | 0.010814145 | 0.006342346 | 2.22E−16 | 0.000893117 | 4.14E−05 | 4.85E−05 |
| C[T > G]C | 0.001949921 | 0.007409385 | 2.23E−16 | 0.001149984 | 0.002296995 | 0.000494858 | 2.22E−16 | 0.001140149 | 2.82E−06 | 0.001210531 |
| C[T > G]G | 0.00469981 | 0.014198821 | 5.35E−05 | 0.000478993 | 0.006864829 | 0.003076793 | 2.22E−16 | 0.000593078 | 2.22E−16 | 0.000435191 |
| C[T > G]T | 0.000573977 | 0.056495311 | 0.001300101 | 0.005229927 | 0.030012461 | 0.005924918 | 0.000133189 | 0.000344045 | 5.55E−05 | 0.000962422 |
| G[C > A]A | 0.024199022 | 0.007719359 | 0.001210094 | 0.00073099 | 0.008417708 | 0.002419072 | 0.000165235 | 0.001980259 | 0.000948958 | 0.002080913 |
| G[C > A]C | 0.026198942 | 0.006679446 | 0.000441034 | 0.002549964 | 0.007434356 | 0.025039732 | 0.000405577 | 0.000417055 | 0.00045098 | 0.022409832 |
| G[C > A]G | 0.002739889 | 0.000170986 | 1.05E−05 | 8.52E−05 | 0.000892212 | 8.73E−05 | 1.39E−05 | 0.000447059 | 0.000163993 | 0.001110487 |
| G[C > A]T | 0.026898913 | 0.00855929 | 0.011100862 | 0.002799961 | 0.012057535 | 0.009889342 | 2.22E−16 | 2.75E−05 | 6.15E−05 | 0.126055306 |
| G[C > G]A | 0.002579896 | 0.001359887 | 1.50E−05 | 1.72E−05 | 0.000170107 | 0.000104301 | 4.68E−05 | 0.000564074 | 0.001499934 | 2.22E−16 |
| G[C > G]C | 0.002389903 | 0.002829765 | 8.31E−05 | 0.000135998 | 0.000388352 | 0.00032515 | 0.000114162 | 0.000289038 | 0.000478979 | 4.33E−05 |
| G[C > G]G | 0.00048798 | 0.000520957 | 2.23E−16 | 2.22E−16 | 3.28E−05 | 1.91E−05 | 3.24E−06 | 9.54E−05 | 0.000240989 | 2.22E−16 |
| G[C > G]T | 0.004859804 | 0.008569289 | 3.54E−05 | 0.000112998 | 0.00015468 | 0.000370065 | 0.000186265 | 0.000504066 | 0.002699881 | 8.03E−05 |
| G[C > T]A | 0.000275989 | 0.006979421 | 4.22E−05 | 0.000362995 | 0.003613867 | 0.001154482 | 0.019728057 | 0.004480587 | 0.007179684 | 0.017107506 |
| G[C > T]C | 0.00024499 | 0.006529458 | 0.012500971 | 0.015099789 | 0.009029907 | 0.005566848 | 0.115163784 | 0.001820238 | 0.000920959 | 0.008163582 |
| G[C > T]G | 0.001909923 | 0.004759605 | 0.014901157 | 0.012599824 | 0.009224204 | 0.002046624 | 0.001782535 | 7.46E−05 | 0.000177992 | 1.92E−05 |
| G[C > T]T | 0.004609814 | 0.010699112 | 0.013501048 | 0.031899553 | 0.010383117 | 0.002248142 | 0.069699125 | 0.00267035 | 0.001269944 | 0.010504609 |
| G[T > A]A | 0.012799483 | 0.00264978 | 3.31E−06 | 2.22E−16 | 0.001902315 | 0.001546366 | 1.54E−06 | 0.000826108 | 6.70E−06 | 5.77E−05 |
| G[T > A]C | 0.013499455 | 0.002119824 | 2.23E−16 | 0.000122998 | 0.006889386 | 0.003352962 | 0.000375534 | 0.001810237 | 0.000129994 | 0.002731198 |
| G[T > A]G | 0.01559937 | 0.000787935 | 7.30E−05 | 2.22E−16 | 0.001930279 | 0.000539586 | 0.000119169 | 0.002510329 | 4.70E−05 | 0.000120053 |
| G[T > A]T | 0.028098865 | 0.002559788 | 0.000426033 | 0.000107998 | 0.012815 | 0.006874777 | 0.000164234 | 0.00091512 | 0.000357984 | 0.000857376 |
| G[T > C]A | 0.002599895 | 0.00951921 | 0.002200171 | 0.014699794 | 0.012739365 | 0.002431649 | 0.000810152 | 0.071109315 | 0.000286987 | 0.011004828 |
| G[T > C]C | 0.002619894 | 0.009779188 | 0.006280488 | 0.005329925 | 0.006069695 | 0.002737737 | 0.000861225 | 0.030403983 | 0.000181992 | 0.006542871 |
| G[T > C]G | 0.002809886 | 0.006309476 | 2.23E−16 | 0.001379981 | 0.012729929 | 0.003533476 | 0.000561799 | 0.041505437 | 0.000194991 | 0.011004828 |
| G[T > C]T | 0.006289746 | 0.009859182 | 0.005290411 | 0.011499839 | 0.010569946 | 0.002166806 | 0.000237338 | 0.046306066 | 2.22E−16 | 0.011204916 |
| G[T > G]A | 0.001309947 | 0.008299311 | 0.000204016 | 0.000480993 | 0.000355061 | 0.000393075 | 9.58E−05 | 0.000412054 | 8.77E−05 | 7.79E−05 |
| G[T > G]C | 0.000655973 | 0.005849514 | 0.000833065 | 0.000941987 | 0.000620308 | 0.00157296 | 2.22E−16 | 0.000213028 | 2.22E−16 | 0.002100922 |
| G[T > G]G | 0.00396984 | 0.003969671 | 2.23E−16 | 0.001729976 | 0.000614245 | 0.000200061 | 0.000130185 | 0.000496065 | 0.000425981 | 0.000781343 |
| G[T > G]T | 0.001289948 | 0.01939839 | 0.002210172 | 0.005159928 | 0.007473674 | 0.000947835 | 0.000154219 | 0.000165022 | 2.89E−06 | 0.002250988 |
| T[C > A]A | 0.029798796 | 0.016998589 | 0.0940073 | 0.000445994 | 0.060185803 | 0.258971765 | 0.000180256 | 0.003480456 | 0.078996521 | 0.000347152 |
| T[C > A]C | 0.025098986 | 0.002459796 | 0.009970774 | 0.003799947 | 0.008543156 | 0.014406759 | 0.000939336 | 0.002880377 | 0.029398705 | 0.024310666 |
| T[C > A]G | 0.001899923 | 0.000444963 | 0.00116009 | 0.000572992 | 0.002657373 | 0.007697783 | 2.22E−16 | 0.000505066 | 0.012099467 | 7.08E−06 |
| T[C > A]T | 0.031898711 | 0.027297734 | 0.67005203 | 0.081598858 | 0.188803967 | 0.341876446 | 4.74E−05 | 0.002350308 | 0.035498437 | 0.151066279 |
| T[C > G]A | 0.001469941 | 0.002689777 | 5.55E−05 | 2.22E−16 | 0.00025875 | 0.000120906 | 1.56E−05 | 0.000969127 | 0.31698604 | 1.97E−05 |
| T[C > G]C | 0.001339946 | 0.001999834 | 2.23E−16 | 2.22E−16 | 0.000395677 | 6.54E−05 | 0.000224319 | 0.001550203 | 0.063497204 | 0.000150066 |
| T[C > G]G | 7.82E−05 | 0.000403966 | 2.23E−16 | 2.22E−16 | 1.04E−05 | 1.96E−05 | 2.22E−16 | 0.000214028 | 0.013799392 | 1.83E−05 |
| T[C > G]T | 0.00024899 | 0.005629533 | 5.45E−05 | 4.34E−05 | 0.000347148 | 0.000168726 | 0.000185263 | 0.0030504 | 0.36898375 | 0.000321141 |
| T[C > T]A | 0.005039796 | 0.004269646 | 0.000366028 | 0.002729962 | 0.002403434 | 0.001332018 | 0.009793929 | 0.005520723 | 0.006929695 | 0.003541554 |
| T[C > T]C | 0.00025199 | 0.007329392 | 0.002760214 | 0.048499321 | 0.00901811 | 0.003584015 | 0.158225025 | 0.002640346 | 0.004479803 | 0.00013706 |
| T[C > T]G | 4.17E−05 | 0.003259729 | 0.002790217 | 0.436993882 | 0.008591415 | 0.005314164 | 2.94E−07 | 8.85E−05 | 0.001189948 | 0.005322335 |
| T[C > T]T | 0.005269787 | 0.005459547 | 0.022801771 | 0.127998208 | 0.012801276 | 0.00811322 | 0.119169481 | 0.006120802 | 0.001419937 | 0.000359158 |
| T[T > A]A | 0.011499535 | 0.025997842 | 0.002040158 | 0.003059957 | 0.009962652 | 0.008925788 | 0.000186265 | 0.003750491 | 0.000295987 | 0.000200088 |
| T[T > A]C | 0.019499212 | 0.001849846 | 0.000360028 | 0.000664991 | 0.002474664 | 0.000761367 | 0.000202288 | 0.006450845 | 0.00023199 | 0.001730759 |
| T[T > A]G | 0.012899479 | 0.016698614 | 0.000693054 | 7.06E−05 | 0.000565805 | 0.003781942 | 0.000201286 | 0.002770363 | 0.000187992 | 0.000357157 |
| T[T > A]T | 0.037898469 | 0.016998589 | 0.005290411 | 0.001519979 | 0.004831271 | 0.006969806 | 0.00067496 | 0.005250688 | 6.20E−05 | 0.001820799 |
| T[T > C]A | 0.002079916 | 0.013998838 | 0.000730057 | 0.008859876 | 0.016998769 | 0.005648334 | 0.000113161 | 0.086111281 | 2.21E−05 | 0.006272752 |
| T[T > C]C | 0.002639893 | 0.013598871 | 0.005170401 | 8.10E−05 | 0.016759702 | 0.006479062 | 0.000467665 | 0.046306066 | 2.49E−05 | 0.002881264 |
| T[T > C]G | 2.22E−16 | 0.009309227 | 0.000363028 | 0.000667991 | 0.015115317 | 0.004968228 | 0.000155221 | 0.040005241 | 9.98E−05 | 0.004311892 |
| T[T > C]T | 0.004569815 | 0.038396813 | 0.003250252 | 2.51E−05 | 0.019920805 | 0.007554899 | 0.000106151 | 0.059707822 | 0.000530977 | 0.002921282 |
| T[T > G]A | 2.22E−16 | 0.065694547 | 0.002690209 | 0.011299842 | 0.008730769 | 0.019897639 | 8.52E−05 | 0.001630214 | 0.000183992 | 9.92E−05 |
| T[T > G]C | 0.001139954 | 0.008619285 | 2.23E−16 | 0.005549922 | 0.000952155 | 0.000738318 | 6.74E−05 | 0.001130148 | 2.22E−16 | 0.001200527 |
| T[T > G]G | 0.003089875 | 0.010899095 | 2.16E−05 | 0.002759961 | 0.00110836 | 0.003147963 | 0.000101144 | 0.000875115 | 3.67E−05 | 0.00011405 |
| T[T > G]T | 0.00099596 | 0.063894697 | 0.018901468 | 0.090898727 | 0.025114706 | 0.014528208 | 5.56E−05 | 0.00221029 | 1.89E−05 | 0.000605266 |
| Type | SBS15 | SBS16 | SBS17a | SBS17b | SBS18 | SBS19 | SBS20 | SBS21 | SBS22 | SBS23 |
| A[C > A]A | 0.000944202 | 0.015995037 | 0.002070261 | 0.000607958 | 0.051533858 | 0.001269382 | 0.000619283 | 0.000156993 | 0.006012763 | 0.000835523 |
| A[C > A]C | 0.000497106 | 0.0028991 | 0.000918116 | 0.000128991 | 0.015810387 | 0.000640688 | 0.001390636 | 0.002359889 | 9.54E−05 | 0.00039925 |
| A[C > A]G | 4.61E−05 | 0.001019684 | 4.76E−05 | 5.82E−05 | 0.002431598 | 0.00024588 | 2.18E−05 | 0.000293986 | 0.000765352 | 9.86E−08 |
| A[C > A]T | 0.001110238 | 0.010596712 | 6.18E−05 | 0.000455969 | 0.021414069 | 0.000570722 | 0.001240568 | 0.000620971 | 0.001720791 | 5.61E−18 |
| A[C > G]A | 0.000113024 | 0.00173946 | 0.001010128 | 0.00014599 | 0.001731137 | 0.003188448 | 0.008774015 | 1.69E−05 | 0.001660763 | 5.74E−18 |
| A[C > G]C | 0.000269058 | 0.002009376 | 0.000569072 | 4.34E−05 | 0.002591703 | 0.001789129 | 0.001510691 | 0.000718966 | 0.00019509 | 4.08E−05 |
| A[C > G]G | 3.07E−05 | 0.000846737 | 0.000152019 | 2.23E−16 | 0.001921262 | 0.0006157 | 0.000469215 | 2.22E−16 | 0.000231106 | 2.86E−18 |
| A[C > G]T | 0.00032507 | 0.011496433 | 1.96E−05 | 0.000318978 | 0.004082682 | 0.002138959 | 0.004422024 | 6.19E−05 | 6.20E−05 | 5.61E−18 |
| A[C > T]A | 0.005051081 | 0.001759454 | 0.001220154 | 0.000570961 | 0.009206048 | 0.027986379 | 0.049322571 | 0.002169898 | 0.001760809 | 0.022614152 |
| A[C > T]C | 0.00691148 | 0.003728843 | 0.001710216 | 7.03E−05 | 0.004653057 | 0.014093141 | 0.013005952 | 0.003919816 | 0.000220101 | 0.080950659 |
| A[C > T]G | 0.074515954 | 0.003268986 | 3.95E−05 | 0.00028698 | 0.012208021 | 0.001549246 | 0.013306089 | 0.007399653 | 0.001360625 | 0.00872546 |
| A[C > T]T | 0.001780381 | 0.005998139 | 0.000737093 | 0.00014699 | 0.00797524 | 0.036782098 | 0.013005952 | 4.17E−05 | 0.000616283 | 0.035021917 |
| A[T > A]A | 0.002040437 | 0.023092834 | 0.001010128 | 6.26E−05 | 0.002491637 | 0.000785618 | 0.000511234 | 0.003459838 | 0.063929382 | 8.29E−18 |
| A[T > A]C | 0.00084118 | 0.007737599 | 0.000997126 | 0.00028898 | 0.000878577 | 0.000254876 | 0.005192376 | 0.005649735 | 0.007013223 | 5.56E−18 |
| A[T > A]G | 0.000540116 | 0.008807267 | 0.000200025 | 6.57E−05 | 0.002601709 | 0.000951537 | 0.000303139 | 0.000247988 | 0.060627865 | 0.000354222 |
| A[T > A]T | 0.009542043 | 0.010996588 | 0.001460184 | 0.000632957 | 0.007845154 | 0.000653682 | 0.02561172 | 0.001599925 | 0.002511154 | 0.000161101 |
| A[T > C]A | 0.003890833 | 0.241924931 | 9.65E−05 | 9.73E−05 | 0.002021328 | 0.00258874 | 0.010704899 | 0.016699216 | 0.025011495 | 0.000886555 |
| A[T > C]C | 0.00196042 | 0.012895998 | 0.012701604 | 0.000251983 | 0.002091374 | 0.000593711 | 0.004552083 | 0.009999531 | 0.000899413 | 0.000178111 |
| A[T > C]G | 0.000240051 | 0.063880178 | 0.000376047 | 2.23E−16 | 0.002081367 | 0.002308876 | 0.018608516 | 0.003879818 | 0.007753564 | 0.000470294 |
| A[T > C]T | 0.000929199 | 0.135957812 | 0.007790984 | 0.000543963 | 0.004242788 | 0.001739154 | 0.000103047 | 0.025598798 | 0.000755347 | 0.000261163 |
| A[T > G]A | 0.0004681 | 0.011196526 | 0.000851107 | 0.001409903 | 0.000721474 | 0.002328867 | 1.05E−05 | 0.000391982 | 0.002331071 | 8.29E−18 |
| A[T > G]C | 0.000415089 | 0.004408632 | 0.000700088 | 0.004479693 | 0.000858564 | 0.001889081 | 2.13E−05 | 0.000436979 | 5.09E−05 | 5.43E−18 |
| A[T > G]G | 0.00018504 | 0.004618567 | 0.000719091 | 0.000989932 | 0.001991308 | 0.001769139 | 2.69E−05 | 0.000186991 | 0.002951356 | 5.68E−18 |
| A[T > G]T | 0.003630777 | 0.007957531 | 0.012501578 | 0.069095261 | 0.00184121 | 0.001619212 | 5.82E−05 | 6.95E−05 | 4.18E−05 | 7.43E−18 |
| C[C > A]A | 0.041808951 | 0.010396774 | 0.000295037 | 0.000270981 | 0.07404865 | 0.001319358 | 0.037617214 | 2.22E−16 | 0.001830841 | 0.000151095 |
| C[C > A]C | 0.005051081 | 0.009027199 | 0.001160146 | 4.11E−05 | 0.019612886 | 0.000766627 | 0.098945279 | 0.002599878 | 0.004432037 | 0.000630395 |
| C[C > A]G | 6.62E−05 | 0.00173946 | 2.22E−16 | 2.23E−16 | 0.012007889 | 0.000235885 | 0.007433402 | 0.001979907 | 0.000136063 | 0.000130081 |
| C[C > A]T | 0.028706146 | 0.002489228 | 0.000137017 | 0.000181988 | 0.036323865 | 3.44E−05 | 0.252115373 | 0.019099103 | 0.000110051 | 0.000580363 |
| C[C > G]A | 8.36E−05 | 0.003548899 | 0.00055307 | 0.000193987 | 0.000613403 | 0.001769139 | 7.69E−05 | 1.97E−05 | 0.001050483 | 0.000174109 |
| C[C > G]C | 1.70E−05 | 0.004538592 | 0.00023503 | 4.66E−05 | 0.002261486 | 0.00180912 | 4.38E−06 | 0.000272987 | 0.000785361 | 0.00025516 |
| C[C > G]G | 5.02E−05 | 0.001459547 | 0.000232029 | 2.23E−16 | 0.003582354 | 4.81E−05 | 0.000188086 | 1.01E−05 | 0.00023911 | 1.86E−18 |
| C[C > G]T | 8.45E−05 | 0.009617016 | 3.90E−05 | 0.000192987 | 0.003792492 | 0.003218434 | 1.01E−05 | 0.000172992 | 0.000320147 | 0.000227142 |
| C[C > T]A | 0.010802313 | 0.007167776 | 0.000821104 | 0.000456969 | 0.011707692 | 0.118942111 | 0.010604853 | 0.001349937 | 0.001210556 | 0.062238949 |
| C[C > T]C | 0.011102377 | 0.002839119 | 0.001720217 | 5.89E−05 | 0.006764444 | 0.091955245 | 0.013005952 | 0.005159758 | 0.003191467 | 0.189118351 |
| C[C > T]G | 0.024805311 | 0.000690786 | 0.00190024 | 0.000542963 | 0.016811045 | 0.015692363 | 0.016507554 | 0.006219708 | 0.000426196 | 0.014208892 |
| C[C > T]T | 0.015603341 | 0.019294013 | 0.00174022 | 0.000135991 | 0.006654372 | 0.257874492 | 0.011705357 | 0.000324985 | 0.001600736 | 0.156097686 |
| C[T > A]A | 0.000348075 | 0.003209004 | 0.000434055 | 9.36E−05 | 0.000542356 | 9.60E−05 | 0.000144066 | 6.19E−06 | 0.128058856 | 0.000462289 |
| C[T > A]C | 0.000244052 | 0.005698232 | 0.040505114 | 0.002239846 | 0.003062012 | 0.000402804 | 0.006482967 | 0.000687968 | 0.055125336 | 4.47E−18 |
| C[T > A]G | 8.20E−05 | 0.00102968 | 0.012401566 | 0.000716951 | 0.00321211 | 0.000293857 | 0.000123056 | 0.000124994 | 0.168077248 | 0.000132083 |
| C[T > A]T | 0.001050225 | 0.006527974 | 0.07230913 | 0.007789466 | 0.002261486 | 0.000217894 | 0.000102047 | 2.22E−16 | 0.060327727 | 0.000375235 |
| C[T > C]A | 0.000750161 | 0.010696681 | 0.011101402 | 0.000646956 | 0.000386254 | 0.00045178 | 0.011705357 | 0.01149946 | 0.006743099 | 6.86E−18 |
| C[T > C]C | 0.00215046 | 0.003169017 | 0.173021846 | 0.006459557 | 0.002461617 | 8.14E−05 | 0.004832211 | 0.002819868 | 0.001340616 | 4.47E−18 |
| C[T > C]G | 0.001390298 | 0.009607019 | 0.087311024 | 0.003919731 | 0.001621065 | 0.000497758 | 0.026111949 | 0.014399324 | 0.002030933 | 0.000389244 |
| C[T > C]T | 0.001750375 | 0.015495192 | 0.426053794 | 0.018298745 | 0.002561683 | 0.00039081 | 0.006903159 | 0.021698981 | 0.000536246 | 4.71E−05 |
| C[T > G]A | 0.000206044 | 0.003958772 | 0.000310039 | 0.002049859 | 0.000111073 | 0.000713653 | 0.000963441 | 4.35E−06 | 0.004912258 | 6.85E−18 |
| C[T > G]C | 0.000157034 | 0.001079665 | 0.001040131 | 0.031397847 | 0.001991308 | 0.002088983 | 0.001600733 | 5.44E−05 | 0.000880405 | 0.000148093 |
| C[T > G]G | 0.000272058 | 0.002239305 | 1.94E−06 | 0.013699061 | 0.002931926 | 0.001329353 | 0.002711241 | 2.01E−05 | 0.008033692 | 6.52E−05 |
| C[T > G]T | 0.002490533 | 0.002539212 | 0.005310671 | 0.549962284 | 0.004142722 | 0.00219893 | 0.00511234 | 0.000184991 | 0.001190547 | 0.000247155 |
| G[C > A]A | 0.00229049 | 0.008607329 | 0.001780225 | 0.000997932 | 0.10907166 | 0.000813604 | 0.010804945 | 0.0021399 | 0.002231025 | 0.000337211 |
| G[C > A]C | 0.002490533 | 0.004248682 | 0.001280162 | 2.78E−06 | 0.017311374 | 0.000271868 | 0.005652587 | 0.007069668 | 0.001730795 | 0.000541339 |
| G[C > A]G | 0.000404087 | 0.001359578 | 0.000106013 | 9.24E−05 | 0.00756497 | 0.0002049 | 0.001570719 | 0.001689921 | 0.000515237 | 0.000119075 |
| G[C > A]T | 0.018203897 | 0.005798201 | 4.70E−05 | 0.000533963 | 0.062340958 | 0.000272867 | 0.00249114 | 0.008899582 | 0.000551253 | 3.09E−05 |
| G[C > G]A | 0.000994213 | 0.001049674 | 0.000878111 | 5.99E−05 | 0.000635417 | 0.001309363 | 0.000669306 | 0.000387982 | 0.001400644 | 4.32E−18 |
| G[C > G]C | 0.00060813 | 0.003089041 | 0.000835105 | 5.55E−05 | 0.001340881 | 0.001289372 | 0.002861309 | 0.001419933 | 0.000930428 | 0.00012708 |
| G[C > G]G | 1.01E−05 | 8.13E−05 | 2.37E−05 | 2.23E−16 | 0.00063942 | 0.00059671 | 0.000692317 | 0.000116995 | 0.000211097 | 1.83E−18 |
| G[C > G]T | 0.000451097 | 0.003228998 | 0.000787099 | 2.23E−16 | 0.001230809 | 0.000319844 | 0.003021383 | 0.00020699 | 0.000455209 | 4.04E−18 |
| G[C > T]A | 0.073715783 | 0.006497984 | 0.001640207 | 6.11E−05 | 0.009686364 | 0.060970326 | 0.04221932 | 0.009319563 | 0.002381094 | 0.053733627 |
| G[C > T]C | 0.129027625 | 0.005288359 | 2.22E−16 | 0.000156989 | 0.008175371 | 0.039680687 | 0.053224357 | 0.01959908 | 0.00187086 | 0.154096434 |
| G[C > T]G | 0.277059318 | 0.001169637 | 0.001610203 | 0.000854941 | 0.01480973 | 0.003208438 | 0.020509386 | 4.34E−05 | 2.22E−16 | 0.012307702 |
| G[C > T]T | 0.077216532 | 0.006717915 | 6.60E−05 | 2.23E−16 | 0.006694398 | 0.108946976 | 0.033115154 | 0.008049622 | 0.001760809 | 0.107067003 |
| G[T > A]A | 0.000511109 | 0.000939708 | 6.46E−05 | 9.73E−05 | 0.001601052 | 0.000188908 | 5.56E−05 | 0.001169945 | 0.053724692 | 6.26E−18 |
| G[T > A]C | 0.000589126 | 0.000607811 | 0.003400429 | 0.000346976 | 0.001951282 | 7.78E−05 | 0.002561172 | 0.003259847 | 0.01020469 | 0.000140088 |
| G[T > A]G | 0.00018704 | 0.005548278 | 0.000695088 | 0.000240983 | 0.001831203 | 0.000485764 | 0.002130975 | 0.000741965 | 0.037517243 | 4.68E−18 |
| G[T > A]T | 0.00084018 | 0.0009657 | 0.004800606 | 0.001579892 | 0.003462275 | 0.000282862 | 0.004301969 | 0.000936956 | 0.009324285 | 5.78E−18 |
| G[T > C]A | 0.012902762 | 0.002059361 | 0.000123016 | 5.95E−05 | 0.000708465 | 0.000953536 | 0.002761264 | 0.222989533 | 0.000721332 | 0.000218137 |
| G[T > C]C | 0.011602484 | 0.001189631 | 0.014901882 | 0.001499897 | 0.002261486 | 0.00037082 | 0.002261035 | 0.066296888 | 0.00124057 | 0.000418262 |
| G[T > C]G | 0.005951274 | 0.004278672 | 0.003570451 | 0.001009931 | 0.003982617 | 0.000952536 | 0.004061859 | 0.116994508 | 0.000581267 | 4.68E−18 |
| G[T > C]T | 0.005861255 | 0.002399256 | 0.005240662 | 0.001469899 | 0.003152071 | 0.002358852 | 0.000928425 | 0.132993757 | 0.000834383 | 0.0007995 |
| G[T > G]A | 0.000203043 | 0.00090172 | 0.000495063 | 0.000280981 | 0.000870572 | 0.000635691 | 2.22E−16 | 0.000285987 | 0.001590731 | 6.26E−18 |
| G[T > G]C | 0.000580124 | 0.000900721 | 0.000204026 | 0.006249571 | 3.70E−05 | 0.000702658 | 6.65E−05 | 0.000532975 | 0.000236109 | 8.70E−05 |
| G[T > G]G | 0.000347074 | 0.000674791 | 0.000835105 | 0.001479899 | 0.005833833 | 0.000509752 | 8.70E−05 | 0.00021299 | 0.000450207 | 0.000157098 |
| G[T > G]T | 0.001390298 | 0.002389259 | 0.007240914 | 0.113992182 | 0.002141407 | 0.000340834 | 0.00356163 | 0.002589878 | 1.96E−05 | 5.78E−18 |
| T[C > A]A | 0.017203683 | 0.01079665 | 0.000190024 | 1.24E−05 | 0.073848518 | 0.001379329 | 0.001710783 | 0.001459931 | 0.00078436 | 0.002131334 |
| T[C > A]C | 0.005811244 | 0.005868179 | 2.59E−05 | 2.23E−16 | 0.043628664 | 0.00112945 | 0.003371543 | 0.000235989 | 0.0017408 | 0.000664416 |
| T[C > A]G | 7.69E−05 | 0.001489538 | 2.22E−16 | 9.51E−05 | 0.012708349 | 0.000284861 | 2.22E−16 | 6.83E−05 | 2.22E−16 | 9.13E−05 |
| T[C > A]T | 0.008051724 | 0.009247131 | 0.000288036 | 0.002569824 | 0.122080207 | 0.001379329 | 0.000904414 | 2.22E−16 | 0.000396182 | 0.00024015 |
| T[C > G]A | 0.000214046 | 0.002509221 | 0.003750474 | 0.000159989 | 0.002141407 | 0.001749149 | 0.000129059 | 0.000653969 | 0.003561637 | 0.000211132 |
| T[C > G]C | 6.12E−05 | 0.005228378 | 0.000723091 | 6.18E−05 | 0.00464305 | 0.003118482 | 2.82E−05 | 0.00063097 | 0.001520699 | 0.000266167 |
| T[C > G]G | 5.84E−05 | 0.000438864 | 0.000380048 | 1.55E−05 | 0.000899591 | 0.000440785 | 0.000124057 | 6.27E−05 | 0.000381175 | 2.56E−18 |
| T[C > G]T | 0.000174037 | 0.028491159 | 0.000283036 | 0.001499897 | 0.003182091 | 0.001609217 | 0.001120513 | 0.000686968 | 0.000388178 | 0.000466292 |
| T[C > T]A | 0.026505675 | 0.006358027 | 0.000532067 | 0.000107993 | 0.011407495 | 0.043878644 | 0.008073695 | 0.003599831 | 0.001590731 | 0.015309581 |
| T[C > T]C | 0.009562047 | 0.003678858 | 9.06E−05 | 0.000603959 | 0.011407495 | 0.024987838 | 0.009194207 | 8.07E−05 | 0.001320607 | 0.043627302 |
| T[C > T]G | 0.02120454 | 0.003019063 | 0.000177022 | 6.24E−05 | 0.011807758 | 0.004317898 | 0.002641209 | 0.000316985 | 3.77E−05 | 0.002871797 |
| T[C > T]T | 0.001740373 | 0.022093144 | 0.000348044 | 0.000894939 | 0.003752465 | 0.062869401 | 5.00E−05 | 7.31E−05 | 2.22E−16 | 0.026316469 |
| T[T > A]A | 0.001090233 | 0.012296184 | 0.000119015 | 0.001189918 | 0.0108071 | 0.00049376 | 0.012005494 | 0.000256988 | 0.071833014 | 7.77E−18 |
| T[T > A]C | 0.000157034 | 0.004668551 | 0.00570072 | 0.000241983 | 0.002731795 | 0.000474769 | 0.000318146 | 0.000794963 | 0.023610852 | 5.27E−18 |
| T[T > A]G | 0.000149032 | 0.005178393 | 0.001830231 | 2.23E−16 | 0.001791177 | 0.000372819 | 0.000176081 | 9.85E−05 | 0.073233658 | 0.000185116 |
| T[T > A]T | 0.000475102 | 0.007157779 | 0.00198025 | 0.00130991 | 0.007104668 | 0.000672673 | 0.006713072 | 7.64E−05 | 0.03011384 | 7.32E−18 |
| T[T > C]A | 0.003420732 | 0.012995967 | 0.002090264 | 0.001059927 | 0.001801183 | 0.001239397 | 0.013806318 | 0.084196048 | 0.004632129 | 7.77E−18 |
| T[T > C]C | 0.005921268 | 0.003668862 | 0.015301932 | 0.001099925 | 0.006184063 | 0.000552731 | 0.0166076 | 0.054797428 | 2.22E−16 | 0.000190119 |
| T[T > C]G | 0.001320283 | 0.007227757 | 0.002350297 | 0.001209917 | 0.003262143 | 0.001079475 | 0.012005494 | 0.027598705 | 0.001220561 | 0.000302189 |
| T[T > C]T | 0.001350289 | 0.008387397 | 0.021002652 | 0.000999931 | 0.003372216 | 0.001469285 | 0.019308836 | 0.04069809 | 2.22E−16 | 7.32E−18 |
| T[T > G]A | 0.000231049 | 0.009776966 | 2.22E−16 | 1.22E−05 | 0.000696458 | 0.001659192 | 2.22E−16 | 0.000464978 | 0.005062327 | 0.000186116 |
| T[T > G]C | 0.000294063 | 0.005508291 | 0.000116015 | 0.0087494 | 0.002101381 | 0.002019017 | 2.22E−16 | 0.000378982 | 0.000752346 | 5.27E−18 |
| T[T > G]G | 0.000148032 | 0.005238375 | 0.000920116 | 0.004779672 | 0.001450953 | 0.002068993 | 0.002791277 | 0.000130994 | 0.005572561 | 0.000182114 |
| T[T > G]T | 0.005991283 | 0.015795099 | 0.004580578 | 0.121991634 | 0.005163392 | 0.004227942 | 0.001510691 | 2.22E−16 | 0.000595274 | 7.32E−18 |
| Type | SBS24 | SBS25 | SBS26 | SBS27 | SBS28 | SBS29 | SBS30 | SBS31 | SBS32 | SBS33 |
| A[C > A]A | 0.0364134 | 0.009898466 | 0.000873118 | 0.005207818 | 0.000783905 | 0.063209262 | 0.001799681 | 0.009534985 | 0.022297772 | 0.00311136 |
| A[C > A]C | 0.026509756 | 0.006998915 | 0.000528071 | 0.004738015 | 0.002529694 | 0.051207503 | 0.00050591 | 0.018490274 | 0.018798122 | 0.002230975 |
| A[C > A]G | 0.014805448 | 0.001449775 | 0.000114015 | 0.000782672 | 0.000352957 | 0.019202814 | 9.13E−05 | 0.001659127 | 0.004459554 | 0.000414181 |
| A[C > A]T | 0.022008099 | 0.00496923 | 0.000619084 | 0.002718861 | 0.00396952 | 0.035605217 | 0.000555901 | 0.006276698 | 0.014598542 | 0.001900831 |
| A[C > G]A | 0.00334123 | 0.008028756 | 0.000427058 | 0.001319447 | 0.000153981 | 0.00314046 | 0.00106981 | 0.008315626 | 0.001989801 | 0.001410616 |
| A[C > G]C | 0.003671351 | 0.001639746 | 0.000378051 | 0.001769259 | 0.000223973 | 0.002630385 | 0.000466917 | 0.003158339 | 0.002029797 | 0.001300568 |
| A[C > G]G | 0.000920339 | 0 | 6.83E−05 | 0 | 2.22E−16 | 0.000896131 | 0.000130977 | 0.002998423 | 0.001129887 | 0.000253111 |
| A[C > G]T | 0.002851049 | 0.003429468 | 0.000728098 | 0.001319447 | 8.33E−05 | 0.002490365 | 0.00073087 | 0.003778013 | 0.001749825 | 0.001300568 |
| A[C > T]A | 0.011804344 | 0.020996746 | 0.002660359 | 0.013694263 | 0.000776906 | 0.005090746 | 0.105981199 | 0.017690695 | 0.08639137 | 0.005312321 |
| A[C > T]C | 0.011204123 | 0.01309797 | 0.002540343 | 0.005417731 | 0.003379591 | 0.004310632 | 0.090383966 | 0.009275121 | 0.122987714 | 0.002130931 |
| A[C > T]G | 0.000514189 | 0.019696947 | 0.00533072 | 0.00670719 | 0.007049147 | 0.020102946 | 0.014397446 | 0.00758601 | 0.036596344 | 0.00716313 |
| A[C > T]T | 0.009153368 | 0.005729112 | 0.002790377 | 0.004478124 | 0.005059388 | 0.001830268 | 0.029294803 | 0.01009469 | 0.151984817 | 0.003071342 |
| A[T > A]A | 0.001580582 | 0.020396838 | 0.001520205 | 0.142940122 | 0.000171979 | 0.000619091 | 0.000362936 | 0.007855868 | 0.000712929 | 0.000868379 |
| A[T > A]C | 0.002250828 | 0.008888622 | 0.003520476 | 0.001459389 | 0.000477942 | 0.002000293 | 0.00039293 | 0.006066809 | 0.000886911 | 0.001140498 |
| A[T > A]G | 0.002000736 | 0.006239033 | 0.000625084 | 0.001229485 | 0.000151982 | 0.002480363 | 0.000637887 | 0.005027356 | 0.000711929 | 0.000696304 |
| A[T > A]T | 0.001800663 | 0.014897691 | 0.003070415 | 0.002538936 | 0.001699794 | 0.001960287 | 0.000489913 | 0.004157813 | 0.000843916 | 0.005072217 |
| A[T > C]A | 0.006402356 | 0.020696792 | 0.084811458 | 0.005047885 | 0.000548934 | 0.001960287 | 0.000610892 | 0.020389275 | 0.000935907 | 0.012505465 |
| A[T > C]C | 0.002180803 | 0.009498528 | 0.026103527 | 0.001849225 | 0.000440947 | 0.002290336 | 0.000457919 | 0.00739611 | 0.00070093 | 0.003751639 |
| A[T > C]G | 0.001760648 | 0.019896916 | 0.078510607 | 0.005687617 | 0.000404951 | 0.002750403 | 0.000276951 | 0.008115731 | 0.001089891 | 0.07343209 |
| A[T > C]T | 0.002440898 | 0.014497753 | 0.037205026 | 0.002588916 | 0.000594928 | 0.001150169 | 0.000544903 | 0.008465547 | 0.000609939 | 0.009214027 |
| A[T > G]A | 0.003211182 | 0.005279182 | 0.002920395 | 0.001139523 | 0.009868806 | 0.00040706 | 3.90E−05 | 0.003678065 | 0.000385961 | 0.000979428 |
| A[T > G]C | 0.001400515 | 0 | 0.002530342 | 0.000958598 | 0.017297907 | 0.00081712 | 0.00010998 | 0.002478696 | 0.000428957 | 0.000302132 |
| A[T > G]G | 0.002060758 | 0.007138893 | 0.002290309 | 6.77E−05 | 0.007679071 | 0.000420062 | 0.000409927 | 0.004017887 | 0.001269873 | 0.000475208 |
| A[T > G]T | 0.004711734 | 0 | 0.005930801 | 0.000255893 | 0.114986087 | 0.000810119 | 0.000793859 | 0.000782588 | 6.45E−18 | 0.000758331 |
| C[C > A]A | 0.025409351 | 0.014797706 | 0.001410191 | 0.005067877 | 0.002029754 | 0.054207943 | 0.000767864 | 0.010694375 | 0.001239876 | 0.00270118 |
| C[C > A]C | 0.034912848 | 0.007818788 | 0.001510204 | 0.002229066 | 0.000108987 | 0.036905408 | 0.000387931 | 0.011593902 | 0.004919509 | 0.001910835 |
| C[C > A]G | 0.01600589 | 0.001279802 | 0.000362049 | 0.000265889 | 2.22E−16 | 0.014802169 | 0.000406928 | 0.000328827 | 1.66E−05 | 0.000371162 |
| C[C > A]T | 0.017106295 | 0.012598047 | 0.007140965 | 0.003098702 | 0.000202975 | 0.051907606 | 0.000296947 | 0.027885332 | 0.002669733 | 0.002531106 |
| C[C > G]A | 0.002610961 | 0.007198884 | 0.000364049 | 0.004728019 | 1.44E−05 | 0.003290482 | 0.001269775 | 0.002048922 | 0.001149885 | 0.001500656 |
| C[C > G]C | 0.005221922 | 0.004599287 | 0.000322044 | 0.001259472 | 7.55E−05 | 0.006340929 | 0.000792859 | 0.001859022 | 0.000823918 | 0.000410179 |
| C[C > G]G | 0.000945348 | 0.000823872 | 4.17E−05 | 0.000360849 | 2.22E−16 | 0.001300191 | 0.000119979 | 0.000853551 | 0.000536946 | 0.000318139 |
| C[C > G]T | 0.004711734 | 0.00019597 | 0.000625084 | 0.000267888 | 2.22E−16 | 0.00669098 | 0.001019819 | 0.003418202 | 0.000494951 | 0.000662289 |
| C[C > T]A | 0.005381981 | 0.014297784 | 0.001690228 | 0.017392714 | 0.000291965 | 0.007611115 | 0.109980489 | 0.04117834 | 0.016898312 | 0.004592007 |
| C[C > T]C | 0.008853258 | 0.005939079 | 0.002830382 | 0.00713701 | 0.000209975 | 0.00164024 | 0.102981731 | 0.163913781 | 0.028397163 | 0.001530669 |
| C[C > T]G | 0.006352338 | 0.011598202 | 0.003340451 | 0.00749686 | 0.002549691 | 0.015302242 | 0.01679702 | 0.008195689 | 0.011198881 | 0.005882571 |
| C[C > T]T | 0.004291579 | 0.013597892 | 0.001650223 | 0.007147006 | 0.003319598 | 0.000673099 | 0.046191806 | 0.14492377 | 0.042795725 | 0.00270118 |
| C[T > A]A | 0.001320486 | 0.026295924 | 0.000944128 | 0.330861402 | 0.000139983 | 0.001950286 | 0.000213962 | 0.02058917 | 0.000324968 | 0.000746326 |
| C[T > A]C | 0.001870688 | 0.015797551 | 0.001450196 | 0.002808823 | 0.001349837 | 0.001410207 | 0.000776862 | 0.021088907 | 0.000649935 | 0.000992434 |
| C[T > A]G | 0.005031852 | 0.062890252 | 0.001140154 | 0.004398158 | 0.000805902 | 0.00198029 | 0.000586896 | 0.018690169 | 0.001409859 | 0.000479209 |
| C[T > A]T | 0.004801767 | 0.036594328 | 0.000642087 | 0.005427726 | 0.001029875 | 0.00198029 | 0.000611891 | 0.030983703 | 0.000662934 | 0.00112049 |
| C[T > C]A | 0.002270836 | 0.009028601 | 0.052007026 | 0.004897948 | 0.00131984 | 0.001180173 | 0.001269775 | 0.008145715 | 0.000592941 | 0.015106602 |
| C[T > C]C | 0.002240825 | 0.010798326 | 0.031204216 | 0.005717605 | 0.002619683 | 0.00184027 | 0.001379755 | 0.00990479 | 0.000926907 | 0.030113159 |
| C[T > C]G | 0.004221554 | 0.013397923 | 0.078710634 | 0.00565763 | 0.002489699 | 0.003910573 | 0.001269775 | 0.004457655 | 0.001839816 | 0.040017488 |
| C[T > C]T | 0.003631336 | 0.008958611 | 0.055507499 | 0.003898367 | 0.000366956 | 0.001780261 | 0.004099273 | 0.013792745 | 0.001089891 | 0.003071342 |
| C[T > G]A | 0.001430526 | 0 | 0.001420192 | 0 | 0.014598234 | 0.000781114 | 0.000197965 | 0.001968964 | 5.81E−05 | 0.000728318 |
| C[T > G]C | 0.000880324 | 0.002889552 | 0.004940667 | 0.00038084 | 0.022097326 | 5.69E−06 | 0.000214962 | 0.001429248 | 0.000449955 | 0.000949415 |
| C[T > G]G | 0.003041119 | 0.011798171 | 0.005560751 | 0.000402831 | 0.015498125 | 0.000332049 | 7.85E−05 | 0.001499211 | 0.001089891 | 0.001070468 |
| C[T > G]T | 0.004711734 | 0.008548675 | 0.007801054 | 0.001439397 | 0.10098778 | 0.000767112 | 0.000264953 | 0.000948501 | 0.000675932 | 0.000192084 |
| G[C > A]A | 0.062222898 | 0.013497908 | 0.000496067 | 0.010795478 | 0.000155981 | 0.09711423 | 0.000942833 | 0.013093113 | 0.004509549 | 0.004411928 |
| G[C > A]C | 0.128047121 | 0.006468997 | 0.00066609 | 0.012394808 | 0.001019877 | 0.090313234 | 0.000493912 | 0.021488697 | 0.003619638 | 0.000968423 |
| G[C > A]G | 0.06592426 | 0.000657898 | 0.000129017 | 0.001629317 | 3.95E−05 | 0.023003371 | 2.22E−16 | 0.000971489 | 0.000876912 | 0.00034215 |
| G[C > A]T | 0.054420027 | 0.010498373 | 0.00081711 | 0.007406897 | 0.000157981 | 0.084612398 | 0.000861847 | 0.003508155 | 0.003269673 | 0.003191395 |
| G[C > G]A | 0.002721001 | 0.004169354 | 0.000395053 | 0.001419405 | 0.000182978 | 0.002270333 | 0.000514909 | 0.001829038 | 0.000875912 | 0.000771337 |
| G[C > G]C | 0.005792132 | 0.00238963 | 0.000579078 | 0.001349435 | 2.22E−16 | 0.00164024 | 0.000255955 | 0.007506052 | 0.00089991 | 0.000776339 |
| G[C > G]G | 0.003141156 | 0.000417935 | 7.38E−05 | 0 | 2.22E−16 | 6.88E−06 | 0.000116979 | 0.00045676 | 6.86E−05 | 4.66E−05 |
| G[C > G]T | 0.003381244 | 0.001989692 | 0.000305041 | 0.004558091 | 2.22E−16 | 0.00321047 | 0.000398929 | 0.003358234 | 0.000227977 | 0.000931407 |
| G[C > T]A | 0.029911007 | 0.00999845 | 0.002750372 | 0.014194054 | 0.003249607 | 0.010401524 | 0.049691185 | 0.006796425 | 0.022597742 | 0.005352339 |
| G[C > T]C | 0.043716088 | 0.006948923 | 0.005450736 | 0.020091584 | 0.000187977 | 0.007961167 | 0.060989181 | 0.011394007 | 0.037896214 | 0.005142247 |
| G[C > T]G | 0.025409351 | 0.010498373 | 0.006680903 | 0.009366077 | 0.000214974 | 0.018302682 | 0.005239071 | 0.001968964 | 0.010398961 | 0.005842553 |
| G[C > T]T | 0.020307473 | 0.009818478 | 0.004980673 | 0.007586822 | 0.000101988 | 0.00512075 | 0.017696861 | 0.005697003 | 0.053194686 | 0.003931718 |
| G[T > A]A | 0.001570578 | 0.03619439 | 0.000783106 | 0.10995394 | 0.000115986 | 0.003430503 | 0.000328942 | 0.001899001 | 0.008789122 | 0.000810354 |
| G[T > A]C | 0.002350865 | 0.005849093 | 0.001150155 | 2.35E−05 | 0.000117986 | 0.000789116 | 0.000181968 | 0.00290847 | 0.008219179 | 0.000362158 |
| G[T > A]G | 0.002490917 | 0.03819408 | 0.000608082 | 0.001569343 | 7.17E−05 | 0.003660536 | 9.86E−05 | 0.005766967 | 0.013098691 | 0.000827362 |
| G[T > A]T | 0.001310482 | 0.020196869 | 0.000834113 | 0 | 0.000323961 | 0.001070157 | 0.000190966 | 0.000788585 | 0.006159385 | 0.001760769 |
| G[T > C]A | 0.003701362 | 0.017497288 | 0.083511282 | 0.004777998 | 0.000490941 | 0.001080158 | 0.000934834 | 0.004527618 | 0.009139087 | 0.012205334 |
| G[T > C]C | 0.003651344 | 0.004049372 | 0.024803351 | 0.001959179 | 0.000586929 | 0.003590526 | 0.000554902 | 0.006566546 | 0.007669234 | 0.007943471 |
| G[T > C]G | 0.002670983 | 0.013397923 | 0.07120962 | 0.001229485 | 0.000730912 | 0.002990438 | 0.000500911 | 0.005127303 | 0.009619039 | 0.207090499 |
| G[T > C]T | 0.003991469 | 0.014197799 | 0.055207459 | 0.00374843 | 0.000850897 | 0.001550227 | 0.000444921 | 0.004687534 | 0.012898711 | 0.009714245 |
| G[T > G]A | 0.001070394 | 0.00554914 | 0.001370185 | 0 | 0.00115986 | 4.49E−05 | 0.000187967 | 0.00030384 | 0.001149885 | 0.000581254 |
| G[T > G]C | 0.001530563 | 0 | 0.001210163 | 5.08E−05 | 0.001929766 | 9.17E−05 | 0.000352937 | 0.000871542 | 0.000739926 | 0.000637278 |
| G[T > G]G | 0.003131152 | 0.008688653 | 0.002350318 | 0.004738015 | 0.003129621 | 0.000770113 | 0.001379755 | 0.002548659 | 0.003009699 | 0.00542237 |
| G[T > G]T | 0.002680987 | 0.002769571 | 0.006050817 | 0.002299037 | 0.020597508 | 0.001150169 | 0.000159972 | 0.002808523 | 0.000784922 | 0.000451197 |
| T[C > A]A | 0.02981097 | 0.006938924 | 0.000938127 | 0.007077035 | 0.01429827 | 0.045206624 | 0.000695877 | 0.001859022 | 0.009729028 | 0.011905203 |
| T[C > A]C | 0.051919106 | 0.010198419 | 0.000630085 | 0.006267375 | 0.000451945 | 0.041106023 | 0.000242957 | 0.009375069 | 0.009289072 | 0.005112234 |
| T[C > A]G | 0.009793604 | 0 | 0.000123017 | 0.001449393 | 0.000128984 | 0.01740255 | 8.26E−05 | 0.000954498 | 0.002379762 | 0.001370599 |
| T[C > A]T | 0.022808393 | 0.024596188 | 0.001400189 | 0.009805892 | 0.000556933 | 0.041106023 | 0.000664882 | 0.00448764 | 0.009639037 | 0.012805596 |
| T[C > G]A | 0.003751381 | 0.009798481 | 0.001310177 | 0.002868798 | 0.000114986 | 0.000107016 | 0.000126977 | 0.000395792 | 4.71E−18 | 0.013005683 |
| T[C > G]C | 0.005582054 | 0.003269493 | 0.000745101 | 0.003108698 | 0.000135984 | 0.006831001 | 0.000985825 | 0.002918465 | 0.00109989 | 0.002511097 |
| T[C > G]G | 0.001350497 | 0.002059681 | 9.41E−05 | 1.28E−05 | 2.46E−05 | 0.000897131 | 0.000105981 | 0.000415781 | 0.0009969 | 8.04E−05 |
| T[C > G]T | 0.004071498 | 0.016397458 | 0.002210299 | 0.002129108 | 0.000142983 | 0.000336049 | 0.000108981 | 0.004237771 | 0.000691931 | 0.020108788 |
| T[C > T]A | 0.002400884 | 0.01109828 | 0.003290445 | 0.018792128 | 0.004839414 | 0.006390936 | 0.097982618 | 0.007476068 | 0.024797523 | 0.014706427 |
| T[C > T]C | 0.012204491 | 0.006449 | 0.002530342 | 0.007986654 | 0.0041295 | 3.61E−18 | 0.133976233 | 0.024587067 | 0.038696134 | 0.003171386 |
| T[C > T]G | 0.00269099 | 0.014097815 | 0.002630355 | 0.00460807 | 0.001739789 | 0.01440211 | 0.014697393 | 0.008405579 | 0.009849016 | 0.003851683 |
| T[C > T]T | 0.004711734 | 0.012398078 | 0.002130288 | 0.011895017 | 0.001989759 | 0.004280627 | 0.052490688 | 0.019489748 | 0.066993307 | 0.009043952 |
| T[T > A]A | 0.004121517 | 0.021796622 | 0.001130153 | 0.052777891 | 0.000187977 | 0.000764112 | 0.00028195 | 0.006696478 | 0.000511949 | 0.004101792 |
| T[T > A]C | 0.003271204 | 0.003409472 | 0.00126017 | 4.71E−05 | 0.001249849 | 0.001930283 | 0.000240957 | 0.003748029 | 0.002449755 | 0.000217095 |
| T[T > A]G | 0.001990733 | 0.010198419 | 0.000278038 | 0 | 0.000320961 | 0.000924135 | 0.000189966 | 0.003268281 | 0.001759824 | 0.000660289 |
| T[T > A]T | 0.002190806 | 0.014497753 | 0.001310177 | 0.001929192 | 0.005719308 | 0.001200176 | 0.000700876 | 0.008875332 | 0.000710929 | 0.002721189 |
| T[T > C]A | 0.001460537 | 0.014497753 | 0.042305716 | 0.00171928 | 0.001309842 | 0.00259038 | 0.000543904 | 0.004587587 | 0.00089691 | 0.011004809 |
| T[T > C]C | 0.003001104 | 0.007408852 | 0.031404243 | 0.003488539 | 0.002049752 | 0.00150022 | 0.000285949 | 0.010294585 | 0.001219878 | 0.012505465 |
| T[T > C]G | 0.002030747 | 0.004889242 | 0.030304094 | 0.001149518 | 0.001409829 | 0.000379056 | 0.000202964 | 0.004007892 | 0.001689831 | 0.320139901 |
| T[T > C]T | 0.003671351 | 0.016997365 | 0.057407756 | 0.001529359 | 0.002059751 | 0.00082212 | 0.000444921 | 0.012093639 | 0.000683932 | 0.009123987 |
| T[T > G]A | 0.000123045 | 0.002079678 | 0.001430193 | 0.001189502 | 0.042294882 | 0.000467068 | 0.000117979 | 0.002248817 | 2.26E−05 | 0.000468205 |
| T[T > G]C | 0.002640972 | 0.00057891 | 0.001720232 | 0.000279883 | 0.035695681 | 9.93E−05 | 9.64E−05 | 0.002378749 | 0.000223978 | 0.000164072 |
| T[T > G]G | 0.003041119 | 0.009428539 | 0.002840384 | 0.002349016 | 0.013998306 | 0.000180026 | 0.000812856 | 0.005347187 | 0.000817918 | 0.000959419 |
| T[T > G]T | 0.003961458 | 0.00786878 | 0.009451277 | 0.000139941 | 0.478942048 | 0.000928136 | 0.008888423 | 0.007296162 | 0.000634937 | 0.002250984 |
| Type | SBS34 | SBS35 | SBS36 | SBS37 | SBS38 | SBS39 | SBS40 | SBS41 | SBS42 | SBS43 |
| A[C > A]A | 0.00486795 | 0.008826949 | 0.025193777 | 0.003950822 | 0.012794869 | 0.011701521 | 0.028203751 | 0.002110193 | 0.001160252 | 0.029506292 |
| A[C > A]C | 0.006957071 | 0.046184034 | 0.008317945 | 0.001450302 | 0.010095952 | 0.00715093 | 0.013401782 | 0.001220112 | 0.020604479 | 0.005901258 |
| A[C > A]G | 5.20E−05 | 0.00138952 | 0.002239447 | 0.001060221 | 0.001899238 | 0.002670347 | 0.002920388 | 6.14E−05 | 3.34E−05 | 0.000626134 |
| A[C > A]T | 0.001229482 | 0.021592535 | 0.01789558 | 0.001850385 | 0.008846453 | 0.007400962 | 0.014801969 | 0.001330122 | 0.007971733 | 0.002070442 |
| A[C > G]A | 0.000761679 | 0.003578763 | 0.001839546 | 0.034307136 | 0.002688922 | 0.04730615 | 0.012201623 | 0.005330488 | 0.000406088 | 0.000361077 |
| A[C > G]C | 0.001129524 | 0.004548428 | 0.001669588 | 0.01120233 | 0.001579367 | 0.022502925 | 0.008681155 | 0.003160289 | 0.00050611 | 3.13E−05 |
| A[C > G]G | 2.22E−16 | 0.00034588 | 0.000499877 | 0.005351113 | 0.000635745 | 0.013601768 | 0.002250299 | 0.001210111 | 1.77E−05 | 0.004430945 |
| A[C > G]T | 0.002029146 | 0.009476724 | 0.001909528 | 0.018803911 | 0.002439022 | 0.043405643 | 0.014601942 | 0.008270758 | 0.000431094 | 0.000354076 |
| A[C > T]A | 0.003518518 | 0.002209236 | 0.005958528 | 0.006241298 | 0.003448617 | 0.009601248 | 0.01970262 | 0.009880905 | 0.022404871 | 2.90E−05 |
| A[C > T]C | 0.003708438 | 0.003988621 | 0.00327919 | 0.003060637 | 0.002149138 | 0.004040525 | 0.01210161 | 0.003360308 | 0.039808654 | 0.000128027 |
| A[C > T]G | 0.00011795 | 0.0005798 | 0.007068254 | 0.006331317 | 0.005177924 | 0.00600078 | 0.000267036 | 0.003430314 | 0.000440096 | 0.00122026 |
| A[C > T]T | 0.003868371 | 0.000899689 | 0.004308936 | 0.005651175 | 0.002439022 | 0.012801664 | 0.016902248 | 0.006350582 | 0.035207654 | 0.000910194 |
| A[T > A]A | 0.102956648 | 0.00488831 | 0.002439397 | 0.002250468 | 0.001989202 | 0.00531069 | 0.006100811 | 0.007460683 | 0.013302892 | 0.015303263 |
| A[T > A]C | 0.044381312 | 0.007357457 | 0.001429647 | 0.001660345 | 0.001939222 | 0.002880374 | 0.004440591 | 0.004300394 | 0.004010872 | 0.003780806 |
| A[T > A]G | 0.034585437 | 0.00132954 | 0.002529375 | 0.002490518 | 0.001079567 | 0.006940902 | 0.006930922 | 0.003030278 | 0.005011089 | 0.001430305 |
| A[T > A]T | 0.315866997 | 0.007367453 | 0.003069242 | 0.002770576 | 0.00271891 | 0.006120796 | 0.010701423 | 0.069606376 | 0.00685149 | 0.004280913 |
| A[T > C]A | 0.004618055 | 0.002039295 | 0.005248704 | 0.07331525 | 0.002788882 | 0.011201456 | 0.018702487 | 0.014401319 | 0.011602522 | 8.88E−06 |
| A[T > C]C | 0.001229482 | 0.000820716 | 0.001689583 | 0.038407989 | 0.001639343 | 0.005640733 | 0.007340976 | 0.00579053 | 0.003510763 | 0.031306676 |
| A[T > C]G | 1.95E−05 | 0.000919682 | 0.003839052 | 0.016803495 | 0.00216913 | 0.009641253 | 0.015502062 | 0.008040737 | 0.008561861 | 0.000586125 |
| A[T > C]T | 0.002339015 | 0.002029298 | 0.005118736 | 0.048009986 | 0.003488601 | 0.013601768 | 0.019402581 | 0.017701621 | 0.002700587 | 0.008801877 |
| A[T > G]A | 0.002818813 | 0.003048946 | 0.001089731 | 0.021704515 | 0.001149539 | 0.003880504 | 0.007230962 | 0.00753069 | 0.001200261 | 0.002630561 |
| A[T > G]C | 0.000461806 | 0.000965666 | 0.000622846 | 0.010602205 | 0.001069571 | 0.002970386 | 0.007050938 | 0.00164015 | 0.000405088 | 0.004070868 |
| A[T > G]G | 0.000196917 | 0.004478452 | 0.000935769 | 0.016803495 | 0.00206917 | 0.007660996 | 0.008061072 | 0.002700247 | 0.000637139 | 0.034907444 |
| A[T > G]T | 0.001149516 | 0.002629091 | 0.000983757 | 0.024705139 | 0.002858854 | 0.006420835 | 0.018002394 | 0.007610697 | 0.000911198 | 0.005641203 |
| C[C > A]A | 0.001949179 | 0.029889667 | 0.055786221 | 0.00322067 | 0.38084728 | 0.010101313 | 0.020802767 | 0.002380218 | 0.011602522 | 0.006631414 |
| C[C > A]C | 3.67E−05 | 0.032988596 | 0.020195012 | 0.000814169 | 0.069472142 | 0.009061178 | 0.014701955 | 0.000919084 | 0.032807132 | 8.20E−05 |
| C[C > A]G | 0.000297875 | 0.00219924 | 0.007098247 | 0.000747155 | 0.050979557 | 0.005740746 | 0.002540338 | 0.000261024 | 0.001880409 | 2.56E−05 |
| C[C > A]T | 0.000619739 | 0.08247149 | 0.06048506 | 0.001770368 | 0.183926246 | 0.006030784 | 0.014701955 | 0.002650243 | 0.021104588 | 5.22E−18 |
| C[C > G]A | 0.00011895 | 0.003858666 | 0.001239694 | 0.000442092 | 0.001689323 | 0.028803744 | 0.007561006 | 0.002080191 | 0.004260926 | 2.09E−05 |
| C[C > G]C | 0.00011795 | 0.006297823 | 0.001369662 | 0.000313065 | 0.001259495 | 0.037704902 | 0.009351244 | 0.00120011 | 0.00662144 | 0.000212045 |
| C[C > G]G | 0.00018992 | 0.000869699 | 0.000418897 | 0.000321067 | 0.000956616 | 0.012301599 | 0.001680223 | 0.000411038 | 0.000470102 | 0.002540542 |
| C[C > G]T | 7.36E−05 | 0.016194402 | 0.002089484 | 0.000581121 | 0.001269491 | 0.040205227 | 0.013901849 | 0.005030461 | 0.006841487 | 0.000853182 |
| C[C > T]A | 0.004748001 | 0.005098238 | 0.006748333 | 0.003610751 | 0.0042383 | 0.009111184 | 0.019602607 | 0.006910633 | 0.018704066 | 7.62E−06 |
| C[C > T]C | 0.000513784 | 0.083171248 | 0.00667835 | 0.002100437 | 0.004728104 | 0.003860502 | 0.017302301 | 0.001850169 | 0.048110459 | 4.35E−18 |
| C[C > T]G | 0.009446023 | 0.002669077 | 0.005328684 | 0.00327068 | 0.003498597 | 0.007570984 | 0.002360314 | 0.010100925 | 0.001920417 | 9.72E−05 |
| C[C > T]T | 3.53E−05 | 0.046983758 | 0.00574858 | 0.006061261 | 0.004098357 | 0.006990909 | 0.021602873 | 0.007730708 | 0.032907154 | 0.000744159 |
| C[T > A]A | 0.023690025 | 0.052581822 | 0.001259689 | 0.001570327 | 0.00089664 | 0.004940642 | 0.001090145 | 0.003620332 | 0.01260274 | 0.00136029 |
| C[T > A]C | 0.000408828 | 0.032988596 | 0.001459639 | 0.001160241 | 0.00201919 | 0.006440837 | 0.007621014 | 0.003190292 | 0.001720374 | 1.21E−05 |
| C[T > A]G | 0.001169508 | 0.058179887 | 0.002139472 | 0.001600333 | 0.001779287 | 0.006800884 | 0.00346046 | 0.003120286 | 0.01490324 | 0.000296063 |
| C[T > A]T | 0.000267887 | 0.066377053 | 0.001959516 | 0.002060429 | 0.002359054 | 0.008271075 | 0.009281234 | 0.009010825 | 0.007571646 | 0.000203043 |
| C[T > C]A | 0.00059375 | 0.00132954 | 0.00149963 | 0.050010402 | 0.001949218 | 0.007340954 | 0.01090145 | 0.010300944 | 0.00354077 | 0.00272058 |
| C[T > C]C | 4.04E−05 | 0.002019302 | 0.001879536 | 0.021804535 | 0.00231907 | 0.007911028 | 0.009871313 | 0.005250481 | 0.003130681 | 0.105022396 |
| C[T > C]G | 0.000810659 | 0.001559461 | 0.001849543 | 0.00995207 | 0.002538982 | 0.00777101 | 0.010501397 | 0.00557051 | 0.003560774 | 0.009251973 |
| C[T > C]T | 0.001589331 | 0.003178901 | 0.002159467 | 0.050410485 | 0.004268288 | 0.008591117 | 0.013601809 | 0.007870721 | 0.003240705 | 0.009452016 |
| C[T > G]A | 0.001059554 | 0.003708718 | 0.000477882 | 0.002600541 | 0.000982606 | 0.005300689 | 0.003350446 | 0.003470318 | 0.000870189 | 0.002730582 |
| C[T > G]C | 2.22E−16 | 0.000517821 | 0.00056586 | 0.002380495 | 0.001409435 | 0.007390961 | 0.005670754 | 0.002410221 | 0.000753164 | 0.007261549 |
| C[T > G]G | 0.000435816 | 0.006357802 | 0.000886781 | 0.003770784 | 0.001589363 | 0.009831278 | 0.005590744 | 0.003040278 | 0.005681235 | 0.046509918 |
| C[T > G]T | 6.55E−07 | 0.002709063 | 0.001069736 | 0.004110855 | 0.006877242 | 0.004940642 | 0.013101743 | 0.010500962 | 0.003480757 | 6.26E−18 |
| G[C > A]A | 0.002808817 | 0.009866589 | 0.067683282 | 0.002040424 | 0.01199519 | 0.006880895 | 0.014201889 | 0.003090283 | 0.007711677 | 0.009301984 |
| G[C > A]C | 0.000467803 | 0.050182652 | 0.019195259 | 0.000629131 | 0.006277483 | 0.006950904 | 0.010201357 | 0.000723066 | 0.026805828 | 0.00169036 |
| G[C > A]G | 0.000226904 | 0.000506825 | 0.004828807 | 0.000506105 | 0.001369451 | 0.004530589 | 0.002550339 | 0.00022002 | 0.00106023 | 0.000177038 |
| G[C > A]T | 1.35E−05 | 0.022892086 | 0.068982961 | 0.001290268 | 0.009086356 | 0.005890766 | 0.010801437 | 0.001950179 | 0.018103936 | 0.001390296 |
| G[C > G]A | 3.34E−05 | 0.003378832 | 0.000885781 | 0.001320275 | 0.001199519 | 0.02400312 | 0.005060673 | 0.002040187 | 0.002400522 | 0.013602901 |
| G[C > G]C | 0.000192919 | 0.021392605 | 0.001079733 | 0.000938195 | 0.001079567 | 0.021302769 | 0.005370714 | 0.002690246 | 0.001420309 | 0.015203242 |
| G[C > G]G | 2.22E−16 | 0.000590796 | 0.000171958 | 0.00057712 | 0.001019591 | 0.009861282 | 0.001190158 | 0.000156014 | 0.000223048 | 0.01280273 |
| G[C > G]T | 2.69E−05 | 0.00873698 | 0.001049741 | 0.001270264 | 0.000916632 | 0.032404213 | 0.006980928 | 0.003560326 | 0.002770602 | 0.018003839 |
| G[C > T]A | 0.000232902 | 0.007287481 | 0.003719081 | 0.002550531 | 0.00236905 | 0.008031044 | 0.014301902 | 0.006010551 | 0.033607306 | 0.000766163 |
| G[C > T]C | 2.22E−16 | 0.017793849 | 0.00339916 | 0.002310481 | 0.002958814 | 0.004500585 | 0.011601543 | 0.008640791 | 0.152033052 | 4.10E−18 |
| G[C > T]G | 0.016692971 | 0.002529126 | 0.005698592 | 0.004320899 | 0.004478204 | 0.005480712 | 3.93E−18 | 0.017101567 | 0.004550989 | 9.66E−06 |
| G[C > T]T | 0.000516782 | 0.006027916 | 0.003629104 | 0.002320483 | 0.003198717 | 0.008611119 | 0.012701689 | 0.00841077 | 0.086518809 | 0.001860397 |
| G[T > A]A | 0.018992003 | 0.000716752 | 0.000879783 | 0.001150239 | 0.000892642 | 0.004960645 | 0.002650352 | 0.002330213 | 0.012002609 | 0.006181318 |
| G[T > A]C | 0.002418981 | 0.005558079 | 0.000630844 | 0.000774161 | 0.001119551 | 0.003520458 | 0.003430456 | 0.00229021 | 0.0018404 | 0.001330284 |
| G[T > A]G | 0.006527252 | 0.001119613 | 0.001169711 | 0.001080225 | 0.001209515 | 0.006670867 | 0.004410587 | 0.001430131 | 0.00777169 | 0.00450096 |
| G[T > A]T | 0.007476852 | 0.002859012 | 0.001119723 | 0.00168035 | 0.001839262 | 0.004090532 | 0.005770768 | 0.004520414 | 0.003250707 | 0.001310279 |
| G[T > C]A | 0.000108954 | 0.000564805 | 0.002339422 | 0.030706387 | 0.001699319 | 0.007470971 | 0.006010799 | 0.006060555 | 0.005821266 | 7.50E−18 |
| G[T > C]C | 0.000645728 | 0.002029298 | 0.001399654 | 0.018203786 | 0.002159134 | 0.006890896 | 0.004580609 | 0.003740343 | 0.00331072 | 0.021904671 |
| G[T > C]G | 0.000439815 | 0.000190934 | 0.001839546 | 0.009832045 | 0.00186925 | 0.005740746 | 0.005650752 | 0.003510322 | 0.003210698 | 0.001200256 |
| G[T > C]T | 2.22E−16 | 0.000420855 | 0.002649346 | 0.040008322 | 0.003048777 | 0.007510976 | 0.007310972 | 0.003890356 | 0.00239052 | 0.00051811 |
| G[T > G]A | 0.000364846 | 0.001759392 | 0.000184954 | 0.011802455 | 0.000503798 | 0.003330433 | 0.002490331 | 0.001800165 | 0.001140248 | 0.061113032 |
| G[T > G]C | 2.22E−16 | 0.005068248 | 0.000312923 | 0.006861427 | 0.000938624 | 0.005800754 | 0.002240298 | 0.000950087 | 7.05E−05 | 0.075616125 |
| G[T > G]G | 5.65E−05 | 0.004038604 | 0.001529622 | 0.009832045 | 0.002159134 | 0.007721004 | 0.004450592 | 0.00120011 | 0.000410089 | 0.241051404 |
| G[T > G]T | 0.000416824 | 0.003348842 | 0.000488879 | 0.017103558 | 0.003338661 | 0.005940772 | 0.005480729 | 0.003470318 | 0.0009202 | 0.074615912 |
| T[C > A]A | 0.004268203 | 0.005268179 | 0.140965182 | 0.002000416 | 0.017692905 | 0.005980778 | 0.026003458 | 0.003310303 | 0.005101109 | 0.006301344 |
| T[C > A]C | 0.001219487 | 0.023092017 | 0.077180936 | 0.001590331 | 0.01149539 | 0.007861022 | 0.021002793 | 0.001720158 | 0.054011742 | 5.37E−06 |
| T[C > A]G | 2.22E−16 | 9.47E−05 | 0.01469637 | 0.001120233 | 0.002379046 | 0.003010391 | 0.003170422 | 0.000207019 | 0.000904197 | 3.07E−18 |
| T[C > A]T | 3.20E−05 | 0.015294713 | 0.207948637 | 0.002440508 | 0.027788857 | 0.006550852 | 0.024103206 | 0.00590054 | 0.027305936 | 6.52E−18 |
| T[C > G]A | 6.18E−07 | 0.006627709 | 0.003979017 | 0.001790372 | 0.002658934 | 0.034704512 | 0.012501663 | 0.019901823 | 0.000559122 | 4.42E−06 |
| T[C > G]C | 0.000139941 | 0.005628054 | 0.002409405 | 0.001110231 | 0.002179126 | 0.036704772 | 0.014101876 | 0.014201301 | 0.001870407 | 0.00103022 |
| T[C > G]G | 2.22E−16 | 0.000154946 | 0.000301925 | 0.000860179 | 0.001099559 | 0.009621251 | 0.001740231 | 0.001930177 | 0.000207045 | 0.001470314 |
| T[C > G]T | 0.002149095 | 0.021192674 | 0.007588126 | 0.002550531 | 0.003948417 | 0.050706592 | 0.023703153 | 0.044604086 | 0.000381083 | 0.000251054 |
| T[C > T]A | 0.001859217 | 0.002958977 | 0.010697358 | 0.004020836 | 0.006447415 | 0.003880504 | 0.030003991 | 0.011701072 | 0.004821048 | 3.72E−05 |
| T[C > T]C | 0.000288878 | 0.012595646 | 0.006858306 | 0.00250052 | 0.007227102 | 0.005280686 | 0.023103073 | 0.006470593 | 0.032707111 | 5.06E−18 |
| T[C > T]G | 0.000299874 | 0.000764736 | 0.003889039 | 0.003430714 | 0.003468609 | 0.004860632 | 0.002120282 | 0.004920451 | 0.000364079 | 3.07E−18 |
| T[C > T]T | 0.00080766 | 0.008037222 | 0.008327943 | 0.005841215 | 0.006577362 | 0.00423055 | 0.02240298 | 0.011501053 | 0.015103283 | 0.000836178 |
| T[T > A]A | 0.271885516 | 0.004298514 | 0.002459393 | 0.00245051 | 0.002439022 | 0.00669087 | 0.007130948 | 0.080607384 | 0.008501848 | 7.63E−05 |
| T[T > A]C | 2.22E−16 | 0.005298168 | 0.001019748 | 0.001130235 | 0.000927628 | 0.00369048 | 0.01030137 | 0.01430131 | 0.002260491 | 0.0009382 |
| T[T > A]G | 0.00776673 | 0.001379523 | 0.000871785 | 0.001280266 | 0.000803678 | 0.00546071 | 0.005020668 | 0.009290851 | 0.005331159 | 5.14E−05 |
| T[T > A]T | 0.045980639 | 0.00540813 | 0.002699333 | 0.001950406 | 0.002498998 | 0.010701391 | 0.021002793 | 0.053504901 | 0.003520765 | 8.77E−18 |
| T[T > C]A | 0.001399411 | 0.000367873 | 0.002969267 | 0.047709924 | 0.002109154 | 0.005940772 | 0.010201357 | 0.063605826 | 0.006821483 | 0.000137029 |
| T[T > C]C | 0.006237374 | 0.000821716 | 0.002279437 | 0.045109383 | 0.002189122 | 0.006440837 | 0.007551004 | 0.026802455 | 0.003030659 | 0.035907657 |
| T[T > C]G | 0.001099537 | 0.000437849 | 0.001379659 | 0.01000208 | 0.00191923 | 0.004980647 | 0.006010799 | 0.020501878 | 0.003410741 | 2.78E−05 |
| T]T > C]T | 0.00707702 | 0.001059634 | 0.002799309 | 0.061412774 | 0.003058773 | 0.009481233 | 0.010201357 | 0.046104223 | 0.002360513 | 0.001840392 |
| T[T > G]A | 2.22E−16 | 0.004288517 | 0.000787805 | 0.018303807 | 0.001169531 | 0.008391091 | 0.008461125 | 0.041903838 | 0.00115025 | 0.000204044 |
| T[T > G]C | 2.22E−16 | 0.00193933 | 0.000743816 | 0.019504057 | 0.000902638 | 0.00508066 | 0.011401516 | 0.01550142 | 0.00027406 | 0.00825176 |
| T[T > G]G | 0.00151936 | 0.004818334 | 0.000901777 | 0.030206283 | 0.001899238 | 0.007280947 | 0.008661152 | 0.019401777 | 0.002930637 | 0.022804863 |
| T[T > G]T | 0.000231902 | 0.003618749 | 0.001809553 | 0.029106054 | 0.00528788 | 0.010901417 | 0.025003325 | 0.087908052 | 0.001740378 | 0.001320282 |
| Type | SBS44 | SBS45 | SBS46 | SBS47 | SBS48 | SBS49 | SBS50 | SBS51 | SBS52 | SBS53 |
| A[C > A]A | 7.68E−18 | 0.009108136 | 0.0044 | 0.067787874 | 0.000855468 | 0.025116627 | 0.119000274 | 0.140963913 | 0.015196277 | 0.00538351 |
| A[C > A]C | 0.000150038 | 0.002849417 | 0.0047 | 0.029694688 | 0.00084146 | 0.007034657 | 0.127000292 | 0.001689567 | 0.006538398 | 0.001961279 |
| A[C > A]G | 9.16E−07 | 0.00165966 | 3.00E−04 | 0.001379753 | 0.000258141 | 0.00711471 | 0.005770013 | 0.004798772 | 0.004138986 | 0.038124854 |
| A[C > A]T | 0.005781464 | 0.009638027 | 0.0034 | 0.011897872 | 3.89E−05 | 0.00797528 | 0.083200191 | 0.021194574 | 0.009237737 | 1.71E−05 |
| A[C > G]A | 0.003180806 | 0.003109364 | 0.0012 | 0.001029816 | 0.000198108 | 0.000288191 | 0.003850009 | 0.003958986 | 0.001729576 | 0.007034586 |
| A[C > G]C | 0.000233059 | 0.001929605 | 0.0015 | 0.00061689 | 0.000114062 | 0.000135089 | 0.001100003 | 0.004538838 | 0.001519628 | 0.005463562 |
| A[C > G]G | 3.14E−05 | 0.000269945 | 0 | 0.000327941 | 8.03E−05 | 0.000204135 | 0.000827002 | 0.001969496 | 0.0008178 | 0.002511637 |
| A[C > G]T | 0.003290833 | 0.001429707 | 0.0022 | 0.001719692 | 0.000686375 | 0.000176117 | 0.001970005 | 0.00089977 | 0.001709581 | 0.003041983 |
| A[C > T]A | 0.089922775 | 0.001329728 | 0.0152 | 0.004269236 | 0.000287157 | 0.000575381 | 0.003520008 | 0.035790838 | 0.002689341 | 0.00722471 |
| A[C > T]C | 0.016904281 | 0.000600877 | 0.0089 | 0.000939832 | 0.00043824 | 0.000303201 | 0.001800004 | 0.005648554 | 0.002099486 | 0.001961279 |
| A[C > T]G | 0.014303623 | 0.005408893 | 0.0046 | 0.003049455 | 0.001210662 | 0.002011332 | 0.013900032 | 0.005648554 | 0.004079001 | 0.006954534 |
| A[C > T]T | 0.030307676 | 0.00121975 | 0.0068 | 0.002489555 | 0.000235129 | 0.000415275 | 0.006920016 | 0.003199181 | 0.001759569 | 0.001941266 |
| A[T > A]A | 0.004781211 | 0.000524893 | 0.0027 | 0.137975319 | 0.000660361 | 0.000129085 | 0.005360012 | 0.112971079 | 0.001769566 | 0.005893842 |
| A[T > A]C | 0.002600659 | 0.000496898 | 0.0018 | 0.007158719 | 0.000266146 | 7.79E−05 | 0.003940009 | 0.001349654 | 0.001179711 | 0.00259169 |
| A[T > A]G | 1.08E−06 | 0.002129564 | 5.00E−04 | 0.007448668 | 0.000465255 | 0.00024116 | 0.000573001 | 0.036790582 | 0.001189709 | 0.002871872 |
| A[T > A]T | 0.030807803 | 0.000604876 | 0.002 | 0.06758791 | 0.000675369 | 0.000354235 | 0.002410006 | 0.02949245 | 0.001189709 | 0.00463302 |
| A[T > C]A | 0.019304889 | 0.00136972 | 0.0185 | 0.002829494 | 0.001100602 | 0.000613406 | 0.002630006 | 0.002589337 | 0.003749081 | 0.011307371 |
| A[T > C]C | 0.003240821 | 0.000417914 | 0.0478 | 0.005668986 | 0.000189103 | 8.15E−05 | 0.009280021 | 0.01929506 | 0.00187954 | 0.003702414 |
| A[T > C]G | 0.027206891 | 0.002069576 | 0.0329 | 0.002089626 | 0.000285156 | 0.000200132 | 0.001130003 | 0.003948989 | 0.002619358 | 0.008255382 |
| A[T > C]T | 0.000267068 | 0.001459701 | 0.0306 | 0.001299767 | 0.001110608 | 0.000482319 | 0.004920011 | 0.001869521 | 0.004918795 | 0.007184684 |
| A[T > G]A | 2.40E−08 | 0.000116976 | 1.00E−04 | 0.000170969 | 0.000517283 | 0.00019613 | 0.000743002 | 0.000722815 | 0.001729576 | 0.004592994 |
| A[T > G]C | 3.26E−05 | 0.00058388 | 0.0008 | 0.001719692 | 4.10E−05 | 8.29E−05 | 0.000607001 | 0.004088953 | 0.00052887 | 0.002901892 |
| A[T > G]G | 7.47E−06 | 0.000983799 | 0.0016 | 0.003679342 | 0.000351192 | 0.000126083 | 0.003940009 | 0.027792885 | 0.001189709 | 0.005063301 |
| A[T > G]T | 0.00142036 | 0.000165966 | 6.00E−04 | 0.008078555 | 0.000199109 | 0.000126083 | 0.000103 | 0.002129455 | 0.001859544 | 0.002061344 |
| C[C > A]A | 2.84E−05 | 0.237951291 | 0.0156 | 0.0245956 | 0.001180646 | 0.229151698 | 0.022500052 | 0.006978214 | 0.05738594 | 0.334217877 |
| C[C > A]C | 0.030307676 | 0.150969097 | 0.0072 | 0.005269057 | 3.57E−05 | 0.028618946 | 0.002890007 | 0.003169189 | 0.018995346 | 0.003552316 |
| C[C > A]G | 0.003740947 | 0.067886104 | 0.0045 | 0.000270952 | 0.137074986 | 0.515341156 | 5.18E−18 | 0.004298899 | 0.013596669 | 0.067744162 |
| C[C > A]T | 0.086721965 | 0.193960296 | 0.0083 | 0.000681878 | 0.000845463 | 0.049032459 | 0.001150003 | 0.005068702 | 0.018795395 | 0.004923209 |
| C[C > G]A | 5.36E−05 | 0.001429707 | 5.00E−04 | 0.000955829 | 9.94E−05 | 0.000216143 | 6.56E−05 | 0.002849271 | 0.001179711 | 0.002721774 |
| C[C > G]C | 2.49E−05 | 0.000799836 | 3.00E−04 | 0.000236958 | 0.000122067 | 0.00025617 | 0.000289001 | 0.000119969 | 0.000834795 | 0.006033934 |
| C[C > G]G | 7.68E−18 | 0.000642868 | 1.00E−04 | 0.00066988 | 3.63E−05 | 0.000238158 | 0.001420003 | 0.00202948 | 0.000567861 | 0.001621057 |
| C[C > G]T | 8.64E−05 | 0.001909609 | 0.0015 | 0.000136975 | 0.000245134 | 0.000191127 | 0.000512001 | 0.002609332 | 0.001199706 | 0.000508331 |
| C[C > T]A | 0.003570904 | 0.002139562 | 0.0284 | 0.000863845 | 1.82E−05 | 0.000470311 | 0.003840009 | 0.003229173 | 0.003269199 | 0.014609524 |
| C[C > T]C | 0.002860725 | 5.07E−05 | 0.0133 | 0.004609176 | 0.000301165 | 0.000493327 | 0.00425001 | 0.004698797 | 0.001819554 | 0.004603001 |
| C[C > T]G | 0.015503927 | 0.00219955 | 0.0083 | 0.001519728 | 1.87E−05 | 0.001380914 | 0.001080002 | 0.020694702 | 0.002379417 | 0.013708937 |
| C[C > T]T | 0.005041277 | 0.002729441 | 0.0064 | 0.002999463 | 0.000479262 | 0.000519344 | 0.011900027 | 0.004058961 | 0.002569371 | 0.016010437 |
| C[T > A]A | 1.24E−05 | 0.000696857 | 0.0042 | 0.013997496 | 8.62E−05 | 7.41E−05 | 0.000193 | 0.006108436 | 0.001219701 | 0.003692407 |
| C[T > A]C | 0.003250823 | 0.001039787 | 0.0033 | 0.000570898 | 0.000102056 | 5.14E−05 | 0.000535001 | 0.001489619 | 0.000230943 | 0.004502935 |
| C[T > A]G | 0.000582147 | 0.003409302 | 0.0043 | 5.82E−05 | 0.00031017 | 0.000232154 | 0.000516001 | 0.006518331 | 0.001229699 | 0.003302153 |
| C[T > A]T | 1.38E−05 | 0.001339726 | 0.0036 | 7.60E−05 | 0.000362198 | 0.00010607 | 0.000209 | 0.003619074 | 0.00085579 | 0.008615617 |
| C[T > C]A | 0.014603699 | 0.001109773 | 0.0223 | 0.001909658 | 0.000145079 | 0.00012108 | 0.000839002 | 0.00417893 | 0.002389415 | 0.010106588 |
| C[T > C]C | 0.003200811 | 0.002869413 | 0.0834 | 0.006278877 | 0.000450246 | 0.000178118 | 0.017700041 | 0.071081803 | 0.001309679 | 0.004072655 |
| C[T > C]G | 0.033808563 | 0.001559681 | 0.0504 | 0.003099446 | 3.21E−05 | 0.000108072 | 0.002420006 | 0.00292925 | 0.001229699 | 0.008495538 |
| C[T > C]T | 0.007711953 | 0.000601877 | 0.0958 | 0.005449025 | 6.77E−05 | 9.75E−05 | 0.001380003 | 0.006638301 | 0.002339427 | 0.005423536 |
| C[T > G]A | 0.000234059 | 0.000101979 | 0 | 0.000136975 | 0.000134073 | 0.00010607 | 0.000894002 | 0.002779289 | 0.000802803 | 0.001490972 |
| C[T > G]C | 0.002530641 | 0.000280942 | 0.0018 | 5.15E−05 | 9.29E−05 | 2.67E−05 | 6.18E−05 | 0.002559345 | 0.00077481 | 0.004242766 |
| C[T > G]G | 0.003470879 | 0.000648867 | 0.0016 | 0.003669344 | 0.000104057 | 0.000112074 | 0.003660008 | 0.005928482 | 0.000795805 | 0.002981944 |
| C[T > G]T | 0.000384097 | 0.000727851 | 0.002 | 0.000106981 | 6.57E−05 | 9.30E−05 | 0.000471001 | 0.002299411 | 0.000862789 | 0.002721774 |
| G[C > A]A | 7.68E−18 | 0.007548455 | 0.0072 | 0.011098015 | 0.000752412 | 0.00788522 | 0.014200033 | 0.003938992 | 0.008237982 | 0.000432282 |
| G[C > A]C | 0.004671183 | 0.005728827 | 0.003 | 0.003479378 | 0.000346189 | 0.002481643 | 0.007580017 | 0.003279161 | 0.007318207 | 0.003442244 |
| G[C > A]G | 0.000605153 | 0.002089572 | 4.00E−04 | 0.00078286 | 0.000739404 | 0.006834524 | 0.000244001 | 0.004278905 | 0.00403901 | 0.050833138 |
| G[C > A]T | 0.011302863 | 0.006988569 | 0.0024 | 0.001409748 | 1.99E−05 | 0.005843869 | 0.002040005 | 0.003449117 | 0.004208969 | 0.001140744 |
| G[C > G]A | 0.000219055 | 0.000332932 | 1.00E−04 | 0.0016797 | 7.84E−05 | 0.000152101 | 0.026700061 | 0.003409127 | 0.001379662 | 0.0013809 |
| G[C > G]C | 0.001190301 | 0.001009793 | 0.0008 | 0.002279592 | 0.000217119 | 0.000110073 | 0.033000076 | 0.001139708 | 0.001449645 | 0.084755252 |
| G[C > G]G | 7.68E−18 | 0.000694858 | 0 | 0.000464917 | 4.31E−05 | 3.67E−05 | 0.002800006 | 0.000266932 | 0.001009753 | 0.003001957 |
| G[C > G]T | 0.001850469 | 0.000931809 | 6.00E−04 | 0.002219603 | 6.53E−05 | 0.000208138 | 0.014300033 | 0.002849271 | 0.00101975 | 0.000573374 |
| G[C > T]A | 0.095524194 | 0.000999795 | 0.0126 | 0.00105981 | 0.000345189 | 0.000184122 | 0.002420006 | 0.004468856 | 0.001779564 | 0.009185988 |
| G[C > T]C | 0.081420622 | 0.000932809 | 0.0045 | 0.001709694 | 7.39E−06 | 0.000118078 | 0.001580004 | 0.003089209 | 0.001219701 | 0.011907763 |
| G[C > T]G | 0.049612566 | 0.001509691 | 0.0015 | 0.001719692 | 0.000303166 | 0.001040689 | 0.005500013 | 0.004278905 | 0.002829307 | 0.005993907 |
| G[C > T]T | 0.066616872 | 0.001989593 | 0.004 | 0.004039277 | 0.00011006 | 0.000308204 | 0.016300037 | 0.005948477 | 0.00101975 | 0.003102022 |
| G[T > A]A | 0.001160294 | 0.000299939 | 0.0061 | 0.015997138 | 0.000130071 | 5.04E−05 | 0.000732002 | 0.020394779 | 0.000523872 | 0.004452903 |
| G[T > A]C | 0.002360598 | 0.000637869 | 0.001 | 0.000227959 | 0.000124068 | 9.11E−05 | 0.001050002 | 0.003849015 | 0.000398902 | 0.002701761 |
| G[T > A]G | 0.00221056 | 0.001029789 | 0.0024 | 1.31E−05 | 0.000143078 | 0.000110073 | 0.000199 | 0.004288902 | 0.001289684 | 0.003962583 |
| G[T > A]T | 0.003180806 | 0.000509896 | 0.0012 | 3.28E−06 | 0.000231126 | 0.000131087 | 0.000786002 | 0.000145963 | 0.000810801 | 0.003112029 |
| G[T > C]A | 0.02700684 | 0.000653866 | 0.0221 | 0.000416925 | 0.000157086 | 6.59E−05 | 0.001380003 | 0.001179698 | 0.002419407 | 0.00561366 |
| G[T > C]C | 0.001160294 | 0.000569883 | 0.0576 | 0.001089805 | 0.000129071 | 0.0001511 | 0.005380012 | 0.002269419 | 0.002049498 | 0.006684358 |
| G[T > C]G | 0.017304383 | 0.000906814 | 0.0353 | 0.003099446 | 4.67E−05 | 6.53E−05 | 0.003710009 | 0.004558833 | 0.000913776 | 0.00256167 |
| G[T > C]T | 0.011102812 | 0.001389716 | 0.0607 | 0.003189429 | 0.00011006 | 0.000175116 | 0.001170003 | 0.004008974 | 0.003109238 | 0.00699456 |
| G[T > G]A | 7.68E−18 | 0.00043991 | 0 | 0.021996065 | 7.64E−06 | 6.61E−05 | 0.00867002 | 0.00218944 | 0.001129723 | 0.004382857 |
| G[T > G]C | 1.83E−05 | 0.000519894 | 1.00E−04 | 0.01039814 | 0.000135074 | 4.37E−05 | 0.009590022 | 0.005298644 | 0.000560863 | 0.001741135 |
| G[T > G]G | 0.000103026 | 0.000178963 | 0.0012 | 0.027195135 | 6.63E−05 | 0.000147097 | 0.035100081 | 0.076180498 | 0.001369664 | 0.004733085 |
| G[T > G]T | 0.002800709 | 0.000495898 | 6.00E−04 | 0.08838419 | 1.15E−05 | 0.000102068 | 0.036500084 | 0.002089465 | 0.000863788 | 0.002031324 |
| T[C > A]A | 0.000853216 | 0.063986902 | 0.0123 | 0.016896977 | 0.12406787 | 0.032921794 | 0.019000044 | 0.035690863 | 0.463886348 | 0.002671742 |
| T[C > A]C | 0.002220562 | 0.064186861 | 0.0071 | 8.11E−05 | 0.003792074 | 0.005113385 | 0.000886002 | 0.002859268 | 0.046788537 | 0.003642374 |
| T[C > A]G | 3.15E−07 | 0.018296255 | 0.0025 | 2.39E−05 | 0.709388064 | 0.040526829 | 4.19E−05 | 0.004538838 | 0.133967178 | 0.000447292 |
| T[C > A]T | 0.016604205 | 0.061987311 | 0.003 | 0.000191966 | 0.000187102 | 0.008755796 | 0.001850004 | 0.000369905 | 0.060985059 | 0.000871568 |
| T[C > G]A | 0.00067317 | 0.003299325 | 0.0028 | 0.000137975 | 0.000137075 | 4.80E−05 | 0.000932002 | 0.002049475 | 0.001979515 | 0.00374244 |
| T[C > G]C | 9.40E−06 | 0.002009589 | 0.0024 | 0.001189787 | 0.000350192 | 0.000153101 | 0.000493001 | 0.003609076 | 0.001439647 | 0.004993255 |
| T[C > G]G | 7.68E−18 | 0.000177964 | 1.00E−04 | 0 | 2.82E−05 | 6.33E−05 | 0.000103 | 0.002159447 | 0.000580858 | 0.00187122 |
| T[C > G]T | 0.002590656 | 0.003289327 | 0.0049 | 0.000923835 | 0.000148081 | 0.00054436 | 0.000421001 | 0.003329148 | 0.002419407 | 0.00374244 |
| T[C > T]A | 0.001400355 | 0.002399509 | 0.0185 | 0.007238705 | 0.000214117 | 0.000187124 | 0.019400045 | 0.001699565 | 0.00146964 | 0.005733738 |
| T[C > T]C | 0.00225057 | 0.002019587 | 0.0126 | 0.037093365 | 0.000317174 | 0.000582386 | 0.104000239 | 0.001769547 | 0.001529625 | 0.005773764 |
| T[C > T]G | 7.68E−18 | 0.001749642 | 0.0037 | 0.001939653 | 0.00040222 | 0.000934619 | 0.003970009 | 0.006488339 | 0.002189464 | 0.010206654 |
| T[C > T]T | 0.003020765 | 0.002949396 | 0.0044 | 0.080885531 | 5.85E−06 | 0.000312207 | 0.081800188 | 0.042389148 | 0.001649596 | 0.007715029 |
| T[T > A]A | 0.006961763 | 0.000406917 | 0.0045 | 0.1559721 | 2.35E−05 | 0.000420278 | 0.001690004 | 0.020094856 | 0.001049743 | 0.003102022 |
| T[T > A]C | 2.07E−05 | 0.000261946 | 0.0043 | 1.23E−05 | 7.64E−06 | 5.70E−05 | 0.000664002 | 0.00257934 | 0.000468885 | 0.003562322 |
| T[T > A]G | 5.02E−06 | 0.000653866 | 5.00E−04 | 0.000451919 | 8.32E−06 | 7.53E−05 | 7.54E−05 | 0.004028969 | 0.000777809 | 0.001601044 |
| T[T > A]T | 0.013903521 | 0.001389716 | 0.0034 | 0.004349222 | 0.000426233 | 0.000331219 | 0.002950007 | 0.006378367 | 0.001339672 | 0.005083314 |
| T[T > C]A | 0.019905041 | 0.001279738 | 0.0093 | 0.001369755 | 0.000605331 | 0.000173115 | 0.001090003 | 0.004458859 | 0.002769322 | 0.007114638 |
| T[T > C]C | 0.01050266 | 0.001099775 | 0.0543 | 0.01749687 | 0.000404221 | 8.48E−05 | 0.029000067 | 0.003928994 | 0.002179466 | 0.005033281 |
| T[T > C]G | 0.019104839 | 0.001239746 | 0.0237 | 0.001579717 | 0.000297163 | 6.31E−05 | 0.001020002 | 0.002469368 | 0.001069738 | 0.004723079 |
| T[T > C]T | 0.037109399 | 0.000544888 | 0.0365 | 0.011497943 | 0.000532291 | 0.000208138 | 0.005110012 | 0.021294549 | 0.00199951 | 0.002191429 |
| T[T > G]A | 7.68E−18 | 0.000284942 | 7.00E−04 | 0.002409569 | 0.000250137 | 0.000150099 | 0.000327001 | 0.002549347 | 0.00142965 | 0.001300848 |
| T[T > G]C | 7.68E−18 | 0.000134972 | 0.001 | 2.25E−05 | 0.000222122 | 0.000138091 | 0.000655002 | 0.003469112 | 0.001119726 | 0.004362844 |
| T[T > G]G | 0.000917232 | 0.000707855 | 0.0009 | 0.001539725 | 0.000350192 | 0.000112074 | 0.00872002 | 0.002759294 | 0.001479637 | 0.006154012 |
| T[T > G]T | 0.00900228 | 0.000723852 | 0.001 | 0.003619353 | 3.20E−07 | 0.000364241 | 0.001600004 | 0.002909255 | 0.00252938 | 0.007054599 |
| Type | SBS54 | SBS55 | SBS56 | SBS57 | SBS58 | SBS59 | SBS60 | SBS84 | SBS85 | SBS86 |
| A[C > A]A | 0.002160358 | 0.005881126 | 0.012597482 | 0.012306325 | 0.058874619 | 0.003588208 | 0.006152707 | 0.003471995 | 0.006080257 | 0.002954301 |
| A[C > A]C | 0.000796132 | 0.002050393 | 0.015696862 | 0.001430735 | 0.006747091 | 0.002368817 | 0.000779343 | 0.005007309 | 0.000879937 | 0.003774501 |
| A[C > A]G | 0.001640271 | 4.49E−05 | 0.000205959 | 0.003331713 | 0.000824644 | 0.000141929 | 2.23E−16 | 0.000452642 | 0.000306011 | 0.00038549 |
| A[C > A]T | 0.000414069 | 0.001170224 | 0.022995403 | 0.008644443 | 0.005577595 | 0.014492763 | 0.000444195 | 0.009295947 | 0.002717279 | 0.003624001 |
| A[C > G]A | 0.001550257 | 0.002850546 | 0.000417916 | 0.01540792 | 0.020591123 | 0.002938533 | 0.000377166 | 0.006758076 | 0.007234927 | 0.052516013 |
| A[C > G]C | 0.000496082 | 0.000168032 | 0.000165967 | 0.00313161 | 0.00173925 | 0.002438782 | 0.000588259 | 0.010811956 | 0.002551355 | 0.078321019 |
| A[C > G]G | 0.001700281 | 0.000510098 | 0.000427914 | 0.002771425 | 0.001689272 | 0.000829586 | 0.000276121 | 4.60E−05 | 0.000422197 | 0.004965301 |
| A[C > G]T | 0.005180857 | 0.001270243 | 0.000122975 | 0.005072607 | 0.002029125 | 0.003828088 | 0.00025011 | 0.029594214 | 0.013019761 | 0.092082022 |
| A[C > T]A | 0.001480245 | 0.005110978 | 0.007398521 | 0.032616765 | 0.080465311 | 0.003918044 | 0.002761215 | 0.053885765 | 0.001374781 | 0.008724302 |
| A[C > T]C | 0.000529088 | 0.000233045 | 0.000981804 | 0.012906634 | 0.006757087 | 0.002858573 | 0.00134059 | 0.042106829 | 0.001478442 | 0.0020495 |
| A[C > T]G | 0.000343057 | 0.005321019 | 0.001519696 | 0.017609051 | 0.008626281 | 0.002558722 | 0.004211853 | 0.002540673 | 0.000443776 | 0.024091006 |
| A[C > T]T | 0.004990826 | 0.000776149 | 0.002839432 | 0.01540792 | 0.00730685 | 0.003388308 | 0.000298131 | 0.05434587 | 0.004563583 | 0.004575101 |
| A[T > A]A | 0.000956158 | 0.004260816 | 5.05E−06 | 0.003281687 | 0.024489443 | 0.001999002 | 0.003971747 | 0.001562339 | 0.111858721 | 0.0018477 |
| A[T > A]C | 0.003190528 | 0.000618118 | 0.004289143 | 0.006433307 | 0.001459371 | 0.002498752 | 0.000223098 | 0.000397461 | 0.013051253 | 0.003799901 |
| A[T > A]G | 0.0042307 | 0.008101551 | 0.000512897 | 0.005802983 | 0.004687979 | 0.002188907 | 0.000352155 | 7.08E−05 | 0.021107257 | 0.0019941 |
| A[T > A]T | 0.00030105 | 0.003920751 | 0.028994204 | 0.006313245 | 0.008766221 | 0.002798603 | 0.00072832 | 0.010652827 | 0.070288411 | 0.007258402 |
| A[T > C]A | 0.029204833 | 0.004350833 | 0.012497502 | 0.026613679 | 0.080465311 | 0.009945034 | 0.001290568 | 0 | 0.029567022 | 0.003610801 |
| A[T > C]C | 0.007751283 | 3.36E−06 | 0.006158769 | 0.009664968 | 0.004927876 | 0.002608697 | 0.000277122 | 0.000323133 | 0.020410487 | 0.0020644 |
| A[T > C]G | 0.202033437 | 0.00402077 | 0.009548091 | 0.010305297 | 0.006747091 | 0.003518243 | 3.52E−06 | 1.15E−05 | 0.00507094 | 0.003521801 |
| A[T > C]T | 0.007831296 | 0.013702623 | 0.012397522 | 0.017909205 | 0.008156484 | 0.01079461 | 3.63E−05 | 0.009382159 | 0.059234082 | 0.004468701 |
| A[T > G]A | 3.87E−05 | 0.00026205 | 0.000120976 | 0.007063631 | 0.013494183 | 0.001789107 | 0.025011002 | 0.000127138 | 0.023838223 | 0.004781101 |
| A[T > G]C | 0.001580262 | 0.000920176 | 7.99E−18 | 0.000789406 | 0.00078766 | 0.001559221 | 0.010504621 | 0.001270956 | 0.00343492 | 7.31E−05 |
| A[T > G]G | 0.00115019 | 0.145027764 | 1.27E−05 | 0.001230633 | 0.002708832 | 0.001699152 | 0.026111486 | 0.000952699 | 0.004485501 | 0.001722 |
| A[T > G]T | 0.000340056 | 0.002280437 | 0.005748851 | 0.00597307 | 0.002079104 | 0.003438283 | 0.016407218 | 0.001764976 | 0.003178596 | 0.0011285 |
| C[C > A]A | 6.32E−05 | 0.006251197 | 0.002479504 | 0.008704474 | 0.006177337 | 0.002228887 | 0.001180519 | 0 | 0.004264235 | 0.006116101 |
| C[C > A]C | 0.002570425 | 0.003750718 | 0.006678665 | 0.003942026 | 0.001829211 | 0.00592704 | 0.000253111 | 0.005218572 | 0.000306235 | 0.003151701 |
| C[C > A]G | 0.005930982 | 0.000641123 | 6.72E−18 | 0.00107055 | 0.001459371 | 0.000814593 | 0.000379167 | 6.92E−05 | 0.000157257 | 0.00055412 |
| C[C > A]T | 0.005530915 | 2.84E−05 | 0.07128575 | 0.008214222 | 0.001959155 | 0.002018992 | 5.41E−05 | 0.005640381 | 0.000860851 | 0.003314601 |
| C[C > G]A | 0.001130187 | 0.002940563 | 0.000203959 | 0.000888457 | 0.000793658 | 0.002078962 | 4.71E−05 | 7.12E−06 | 0.001223945 | 0.021218005 |
| C[C > G]C | 0.000883146 | 0.002860548 | 0.000224955 | 0.002871476 | 0.001279448 | 0.001729137 | 0.000142062 | 0.003440786 | 0.000210179 | 0.059117014 |
| C[C > G]G | 0.000434072 | 0.000264051 | 6.66E−05 | 0.000763392 | 5.11E−05 | 0.000689656 | 0.000337148 | 2.20E−06 | 4.02E−05 | 0.003156001 |
| C[C > G]T | 0.00133022 | 0.003310634 | 0.000768846 | 0.002871476 | 0.001249461 | 0.003458273 | 0.000146064 | 0.013308248 | 0.000484084 | 0.04023301 |
| C[C > T]A | 0.003370558 | 0.007251388 | 0.00029794 | 0.014907663 | 0.008086514 | 0.002868568 | 0.001520669 | 0.018028129 | 0.001636999 | 0.003542601 |
| C[C > T]C | 0.000226037 | 0.004000766 | 0.000530894 | 0.012106223 | 0.009915725 | 0.000408796 | 0.001880827 | 0.0406213 | 0.000369373 | 0.004840501 |
| C[C > T]G | 0.028304684 | 6.59E−05 | 0.007948411 | 0.006403291 | 0.007986557 | 0.001099451 | 0.004061787 | 0 | 2.41E−05 | 0.005914801 |
| C[C > T]T | 0.003710614 | 0.000719138 | 0.000507898 | 0.032016456 | 0.027688064 | 0.002418792 | 0.002511105 | 0.042237292 | 0.002350306 | 0.002358401 |
| C[T > A]A | 7.45E−18 | 7.39E−05 | 0.001189762 | 0.006393286 | 0.002478931 | 7.03E−05 | 6.64E−05 | 0.00013655 | 0.00989632 | 0.002118001 |
| C[T > A]C | 0.000732121 | 1.10E−06 | 0.000973805 | 0.006593389 | 0.022290391 | 0.001639181 | 0.000514226 | 0 | 0.001136017 | 0.00050799 |
| C[T > A]G | 0.001920318 | 0.016003064 | 0.001589682 | 0.001290663 | 0.00085763 | 0.000138931 | 3.81E−07 | 0.000125176 | 0.000178583 | 0.002623201 |
| C[T > A]T | 6.66E−05 | 0.005231001 | 6.09E−06 | 0.010705503 | 0.00129944 | 0.003708148 | 0.000132058 | 0 | 0.010551155 | 0.003334101 |
| C[T > C]A | 0.011401887 | 0.0041808 | 0.003369326 | 0.012006171 | 0.003918311 | 0.002198902 | 0.000879387 | 0.002269666 | 0.007184034 | 0.001284 |
| C[T > C]C | 0.017002814 | 0.004980954 | 0.002639472 | 0.004562345 | 0.009495906 | 0.00572714 | 0.002791228 | 3.25E−06 | 0.006209066 | 0.002951901 |
| C[T > C]G | 0.193031947 | 0.000949182 | 0.000480904 | 0.008304268 | 0.005927445 | 0.001639181 | 0.000580255 | 0.003628315 | 0.000839952 | 0.00055781 |
| C[T > C]T | 0.026904453 | 0.000684131 | 0.000205959 | 0.024312497 | 0.028187848 | 0.012193911 | 0.002100924 | 0.000918582 | 0.016913141 | 0.0017926 |
| C[T > G]A | 0.004360722 | 0.001650316 | 0.005818837 | 0.000140072 | 0.000502783 | 0.001009496 | 0.003231421 | 0.000897193 | 0.003736475 | 0.004071701 |
| C[T > G]C | 0.018903128 | 0.000466089 | 3.93E−07 | 0.000847436 | 0.001039552 | 0.002618692 | 0.004161831 | 0.002148873 | 0.00247817 | 0.00016409 |
| C[T > G]G | 0.022103658 | 0.113021637 | 0.004449111 | 0.002191126 | 0.000698699 | 0.002018992 | 0.007113129 | 0.000788671 | 0.001120299 | 0.0017689 |
| C[T > G]T | 0.000886147 | 0.001180226 | 6.78E−18 | 0.005162654 | 0.000472796 | 0.021189419 | 1.80E−05 | 0 | 0.00175687 | 0.002421301 |
| G[C > A]A | 0.005190859 | 0.000855164 | 0.000216957 | 0.001890972 | 0.002738819 | 0.102948593 | 0.000517228 | 0.002703472 | 0.001439703 | 0.004842501 |
| G[C > A]C | 0.009131511 | 0.000192037 | 0.007338533 | 0.000752387 | 0.001079535 | 0.128935616 | 0.000866381 | 0.010228858 | 0.000504268 | 0.003629101 |
| G[C > A]G | 0.005640934 | 7.72E−05 | 9.79E−06 | 6.57E−18 | 0.002728824 | 0.012093961 | 0.000390172 | 0.000763601 | 0.000362283 | 0.00022304 |
| G[C > A]T | 0.006731114 | 0.000738141 | 0.015696862 | 0.001951003 | 0.000349849 | 0.498750949 | 0.000133059 | 0.010179165 | 0.000762947 | 0.008370602 |
| G[C > G]A | 0.002190363 | 0.000851163 | 0.000186963 | 0.003791949 | 0.002119086 | 0.001439281 | 0.000163072 | 0.011151059 | 0.001595924 | 0.005558801 |
| G[C > G]C | 0.000220036 | 0.00141027 | 9.83E−05 | 0.002571322 | 0.001489358 | 0.001719142 | 0.000135059 | 0.015758341 | 0.005060362 | 0.109340026 |
| G[C > G]G | 0.001590263 | 0.005721095 | 5.62E−06 | 0.001860957 | 0.001709263 | 0.000358821 | 0.000202089 | 0.001697967 | 2.68E−05 | 0.0007638 |
| G[C > G]T | 0.004940818 | 6.82E−05 | 1.75E−06 | 0.000436224 | 0.001079535 | 0.002258872 | 6.43E−05 | 0.076057555 | 0.003186104 | 0.012454003 |
| G[C > T]A | 0.000222037 | 0.006501245 | 9.49E−06 | 0.005142643 | 0.006777078 | 0.002438782 | 0.001240546 | 0.089177554 | 0.000887033 | 0.004533601 |
| G[C > T]C | 0.013102168 | 0.001510289 | 0.000505899 | 0.006933564 | 0.003148643 | 0.002348827 | 0.001440634 | 0.111629543 | 0.000484366 | 0.003374301 |
| G[C > T]G | 0.019103162 | 0.011902279 | 0.002059588 | 0.00844434 | 0.007356828 | 3.24E−18 | 0.003361479 | 0.009223924 | 0.000116424 | 0.007972002 |
| G[C > T]T | 0.000248041 | 0.005291013 | 0.000829834 | 0.006183178 | 0.005027833 | 0.002958523 | 0.000894393 | 0.180440442 | 0.004167105 | 0.007136002 |
| G[T > A]A | 0.002330386 | 0.002430465 | 0.000419916 | 0.003331713 | 0.002069108 | 0.000254873 | 0.000109048 | 0.001098639 | 0.017239234 | 0.0012592 |
| G[T > A]C | 0.008991488 | 0.000185035 | 0.002369526 | 0.000597307 | 0.00062573 | 0.001979012 | 0.000110048 | 6.56E−05 | 0.001005109 | 0.0003565 |
| G[T > A]G | 0.002640437 | 0.012502393 | 0.000435913 | 0.002461265 | 0.020391209 | 0.000504748 | 0.000360158 | 0.000337381 | 0.003520166 | 0.003061901 |
| G[T > A]T | 0.001850306 | 0.00512098 | 0.005368927 | 0.007934078 | 0.002768806 | 0.001509246 | 0.00063728 | 0.000135757 | 0.011658501 | 0.002640301 |
| G[T > C]A | 0.011401887 | 0.0026105 | 0.000729854 | 0.003861985 | 0.002179061 | 0.000709646 | 0.000761335 | 0 | 0.021590708 | 0.004448301 |
| G[T > C]C | 0.004430733 | 0.003170607 | 2.73E−05 | 0.002291178 | 0.000779664 | 0.003178413 | 0.000635279 | 0.000865945 | 0.011606135 | 0.006860402 |
| G[T > C]G | 0.121020029 | 0.006011151 | 0.00164967 | 0.004722427 | 0.011095217 | 0.000985508 | 0.00050022 | 0.000197907 | 0.006462688 | 0.002680701 |
| G[T > C]T | 0.005410896 | 0.012602413 | 0.002579484 | 0.005362756 | 0.00201913 | 0.001189406 | 0.000696306 | 0.003662473 | 0.054089003 | 0.008549902 |
| G[T > G]A | 0.000170028 | 0.005671086 | 6.72E−18 | 0.00140072 | 0.008066523 | 0.001229386 | 0.028212411 | 1.89E−05 | 0.007699624 | 0.0013954 |
| G[T > G]C | 0.001530253 | 0.003200613 | 9.19E−07 | 0.000171088 | 0.002688841 | 0.001649176 | 0.028812675 | 0.001229436 | 0.000800068 | 0.00068724 |
| G[T > G]G | 7.45E−18 | 0.299057252 | 6.72E−18 | 0.000861443 | 0.023389917 | 0.002288857 | 0.662291341 | 0.000212751 | 0.001946514 | 0.00045448 |
| G[T > G]T | 1.21E−05 | 0.001440276 | 0.000113977 | 0.002831455 | 0.016892718 | 0.007446282 | 0.04742086 | 0.001340079 | 0.002927906 | 0.002207901 |
| T[C > A]A | 0.000669111 | 0.001740333 | 0.21895623 | 0.014307354 | 0.007366824 | 2.37E−05 | 0.002020889 | 0.002083564 | 0.001944387 | 0.008561702 |
| T[C > A]C | 4.05E−05 | 8.28E−18 | 0.013197362 | 0.000709365 | 0.000428815 | 0.020189918 | 0.000163072 | 9.06E−06 | 0.000169428 | 0.003948601 |
| T[C > A]G | 0.000828137 | 9.91E−05 | 0.005458909 | 0.000223115 | 0.002728824 | 0.000633684 | 0.00020509 | 0 | 3.99E−09 | 0.0010775 |
| T[C > A]T | 0.007201192 | 0.004060777 | 0.401919655 | 0.006193183 | 0.014693666 | 0.003828088 | 0.000406179 | 0.001203779 | 0.002057492 | 0.009082702 |
| T[C > G]A | 0.004690776 | 0.007061352 | 0.001129774 | 0.002281173 | 0.002129082 | 2.35E−06 | 0.000529233 | 0 | 0.000947995 | 0.04222501 |
| T[C > G]C | 0.000517086 | 0.003280628 | 0.000293941 | 0.00280144 | 0.001799224 | 0.002088957 | 0.000348153 | 0.000113943 | 0.001869405 | 0.047387011 |
| T[C > G]G | 0.001230204 | 0.000229044 | 8.39E−05 | 0.001330684 | 0.000629729 | 0.000404798 | 6.59E−05 | 7.21E−07 | 0.000169997 | 0.004635901 |
| T[C > G]T | 0.000311051 | 0.002110404 | 0.000410918 | 0.012906634 | 0.0180922 | 0.001519241 | 0.000761335 | 0.004953383 | 0.006244143 | 0.110840027 |
| T[C > T]A | 7.45E−18 | 5.82E−11 | 0.00019996 | 0.008144186 | 0.004288151 | 0.00016092 | 4.07E−05 | 7.08E−05 | 0.003823611 | 0.008589702 |
| T[C > T]C | 0.000661109 | 0.004110787 | 0.000183963 | 0.017308897 | 0.015293407 | 0.00027986 | 0.000958422 | 0.026909332 | 0.002998121 | 0.008121902 |
| T[C > T]G | 1.19E−13 | 0.004810921 | 0.000153969 | 0.004602366 | 0.003338561 | 4.54E−18 | 0.001730761 | 6.92E−06 | 0.000157959 | 0.013691003 |
| T[C > T]T | 0.000236039 | 0.031406012 | 8.20E−05 | 0.037219131 | 0.178922866 | 0.002088957 | 0.003911721 | 0.016839668 | 0.038650891 | 0.012985003 |
| T[T > A]A | 0.001550257 | 0.003610691 | 0.009418117 | 0.003021553 | 0.011894872 | 0.002088957 | 0.001130497 | 0.006569907 | 0.060179904 | 0.003922301 |
| T[T > A]C | 0.00017903 | 6.95E−05 | 0.000804839 | 0.009234747 | 0.000897613 | 0.001859072 | 2.23E−16 | 0.000864507 | 0.007794895 | 0.0017123 |
| T[T > A]G | 0.000329054 | 0.009481815 | 0.006358729 | 0.004402263 | 0.000828643 | 0.000452774 | 4.24E−05 | 0.000784032 | 0.0067334 | 0.0013925 |
| T]T > A]T | 7.45E−18 | 0.002100402 | 0.000743851 | 0.028914862 | 0.014593709 | 0.002988508 | 0.000530233 | 0.007059955 | 0.062798282 | 0.002365301 |
| T[T > C]A | 0.004730783 | 0.004880934 | 0.000567886 | 0.00490252 | 0.003308574 | 0.002048977 | 0.000240106 | 0.000432939 | 0.012963549 | 0.0015339 |
| T[T > C]C | 0.008191356 | 0.000980188 | 0.001319736 | 0.026713731 | 0.019591554 | 0.002238882 | 0.00109048 | 0.000538465 | 0.023324449 | 0.001995 |
| T[T > C]G | 0.088414633 | 0.004160797 | 0.00184963 | 0.002961522 | 0.002159069 | 0.001349326 | 0.000109048 | 2.24E−05 | 0.003823535 | 0.0018459 |
| T[T > C]T | 0.012502069 | 0.00088717 | 0.000284943 | 0.195100282 | 0.031986211 | 0.002608697 | 0.001320581 | 0.009820763 | 0.09405438 | 0.003439301 |
| T[T > G]A | 3.24E−05 | 0.00606116 | 0.009458109 | 0.003221656 | 0.000797656 | 0.001599201 | 0.001610709 | 0.001875698 | 0.007106411 | 0.007135502 |
| T[T > G]C | 0.000706117 | 0.005651082 | 1.00E−06 | 0.003962036 | 0.001589315 | 0.002328837 | 0.010604665 | 0.000737917 | 0.006404454 | 0.002368501 |
| T[T > G]G | 0.007691273 | 0.079515222 | 0.00010098 | 0.005402777 | 0.006737096 | 0.001959022 | 0.023910518 | 0 | 0.00341 | 0.0020107 |
| T[T > G]T | 0.000499083 | 0.041207889 | 0.028994204 | 0.119061197 | 0.021690649 | 1.30E−17 | 0.031813995 | 0.006603134 | 0.012708762 | 0.004887201 |

| Type | SBS87 | SBS88 | SBS89 | SBS90 | SBS91 | SBS92 | SBS93 | SBS94 |
|---|---|---|---|---|---|---|---|---|
| A[C > A]A | 0.008972551 | 0 | 0.032168854 | 0.00220165 | 0.00294455 | 0.01132934 | 0.011573228 | 0.01557967 |
| A[C > A]C | 0.004572575 | 0 | 0.017693728 | 0.000708223 | 0.052997088 | 0.00974473 | 0.008096344 | 0.024746217 |
| A[C > A]G | 0.006208566 | 0 | 0.009670834 | 0.000138566 | 0.0002042 | 0.00469657 | 0.001760621 | 0.001574271 |
| A[C > A]T | 0.004957473 | 0.001731102 | 0.020743819 | 0.001755161 | 0.00013081 | 0.00775798 | 0.008420819 | 0.011076145 |
| A[C > G]A | 0.007866457 | 0 | 0.014817169 | 0.000508073 | 0.000243319 | 0.00305586 | 0.008856798 | 0.007003872 |
| A[C > G]C | 0.003933979 | 0.004039238 | 0.005883213 | 0.00027713 | 0.000124574 | 0.00168986 | 0.005418101 | 0.003990091 |
| A[C > G]G | 0.030661834 | 0 | 0.006050562 | 4.62E−05 | 4.73E−05 | 0.0009073 | 0.001631566 | 0.000550342 |
| A[C > G]T | 0.009720347 | 0.004039238 | 0.010900686 | 0.000923769 | 8.09E−05 | 0.00411591 | 0.013901695 | 0.007123635 |
| A[C > T]A | 0.009425049 | 0.010386613 | 0.049898108 | 0.000708019 | 0.01576341 | 0.023766369 | 0.008941007 | 0.007530362 |
| A[C > T]C | 0.003555581 | 0 | 0.013521816 | 0.000292527 | 0.001214934 | 0.014097539 | 0.005071208 | 0.014290105 |
| A[C > T]G | 0.121719339 | 0.012694749 | 0.006105628 | 9.24E−05 | 0.002227541 | 0.00978578 | 0.017637736 | 0.02309937 |
| A[C > T]T | 0.010701942 | 0 | 0.029636527 | 0.000646638 | 0.002326193 | 0.016452069 | 0.006509024 | 0.01651 |
| A[T > A]A | 0.009897046 | 0.047893826 | 0.011516257 | 0.061353668 | 0.000215265 | 0.01007203 | 0.010413532 | 0.010524831 |
| A[T > A]C | 0.000694526 | 0.023658396 | 0.003067675 | 0.000354111 | 0.000169236 | 0.00358815 | 0.005788866 | 0.005608619 |
| A[T > A]G | 0.003843379 | 0.000577034 | 0.010670567 | 0.000354111 | 0.0001602 | 0.00701173 | 0.005317446 | 0.004028931 |
| A[T > A]T | 0.004206177 | 0.042700519 | 0.004448487 | 0.023463736 | 0.000373855 | 0.00918124 | 0.013276356 | 0.015910259 |
| A[T > C]A | 0.00921505 | 0.111367571 | 0.015782305 | 0.005711972 | 0.000299841 | 0.089188046 | 0.034444397 | 0.013633952 |
| A[T > C]C | 0.003118983 | 0.04558569 | 0.003453395 | 0.000184754 | 0.000148826 | 0.00463736 | 0.003291743 | 0.005153844 |
| A[T > C]G | 0.000973215 | 0.006347374 | 0.014690391 | 0.000107773 | 0.000610604 | 0.040111988 | 0.006292806 | 0.002828782 |
| A[T > C]T | 0.00557557 | 0.16964801 | 0.011812088 | 0.002863684 | 0.000235212 | 0.029566939 | 0.010676658 | 0.022937681 |
| A[T > G]A | 0.007955057 | 0.008078477 | 0.00523782 | 0.005434842 | 0.002388151 | 0.00206966 | 0.025483724 | 0.003719629 |
| A[T > G]C | 0.000673416 | 0.005193306 | 0.001300242 | 4.62E−05 | 0.000131587 | 0.00035897 | 0.002393023 | 0.000917381 |
| A[T > G]G | 0.002687785 | 0.002308136 | 0.005110413 | 0.000230942 | 0.001220499 | 0.00123837 | 0.005138721 | 0.002077109 |
| A[T > G]T | 0.002173988 | 0.007501443 | 0.003994217 | 0.002047688 | 0.000213945 | 0.0035412 | 0.007962999 | 0.002289645 |
| C[C > A]A | 0.008557454 | 0 | 0.024671469 | 0.001185504 | 0.008191134 | 0.018550349 | 0.006498487 | 0.079925512 |
| C[C > A]C | 0.006040367 | 0 | 0.014514463 | 0.000446489 | 0.004579504 | 0.013955519 | 0.004795732 | 0.07599413 |
| C[C > A]G | 0.020006891 | 0 | 0.003289875 | 0.000216006 | 0.002326351 | 0.00528203 | 0.001508262 | 0.00622497 |
| C[C > A]T | 0.004885573 | 0 | 0.016733698 | 0.001016146 | 0.003239626 | 0.012543099 | 0.010062158 | 0.039904051 |
| C[C > G]A | 0.006765663 | 0.004616272 | 0.015425429 | 0.000338715 | 6.64E−05 | 0.00240959 | 0.00337806 | 0.009195761 |
| C[C > G]C | 0.003339182 | 0.001154068 | 0.010618496 | 7.70E−05 | 0.000202057 | 0.00396222 | 0.003208406 | 0.015074767 |
| C[C > G]G | 0.085984533 | 0 | 0.004169842 | 1.54E−05 | 5.34E−05 | 0.00160101 | 0.000775635 | 0.001842681 |
| C[C > G]T | 0.009610648 | 0.00288517 | 0.015833619 | 0.000292527 | 0.000100649 | 0.005366 | 0.006051846 | 0.008551437 |
| C[C > T]A | 0.008383154 | 0 | 0.009444215 | 0.000615846 | 0.036614087 | 0.036607349 | 0.008701419 | 0.013657465 |
| C[C > T]C | 0.008045256 | 0 | 0.011558976 | 0.000307923 | 0.000270783 | 0.034761309 | 0.003991478 | 0.017945836 |
| C[C > T]G | 0.186898985 | 0 | 0.00233382 | 0.00020015 | 0.001276567 | 0.01082396 | 0.007245249 | 0.018825229 |
| C[C > T]T | 0.006962062 | 0.004039238 | 0.01288627 | 0.000369508 | 0.001221976 | 0.045323478 | 0.006905849 | 0.025048186 |
| C[T > A]A | 0.008352155 | 0 | 0.03155286 | 0.102061094 | 0.000317267 | 0.00704592 | 0.010633058 | 0.011932627 |
| C[T > A]C | 0.004910873 | 0.003462204 | 0.017709755 | 0.000354111 | 0.000141907 | 0.00393189 | 0.00851737 | 0.008788044 |
| C[T > A]G | 0.002767285 | 0 | 0.036874595 | 0.000369508 | 0.000246237 | 0.01024476 | 0.007504242 | 0.007734639 |
| C[T > A]T | 0.010191945 | 0 | 0.019947678 | 0.10052148 | 0.000460322 | 0.00644868 | 0.024588409 | 0.014440581 |
| C[T > C]A | 0.005121972 | 0 | 0.004889623 | 0.004772807 | 0.000154628 | 0.030764589 | 0.029558831 | 0.006294826 |
| C[T > C]C | 0.00183399 | 0.001731102 | 0.004215919 | 0.000215546 | 0.000146082 | 0.00401078 | 0.008356107 | 0.014926472 |
| C[T > C]G | 0.000836095 | 0 | 0.008097547 | 0.00020015 | 0.000150463 | 0.014122809 | 0.002378116 | 0.007044152 |
| C[T > C]T | 0.008857152 | 0 | 0.004313727 | 0.006358611 | 0.00016344 | 0.013670349 | 0.014858319 | 0.016106356 |
| C[T > G]A | 0.008515354 | 0 | 0.006903017 | 0.008945164 | 0.000758217 | 0.0009284 | 0.009320692 | 0.002228081 |
| C[T > G]C | 0.001083894 | 0.002308136 | 0.003219819 | 4.62E−05 | 5.88E−05 | 0.00041878 | 0.006366696 | 0.002052565 |
| C[T > G]G | 0.001958989 | 0.002308136 | 0.009030111 | 0.000123169 | 0.00042886 | 0.00209052 | 0.002552462 | 0.00251354 |
| C[T > G]T | 0.003082183 | 0 | 0.004179842 | 0.009700255 | 0.000122273 | 0.00054519 | 0.038220293 | 0.002796927 |
| G[C > A]A | 0.007102761 | 0.008655511 | 0.006781995 | 0.000969957 | 0.003524665 | 0.00893931 | 0.002242206 | 0.013892654 |
| G[C > A]C | 0.004665975 | 0 | 0.006025706 | 0.000415696 | 0.005870289 | 0.00782492 | 0.001700249 | 0.030449959 |
| G[C > A]G | 0.008287855 | 0.000577034 | 0.001679936 | 0.000123003 | 5.91E−05 | 0.00445574 | 0.000295683 | 0.001764505 |
| G[C > A]T | 0.001067494 | 0.003462204 | 0.007237208 | 0.0004003 | 0.001167017 | 0.00655609 | 0.003100581 | 0.011344232 |
| G[C > G]A | 0.002617586 | 0 | 0.006521988 | 0.000431092 | 3.76E−05 | 0.00234881 | 0.003532121 | 0.003114008 |
| G[C > G]C | 0.001198293 | 0.001731102 | 0.004590414 | 0.000292527 | 0.000189376 | 0.00216132 | 0.004664741 | 0.006887041 |
| G[C > G]G | 0.00545547 | 0.000577034 | 0.002079921 | 4.62E−05 | 6.39E−05 | 0.00089337 | 0.000911111 | 0.000704123 |
| G[C > G]T | 0.004499176 | 0.006924409 | 0.005716567 | 0.000261735 | 0.000157899 | 0.00152748 | 0.005673738 | 0.003691821 |
| G[C > T]A | 0.00188169 | 0.000577034 | 0.006853693 | 0.000446489 | 0.011307255 | 0.012825829 | 0.006114246 | 0.005591358 |
| G[C > T]C | 0.002618086 | 0 | 0.00852926 | 0.000323319 | 0.000131455 | 0.013510909 | 0.005132146 | 0.016772937 |
| G[C > T]G | 0.017708904 | 0.006347374 | 0.001879929 | 0.000169004 | 0.001191861 | 0.00593888 | 0.009014056 | 0.016933011 |
| G[C > T]T | 0.001446392 | 0.003462204 | 0.009225851 | 0.000508073 | 0.00015044 | 0.016846129 | 0.004511933 | 0.008438094 |
| G[T > A]A | 0.009401649 | 0 | 0.005810454 | 0.038967661 | 0.000273732 | 0.00562396 | 0.005778544 | 0.005669559 |
| G[T > A]C | 0.003398982 | 0.005193306 | 0.002521202 | 0.000169357 | 0.000318247 | 0.00192496 | 0.00437304 | 0.001491142 |
| G[T > A]G | 0.006810563 | 0.00288517 | 0.009132422 | 0.000153961 | 0.000119027 | 0.0059962 | 0.004438423 | 0.002225964 |
| G[T > A]T | 0.006955462 | 0.015002885 | 0.003874896 | 0.016627845 | 0.000727982 | 0.00411785 | 0.005196632 | 0.007256276 |
| G[T > C]A | 0.003009584 | 0 | 0.005049809 | 0.003280086 | 0.000171407 | 0.029209339 | 0.011400838 | 0.004144722 |
| G[T > C]C | 0.003894779 | 0.003462204 | 0.001644875 | 9.24E−05 | 0.000135452 | 0.00268576 | 0.00281096 | 0.001138667 |
| G[T > C]G | 0.003961778 | 0.000577034 | 0.007259725 | 7.70E−05 | 0.000144829 | 0.00925981 | 0.000207275 | 0.000947577 |
| G[T > C]T | 0.006540964 | 0.013271783 | 0.00405927 | 0.001324069 | 0.000152534 | 0.012672949 | 0.005878906 | 0.010745671 |
| G[T > G]A | 0.003527981 | 0 | 0.012761223 | 0.006358611 | 0.00010075 | 0.00055049 | 0.003739788 | 0.001278935 |
| G[T > G]C | 0.001238693 | 0.001731102 | 0.001270164 | 4.62E−05 | 0.00139143 | 0.00029423 | 0.002704224 | 0.000632935 |
| G[T > G]G | 0.003236482 | 0.00288517 | 0.048482879 | 0.000230942 | 7.33E−05 | 0.00154435 | 0.000865989 | 0.001974416 |
| G[T > G]T | 0.002460587 | 0.013271783 | 0.003769857 | 0.002880076 | 5.39E−05 | 0.0001996 | 0.00614793 | 0.002121497 |
| T[C > A]A | 0.004005478 | 0 | 0.015740016 | 0.002016896 | 0.333883761 | 0.014848579 | 0.009890657 | 0.025239421 |
| T[C > A]C | 0.004178977 | 0.002308136 | 0.016784775 | 0.000785204 | 0.045238573 | 0.012831779 | 0.004037422 | 0.020303696 |
| T[C > A]G | 0.003172283 | 0.000577034 | 0.001949926 | 7.70E−05 | 0.31841755 | 0.00239317 | 0.000174699 | 0.002607667 |
| T[C > A]T | 0.004133778 | 0.010386613 | 0.018713928 | 0.001447238 | 0.076489253 | 0.013186259 | 0.008748807 | 0.008152825 |
| T[C > G]A | 0.000639537 | 0 | 0.022471653 | 0.000646638 | 4.88E−05 | 0.019451159 | 0.0128107 | 0.006092674 |
| T[C > G]C | 0.002841185 | 0 | 0.016591053 | 0.000323319 | 0.000153375 | 0.0040875 | 0.007062306 | 0.005348828 |
| T[C > G]G | 0.026116858 | 0.000577034 | 0.003639862 | 6.16E−05 | 2.58E−05 | 0.0002984 | 0.000700167 | 0.001042688 |
| T[C > G]T | 0.012630931 | 0.004039238 | 0.026080078 | 0.001431843 | 0.000267231 | 0.022989279 | 0.025727117 | 0.012610709 |
| T[C > T]A | 8.72E−06 | 0 | 0.009469641 | 0.002110055 | 0.036454398 | 0.017104589 | 0.008564191 | 0.009482037 |
| T[C > T]C | 0.006175266 | 0 | 0.010544089 | 0.000985354 | 0.000133887 | 0.017369399 | 0.007826701 | 0.019746357 |
| T[C > T]G | 0.059555677 | 0.009232545 | 0.002440703 | 0.000292527 | 0.002047499 | 0.00359965 | 0.009680071 | 0.005085538 |
| T[C > T]T | 0.010947941 | 0 | 0.009482236 | 0.002771308 | 0.00013978 | 0.024582749 | 0.010452124 | 0.012927103 |
| T[T > A]A | 0.008279655 | 0.02135026 | 0.007089731 | 0.357009385 | 0.002293579 | 0.00817815 | 0.020663804 | 0.010310468 |
| T[T > A]C | 0.00372258 | 0 | 0.00316471 | 0.000877581 | 0.000224179 | 0.00284712 | 0.010370812 | 0.004183892 |
| T[T > A]G | 0.004866274 | 0.004039238 | 0.010062615 | 0.00060045 | 0.00074177 | 0.00675146 | 0.010316638 | 0.004282786 |
| T[T > A]T | 0.005260771 | 0.074437392 | 0.00635055 | 0.162675746 | 0.000508384 | 0.00604418 | 0.030986782 | 0.017322773 |
| T[T > C]A | 0.009768947 | 0.004039238 | 0.007517012 | 0.010115272 | 0.000318737 | 0.034536139 | 0.094342421 | 0.008883495 |
| T[T > C]C | 0.007158861 | 0.002308136 | 0.002556836 | 0.000446489 | 0.000177915 | 0.0043502 | 0.013289549 | 0.004857044 |
| T[T > C]G | 0.001711291 | 0 | 0.008690558 | 0.000138566 | 0.000150278 | 0.012935279 | 0.010955607 | 0.002100277 |
| T[T > C]T | 0.006587764 | 0.096364686 | 0.006789743 | 0.004000105 | 0.001273762 | 0.014286909 | 0.014080093 | 0.016765958 |
| T[T > G]A | 0.008204455 | 0.030582804 | 0.006865512 | 0.018583156 | 0.005954684 | 0.00220843 | 0.053673548 | 0.004071805 |
| T[T > G]C | 0.002497286 | 0.020773226 | 0.002929014 | 0.000184754 | 0.000143443 | 0.00030058 | 0.013276016 | 0.001235435 |
| T[T > G]G | 0.001995989 | 0 | 0.005037266 | 0.000292527 | 0.000628245 | 0.00174284 | 0.012705223 | 0.003048468 |
| T[T > G]T | 0.006250766 | 0.102135026 | 0.005689784 | 0.008330219 | 0.001008682 | 0.00179435 | 0.054953589 | 0.003333374 |

TABLE 10
COSMIC DATA (COSMIC_v3.2_SBS_GRCh38)

| Type | SBS1 | SBS2 | SBS3 | SBS4 | SBS5 | SBS6 | SBS7a | SBS7b | SBS7c | SBS7d |
|---|---|---|---|---|---|---|---|---|---|---|
| A[C > | 0.000876023 | 5.79E−07 | 0.020919749 | 0.042450736 | 0.012052048 | 0.000425146 | 6.72E−05 | 0.002344281 | 0.004840988 | 4.04E−05 |
| A]A | | | | | | | | | | |
| A[C > | 0.00222012 | 0.000145505 | 0.016343192 | 0.032989582 | 0.009337087 | 0.000516226 | 0.00017673 | 0.000456788 | 0.001135127 | 0.000753958 |
| A]C | | | | | | | | | | |
| A[C > | 0.000179727 | 5.36E−05 | 0.001807549 | 0.016115961 | 0.00190814 | 5.34E−05 | 7.33E−05 | 0.000192188 | 0.000388049 | 0.000256936 |
| A]G | | | | | | | | | | |
| A[C > | 0.001265053 | 9.76E−05 | 0.012265066 | 0.02966277 | 0.006635872 | 0.000179986 | 0.000248522 | 0.000714051 | 0.001963631 | 0.004051334 |
| A]T | | | | | | | | | | |
| A[C > | 0.001839055 | 2.23E−16 | 0.019813416 | 0.006930938 | 0.010143807 | 0.000471161 | 6.51E−05 | 8.61E−06 | 0.001122548 | 0.001180887 |
| G]A | | | | | | | | | | |
| A[C > | 0.001187959 | 0.000130757 | 0.011588809 | 0.002813526 | 0.00563786 | 0.000284712 | 2.13E−05 | 0.000187273 | 0.000314874 | 0.002513195 |
| G]C | | | | | | | | | | |
| A[C > | 0.000116772 | 1.56E−05 | 0.00026132 | 0.001322335 | 0.001774054 | 2.28E−16 | 5.23E−05 | 0.0005218 | 2.29E−16 | 0.000931136 |
| G]G | | | | | | | | | | |
| A[C > | 0.001126688 | 9.10E−05 | 0.017492799 | 0.003569588 | 0.010139532 | 0.00025398 | 9.58E−05 | 0.000286626 | 0.001933575 | 0.002910959 |
| G]T | | | | | | | | | | |
| A[C > | 0.024718478 | 6.10E−05 | 0.014281752 | 0.008751692 | 0.032741397 | 0.064121962 | 0.000122309 | 0.001418642 | 0.001333026 | 0.003332502 |
| T]A | | | | | | | | | | |
| A[C > | 0.006154016 | 0.001356731 | 0.012282156 | 0.004141035 | 0.017704858 | 0.022560258 | 0.01747552 | 0.037751869 | 0.007580675 | 0.001379793 |
| T]C | | | | | | | | | | |
| A[C > | 0.370623899 | 3.35E−05 | 0.002654514 | 0.000808897 | 0.006374218 | 0.12327893 | 2.30E−16 | 0.000561064 | 0.000954169 | 0.00124357 |
| T]G | | | | | | | | | | |
| A[C > | 0.009468128 | 0.001856029 | 0.012164532 | 0.00427345 | 0.022086109 | 0.021298317 | 0.007435631 | 0.010861613 | 0.007493856 | 0.03291084 |
| T]T | | | | | | | | | | |
| A[T > | 0.000783812 | 9.49E−05 | 0.005471488 | 0.009389959 | 0.007344732 | 9.73E−05 | 0.001927253 | 0.002482522 | 0.0123154 | 8.82E−05 |
| A]A | | | | | | | | | | |
| A[T > | 0.002201314 | 0.000875074 | 0.007239755 | 0.003545218 | 0.006948776 | 0.000545302 | 0.000670606 | 0.000469101 | 0.003422224 | 0.001079059 |
| A]C | | | | | | | | | | |
| A[T > | 0.001117281 | 2.52E−05 | 0.009610497 | 0.012364317 | 0.009079253 | 2.20E−16 | 0.000583325 | 0.001555801 | 0.000209626 | 0.003670317 |
| A]G | | | | | | | | | | |
| A[T > | 0.000179456 | 2.21E−16 | 0.006094787 | 0.004200288 | 0.006175832 | 0.001646952 | 0.003688885 | 0.008272417 | 0.050398663 | 0.040793614 |
| A]T | | | | | | | | | | |
| A[T > | 0.001067943 | 6.10E−05 | 0.016444364 | 0.007924647 | 0.045979219 | 0.002190691 | 0.00048678 | 0.001016937 | 0.000286035 | 0.004323671 |
| C]A | | | | | | | | | | |
| A[T > | 0.003000895 | 2.14E−05 | 0.007792024 | 0.000718082 | 0.013436305 | 0.001128555 | 0.000227205 | 0.000458051 | 0.000533347 | 0.003926577 |
| C]C | | | | | | | | | | |
| A[T > | 0.000103887 | 1.31E−05 | 0.012262356 | 0.004985612 | 0.038029328 | 0.004204267 | 0.000185829 | 0.001495962 | 0.001063032 | 0.009116273 |
| C]G | | | | | | | | | | |
| A[T > | 0.005628826 | 0.000153466 | 0.017256926 | 0.001556401 | 0.038150707 | 0.000498054 | 0.0006115 | 0.00214544 | 0.00263425 | 0.050619813 |
| C]T | | | | | | | | | | |
| A[T > | 0.00016852 | 0.000235436 | 0.003936681 | 0.001046651 | 0.003841554 | 2.55E−05 | 0.000469892 | 8.41E−05 | 0.011818812 | 0.00366917 |
| G]A | | | | | | | | | | |
| A[T > | 0.000204337 | 7.44E−05 | 0.00261073 | 0.000159685 | 0.002607044 | 0.000203739 | 0.000247223 | 0.000936192 | 0.001050683 | 0.002657683 |
| G]C | | | | | | | | | | |
| A[T > | 0.000262659 | 2.03E−06 | 0.006280719 | 0.001415914 | 0.007904525 | 0.000106098 | 0.000274272 | 0.001466043 | 0.000600067 | 0.010514962 |
| G]G | | | | | | | | | | |
| A[T > | 0.000109831 | 3.72E−06 | 0.003960115 | 0.000215502 | 0.004811173 | 0.000382966 | 0.000307241 | 0.000731445 | 2.21E−16 | 0.014193399 |
| G]T | | | | | | | | | | |
| C[C > | 0.000305026 | 0.000205314 | 0.022375696 | 0.080268881 | 0.007378521 | 0.001800201 | 0.000451034 | 0.001134121 | 0.000108023 | 0.014348122 |
| A]A | | | | | | | | | | |
| C[C > | 0.001738259 | 9.34E−05 | 0.024991558 | 0.078742784 | 0.006056583 | 0.004018387 | 0.000236313 | 0.001531669 | 0.00304176 | 0.003164912 |
| A]C | | | | | | | | | | |
| C[C > | 9.20E−05 | 2.22E−16 | 0.002519081 | 0.024593149 | 0.003467616 | 0.001647055 | 2.23E−16 | 0.000409628 | 0.001910273 | 8.50E−05 |
| A]G | | | | | | | | | | |
| C[C > | 2.18E−16 | 0.000415577 | 0.015315381 | 0.068633445 | 0.006445227 | 0.009495852 | 0.000368769 | 0.001482361 | 0.013379336 | 0.000115778 |
| A]T | | | | | | | | | | |
| C[C > | 2.36E−05 | 2.20E−16 | 0.019193375 | 0.00778817 | 0.006911778 | 4.07E−05 | 2.21E−16 | 0.000455638 | 0.000204153 | 0.001306174 |
| G]A | | | | | | | | | | |
| C[C | 7.46E−05 | 0.000344145 | 0.013928101 | 0.00678749 | 0.008946776 | 3.65E−05 | 0.000128988 | 0.000948647 | 0.002224718 | 0.000281107 |
| G]C | | | | | | | | | | |
| C[C > | 0.000347296 | 4.85E−05 | 0.001505427 | 0.003412927 | 0.002495481 | 8.41E−05 | 7.75E−05 | 0.000794155 | 0.000262037 | 0.001318182 |
| G]G | | | | | | | | | | |
| C[C > | 2.18E−16 | 6.28E−05 | 0.022177467 | 0.00678377 | 0.009464255 | 1.42E−05 | 9.48E−05 | 0.000885437 | 0.004202102 | 2.20E−16 |
| G]T | | | | | | | | | | |
| C[C > | 0.001955296 | 0.00426422 | 0.016011054 | 0.012632154 | 0.019265587 | 0.013452053 | 0.049564181 | 0.106448207 | 0.002685697 | 0.035227113 |
| T]A | | | | | | | | | | |
| C[C > | 0.000262195 | 0.00016668 | 0.019953734 | 0.011361882 | 0.018741868 | 0.014540862 | 0.054450495 | 0.179847635 | 0.005876799 | 0.002555519 |
| T]C | | | | | | | | | | |
| C[C > | 0.19338067 | 0.002759371 | 0.002248104 | 0.003322585 | 0.01753852 | 0.121782217 | 0.006712663 | 0.022188156 | 0.008941279 | 0.002636364 |
| T]G | | | | | | | | | | |
| C[C > | 0.000191625 | 0.001431321 | 0.02287362 | 0.015915002 | 0.02294064 | 0.013650287 | 0.056108337 | 0.125353987 | 0.020415875 | 0.000136559 |
| T]T | | | | | | | | | | |
| C[T > | 4.23E−05 | 0.000241151 | 0.007938556 | 0.014400022 | 0.00353436 | 6.62E−05 | 0.0001764 | 0.00112021 | 0.003457357 | 0.001601695 |
| A]A | | | | | | | | | | |
| C[T > | 0.000387449 | 0.000242876 | 0.017850616 | 0.012236935 | 0.005197411 | 0.000549592 | 0.000260902 | 0.000961079 | 0.013391576 | 8.11E−05 |
| A]C | | | | | | | | | | |
| C[T > | 0.000318465 | 0.000147869 | 0.013197832 | 0.03250062 | 0.005002106 | 0.000114362 | 0.000111622 | 0.000883181 | 0.000957515 | 0.001193831 |
| A]G | | | | | | | | | | |
| C[T > | 0.000257226 | 2.23E−16 | 0.012780765 | 0.011877219 | 0.004321237 | 0.000179169 | 0.000372161 | 0.000112754 | 0.048840016 | 0.001151553 |
| A]T | | | | | | | | | | |
| C[T > | 2.19E−16 | 0.000299702 | 0.008828394 | 0.005180008 | 0.013278809 | 0.001650763 | 0.000449471 | 0.001530287 | 0.007542418 | 0.010147387 |
| C]A | | | | | | | | | | |
| C[T > | 0.002464687 | 2.84E−05 | 0.014541232 | 0.002367145 | 0.01041485 | 0.001027368 | 0.000258903 | 0.0008427 | 0.00357775 | 8.87E−05 |
| C]C | | | | | | | | | | |
| C[T > | 0.00035385 | 2.21E−16 | 0.010098341 | 0.005500105 | 0.020767225 | 0.00529049 | 0.000103649 | 0.00073015 | 0.002162131 | 0.000930193 |
| C]G | | | | | | | | | | |
| C[T > | 4.21E−05 | 0.000185792 | 0.015900479 | 0.002576753 | 0.014973588 | 0.001271196 | 0.001825696 | 0.005768572 | 0.043223916 | 0.039753617 |
| C]T | | | | | | | | | | |
| C[T > | 3.49E−05 | 0.000151836 | 0.004369205 | 0.000870001 | 0.002585873 | 4.92E−05 | 8.39E−05 | 0.000359067 | 0.003268049 | 0.001472366 |
| G]A | | | | | | | | | | |
| C[T > | 0.000209005 | 0.000154286 | 0.007060019 | 0.000442335 | 0.004616582 | 0.000781997 | 0.000171935 | 0.000794546 | 0.005576492 | 4.97E−05 |
| G]C | | | | | | | | | | |
| C[T > | 0.000125813 | 0.000120081 | 0.010698242 | 0.003790072 | 0.006120341 | 0.000918875 | 2.22E−16 | 0.001290265 | 0.001185685 | 2.57E−05 |
| G]G | | | | | | | | | | |
| C[T > | 0.000169176 | 0.000202773 | 0.007074707 | 0.001207853 | 0.007336053 | 0.001301224 | 2.66E−05 | 3.70E−06 | 0.003409775 | 0.000156211 |
| G]T | | | | | | | | | | |
| G[C > | 0.00155627 | 8.57E−05 | 0.00714379 | 0.031666925 | 0.010205301 | 0.000425523 | 4.05E−05 | 0.000747719 | 0.001048388 | 0.000167487 |
| A]A | | | | | | | | | | |
| G[C > | 0.000329319 | 2.19E−16 | 0.01077098 | 0.034394391 | 0.007548778 | 0.000897336 | 0.000135929 | 0.000212534 | 0.000730679 | 2.18E−16 |
| A]C | | | | | | | | | | |
| G[C > | 0.000582768 | 1.39E−05 | 0.001444126 | 0.015453974 | 0.00235978 | 0.000358586 | 4.28E−05 | 7.36E−05 | 2.23E−16 | 1.83E−05 |
| A]G | | | | | | | | | | |
| G[C > | 2.19E−16 | 5.07E−05 | 0.009968801 | 0.021137734 | 0.006789125 | 0.001219561 | 2.22E−16 | 0.000146596 | 0.000360615 | 7.34E−05 |
| A]T | | | | | | | | | | |
| G[C > | 9.45E−06 | 6.50E−05 | 0.012724563 | 0.005301204 | 0.004682432 | 1.93E−05 | 1.96E−05 | 5.12E−05 | 0.000181721 | 0.000195402 |
| G]A | | | | | | | | | | |
| G[C > | 0.000159317 | 0.000255995 | 0.00852785 | 0.005682981 | 0.005012783 | 0.000673248 | 3.27E−05 | 0.000446815 | 0.000465783 | 2.18E−16 |
| G]C | | | | | | | | | | |
| G[C > | 0.000164803 | 2.24E−16 | 0.002443906 | 0.00333321 | 0.001522764 | 2.23E−16 | 4.54E−05 | 0.000340455 | 0.000181149 | 0.000547644 |
| G]G | | | | | | | | | | |
| G[C > | 2.19E−16 | 0.00013259 | 0.014654138 | 0.003031071 | 0.006689577 | 0.000318276 | 4.17E−05 | 0.000276238 | 0.001271589 | 0.000874872 |
| G]T | | | | | | | | | | |
| G[C > | 0.004373315 | 2.22E−16 | 0.01643172 | 0.004098662 | 0.019410082 | 0.093276158 | 8.48E−05 | 0.00085697 | 0.000156759 | 0.000363886 |
| T]A | | | | | | | | | | |
| G[C > | 9.01E−05 | 1.27E−07 | 0.013241389 | 0.004408017 | 0.021314196 | 0.109095629 | 0.011524404 | 0.025998304 | 9.36E−06 | 0.000177967 |
| T]C | | | | | | | | | | |
| G[C > | 0.216428366 | 2.24E−16 | 0.000533216 | 0.000486851 | 0.01240397 | 0.178790626 | 5.56E−06 | 0.000177804 | 3.45E−05 | 0.000157762 |
| T]G | | | | | | | | | | |
| G[C > | 3.76E−05 | 4.99E−05 | 0.011264745 | 0.00379881 | 0.018515795 | 0.073768594 | 0.015998258 | 0.00537517 | 0.000753019 | 0.041759766 |
| T]T | | | | | | | | | | |
| G[T > | 7.93E−05 | 2.20E−16 | 0.00701785 | 0.009166635 | 0.003642939 | 5.04E−05 | 0.000397327 | 0.000523053 | 0.001713717 | 2.34E−05 |
| A]A | | | | | | | | | | |
| G[T > | 0.000126346 | 0.000129544 | 0.007452207 | 0.003587281 | 0.002895095 | 0.00048456 | 0.000222451 | 0.000255143 | 0.002253735 | 0.000111028 |
| A]C | | | | | | | | | | |
| G[T > | 0.000242933 | 6.36E−05 | 0.011652531 | 0.013161737 | 0.004122784 | 0.000194829 | 0.000290378 | 0.000659214 | 2.22E−16 | 0.001349364 |
| A]G | | | | | | | | | | |
| G[T > | 0.000257268 | 6.32E−05 | 0.012476183 | 0.005600095 | 0.003261512 | 0.000250198 | 0.000309387 | 5.67E−05 | 0.020519482 | 0.008841252 |
| A]T | | | | | | | | | | |
| G[T > | 0.001026071 | 2.20E−16 | 0.009920417 | 0.002883215 | 0.013996034 | 0.002540903 | 0.000100075 | 0.00026451 | 2.20E−16 | 0.000791266 |
| C]A | | | | | | | | | | |
| G[T > | 0.00186091 | 2.71E−05 | 0.005619043 | 0.001175831 | 0.00789933 | 0.001099922 | 7.36E−05 | 2.21E−16 | 0.000419969 | 0.002299875 |
| C]C | | | | | | | | | | |
| G[T > | 0.001155412 | 0.000106686 | 0.010949361 | 0.002381169 | 0.015046656 | 0.002447845 | 7.26E−05 | 0.000531592 | 0.000232244 | 0.002568789 |
| C]G | | | | | | | | | | |
| G[T > | 7.11E−05 | 1.26E−05 | 0.010346103 | 0.000765955 | 0.01387662 | 0.00091403 | 0.000819976 | 0.006950687 | 0.00714644 | 0.320949556 |
| C]T | | | | | | | | | | |
| G[T > | 2.18E−16 | 0.000206209 | 0.004393612 | 0.000594539 | 0.002501419 | 4.30E−05 | 8.98E−05 | 0.00032119 | 0.002456658 | 7.40E−05 |
| G]A | | | | | | | | | | |
| G[T > | 2.18E−16 | 0.000131522 | 0.002331305 | 0.000252106 | 0.001701241 | 0.000211066 | 0.000120163 | 0.000319925 | 0.001648106 | 2.21E−05 |
| G]C | | | | | | | | | | |
| G[T > | 0.000343661 | 3.70E−05 | 0.010949361 | 0.002441452 | 0.005316485 | 0.000273759 | 0.000325424 | 0.002421809 | 0.006036334 | 0.003798209 |
| G]G | | | | | | | | | | |
| G[T > | 1.46E−05 | 2.25E−16 | 0.005913508 | 0.000255656 | 0.002380296 | 0.000793975 | 0.000844242 | 0.001856899 | 0.00682298 | 0.006903443 |
| G]T | | | | | | | | | | |
| T[C > | 6.51E−05 | 0.000637963 | 0.00845919 | 0.040040133 | 0.007894812 | 0.000356154 | 0.000391022 | 0.002918041 | 0.002525963 | 0.011409607 |
| A]A | | | | | | | | | | |
| T[C > | 0.00249718 | 0.000169415 | 0.016867475 | 0.047097004 | 0.009193833 | 0.000497308 | 0.000636505 | 0.003535444 | 0.01420767 | 0.007752389 |
| A]C | | | | | | | | | | |
| T[C > | 2.24E−16 | 8.65E−05 | 0.001440153 | 0.011135139 | 0.002345869 | 0.000203177 | 3.50E−05 | 0.000306529 | 2.26E−16 | 2.26E−16 |
| A]G | | | | | | | | | | |
| T[C > | 5.84E−06 | 0.000441166 | 0.010071242 | 0.041727037 | 0.012810853 | 0.000750523 | 0.000617237 | 0.001424845 | 0.003754831 | 0.000191979 |
| A]T | | | | | | | | | | |
| T[C > | 2.21E−16 | 2.23E−16 | 0.013377792 | 0.0036016 | 0.007844591 | 4.97E−05 | 2.24E−16 | 0.00025558 | 0.001944591 | 0.003743152 |
| G]A | | | | | | | | | | |
| T[C > | 0.000200367 | 9.84E−05 | 0.020381532 | 0.005974993 | 0.007619752 | 0.000112843 | 5.09E−05 | 0.001627108 | 0.00461249 | 0.000587423 |
| G]C | | | | | | | | | | |
| T[C > | 6.97E−05 | 4.18E−05 | 0.00053112 | 0.001869477 | 0.001744103 | 1.98E−06 | 6.46E−06 | 0.000371922 | 8.91E−06 | 0.000887231 |
| G]G | | | | | | | | | | |
| T[C > | 0.001549177 | 0.001183128 | 0.023738634 | 0.003414949 | 0.012609107 | 0.000128604 | 0.000282942 | 0.000721517 | 0.012381882 | 8.57E−05 |
| G]T | | | | | | | | | | |
| T[C > | 0.001097601 | 0.535130188 | 0.00579369 | 0.003762565 | 0.02038991 | 0.012005208 | 0.238623739 | 0.00665112 | 0.017340939 | 0.000207174 |
| T]A | | | | | | | | | | |
| T[C > | 3.68E−05 | 0.096965111 | 0.013353418 | 0.007019362 | 0.023059777 | 0.010485418 | 0.331262809 | 0.211925778 | 0.013707399 | 0.005644458 |
| T]C | | | | | | | | | | |
| T[C > | 0.110451284 | 0.044809865 | 0.00101015 | 0.000287062 | 0.016217097 | 0.052826092 | 0.075441724 | 0.058649178 | 4.72E−05 | 0.001778527 |
| T]G | | | | | | | | | | |
| T[C > | 2.21E−16 | 0.300795253 | 0.008252963 | 0.007294654 | 0.021183301 | 0.007022968 | 0.107739642 | 0.103073866 | 0.002335445 | 0.050256343 |
| T]T | | | | | | | | | | |
| T[T > | 0.006525361 | 0.000413734 | 0.006430248 | 0.008772822 | 0.006223899 | 0.000446024 | 0.00035051 | 0.003201491 | 0.015158671 | 0.016413311 |
| A]A | | | | | | | | | | |
| T[T > | 2.23E−16 | 0.000194253 | 0.010702694 | 0.004394007 | 0.004213962 | 0.000115575 | 0.000395237 | 0.002284086 | 0.034739505 | 0.001247504 |
| A]C | | | | | | | | | | |
| T[T > | 2.77E−05 | 5.01E−05 | 0.006762433 | 0.00961631 | 0.004820615 | 2.96E−05 | 2.24E−16 | 0.001713918 | 0.012252762 | 0.005615669 |
| A]G | | | | | | | | | | |
| T[T > | 0.002221005 | 0.000204312 | 0.013053346 | 0.005644097 | 0.008773503 | 0.000425445 | 0.000798703 | 2.23E−16 | 0.279174925 | 0.027275591 |
| A]T | | | | | | | | | | |
| T[T > | 0.000247614 | 2.19E−16 | 0.015013789 | 0.002380912 | 0.017754387 | 0.001453999 | 0.002116845 | 0.005928687 | 0.029726745 | 0.079806038 |
| C]A | | | | | | | | | | |
| T[T > | 0.003396973 | 4.40E−05 | 0.009163545 | 0.000512804 | 0.009883472 | 0.00110506 | 0.000605556 | 0.001764048 | 0.021229698 | 0.014503502 |
| C]C | | | | | | | | | | |
| T[T > | 0.000412157 | 0.000117038 | 0.006984152 | 0.001753918 | 0.012781171 | 0.002135088 | 0.000320461 | 0.003841192 | 0.012955789 | 0.022863795 |
| C]G | | | | | | | | | | |
| T[T > | 0.004274201 | 3.57E−05 | 0.013957039 | 0.000638727 | 0.018549692 | 0.001737734 | 0.000812715 | 0.00298329 | 0.088155236 | 0.043061463 |
| C]T | | | | | | | | | | |
| T[T > | 2.17E−16 | 1.64E−05 | 0.007161182 | 0.00037245 | 0.005148772 | 0.000103155 | 0.000126026 | 0.000943649 | 0.018997555 | 0.000207378 |
| G]A | | | | | | | | | | |
| T[T > | 5.52E−05 | 7.12E−05 | 0.00640123 | 0.000177391 | 0.006677196 | 0.000290965 | 0.00011786 | 0.001580506 | 0.017674485 | 0.000116637 |
| G]C | | | | | | | | | | |
| T[T > | 0.000577614 | 9.54E−05 | 0.008112904 | 0.002338558 | 0.006984357 | 0.000324774 | 2.24E−16 | 0.001361052 | 0.007673041 | 0.00012535 |
| G]G | | | | | | | | | | |
| T[T > | 2.20E−16 | 2.22E−16 | 0.010543087 | 0.000570435 | 0.013536262 | 0.001008685 | 8.30E−05 | 0.00177792 | 0.021713605 | 0.000165852 |
| G]T | | | | | | | | | | |
| Type | SBS8 | SBS9 | SBS10a | SBS10b | SBS10c | SBS10d | SBS11 | SBS12 | SBS13 | SBS14 |
| A[C > | 0.044310641 | 0.000560639 | 0.002185262 | 0.000180915 | 0.004342639 | 0.010124729 | 0.000147337 | 0.004542368 | 0.001816879 | 0.001126093 |
| A]A | | | | | | | | | | |
| A[C > | 0.047299564 | 0.004046991 | 0.001739372 | 0.006402357 | 0.014642916 | 0.018185496 | 0.000548604 | 0.001118361 | 0.000708842 | 0.01297141 |
| A]C | | | | | | | | | | |
| A[C > | 0.004767276 | 0.000439559 | 0.000153713 | 5.46E−05 | 0.000676583 | 0.000746925 | 9.75E−05 | 0.00055731 | 0.000270656 | 0.000426447 |
| A]G | | | | | | | | | | |
| A[C > | 0.047204587 | 0.003063133 | 0.016956073 | 1.62E−05 | 0.013156119 | 0.014205764 | 0.000268324 | 0.001225521 | 0.000347257 | 0.083014325 |
| A]T | | | | | | | | | | |
| A[C > | 0.004350682 | 0.00486289 | 2.23E−16 | 0.000303181 | 0.000348532 | 0.000129034 | 0.000171557 | 0.001226037 | 0.003863364 | 2.23E−16 |
| G]A | | | | | | | | | | |
| A[C > | 0.002919115 | 0.001939389 | 2.39E−05 | 0.00013118 | 0.000321025 | 0.000287969 | 0.000288216 | 0.000635388 | 0.000934964 | 5.72E−06 |
| G]C | | | | | | | | | | |
| A[C > | 0.000295117 | 0.001403288 | 2.29E−16 | 2.27E−16 | 4.37E−05 | 2.77E−05 | 6.18E−05 | 7.75E−05 | 0.000270656 | 4.04E−05 |
| G]G | | | | | | | | | | |
| A[C > | 0.005734855 | 0.0064376 | 2.22E−16 | 2.21E−16 | 0.000517421 | 0.000106649 | 0.000539673 | 0.001446516 | 0.003991458 | 0.000205023 |
| G]T | | | | | | | | | | |
| A[C > | 0.006842527 | 0.008138308 | 0.00103775 | 0.000545726 | 0.004879637 | 0.001369357 | 0.026036346 | 0.005537267 | 0.007167688 | 0.012467454 |
| T]A | | | | | | | | | | |
| A[C > | 0.002790476 | 0.005363005 | 0.000819569 | 0.005139507 | 0.005183137 | 0.001935687 | 0.14609571 | 0.002098165 | 2.18E−16 | 0.010000858 |
| T]C | | | | | | | | | | |
| A[C > | 0.003332965 | 0.004519413 | 0.005707862 | 0.000422631 | 0.013405701 | 0.000722658 | 0.007855763 | 0.000121782 | 0.000310639 | 0.027362816 |
| T]G | | | | | | | | | | |
| A[C > | 0.012554412 | 0.006517945 | 0.002982274 | 0.007879387 | 0.006169511 | 0.002509331 | 0.109952152 | 0.00239077 | 0.000317321 | 0.016180758 |
| T]T | | | | | | | | | | |
| A[T > | 0.019415298 | 0.012644242 | 5.01E−05 | 0.001792725 | 0.003993857 | 0.0018887 | 0.000417 | 0.005148426 | 2.20E−16 | 0.00061074 |
| A]A | | | | | | | | | | |
| A[T > | 0.021367046 | 0.002828744 | 0.000193267 | 0.000334447 | 0.013655428 | 0.003184847 | 0.000444318 | 0.002718996 | 6.50E−05 | 0.000373417 |
| A]C | | | | | | | | | | |
| A[T > | 0.022708056 | 0.002748741 | 2.21E−16 | 2.31E−05 | 0.001222456 | 0.000655356 | 3.93E−05 | 0.003107951 | 0.000583825 | 0.000352805 |
| A]G | | | | | | | | | | |
| A[T > | 0.024913437 | 0.018833676 | 0.0065911 | 0.002809774 | 0.071372031 | 0.02200544 | 3.40E−06 | 0.002810718 | 0.0006 | 0.001844812 |
| A]T | | | | | | | | | | |
| A[T > | 0.009727562 | 0.020210875 | 0.000736641 | 0.009209879 | 0.018103772 | 0.005172956 | 0.001140001 | 0.073292866 | 0.001889418 | 0.012553552 |
| C]A | | | | | | | | | | |
| A[T > | 0.00502577 | 0.010532557 | 0.003606313 | 0.000506137 | 0.009088248 | 0.003226151 | 0.000945058 | 0.040433783 | 0.000141527 | 0.006324003 |
| C]C | | | | | | | | | | |
| A[T > | 0.006951852 | 0.014241666 | 0.002116649 | 0.000523206 | 0.016837866 | 0.008489886 | 0.0003201 | 0.070227738 | 0.00042352 | 0.012358149 |
| C]G | | | | | | | | | | |
| A[T > | 0.014848409 | 0.028898233 | 0.00332524 | 0.000147883 | 0.012706936 | 0.005640828 | 0.001191054 | 0.063589993 | 0.000949505 | 0.005953257 |
| C]T | | | | | | | | | | |
| A[T > | 0.001632876 | 0.044901994 | 0.001898457 | 3.42E−05 | 0.002502817 | 0.00687662 | 0.000218 | 0.002001612 | 6.07E−05 | 0.000211218 |
| G]A | | | | | | | | | | |
| A[T > | 0.000670103 | 0.006951488 | 0.001235312 | 5.29E−05 | 0.000979805 | 0.000613297 | 4.71E−05 | 0.000666204 | 2.21E−16 | 0.002981316 |
| G]C | | | | | | | | | | |
| A[T > | 0.004750764 | 0.005308257 | 0.000316508 | 0.000515323 | 0.00103232 | 0.000932459 | 0.000179056 | 0.00080488 | 8.16E−05 | 0.000706607 |
| G]G | | | | | | | | | | |
| A[T > | 0.000737438 | 0.02381613 | 0.009124616 | 2.19E−16 | 0.012982246 | 0.004821013 | 0.000285252 | 0.001275787 | 0.000316832 | 0.01106887 |
| G]T | | | | | | | | | | |
| C[C > | 0.039839576 | 0.004768604 | 0.003137527 | 0.000512084 | 0.017281904 | 0.009901992 | 0.000616666 | 0.005385724 | 0.001381922 | 0.016204834 |
| A]A | | | | | | | | | | |
| C[C > | 0.0382896 | 0.001894654 | 0.000197966 | 0.003104625 | 0.009522375 | 0.014599205 | 0.000977275 | 0.004224422 | 0.000949095 | 0.01945369 |
| A]C | | | | | | | | | | |
| C[C > | 0.003419 | 0.001373551 | 4.62E−05 | 8.31E−05 | 0.002405688 | 0.001018521 | 9.26E−06 | 0.001173289 | 4.31E−06 | 0.001384556 |
| A]G | | | | | | | | | | |
| C[C > | 0.03278667 | 0.003040076 | 0.017069448 | 0.008187688 | 0.044499848 | 0.018361956 | 0.000149681 | 0.001093076 | 2.19E−16 | 0.258489774 |
| A]T | | | | | | | | | | |
| C[C > | 0.004967528 | 0.000887159 | 4.51E−05 | 2.94E−05 | 0.000109247 | 8.09E−05 | 0.000260436 | 0.001331526 | 0.00553756 | 8.50E−05 |
| G]A | | | | | | | | | | |
| C[C > | 0.005388176 | 0.001519671 | 2.19E−16 | 2.17E−16 | 0.000204405 | 0.000122755 | 0.000175434 | 0.00087943 | 0.000635345 | 7.76E−05 |
| G]C | | | | | | | | | | |
| C[C > | 1.05E−05 | 0.000438133 | 2.00E−05 | 2.20E−16 | 0.000180304 | 2.90E−05 | 1.13E−05 | 0.000173486 | 0.000486127 | 1.92E−05 |
| G]G | | | | | | | | | | |
| C[C > | 0.00545451 | 0.003010271 | 2.97E−05 | 2.18E−16 | 0.000236211 | 0.000228466 | 9.62E−06 | 0.001550181 | 0.006317548 | 0.000176966 |
| G]T | | | | | | | | | | |
| C[C > | 0.00265266 | 0.007192644 | 3.63E−05 | 0.000148416 | 0.003728588 | 0.000898461 | 0.004061212 | 0.006548325 | 1.91E−05 | 0.002714061 |
| T]A | | | | | | | | | | |
| C[C > | 0.003967118 | 0.00864436 | 2.19E−16 | 8.24E−05 | 0.006542111 | 0.002165791 | 0.124885075 | 0.000250702 | 0.001323635 | 0.001362746 |
| T]C | | | | | | | | | | |
| C[C > | 0.001493933 | 0.006526876 | 2.22E−16 | 0.003670112 | 0.009475857 | 0.000591316 | 0.000926453 | 4.10E−05 | 0.001982362 | 0.011638295 |
| T]G | | | | | | | | | | |
| C[C > | 0.014406264 | 0.011027727 | 0.000101627 | 0.000584835 | 0.009752328 | 0.00251532 | 0.064961381 | 0.003358726 | 0.00143132 | 0.0013521 |
| T]T | | | | | | | | | | |
| C[T > | 0.018978021 | 0.004784235 | 2.21E−16 | 7.01E−05 | 0.000670929 | 0.001210228 | 0.000184589 | 0.000545462 | 0.00020741 | 0.000154923 |
| A]A | | | | | | | | | | |
| C[T > | 0.029254478 | 0.003135693 | 2.22E−16 | 0.000105063 | 0.0027907 | 0.000613847 | 0.000213322 | 0.00561141 | 0.000340425 | 0.002957455 |
| A]C | | | | | | | | | | |
| C]T > | 0.027568557 | 0.005303702 | 2.21E−16 | 2.19E−16 | 0.000897199 | 0.000998974 | 0.000305982 | 0.001408635 | 0.000224284 | 0.000316848 |
| A]G | | | | | | | | | | |
| C[T > | 0.042024805 | 0.00446367 | 0.000375411 | 0.000232744 | 0.003363841 | 0.002112612 | 0.000163582 | 0.001508326 | 0.000522416 | 0.001388338 |
| A]T | | | | | | | | | | |
| C[T > | 0.002387235 | 0.01368351 | 0.000121017 | 0.00130438 | 0.011931533 | 0.002586658 | 0.00032905 | 0.045355256 | 4.70E−05 | 0.003158428 |
| C]A | | | | | | | | | | |
| C[T > | 0.00701306 | 0.015327826 | 4.10E−05 | 0.000122903 | 0.009780141 | 0.002261641 | 0.000717447 | 0.049801261 | 0.000121438 | 0.005353495 |
| C]C | | | | | | | | | | |
| C[T > | 0.001258565 | 0.015381735 | 0.00104156 | 2.50E−05 | 0.014579222 | 0.003301465 | 0.000131422 | 0.037363797 | 2.20E−16 | 0.004467856 |
| C]G | | | | | | | | | | |
| C[T > | 0.006494743 | 0.03307539 | 2.44E−06 | 0.000810625 | 0.019310258 | 0.004754822 | 8.53E−05 | 0.046154767 | 0.000111875 | 0.002072446 |
| C]T | | | | | | | | | | |
| C[T > | 0.001218589 | 0.025968706 | 0.000153751 | 0.000224314 | 0.010778052 | 0.006311601 | 2.23E−16 | 0.00089212 | 4.11E−05 | 4.85E−05 |
| G]A | | | | | | | | | | |
| C[T > | 0.001953638 | 0.007423477 | 2.22E−16 | 0.00113983 | 0.002296256 | 0.000493949 | 2.23E−16 | 0.001142323 | 2.81E−06 | 0.001213058 |
| G]C | | | | | | | | | | |
| C[T > | 0.004694646 | 0.014183158 | 5.31E−05 | 0.00047334 | 0.006842037 | 0.003061932 | 2.23E−16 | 0.000592426 | 2.20E−16 | 0.000434791 |
| G]G | | | | | | | | | | |
| C]T > | 0.000577087 | 0.056801201 | 0.001297964 | 0.005201924 | 0.030107992 | 0.005934772 | 0.000134299 | 0.000345909 | 5.54E−05 | 0.000967812 |
| G]T | | | | | | | | | | |
| G[C > | 0.024223162 | 0.007727027 | 0.001202793 | 0.000723879 | 0.008407369 | 0.00241244 | 0.000165879 | 0.001982235 | 0.000943772 | 0.002083365 |
| A]A | | | | | | | | | | |
| G[C > | 0.025864633 | 0.006594185 | 0.000432348 | 0.002490452 | 0.007323171 | 0.02462788 | 0.00040156 | 0.000411733 | 0.000442351 | 0.022127875 |
| A]C | | | | | | | | | | |
| G[C > | 0.002764369 | 0.000172513 | 1.05E−05 | 8.50E−05 | 0.000898181 | 8.78E−05 | 1.41E−05 | 0.000451053 | 0.00016439 | 0.001120611 |
| A]G | | | | | | | | | | |
| G[C > | 0.026789926 | 0.008524573 | 0.010978228 | 0.002758736 | 0.011981979 | 0.009812483 | 2.22E−16 | 2.74E−05 | 6.09E−05 | 0.125567264 |
| A]T | | | | | | | | | | |
| G[C > | 0.002582469 | 0.001361238 | 1.49E−05 | 1.70E−05 | 0.000169898 | 0.000104015 | 4.69E−05 | 0.000564637 | 0.001491736 | 2.22E−16 |
| G]A | | | | | | | | | | |
| G[C > | 0.002359407 | 0.002793644 | 8.15E−05 | 0.000132824 | 0.000382544 | 0.000319802 | 0.000113032 | 0.00028535 | 0.000469814 | 4.28E−05 |
| G]C | | | | | | | | | | |
| G[C > | 0.00049234 | 0.000525609 | 2.23E−16 | 2.22E−16 | 3.31E−05 | 1.92E−05 | 3.28E−06 | 9.63E−05 | 0.000241573 | 2.24E−16 |
| G]G | | | | | | | | | | |
| G[C > | 0.004840113 | 0.008534532 | 3.50E−05 | 0.000111335 | 0.00015371 | 0.000367189 | 0.000186047 | 0.000502024 | 0.00267158 | 8.00E−05 |
| G]T | | | | | | | | | | |
| G[C > | 0.000276264 | 0.006986353 | 4.19E−05 | 0.000359464 | 0.003609428 | 0.001151317 | 0.019804897 | 0.004485057 | 0.007140443 | 0.017127668 |
| T]A | | | | | | | | | | |
| G[C > | 0.000241864 | 0.006446112 | 0.012254767 | 0.01474738 | 0.008894859 | 0.005475285 | 0.114023342 | 0.001797012 | 0.000903337 | 0.008060869 |
| T]C | | | | | | | | | | |
| G[C > | 0.001926987 | 0.004802109 | 0.01492869 | 0.012576183 | 0.009285922 | 0.002057197 | 0.001803667 | 7.53E−05 | 0.000178423 | 1.94E−05 |
| T]G | | | | | | | | | | |
| G[C > | 0.004591136 | 0.010655717 | 0.013351898 | 0.031429887 | 0.010318053 | 0.00223067 | 0.069617651 | 0.00265953 | 0.001256632 | 0.010463939 |
| T]T | | | | | | | | | | |
| G[T > | 0.012711174 | 0.002631487 | 3.26E−06 | 2.18E−16 | 0.001884989 | 0.001529961 | 1.54E−06 | 0.000820409 | 6.61E−06 | 5.73E−05 |
| A]A | | | | | | | | | | |
| G[T > | 0.013436723 | 0.002109964 | 2.20E−16 | 0.000121115 | 0.006842122 | 0.003324915 | 0.000374871 | 0.001801825 | 0.000128555 | 0.002718998 |
| A]C | | | | | | | | | | |
| G[T > | 0.015655364 | 0.00079076 | 7.28E−05 | 2.20E−16 | 0.0019392 | 0.0005395 | 0.000119943 | 0.00251934 | 4.69E−05 | 0.000120505 |
| A]G | | | | | | | | | | |
| G[T > | 0.028474707 | 0.002594015 | 0.000428699 | 0.00010827 | 0.012957532 | 0.006940709 | 0.000166912 | 0.00092736 | 0.00036043 | 0.000869001 |
| A]T | | | | | | | | | | |
| G[T > | 0.002581957 | 0.009453492 | 0.002169644 | 0.014441953 | 0.012623338 | 0.002405852 | 0.000806891 | 0.070618703 | 0.000283167 | 0.010930877 |
| C]A | | | | | | | | | | |
| G[T > | 0.00260772 | 0.009733703 | 0.006207393 | 0.005248313 | 0.006028056 | 0.002714836 | 0.000859704 | 0.030262697 | 0.000179977 | 0.006513643 |
| C]C | | | | | | | | | | |
| G[T > | 0.002819973 | 0.006332097 | 2.22E−16 | 0.001370095 | 0.012747215 | 0.003532914 | 0.000565448 | 0.041654422 | 0.000194428 | 0.011046327 |
| C]G | | | | | | | | | | |
| G[T > | 0.006373876 | 0.009991012 | 0.005323517 | 0.011528789 | 0.010687509 | 0.002187587 | 0.000241208 | 0.046925443 | 2.24E−16 | 0.011356843 |
| C]T | | | | | | | | | | |
| G]T > | 0.001300909 | 0.008242015 | 0.000201185 | 0.000472556 | 0.000351827 | 0.000388904 | 9.55E−05 | 0.000409211 | 8.65E−05 | 7.74E−05 |
| G]A | | | | | | | | | | |
| G[T > | 0.000652925 | 0.005822307 | 0.000823369 | 0.000927563 | 0.000616053 | 0.001559803 | 2.22E−16 | 0.000212038 | 2.20E−16 | 0.002091537 |
| G]C | | | | | | | | | | |
| G[T > | 0.003984089 | 0.003983903 | 2.22E−16 | 0.001717582 | 0.000615079 | 0.000200029 | 0.000131031 | 0.000497846 | 0.00042475 | 0.000784289 |
| G]G | | | | | | | | | | |
| G[T > | 0.001307202 | 0.019657773 | 0.002224002 | 0.005172917 | 0.007556799 | 0.000959256 | 0.000156734 | 0.000167229 | 2.91E−06 | 0.002281509 |
| G]T | | | | | | | | | | |
| T[C > | 0.029945072 | 0.017081958 | 0.093805211 | 0.000443381 | 0.060346754 | 0.259270928 | 0.000181666 | 0.003497541 | 0.078871738 | 0.000348919 |
| A]A | | | | | | | | | | |
| T[C > | 0.025176237 | 0.002467356 | 0.009931212 | 0.003770801 | 0.008550395 | 0.014397123 | 0.000944954 | 0.002889243 | 0.029298787 | 0.0243899 |
| A]C | | | | | | | | | | |
| T[C > | 0.001938739 | 0.000454052 | 0.001175476 | 0.000578433 | 0.002705634 | 0.00782571 | 2.28E−16 | 0.000515385 | 0.012266944 | 7.23E−06 |
| A]G | | | | | | | | | | |
| T[C > | 0.03219251 | 0.027549039 | 0.671473651 | 0.081468029 | 0.190119224 | 0.343736498 | 4.79E−05 | 0.002371955 | 0.035594077 | 0.152485221 |
| A]T | | | | | | | | | | |
| T[C > | 0.001477156 | 0.002702969 | 5.54E−05 | 2.21E−16 | 0.000259442 | 0.000121046 | 1.57E−05 | 0.000973884 | 0.316485329 | 1.98E−05 |
| G]A | | | | | | | | | | |
| T[C > | 0.00134407 | 0.002005981 | 2.22E−16 | 2.20E−16 | 0.000396012 | 6.54E−05 | 0.000225661 | 0.001554974 | 0.063281395 | 0.000150555 |
| G]C | | | | | | | | | | |
| T[C > | 7.98E−05 | 0.000412218 | 2.26E−16 | 2.24E−16 | 1.06E−05 | 1.99E−05 | 2.28E−16 | 0.000218401 | 0.013990399 | 1.87E−05 |
| G]G | | | | | | | | | | |
| T[C > | 0.000251283 | 0.005681359 | 5.46E−05 | 4.33E−05 | 0.000349567 | 0.000169644 | 0.000187511 | 0.003078495 | 0.36997787 | 0.000324157 |
| G]T | | | | | | | | | | |
| T[C > | 0.005064536 | 0.004290586 | 0.000365242 | 0.002713967 | 0.002409862 | 0.001333557 | 0.009870493 | 0.005547823 | 0.006918749 | 0.003559582 |
| T]A | | | | | | | | | | |
| T[C > | 0.000252765 | 0.007351919 | 0.002749262 | 0.048127325 | 0.009025752 | 0.003581617 | 0.159171412 | 0.002648473 | 0.004464577 | 0.000137507 |
| T]C | | | | | | | | | | |
| T[C > | 4.26E−05 | 0.003326313 | 0.002827223 | 0.441143755 | 0.008747446 | 0.005402479 | 3.01E−07 | 9.03E−05 | 0.001206419 | 0.005432054 |
| T]G | | | | | | | | | | |
| T[C > | 0.005318324 | 0.005509808 | 0.022850148 | 0.127792987 | 0.012890453 | 0.008157361 | 0.120615191 | 0.006177177 | 0.001423763 | 0.000362531 |
| T]T | | | | | | | | | | |
| T[T > | 0.01134804 | 0.025655235 | 0.00199914 | 0.00298729 | 0.009809541 | 0.008775299 | 0.000184343 | 0.003701082 | 0.000290202 | 0.000197488 |
| A]A | | | | | | | | | | |
| T[T > | 0.01985705 | 0.001883786 | 0.00036406 | 0.000669938 | 0.002514474 | 0.000772443 | 0.000206596 | 0.006569227 | 0.000234722 | 0.00176284 |
| A]C | | | | | | | | | | |
| T[T > | 0.012988125 | 0.016813297 | 0.000692915 | 7.03E−05 | 0.000568426 | 0.003793709 | 0.000203256 | 0.002789401 | 0.000188061 | 0.000359676 |
| A]G | | | | | | | | | | |
| T[T > | 0.038018415 | 0.017052316 | 0.005269877 | 0.001508451 | 0.004835785 | 0.006965748 | 0.000679056 | 0.005267306 | 6.18E−05 | 0.001826892 |
| A]T | | | | | | | | | | |
| T[T > | 0.002052515 | 0.013814357 | 0.000715378 | 0.008649472 | 0.016737525 | 0.005553103 | 0.000111993 | 0.084976846 | 2.17E−05 | 0.006191234 |
| C]A | | | | | | | | | | |
| T[T > | 0.002688339 | 0.01384837 | 0.005228302 | 8.16E−05 | 0.017029315 | 0.006573317 | 0.000477626 | 0.047155848 | 2.52E−05 | 0.00293467 |
| C]C | | | | | | | | | | |
| T[T > | 2.24E−16 | 0.009373161 | 0.000362956 | 0.000665374 | 0.015185349 | 0.004983685 | 0.00015674 | 0.040280162 | 9.98E−05 | 0.004342309 |
| C]G | | | | | | | | | | |
| T[T > | 0.004584279 | 0.038518172 | 0.003237637 | 2.49E−05 | 0.019939417 | 0.007550501 | 0.000106795 | 0.059896794 | 0.000529218 | 0.002931057 |
| C]T | | | | | | | | | | |
| T[T > | 2.19E−16 | 0.064828805 | 0.002636121 | 0.011031494 | 0.008596591 | 0.019562162 | 8.43E−05 | 0.001608737 | 0.000180396 | 9.80E−05 |
| G]A | | | | | | | | | | |
| T[T > | 0.001160874 | 0.008777423 | 2.26E−16 | 0.005591212 | 0.000967472 | 0.000749058 | 6.88E−05 | 0.001150888 | 2.25E−16 | 0.001222779 |
| G]C | | | | | | | | | | |
| T[T > | 0.003111109 | 0.010973948 | 2.16E−05 | 0.002749152 | 0.001113495 | 0.003157757 | 0.000102134 | 0.000881129 | 3.67E−05 | 0.000114855 |
| G]G | | | | | | | | | | |
| T[T > | 0.000999112 | 0.064096645 | 0.018828105 | 0.090209354 | 0.02513817 | 0.014519751 | 5.59E−05 | 0.002217285 | 1.88E−05 | 0.000607291 |
| G]T | | | | | | | | | | |
| Type | SBS15 | SBS16 | SBS17a | SBS17b | SBS18 | SBS19 | SBS20 | SBS21 | SBS22 | SBS23 |
| A[C > | 0.00094603 | 0.016094373 | 0.002072799 | 0.000607769 | 0.05168832 | 0.001279613 | 0.000624248 | 0.000157722 | 0.006046172 | 0.000844507 |
| A]A | | | | | | | | | | |
| A[C > | 0.000490511 | 0.002872843 | 0.000905293 | 0.000126994 | 0.015617161 | 0.000636052 | 0.001380514 | 0.002334882 | 9.45E−05 | 0.00039742 |
| A]C | | | | | | | | | | |
| A[C > | 4.74E−05 | 0.001053691 | 4.89E−05 | 5.97E−05 | 0.002504669 | 0.000254548 | 2.26E−05 | 0.000303319 | 0.000790362 | 1.02E−07 |
| A]G | | | | | | | | | | |
| A[C > | 0.001111918 | 0.010658028 | 6.19E−05 | 0.000455634 | 0.0214692 | 0.00057508 | 0.001249985 | 0.000623594 | 0.001729623 | 5.67E−18 |
| A]T | | | | | | | | | | |
| A[C > | 0.000113243 | 0.001750263 | 0.001011366 | 0.000145944 | 0.001736326 | 0.003214147 | 0.008844347 | 1.70E−05 | 0.001669991 | 5.81E−18 |
| G]A | | | | | | | | | | |
| A[C > | 0.000265488 | 0.001991177 | 0.000561124 | 4.27E−05 | 0.002560028 | 0.001776184 | 0.001499695 | 0.000711347 | 0.000193197 | 4.06E−05 |
| G]C | | | | | | | | | | |
| A[C > | 3.16E−05 | 0.000874976 | 0.000156311 | 2.29E−16 | 0.001979988 | 0.000637404 | 0.000485733 | 2.29E−16 | 0.000238658 | 2.97E−18 |
| G]G | | | | | | | | | | |
| A[C > | 0.000325562 | 0.011562955 | 1.96E−05 | 0.000318744 | 0.004091933 | 0.00215529 | 0.004455591 | 6.22E−05 | 6.23E−05 | 5.67E−18 |
| G]T | | | | | | | | | | |
| A[C > | 0.00506086 | 0.001770381 | 0.00122165 | 0.000570783 | 0.009233642 | 0.02821195 | 0.049717936 | 0.002179982 | 0.001770593 | 0.022857308 |
| T]A | | | | | | | | | | |
| A[C > | 0.006819787 | 0.00369507 | 0.001686331 | 6.92E−05 | 0.00459619 | 0.013991169 | 0.012911281 | 0.003878278 | 0.000217966 | 0.080579573 |
| T]C | | | | | | | | | | |
| A[C > | 0.076673991 | 0.003378008 | 4.06E−05 | 0.000294629 | 0.01257488 | 0.001603857 | 0.013774526 | 0.007634554 | 0.001405089 | 0.009057158 |
| T]G | | | | | | | | | | |
| A[C > | 0.001783076 | 0.006032846 | 0.000737686 | 0.000146882 | 0.007995772 | 0.037062935 | 0.01310468 | 4.19E−05 | 0.000619446 | 0.035383565 |
| T]T | | | | | | | | | | |
| A[T > | 0.002025831 | 0.02302534 | 0.001002186 | 6.20E−05 | 0.002476421 | 0.000784761 | 0.000510654 | 0.003444365 | 0.063701089 | 8.30E−18 |
| A]A | | | | | | | | | | |
| A[T > | 0.000841442 | 0.007773032 | 0.00099673 | 0.000288422 | 0.000879782 | 0.000256514 | 0.005225514 | 0.005666788 | 0.007040759 | 5.61E−18 |
| A]C | | | | | | | | | | |
| A[T > | 0.000536417 | 0.00878427 | 0.000198515 | 6.51E−05 | 0.002586629 | 0.000950797 | 0.00030289 | 0.000246956 | 0.060430237 | 0.000354891 |
| A]G | | | | | | | | | | |
| A[T > | 0.009482122 | 0.010974152 | 0.001449986 | 0.000627571 | 0.007804147 | 0.000653547 | 0.025605328 | 0.00159418 | 0.002504401 | 0.000161498 |
| A]T | | | | | | | | | | |
| A[T > | 0.003862981 | 0.241217849 | 9.58E−05 | 9.64E−05 | 0.002008984 | 0.002585919 | 0.010692763 | 0.016624537 | 0.024922179 | 0.000887954 |
| C]A | | | | | | | | | | |
| A[T > | 0.001961031 | 0.012955054 | 0.012696559 | 0.000251496 | 0.002094242 | 0.000597526 | 0.004581134 | 0.010029714 | 0.000902945 | 0.000179735 |
| C]C | | | | | | | | | | |
| A[T > | 0.000238407 | 0.063713374 | 0.000373208 | 2.21E−16 | 0.002069304 | 0.00230708 | 0.018593228 | 0.003863674 | 0.007728289 | 0.000471184 |
| C]G | | | | | | | | | | |
| A[T > | 0.000923364 | 0.135680423 | 0.007736572 | 0.000539334 | 0.00422061 | 0.001738796 | 0.000103021 | 0.025506876 | 0.000753316 | 0.000261807 |
| C]T | | | | | | | | | | |
| A[T > | 0.000464749 | 0.011163801 | 0.000844416 | 0.00139667 | 0.000717068 | 0.002326328 | 1.05E−05 | 0.000390229 | 0.002322747 | 8.30E−18 |
| G]A | | | | | | | | | | |
| A[T > | 0.000415218 | 0.004428821 | 0.00069981 | 0.004471037 | 0.000859742 | 0.00190122 | 2.14E−05 | 0.000438298 | 5.11E−05 | 5.48E−18 |
| G]C | | | | | | | | | | |
| A[T > | 0.000183772 | 0.004606507 | 0.00071366 | 0.000980947 | 0.001979766 | 0.001767763 | 2.69E−05 | 0.000186213 | 0.002941736 | 5.69E−18 |
| G]G | | | | | | | | | | |
| A[T > | 0.003607977 | 0.007941295 | 0.012414268 | 0.068507332 | 0.001831586 | 0.001618879 | 5.82E−05 | 6.92E−05 | 4.17E−05 | 7.45E−18 |
| G]T | | | | | | | | | | |
| C[C > | 0.041420004 | 0.010343995 | 0.000292085 | 0.000267858 | 0.073437487 | 0.001315073 | 0.037493408 | 2.21E−16 | 0.001820363 | 0.000151006 |
| A]A | | | | | | | | | | |
| C[C > | 0.004970545 | 0.008921163 | 0.001140839 | 4.04E−05 | 0.019320614 | 0.000759015 | 0.097958501 | 0.002565347 | 0.004377129 | 0.000625802 |
| A]C | | | | | | | | | | |
| C[C > | 6.62E−05 | 0.00174654 | 2.22E−16 | 2.22E−16 | 0.012018263 | 0.000237281 | 0.00747705 | 0.001984877 | 0.000136528 | 0.0001312 |
| A]G | | | | | | | | | | |
| C[C > | 0.028439947 | 0.002476665 | 0.00013565 | 0.000179895 | 0.036025146 | 3.43E−05 | 0.251293147 | 0.018973192 | 0.000109424 | 0.000580041 |
| A]T | | | | | | | | | | |
| C[C > | 8.28E−05 | 0.003530883 | 0.000547536 | 0.000191751 | 0.00060834 | 0.001763394 | 7.67E−05 | 1.96E−05 | 0.001044471 | 0.000174007 |
| G]A | | | | | | | | | | |
| C[C > | 1.67E−05 | 0.00448528 | 0.000231118 | 4.58E−05 | 0.002227785 | 0.001791156 | 4.34E−06 | 0.000269361 | 0.000775631 | 0.000253301 |
| G]C | | | | | | | | | | |
| C[C > | 5.02E−05 | 0.001465488 | 0.00023182 | 2.22E−16 | 0.003585448 | 4.84E−05 | 0.00018919 | 1.01E−05 | 0.000239927 | 1.88E−18 |
| G]G | | | | | | | | | | |
| C[C > | 8.37E−05 | 0.009568483 | 3.86E−05 | 0.000190768 | 0.003761303 | 0.003208078 | 1.01E−05 | 0.000171851 | 0.000318324 | 0.000227016 |
| G]T | | | | | | | | | | |
| C[C > | 0.010701819 | 0.007131389 | 0.000812888 | 0.000451702 | 0.011611062 | 0.118555837 | 0.01056995 | 0.001340997 | 0.001203628 | 0.062202512 |
| T]A | | | | | | | | | | |
| C[C > | 0.010925356 | 0.00280577 | 0.00169159 | 5.78E−05 | 0.00666364 | 0.091042165 | 0.012876244 | 0.005091227 | 0.003151928 | 0.187740562 |
| T]C | | | | | | | | | | |
| C[C > | 0.02480047 | 0.000693597 | 0.001898523 | 0.000541639 | 0.016825568 | 0.015785197 | 0.016604484 | 0.00623532 | 0.000427652 | 0.014331124 |
| T]G | | | | | | | | | | |
| C[C > | 0.015458647 | 0.019196644 | 0.00172286 | 0.000134427 | 0.006599648 | 0.25704474 | 0.011667182 | 0.000322842 | 0.001591622 | 0.156010982 |
| T]T | | | | | | | | | | |
| C[T > | 0.000346689 | 0.003209867 | 0.000432021 | 9.30E−05 | 0.00054077 | 9.62E−05 | 0.000144363 | 6.18E−06 | 0.128009986 | 0.000464501 |
| A]A | | | | | | | | | | |
| C]T > | 0.000243816 | 0.005717011 | 0.040437283 | 0.002232661 | 0.003062293 | 0.000404874 | 0.006516002 | 0.000689162 | 0.055271043 | 4.51E−18 |
| A]C | | | | | | | | | | |
| C[T > | 8.17E−05 | 0.001029975 | 0.012343664 | 0.000712508 | 0.00320277 | 0.000294481 | 0.000123312 | 0.000124836 | 0.168016059 | 0.000132717 |
| A]G | | | | | | | | | | |
| C[T > | 0.001052888 | 0.006572449 | 0.072441123 | 0.0077917 | 0.002269622 | 0.000219782 | 0.000102926 | 2.23E−16 | 0.060699247 | 0.000379497 |
| A]T | | | | | | | | | | |
| C[T > | 0.000747175 | 0.010699555 | 0.011049376 | 0.000642935 | 0.000385124 | 0.000452732 | 0.011729511 | 0.011484678 | 0.006740526 | 6.90E−18 |
| C]A | | | | | | | | | | |
| C[T > | 0.002148382 | 0.00317946 | 0.172732099 | 0.006438836 | 0.002461843 | 8.18E−05 | 0.004856835 | 0.002824765 | 0.00134416 | 4.51E−18 |
| C]C | | | | | | | | | | |
| C[T > | 0.001384788 | 0.00960977 | 0.086903374 | 0.003895439 | 0.001616351 | 0.000498815 | 0.026166292 | 0.014381067 | 0.002030194 | 0.000391113 |
| C]G | | | | | | | | | | |
| C[T > | 0.001754814 | 0.01560076 | 0.42683151 | 0.018303995 | 0.0025709 | 0.000394196 | 0.006962661 | 0.02181287 | 0.000539549 | 4.77E−05 |
| C]T | | | | | | | | | | |
| C[T > | 0.000205224 | 0.003959835 | 0.000308586 | 0.00203712 | 0.000110748 | 0.000715157 | 0.000965429 | 4.34E−06 | 0.004910383 | 6.89E−18 |
| G]A | | | | | | | | | | |
| C[T > | 0.000156882 | 0.001083223 | 0.001038389 | 0.03129713 | 0.001991491 | 0.00209972 | 0.001608889 | 5.45E−05 | 0.000882732 | 0.000149251 |
| G]C | | | | | | | | | | |
| C[T > | 0.00027098 | 0.002239946 | 1.93E−06 | 0.013614161 | 0.002923401 | 0.001332178 | 0.002716883 | 2.01E−05 | 0.008030768 | 6.56E−05 |
| G]G | | | | | | | | | | |
| C[T > | 0.002496849 | 0.002556512 | 0.005320365 | 0.550120054 | 0.004157627 | 0.002217981 | 0.005156405 | 0.000185962 | 0.001197879 | 0.000249962 |
| G]T | | | | | | | | | | |
| G[C > | 0.002286201 | 0.008627863 | 0.001775632 | 0.000993828 | 0.108982738 | 0.000817044 | 0.010850155 | 0.002141672 | 0.002234894 | 0.000339541 |
| A]A | | | | | | | | | | |
| G[C > | 0.002451703 | 0.004200283 | 0.001259309 | 2.73E−06 | 0.017059523 | 0.000269265 | 0.005598223 | 0.006978275 | 0.001709967 | 0.000537588 |
| A]C | | | | | | | | | | |
| G[C > | 0.000406528 | 0.001373627 | 0.000106578 | 9.27E−05 | 0.007618736 | 0.000207398 | 0.001589797 | 0.001704731 | 0.000520223 | 0.000120848 |
| A]G | | | | | | | | | | |
| G[C > | 0.018078157 | 0.005782716 | 4.66E−05 | 0.000529085 | 0.061975927 | 0.000272639 | 0.002488945 | 0.008862024 | 0.000549424 | 3.10E−05 |
| A]T | | | | | | | | | | |
| G]C > | 0.000992351 | 0.001052178 | 0.000875845 | 5.96E−05 | 0.000634899 | 0.001314899 | 0.000672107 | 0.000388303 | 0.001403072 | 4.35E−18 |
| G]A | | | | | | | | | | |
| G[C > | 0.000598649 | 0.003053853 | 0.000821503 | 5.45E−05 | 0.001321373 | 0.001277028 | 0.002833791 | 0.001401577 | 0.000919231 | 0.000126199 |
| G]C | | | | | | | | | | |
| G[C > | 1.02E−05 | 8.21E−05 | 2.38E−05 | 2.24E−16 | 0.000643965 | 0.000603984 | 0.000700726 | 0.00011802 | 0.00021314 | 1.86E−18 |
| G]G | | | | | | | | | | |
| G[C > | 0.000447981 | 0.003220375 | 0.000781109 | 2.21E−16 | 0.001223602 | 0.000319577 | 0.003018721 | 0.000206117 | 0.000453698 | 4.05E−18 |
| G]T | | | | | | | | | | |
| G[C > | 0.073577745 | 0.006513486 | 0.001635975 | 6.08E−05 | 0.009678467 | 0.061228123 | 0.042395977 | 0.009327281 | 0.002385223 | 0.054104944 |
| T]A | | | | | | | | | | |
| G[C > | 0.127015951 | 0.005228118 | 2.18E−16 | 0.000154195 | 0.008056434 | 0.039300781 | 0.052712474 | 0.019345714 | 0.001848346 | 0.153028724 |
| T]C | | | | | | | | | | |
| G[C > | 0.278733196 | 0.001181724 | 0.001618783 | 0.000858177 | 0.014914987 | 0.003247552 | 0.020758502 | 4.38E−05 | 2.24E−16 | 0.012491015 |
| T]G | | | | | | | | | | |
| G[C > | 0.07668317 | 0.006699974 | 6.55E−05 | 2.21E−16 | 0.0066552 | 0.108855751 | 0.033085977 | 0.008015651 | 0.001754965 | 0.107263069 |
| T]T | | | | | | | | | | |
| G[T > | 0.000506128 | 0.000934519 | 6.39E−05 | 9.61E−05 | 0.001587126 | 0.00018821 | 5.54E−05 | 0.001161677 | 0.053393272 | 6.26E−18 |
| A]A | | | | | | | | | | |
| G[T > | 0.000584707 | 0.000605826 | 0.003372531 | 0.000343601 | 0.001938697 | 7.77E−05 | 0.002557386 | 0.003244149 | 0.010164741 | 0.00014026 |
| A]C | | | | | | | | | | |
| G[T > | 0.000187173 | 0.005575916 | 0.00069509 | 0.000240614 | 0.001834448 | 0.000489081 | 0.002145433 | 0.000744502 | 0.03767961 | 4.73E−18 |
| A]G | | | | | | | | | | |
| G[T > | 0.000848977 | 0.000979974 | 0.004847431 | 0.001592852 | 0.003502231 | 0.000287571 | 0.004373389 | 0.000949328 | 0.009455955 | 5.90E−18 |
| A]T | | | | | | | | | | |
| G[T > | 0.012777001 | 0.002047989 | 0.00012173 | 5.88E−05 | 0.000702303 | 0.000950013 | 0.002750942 | 0.221413557 | 0.000716882 | 0.000217911 |
| C]A | | | | | | | | | | |
| G[T > | 0.011515455 | 0.001185745 | 0.014779621 | 0.001485307 | 0.0022469 | 0.000370288 | 0.002257692 | 0.06597764 | 0.001235714 | 0.000418777 |
| C]C | | | | | | | | | | |
| G[T > | 0.005955511 | 0.004299985 | 0.00357046 | 0.001008383 | 0.003989674 | 0.000959041 | 0.004089416 | 0.117394591 | 0.000583783 | 4.73E−18 |
| C]G | | | | | | | | | | |
| G[T > | 0.005922623 | 0.002434719 | 0.005291779 | 0.001481957 | 0.003188447 | 0.002398118 | 0.000943838 | 0.134749835 | 0.000846166 | 0.00081498 |
| C]T | | | | | | | | | | |
| G[T > | 0.000201064 | 0.000896741 | 0.00048989 | 0.000277618 | 0.000863 | 0.000633342 | 2.21E−16 | 0.000283965 | 0.001580918 | 6.26E−18 |
| G]A | | | | | | | | | | |
| G[T > | 0.000575773 | 0.000897778 | 0.000202352 | 0.006188779 | 3.68E−05 | 0.00070165 | 6.64E−05 | 0.000530408 | 0.000235184 | 8.71E−05 |
| G]C | | | | | | | | | | |
| G[T > | 0.000347321 | 0.000678152 | 0.000835108 | 0.00147763 | 0.00584417 | 0.000513233 | 8.76E−05 | 0.000213718 | 0.000452155 | 0.000158594 |
| G]G | | | | | | | | | | |
| G[T > | 0.001404854 | 0.002424574 | 0.007311542 | 0.114927286 | 0.00216612 | 0.000346508 | 0.003620759 | 0.002624076 | 1.99E−05 | 5.90E−18 |
| G]T | | | | | | | | | | |
| T[C > | 0.017238563 | 0.010864694 | 0.000190274 | 1.24E−05 | 0.074076629 | 0.001390573 | 0.001724654 | 0.00146685 | 0.000788791 | 0.002154447 |
| A]A | | | | | | | | | | |
| T[C > | 0.005812417 | 0.005894403 | 2.59E−05 | 2.23E−16 | 0.043683692 | 0.001136583 | 0.003392686 | 0.000236675 | 0.001747443 | 0.000670397 |
| A]C | | | | | | | | | | |
| T[C > | 7.83E−05 | 0.001522077 | 2.26E−16 | 9.65E−05 | 0.0129445 | 0.000291619 | 2.27E−16 | 6.97E−05 | 2.27E−16 | 9.37E−05 |
| A]G | | | | | | | | | | |
| T[C > | 0.008102584 | 0.009345241 | 0.00028965 | 0.002580255 | 0.122981487 | 0.001396526 | 0.00091565 | 2.24E−16 | 0.000400125 | 0.000243794 |
| A]T | | | | | | | | | | |
| T[C > | 0.00021448 | 0.002525035 | 0.003755414 | 0.000159954 | 0.002148021 | 0.001763408 | 0.000130105 | 0.000657068 | 0.003581753 | 0.000213422 |
| G]A | | | | | | | | | | |
| T[C > | 6.12E−05 | 0.005251742 | 0.000722725 | 6.17E−05 | 0.004648907 | 0.003138176 | 2.84E−05 | 0.000632805 | 0.001526502 | 0.000268563 |
| G]C | | | | | | | | | | |
| T[C > | 5.94E−05 | 0.000448451 | 0.000386426 | 1.57E−05 | 0.000916308 | 0.000451243 | 0.000126994 | 6.40E−05 | 0.000389249 | 2.63E−18 |
| G]G | | | | | | | | | | |
| T[C > | 0.000175137 | 0.028793447 | 0.000284622 | 0.001505985 | 0.003205583 | 0.00162928 | 0.001134433 | 0.000693178 | 0.000392042 | 0.000473366 |
| G]T | | | | | | | | | | |
| T[C > | 0.026559414 | 0.006398097 | 0.000532768 | 0.000107969 | 0.011442731 | 0.044236348 | 0.008139156 | 0.00361689 | 0.001599716 | 0.015476085 |
| T]A | | | | | | | | | | |
| T[C > | 0.009563977 | 0.003695299 | 9.06E−05 | 0.000602725 | 0.011421883 | 0.025145643 | 0.009251866 | 8.09E−05 | 0.001325646 | 0.044020076 |
| T]C | | | | | | | | | | |
| T[C > | 0.021575714 | 0.003085016 | 0.000179993 | 6.33E−05 | 0.012027173 | 0.004420335 | 0.00270375 | 0.000323406 | 3.85E−05 | 0.002947779 |
| T]G | | | | | | | | | | |
| T[C > | 0.001751366 | 0.02232755 | 0.000349994 | 0.000898571 | 0.003780169 | 0.06365323 | 5.06E−05 | 7.38E−05 | 2.24E−16 | 0.026715734 |
| T]T | | | | | | | | | | |
| T[T > | 0.001072786 | 0.01215102 | 0.000117027 | 0.001168249 | 0.010645413 | 0.000488827 | 0.011885049 | 0.000253559 | 0.070938828 | 7.72E−18 |
| A]A | | | | | | | | | | |
| T[T > | 0.000159457 | 0.004760819 | 0.005784588 | 0.000245166 | 0.002776889 | 0.000485042 | 0.000325015 | 0.000809414 | 0.024061831 | 5.40E−18 |
| A]C | | | | | | | | | | |
| T[T > | 0.000149626 | 0.00522121 | 0.001836222 | 2.23E−16 | 0.00180022 | 0.000376592 | 0.000177855 | 9.92E−05 | 0.073791171 | 0.000187489 |
| A]G | | | | | | | | | | |
| T[T > | 0.000475239 | 0.00719039 | 0.001979418 | 0.001307349 | 0.007114246 | 0.000676979 | 0.006755757 | 7.66E−05 | 0.030231372 | 7.39E−18 |
| A]T | | | | | | | | | | |
| T[T > | 0.003365989 | 0.012842542 | 0.002055354 | 0.001040625 | 0.001774235 | 0.001227016 | 0.013667807 | 0.083072782 | 0.004574468 | 7.72E−18 |
| C]A | | | | | | | | | | |
| T[T > | 0.006012642 | 0.003741371 | 0.015527051 | 0.001114392 | 0.006286144 | 0.000564691 | 0.016966214 | 0.055793593 | 2.26E−16 | 0.000194752 |
| C]C | | | | | | | | | | |
| T[T > | 0.001325544 | 0.007287519 | 0.002357991 | 0.001212014 | 0.003278613 | 0.001090401 | 0.01212648 | 0.027783663 | 0.001229853 | 0.000306063 |
| C]G | | | | | | | | | | |
| T[T > | 0.001350679 | 0.008425611 | 0.020993823 | 0.000997976 | 0.003376762 | 0.001478692 | 0.019431612 | 0.040819987 | 2.23E−16 | 7.39E−18 |
| C]T | | | | | | | | | | |
| T[T > | 0.000227352 | 0.009661543 | 2.18E−16 | 1.20E−05 | 0.000686038 | 0.001642619 | 2.20E−16 | 0.000458775 | 0.00499931 | 0.000184749 |
| G]A | | | | | | | | | | |
| T[T > | 0.000298601 | 0.005617154 | 0.000117721 | 0.008864485 | 0.002136068 | 0.002062705 | 2.27E−16 | 0.000385872 | 0.000766716 | 5.40E−18 |
| G]C | | | | | | | | | | |
| T[T > | 0.000148622 | 0.005281687 | 0.000923128 | 0.004787955 | 0.001458279 | 0.002089935 | 0.002819407 | 0.000131872 | 0.005614984 | 0.000184449 |
| G]G | | | | | | | | | | |
| T[T > | 0.005993012 | 0.015867062 | 0.004578653 | 0.121753089 | 0.005170354 | 0.004255012 | 0.001520297 | 2.23E−16 | 0.000597597 | 7.39E−18 |
| G]T | | | | | | | | | | |
| Type | SBS24 | SBS25 | SBS26 | SBS27 | SBS28 | SBS29 | SBS30 | SBS31 | SBS32 | SBS33 |
| A[C > | 0.036623727 | 0.009935785 | 0.000876875 | 0.005244791 | 0.000785671 | 0.063497597 | 0.001810502 | 0.009609122 | 0.022378921 | 0.003115413 |
| A]A | | | | | | | | | | |
| A[C > | 0.026258314 | 0.006918705 | 0.000522296 | 0.00469925 | 0.002496922 | 0.050660559 | 0.00050123 | 0.018351301 | 0.018580266 | 0.002199986 |
| A]C | | | | | | | | | | |
| A[C > | 0.015292613 | 0.001494493 | 0.000117594 | 0.000809489 | 0.000363294 | 0.019810721 | 9.43E−05 | 0.001717126 | 0.004596508 | 0.000425907 |
| A]G | | | | | | | | | | |
| A[C > | 0.02212589 | 0.004985862 | 0.000621485 | 0.002737009 | 0.003976784 | 0.035752558 | 0.000559008 | 0.006322835 | 0.014645495 | 0.001902505 |
| A]T | | | | | | | | | | |
| A[C > | 0.003360529 | 0.008059025 | 0.000428895 | 0.001328814 | 0.000154328 | 0.003154786 | 0.001076243 | 0.008380282 | 0.001997043 | 0.001412454 |
| G]A | | | | | | | | | | |
| A[C > | 0.003636529 | 0.001620954 | 0.000373917 | 0.001754783 | 0.000221071 | 0.00260229 | 0.000462597 | 0.003134601 | 0.002006273 | 0.001282503 |
| G]C | | | | | | | | | | |
| A[C > | 0.000950622 | 0 | 7.05E−05 | 0 | 2.28E−16 | 0.0009245 | 0.000135318 | 0.00310324 | 0.001164586 | 0.000260276 |
| G]G | | | | | | | | | | |
| A[C > | 0.002866308 | 0.003440947 | 0.000730923 | 0.001328254 | 8.34E−05 | 0.00250067 | 0.000734955 | 0.003805783 | 0.001755453 | 0.001301714 |
| G]T | | | | | | | | | | |
| A[C > | 0.011872527 | 0.021075907 | 0.002671806 | 0.013791484 | 0.000778656 | 0.005113968 | 0.106618463 | 0.017828245 | 0.086705775 | 0.005319243 |
| T]A | | | | | | | | | | |
| A[C > | 0.011097853 | 0.012947862 | 0.002512562 | 0.005373404 | 0.003335809 | 0.00426459 | 0.089547772 | 0.009205409 | 0.121562381 | 0.002101332 |
| T]C | | | | | | | | | | |
| A[C > | 0.000531108 | 0.020304488 | 0.005498058 | 0.006937001 | 0.007255587 | 0.020739348 | 0.014874689 | 0.007851198 | 0.037720219 | 0.007365922 |
| T]G | | | | | | | | | | |
| A[C > | 0.009202359 | 0.005748288 | 0.002801202 | 0.004508015 | 0.005068647 | 0.001837842 | 0.029458531 | 0.010168891 | 0.152473644 | 0.003074047 |
| T]T | | | | | | | | | | |
| A[T > | 0.001575282 | 0.020287902 | 0.001512888 | 0.142648256 | 0.000170802 | 0.00061627 | 0.000361804 | 0.007845089 | 0.000709029 | 0.000861618 |
| A]A | | | | | | | | | | |
| A[T > | 0.00226016 | 0.008907671 | 0.003529892 | 0.001467367 | 0.000478242 | 0.00200616 | 0.000394652 | 0.006104069 | 0.000888696 | 0.001140133 |
| A]C | | | | | | | | | | |
| A[T > | 0.001994651 | 0.00620765 | 0.00062227 | 0.001227358 | 0.000150989 | 0.002469833 | 0.000636096 | 0.005022026 | 0.000708255 | 0.000691099 |
| A]G | | | | | | | | | | |
| A[T > | 0.001796213 | 0.01483124 | 0.003058341 | 0.002535995 | 0.001689654 | 0.001953082 | 0.000488818 | 0.004155783 | 0.000840042 | 0.00503718 |
| A]T | | | | | | | | | | |
| A[T > | 0.006380888 | 0.020586254 | 0.084403238 | 0.005037578 | 0.000545176 | 0.001951355 | 0.000608987 | 0.0203613 | 0.000930787 | 0.0124081 |
| C]A | | | | | | | | | | |
| A[T > | 0.002189844 | 0.009518884 | 0.026173344 | 0.001859335 | 0.000441224 | 0.002297054 | 0.000459925 | 0.007441534 | 0.000702341 | 0.003750438 |
| C]C | | | | | | | | | | |
| A[T > | 0.001755292 | 0.019796833 | 0.078157127 | 0.005677777 | 0.000402305 | 0.002738726 | 0.000276173 | 0.008107128 | 0.001084268 | 0.072883131 |
| C]G | | | | | | | | | | |
| A[T > | 0.002434867 | 0.014433086 | 0.037058719 | 0.002585916 | 0.000591379 | 0.001145941 | 0.000543685 | 0.008461414 | 0.000607139 | 0.00915038 |
| C]T | | | | | | | | | | |
| A[T > | 0.003200414 | 0.005250987 | 0.002906338 | 0.001137196 | 0.009801257 | 0.000405205 | 3.89E−05 | 0.003673019 | 0.00038385 | 0.000971802 |
| G]A | | | | | | | | | | |
| A[T > | 0.001406322 | 0 | 0.00253711 | 0.000963839 | 0.017308769 | 0.000819517 | 0.000110462 | 0.00249392 | 0.00042982 | 0.000302035 |
| G]C | | | | | | | | | | |
| A[T > | 0.00205449 | 0.007102984 | 0.002279998 | 6.76E−05 | 0.007628893 | 0.000418278 | 0.000408777 | 0.004013627 | 0.001263321 | 0.000471655 |
| G]G | | | | | | | | | | |
| A[T > | 0.004700091 | 0 | 0.005907479 | 0.000255596 | 0.114300116 | 0.000807141 | 0.000792084 | 0.000782206 | 6.42E−18 | 0.000753093 |
| G]T | | | | | | | | | | |
| C[C > | 0.025269449 | 0.014686882 | 0.001400372 | 0.005046605 | 0.002011507 | 0.053844382 | 0.000763816 | 0.010656633 | 0.00123043 | 0.00267436 |
| A]A | | | | | | | | | | |
| C[C > | 0.03448786 | 0.007708208 | 0.001489635 | 0.002204829 | 0.000107283 | 0.036412144 | 0.000383299 | 0.011475536 | 0.0048493 | 0.00187918 |
| A]C | | | | | | | | | | |
| C[C > | 0.0160641 | 0.001281894 | 0.000362833 | 0.000267207 | 2.22E−16 | 0.014838063 | 0.000408504 | 0.000330679 | 1.66E−05 | 0.000370855 |
| A]G | | | | | | | | | | |
| C[C > | 0.01701262 | 0.012504072 | 0.007091456 | 0.003085788 | 0.000201157 | 0.05156102 | 0.000295391 | 0.027787755 | 0.002649473 | 0.00250605 |
| A]T | | | | | | | | | | |
| C[C > | 0.002596585 | 0.007144969 | 0.000361514 | 0.004708174 | 1.43E−05 | 0.003268414 | 0.001263081 | 0.002041691 | 0.001141124 | 0.001485756 |
| G]A | | | | | | | | | | |
| C[C > | 0.005158356 | 0.00453424 | 0.000317657 | 0.001245778 | 7.43E−05 | 0.006256179 | 0.000783392 | 0.001840043 | 0.000812159 | 0.000403384 |
| G]C | | | | | | | | | | |
| C[C > | 0.000948786 | 0.000825219 | 4.18E−05 | 0.000362638 | 2.22E−16 | 0.001303343 | 0.000120443 | 0.000858358 | 0.000537754 | 0.000317876 |
| G]G | | | | | | | | | | |
| C[C > | 0.004685932 | 0.000194508 | 0.000620751 | 0.000266771 | 2.20E−16 | 0.006646305 | 0.001014473 | 0.003406241 | 0.000491194 | 0.000655733 |
| G]T | | | | | | | | | | |
| C[C > | 0.005352348 | 0.014190703 | 0.001678459 | 0.017319708 | 0.00028934 | 0.007560069 | 0.109400708 | 0.041033016 | 0.016769568 | 0.004546413 |
| T]A | | | | | | | | | | |
| C[C > | 0.008745489 | 0.005855083 | 0.002791832 | 0.007059408 | 0.000206692 | 0.001618317 | 0.101752115 | 0.162240338 | 0.027991894 | 0.001505312 |
| T]C | | | | | | | | | | |
| C[C > | 0.00637544 | 0.011617167 | 0.003347688 | 0.007534023 | 0.002549999 | 0.015339348 | 0.016862078 | 0.008241845 | 0.011215731 | 0.005877706 |
| T]G | | | | | | | | | | |
| C[C > | 0.004268078 | 0.013496458 | 0.001638782 | 0.00711722 | 0.003289854 | 0.000668604 | 0.045949677 | 0.144416648 | 0.042470951 | 0.002674441 |
| T]T | | | | | | | | | | |
| C[T > | 0.001320271 | 0.026239201 | 0.000942591 | 0.331242688 | 0.00013947 | 0.001947613 | 0.000213978 | 0.020626732 | 0.000324224 | 0.000742886 |
| A]A | | | | | | | | | | |
| C[T > | 0.001876043 | 0.015811174 | 0.001452216 | 0.00282057 | 0.001348958 | 0.001412536 | 0.000779269 | 0.021191312 | 0.000650411 | 0.000990848 |
| A]C | | | | | | | | | | |
| C]T > | 0.00503112 | 0.062755694 | 0.001138318 | 0.004403303 | 0.000802962 | 0.001977611 | 0.000586949 | 0.018724595 | 0.001406659 | 0.000477009 |
| A]G | | | | | | | | | | |
| C[T > | 0.004832395 | 0.036754291 | 0.000645236 | 0.005469533 | 0.001032813 | 0.001990515 | 0.000615939 | 0.031243308 | 0.000665745 | 0.001122621 |
| A]T | | | | | | | | | | |
| C[T > | 0.002270465 | 0.009009125 | 0.051922366 | 0.004903593 | 0.001315002 | 0.001178556 | 0.001269867 | 0.008160576 | 0.000591585 | 0.015036962 |
| C]A | | | | | | | | | | |
| C[T > | 0.002247239 | 0.010807638 | 0.031247689 | 0.005741515 | 0.002617978 | 0.001843309 | 0.001384031 | 0.009952886 | 0.000927586 | 0.030065044 |
| C]C | | | | | | | | | | |
| C[T > | 0.004220939 | 0.013369258 | 0.078583886 | 0.005664249 | 0.002480616 | 0.003905283 | 0.001269889 | 0.004465866 | 0.00183564 | 0.039833713 |
| C]G | | | | | | | | | | |
| C[T > | 0.003654498 | 0.008997772 | 0.055779707 | 0.003928394 | 0.000368002 | 0.001789453 | 0.004126391 | 0.013908311 | 0.001094513 | 0.003077185 |
| C]T | | | | | | | | | | |
| C[T > | 0.001430293 | 0 | 0.00141788 | 0 | 0.01454472 | 0.000780044 | 0.000197979 | 0.001972556 | 5.80E−05 | 0.000724961 |
| G]A | | | | | | | | | | |
| C[T > | 0.000882844 | 0.002892044 | 0.004947551 | 0.000382433 | 0.022082943 | 5.70E−06 | 0.000215628 | 0.001436188 | 0.000450284 | 0.000947898 |
| G]C | | | | | | | | | | |
| C[T > | 0.003040677 | 0.011772928 | 0.005551797 | 0.000403303 | 0.015441584 | 0.000331599 | 7.85E−05 | 0.001501973 | 0.001087417 | 0.001065552 |
| G]G | | | | | | | | | | |
| C[T > | 0.004741787 | 0.008586043 | 0.00783931 | 0.001450484 | 0.101275871 | 0.000771073 | 0.000266706 | 0.000956448 | 0.000678799 | 0.000192449 |
| G]T | | | | | | | | | | |
| G[C > | 0.062344417 | 0.013497296 | 0.000496308 | 0.010830792 | 0.000155738 | 0.097186394 | 0.000944897 | 0.01314476 | 0.004508757 | 0.004400884 |
| A]A | | | | | | | | | | |
| G[C > | 0.12653385 | 0.006379797 | 0.000657254 | 0.01226444 | 0.001004293 | 0.089138138 | 0.00048819 | 0.02127695 | 0.003569262 | 0.000952722 |
| A]C | | | | | | | | | | |
| G[C > | 0.066576742 | 0.000663084 | 0.000130103 | 0.001647608 | 3.97E−05 | 0.023202993 | 2.24E−16 | 0.000983054 | 0.00088371 | 0.000343999 |
| A]G | | | | | | | | | | |
| G[C > | 0.054251263 | 0.010444943 | 0.000813383 | 0.007393642 | 0.000156939 | 0.08424815 | 0.000859377 | 0.003504227 | 0.003252609 | 0.003167348 |
| A]T | | | | | | | | | | |
| G[C > | 0.002726315 | 0.004169165 | 0.000395245 | 0.001424049 | 0.000182693 | 0.00227202 | 0.000516036 | 0.001836253 | 0.000875759 | 0.000769406 |
| G]A | | | | | | | | | | |
| G[C > | 0.00572368 | 0.002356679 | 0.000571396 | 0.001335241 | 2.19E−16 | 0.001618899 | 0.000252989 | 0.007432088 | 0.000887386 | 0.000763752 |
| G]C | | | | | | | | | | |
| G[C > | 0.003172245 | 0.00042123 | 7.44E−05 | 0 | 2.23E−16 | 6.94E−06 | 0.000118165 | 0.000462197 | 6.91E−05 | 4.69E−05 |
| G]G | | | | | | | | | | |
| G[C > | 0.003370759 | 0.001979565 | 0.00030365 | 0.004549934 | 2.21E−16 | 0.00319665 | 0.000397786 | 0.003354474 | 0.000226787 | 0.000924389 |
| G]T | | | | | | | | | | |
| G[C > | 0.029969422 | 0.009997997 | 0.002751706 | 0.014240486 | 0.003244547 | 0.010409253 | 0.049799957 | 0.006823234 | 0.022593772 | 0.005338941 |
| T]A | | | | | | | | | | |
| G[C > | 0.043199447 | 0.006853105 | 0.005378428 | 0.019880261 | 0.000185105 | 0.007857581 | 0.060282601 | 0.011281732 | 0.037368795 | 0.005058876 |
| T]C | | | | | | | | | | |
| G[C > | 0.025660838 | 0.010581135 | 0.006737142 | 0.009471221 | 0.000216341 | 0.018461512 | 0.00529217 | 0.001992405 | 0.010479573 | 0.005874137 |
| T]G | | | | | | | | | | |
| G[C > | 0.020244497 | 0.009768509 | 0.004957953 | 0.007573245 | 0.000101315 | 0.005098706 | 0.017646136 | 0.005690625 | 0.052917061 | 0.003902093 |
| T]T | | | | | | | | | | |
| G[T > | 0.001561231 | 0.035907221 | 0.000777305 | 0.109443346 | 0.000114892 | 0.003405968 | 0.000327061 | 0.001891451 | 0.008718252 | 0.000801949 |
| A]A | | | | | | | | | | |
| G[T > | 0.002342174 | 0.005815847 | 0.001144225 | 2.34E−05 | 0.000117138 | 0.000785249 | 0.000181338 | 0.002903477 | 0.008171396 | 0.000359215 |
| A]C | | | | | | | | | | |
| G[T > | 0.002502244 | 0.038291244 | 0.000609952 | 0.001578553 | 7.18E−05 | 0.003672742 | 9.91E−05 | 0.005804707 | 0.013130302 | 0.000827427 |
| A]G | | | | | | | | | | |
| G[T > | 0.001329278 | 0.020445694 | 0.000844837 | 0 | 0.000327456 | 0.001084195 | 0.000193751 | 0.000801486 | 0.006234455 | 0.001778081 |
| A]T | | | | | | | | | | |
| G[T > | 0.003679333 | 0.017358463 | 0.082892641 | 0.004755811 | 0.000486309 | 0.001072433 | 0.000929489 | 0.004509618 | 0.009065395 | 0.012078733 |
| C]A | | | | | | | | | | |
| G[T > | 0.003637845 | 0.004026356 | 0.02467545 | 0.001954504 | 0.000582711 | 0.003572932 | 0.00055298 | 0.006555274 | 0.007624648 | 0.007878906 |
| C]C | | | | | | | | | | |
| G[T > | 0.002683129 | 0.013432007 | 0.071428639 | 0.001236701 | 0.000731663 | 0.003000409 | 0.000503308 | 0.005160857 | 0.009642252 | 0.207106992 |
| C]G | | | | | | | | | | |
| G[T > | 0.004048718 | 0.014372715 | 0.055917254 | 0.003807196 | 0.000860078 | 0.001570563 | 0.000451409 | 0.004764218 | 0.01305592 | 0.009809752 |
| C]T | | | | | | | | | | |
| G[T > | 0.001064023 | 0.005505113 | 0.001360035 | 0 | 0.001148918 | 4.46E−05 | 0.000186892 | 0.000302632 | 0.001140613 | 0.000575225 |
| G]A | | | | | | | | | | |
| G[T > | 0.001524905 | 0 | 0.001203923 | 5.07E−05 | 0.001915897 | 9.13E−05 | 0.000351715 | 0.000870045 | 0.000735624 | 0.000632099 |
| G]C | | | | | | | | | | |
| G[T > | 0.003145391 | 0.008710757 | 0.002357546 | 0.004765823 | 0.003132839 | 0.000772681 | 0.001386356 | 0.002565338 | 0.003016962 | 0.005422801 |
| G]G | | | | | | | | | | |
| G[T > | 0.00271944 | 0.002803692 | 0.006128612 | 0.00233508 | 0.020819742 | 0.001165257 | 0.000162304 | 0.002854468 | 0.000794488 | 0.000455633 |
| G]T | | | | | | | | | | |
| T[C > | 0.029985899 | 0.006965722 | 0.000942249 | 0.007127929 | 0.014331786 | 0.045416986 | 0.000700125 | 0.001873648 | 0.009765327 | 0.011921802 |
| A]A | | | | | | | | | | |
| T[C > | 0.052128613 | 0.010219151 | 0.000631701 | 0.006300944 | 0.000452179 | 0.04122206 | 0.000243995 | 0.00943161 | 0.009306742 | 0.005110035 |
| A]C | | | | | | | | | | |
| T[C > | 0.010003229 | 0 | 0.000125466 | 0.001482364 | 0.000131284 | 0.017753575 | 8.44E−05 | 0.000976866 | 0.002425535 | 0.001393709 |
| A]G | | | | | | | | | | |
| T[C > | 0.023040438 | 0.024796867 | 0.001412362 | 0.009918686 | 0.000560628 | 0.041474079 | 0.000671805 | 0.004542306 | 0.009716415 | 0.012878343 |
| A]T | | | | | | | | | | |
| T[C > | 0.003773393 | 0.009836321 | 0.001315934 | 0.002889429 | 0.000115256 | 0.000107514 | 0.000127753 | 0.000398906 | 4.73E−18 | 0.013023817 |
| G]A | | | | | | | | | | |
| T[C > | 0.005604579 | 0.00327614 | 0.000747011 | 0.003125349 | 0.000136054 | 0.006850284 | 0.000990036 | 0.002936066 | 0.001101982 | 0.002510017 |
| G]C | | | | | | | | | | |
| T[C > | 0.001379403 | 0.002099571 | 9.60E−05 | 1.31E−05 | 2.50E−05 | 0.000915227 | 0.000108275 | 0.000425525 | 0.001016075 | 8.18E−05 |
| G]G | | | | | | | | | | |
| T[C > | 0.00411292 | 0.016531244 | 0.002229514 | 0.002153599 | 0.000143931 | 0.000339058 | 0.000110115 | 0.004289394 | 0.000697485 | 0.020223022 |
| G]T | | | | | | | | | | |
| T[C > | 0.002414972 | 0.01114114 | 0.003304904 | 0.018927268 | 0.004850758 | 0.006420676 | 0.098580788 | 0.007534884 | 0.024890042 | 0.014726932 |
| T]A | | | | | | | | | | |
| T[C > | 0.012253739 | 0.00646211 | 0.00253683 | 0.008029433 | 0.004131639 | 3.62E−18 | 0.134548545 | 0.024735352 | 0.038769741 | 0.003170021 |
| T]C | | | | | | | | | | |
| T[C > | 0.002748589 | 0.01437085 | 0.00268272 | 0.004712895 | 0.001770803 | 0.014692614 | 0.015015516 | 0.00860256 | 0.010038455 | 0.003916629 |
| T]G | | | | | | | | | | |
| T[C > | 0.004759669 | 0.012499234 | 0.002148808 | 0.012031842 | 0.002002961 | 0.004318955 | 0.053037198 | 0.019727164 | 0.067531097 | 0.009095329 |
| T]T | | | | | | | | | | |
| T[T > | 0.004071102 | 0.021487062 | 0.001114693 | 0.052200891 | 0.000180275 | 0.000753854 | 0.000278567 | 0.006627713 | 0.000504612 | 0.004033599 |
| A]A | | | | | | | | | | |
| T[T > | 0.003334414 | 0.003468423 | 0.001282639 | 4.81E−05 | 0.001269537 | 0.001965207 | 0.000245671 | 0.003828047 | 0.002491788 | 0.000220306 |
| A]C | | | | | | | | | | |
| T[T > | 0.002006326 | 0.010257806 | 0.000279805 | 0 | 0.000322342 | 0.00093025 | 0.000191499 | 0.003300429 | 0.001769841 | 0.000662501 |
| A]G | | | | | | | | | | |
| T[T > | 0.002199838 | 0.014528485 | 0.001313651 | 0.001939693 | 0.005722766 | 0.001203668 | 0.000703931 | 0.008929634 | 0.000712343 | 0.002720255 |
| A]T | | | | | | | | | | |
| T[T > | 0.001442672 | 0.014291853 | 0.041727003 | 0.001700484 | 0.001289287 | 0.002555604 | 0.000537377 | 0.004540478 | 0.000884057 | 0.010821851 |
| C]A | | | | | | | | | | |
| T[T > | 0.003059096 | 0.007536954 | 0.031964181 | 0.003560627 | 0.00208204 | 0.001527363 | 0.000291543 | 0.010514369 | 0.001240809 | 0.012690419 |
| C]C | | | | | | | | | | |
| T[T > | 0.002046654 | 0.004917713 | 0.030496725 | 0.001160047 | 0.001415895 | 0.000381563 | 0.000204602 | 0.004047315 | 0.00169945 | 0.32121261 |
| C]G | | | | | | | | | | |
| T[T > | 0.003686486 | 0.017033397 | 0.057559963 | 0.001537684 | 0.002060996 | 0.000824513 | 0.00044686 | 0.012167632 | 0.000685292 | 0.009120854 |
| C]T | | | | | | | | | | |
| T[T > | 0.00012154 | 0.002050142 | 0.001410629 | 0.001176497 | 0.041631164 | 0.000460798 | 0.000116563 | 0.002225725 | 2.23E−05 | 0.000460421 |
| G]A | | | | | | | | | | |
| T[T > | 0.002692004 | 0.00058892 | 0.001750904 | 0.000285666 | 0.036257973 | 0.000101111 | 9.83E−05 | 0.002429534 | 0.000227821 | 0.000166498 |
| G]C | | | | | | | | | | |
| T[T > | 0.003064941 | 0.009483442 | 0.002858439 | 0.002370531 | 0.014058532 | 0.000181217 | 0.000819416 | 0.005399785 | 0.000822574 | 0.000962634 |
| G]G | | | | | | | | | | |
| T[T > | 0.003977789 | 0.007885461 | 0.009476335 | 0.000140703 | 0.479231654 | 0.000930837 | 0.008927167 | 0.007340803 | 0.0006362 | 0.002250211 |
| G]T | | | | | | | | | | |
| Type | SBS34 | SBS35 | SBS36 | SBS37 | SBS38 | SBS39 | SBS40 | SBS41 | SBS42 | SBS43 |
| A[C > | 0.004914518 | 0.0088988 | 0.025256687 | 0.00396339 | 0.012902367 | 0.011747931 | 0.028323239 | 0.002119925 | 0.001170674 | 0.029584361 |
| A]A | | | | | | | | | | |
| A[C > | 0.006917052 | 0.045853502 | 0.00821219 | 0.001432839 | 0.010026297 | 0.007070358 | 0.013254349 | 0.00120714 | 0.020474114 | 0.005827094 |
| A]C | | | | | | | | | | |
| A[C > | 5.39E−05 | 0.001438614 | 0.002305593 | 0.001092281 | 0.001966853 | 0.00275325 | 0.003011865 | 6.34E−05 | 3.46E−05 | 0.000644723 |
| A]G | | | | | | | | | | |
| A[C > | 0.001240721 | 0.021759123 | 0.017932704 | 0.001855489 | 0.008917017 | 0.007427184 | 0.014858413 | 0.001335693 | 0.00803995 | 0.002075045 |
| A]T | | | | | | | | | | |
| A[C > | 0.000768966 | 0.003607894 | 0.001844139 | 0.034416274 | 0.002711513 | 0.047493773 | 0.012253316 | 0.005355071 | 0.000409736 | 0.000362032 |
| G]A | | | | | | | | | | |
| A[C > | 0.001123027 | 0.004515875 | 0.00164836 | 0.01106745 | 0.00156847 | 0.022249377 | 0.008585653 | 0.003126691 | 0.000502908 | 3.09E−05 |
| G]C | | | | | | | | | | |
| A[C > | 2.30E−16 | 0.000358101 | 0.000514641 | 0.005512928 | 0.000658378 | 0.014024044 | 0.002320786 | 0.001248482 | 1.83E−05 | 0.004562499 |
| G]G | | | | | | | | | | |
| A[C > | 0.002047694 | 0.009549837 | 0.00191349 | 0.018855779 | 0.002458477 | 0.043559428 | 0.014657624 | 0.008305398 | 0.000434783 | 0.000354863 |
| G]T | | | | | | | | | | |
| A[C > | 0.003552178 | 0.002227219 | 0.005973407 | 0.006261153 | 0.003477591 | 0.009639328 | 0.019786093 | 0.009926473 | 0.022606124 | 2.91E−05 |
| T]A | | | | | | | | | | |
| A[C > | 0.003687107 | 0.003960075 | 0.003237498 | 0.003023785 | 0.002134311 | 0.003994999 | 0.01196848 | 0.003324582 | 0.039556783 | 0.000126418 |
| T]C | | | | | | | | | | |
| A[C > | 0.000122291 | 0.000600285 | 0.007277029 | 0.006522773 | 0.005362262 | 0.006187078 | 0.0002754 | 0.003539085 | 0.000456026 | 0.001256489 |
| T]G | | | | | | | | | | |
| A[C > | 0.003903731 | 0.00090663 | 0.004317875 | 0.005666763 | 0.002458477 | 0.01284702 | 0.016966702 | 0.00637718 | 0.035508936 | 0.000912218 |
| T]T | | | | | | | | | | |
| A[T > | 0.102998106 | 0.00488337 | 0.002423292 | 0.002237135 | 0.001987708 | 0.005283358 | 0.006071048 | 0.007427058 | 0.013300554 | 0.015204481 |
| A]A | | | | | | | | | | |
| A[T > | 0.044733248 | 0.007405323 | 0.001430894 | 0.001662927 | 0.001952345 | 0.002887111 | 0.004452175 | 0.004313223 | 0.00404034 | 0.003784665 |
| A]C | | | | | | | | | | |
| A[T > | 0.034610174 | 0.001328612 | 0.00251346 | 0.002476537 | 0.001079093 | 0.006907338 | 0.006899263 | 0.003017563 | 0.005011774 | 0.001421516 |
| A]G | | | | | | | | | | |
| A[T > | 0.316273863 | 0.007366521 | 0.003051676 | 0.0027566 | 0.002719271 | 0.006094684 | 0.01065864 | 0.069353991 | 0.006856348 | 0.004257044 |
| A]T | | | | | | | | | | |
| A[T > | 0.004619915 | 0.002037234 | 0.00521405 | 0.072880896 | 0.002786786 | 0.011143806 | 0.018611245 | 0.014336413 | 0.011600483 | 8.82E−06 |
| C]A | | | | | | | | | | |
| A[T > | 0.001239232 | 0.000826056 | 0.001691056 | 0.038467716 | 0.001650436 | 0.005653925 | 0.007360127 | 0.005807805 | 0.003536557 | 0.031338627 |
| C]C | | | | | | | | | | |
| A[T > | 1.95E−05 | 0.00091904 | 0.003814896 | 0.016709163 | 0.002168177 | 0.00959463 | 0.015431253 | 0.008006998 | 0.008563031 | 0.000582524 |
| C]G | | | | | | | | | | |
| A[T > | 0.002342028 | 0.002029042 | 0.00508944 | 0.047767793 | 0.003489065 | 0.013543741 | 0.019325011 | 0.017637437 | 0.002702502 | 0.008752801 |
| C]T | | | | | | | | | | |
| A[T > | 0.002819948 | 0.003045864 | 0.001082536 | 0.021575927 | 0.001148675 | 0.003860533 | 0.007195685 | 0.007496749 | 0.00120005 | 0.002613581 |
| G]A | | | | | | | | | | |
| A[T > | 0.000465468 | 0.000971949 | 0.000623389 | 0.010618692 | 0.001076809 | 0.002977333 | 0.007069332 | 0.001645043 | 0.000408064 | 0.004075023 |
| G]C | | | | | | | | | | |
| A[T > | 0.000197058 | 0.004475323 | 0.000929881 | 0.016709163 | 0.002068261 | 0.007623949 | 0.008024251 | 0.002688917 | 0.000637226 | 0.034692954 |
| G]G | | | | | | | | | | |
| A[T > | 0.001150997 | 0.002628758 | 0.000978127 | 0.02458051 | 0.002859234 | 0.006393443 | 0.017930423 | 0.007583102 | 0.000911844 | 0.00560975 |
| G]T | | | | | | | | | | |
| C[C > | 0.001945752 | 0.029794961 | 0.055298194 | 0.003194674 | 0.379739071 | 0.010027619 | 0.020656562 | 0.002364372 | 0.011575426 | 0.006574377 |
| A]A | | | | | | | | | | |
| C[C > | 3.64E−05 | 0.032663622 | 0.019884143 | 0.000802184 | 0.068805615 | 0.00893477 | 0.014500762 | 0.000906845 | 0.032511096 | 8.08E−05 |
| A]C | | | | | | | | | | |
| C[C > | 0.000300084 | 0.002212426 | 0.007100836 | 0.000747938 | 0.051298523 | 0.005751256 | 0.002545674 | 0.00026167 | 0.001893264 | 2.56E−05 |
| A]G | | | | | | | | | | |
| C[C > | 0.000618668 | 0.082212643 | 0.059957727 | 0.001756131 | 0.183396552 | 0.005986966 | 0.014599066 | 0.002632678 | 0.021055933 | 5.18E−18 |
| A]T | | | | | | | | | | |
| C[C > | 0.000118741 | 0.00384644 | 0.001228849 | 0.000438524 | 0.001684407 | 0.028593605 | 0.007507866 | 0.002066342 | 0.004250975 | 2.07E−05 |
| G]A | | | | | | | | | | |
| C[C > | 0.000116954 | 0.006235782 | 0.001348578 | 0.000308456 | 0.001247411 | 0.037178901 | 0.009223274 | 0.001184129 | 0.006561691 | 0.000208812 |
| G]C | | | | | | | | | | |
| C[C > | 0.000191329 | 0.000874914 | 0.000419049 | 0.000321403 | 0.000962602 | 0.01232412 | 0.001683753 | 0.000412055 | 0.000473316 | 0.002541846 |
| G]G | | | | | | | | | | |
| C[C > | 7.34E−05 | 0.016143574 | 0.002071267 | 0.000576448 | 0.001265835 | 0.039913105 | 0.013804559 | 0.004997122 | 0.006825715 | 0.000845869 |
| G]T | | | | | | | | | | |
| C[C > | 0.004739653 | 0.005082084 | 0.006689298 | 0.003581606 | 0.004225968 | 0.009044713 | 0.019464838 | 0.006864627 | 0.018660385 | 7.56E−06 |
| T]A | | | | | | | | | | |
| C[C > | 0.000509442 | 0.082351919 | 0.006575548 | 0.002069516 | 0.004682742 | 0.003806646 | 0.017065522 | 0.001825532 | 0.047676332 | 4.28E−18 |
| T]C | | | | | | | | | | |
| C[C > | 0.009516102 | 0.00268508 | 0.005330627 | 0.003274106 | 0.003520487 | 0.007584845 | 0.002365272 | 0.010125924 | 0.001933546 | 9.73E−05 |
| T]G | | | | | | | | | | |
| C[C > | 3.52E−05 | 0.046836293 | 0.005698462 | 0.006012517 | 0.004086554 | 0.006940115 | 0.021451689 | 0.007679473 | 0.032831288 | 0.00073778 |
| T]T | | | | | | | | | | |
| C[T > | 0.023775422 | 0.052696813 | 0.001255377 | 0.00156602 | 0.000898834 | 0.004930947 | 0.001088299 | 0.003615551 | 0.012640857 | 0.001355835 |
| A]A | | | | | | | | | | |
| C[T > | 0.000411543 | 0.033160779 | 0.001459045 | 0.00116056 | 0.002030256 | 0.00644765 | 0.00763113 | 0.00319572 | 0.001730799 | 1.21E−05 |
| A]C | | | | | | | | | | |
| C[T > | 0.001173744 | 0.058308145 | 0.002132186 | 0.001595972 | 0.001783672 | 0.006787658 | 0.003454661 | 0.00311622 | 0.014948578 | 0.000295099 |
| A]G | | | | | | | | | | |
| C[T > | 0.000270612 | 0.066957433 | 0.001965585 | 0.002068221 | 0.002380298 | 0.008308852 | 0.009326137 | 0.009057801 | 0.007644234 | 0.000203702 |
| A]T | | | | | | | | | | |
| C[T > | 0.00059589 | 0.001332448 | 0.001494497 | 0.049873243 | 0.001953988 | 0.007326549 | 0.010882989 | 0.010287341 | 0.003551479 | 0.002711671 |
| C]A | | | | | | | | | | |
| C[T > | 4.07E−05 | 0.002029842 | 0.001878771 | 0.021810533 | 0.002331779 | 0.007919396 | 0.009884416 | 0.005259414 | 0.003149651 | 0.104995223 |
| C]C | | | | | | | | | | |
| C[T > | 0.000813595 | 0.001562899 | 0.001843245 | 0.00992495 | 0.002545239 | 0.007755898 | 0.010483798 | 0.005563252 | 0.003571606 | 0.009221837 |
| C]G | | | | | | | | | | |
| C[T > | 0.001605496 | 0.003206696 | 0.002166155 | 0.050601135 | 0.004306726 | 0.008630356 | 0.013667614 | 0.007911753 | 0.003271772 | 0.009482699 |
| C]T | | | | | | | | | | |
| C[T > | 0.001063373 | 0.003716828 | 0.000476246 | 0.002593409 | 0.00098501 | 0.005290288 | 0.003344772 | 0.003465735 | 0.000872821 | 0.00272164 |
| G]A | | | | | | | | | | |
| C[T > | 2.23E−16 | 0.000520524 | 0.00056563 | 0.00238115 | 0.001417159 | 0.007398779 | 0.005678282 | 0.002414322 | 0.000757728 | 0.00725967 |
| G]C | | | | | | | | | | |
| C[T > | 0.000437395 | 0.006371818 | 0.000883761 | 0.003760509 | 0.00159328 | 0.009812159 | 0.005581374 | 0.003036317 | 0.005698518 | 0.046358422 |
| G]G | | | | | | | | | | |
| C[T > | 6.61E−07 | 0.002732751 | 0.001073049 | 0.004126402 | 0.006939175 | 0.004963208 | 0.013165128 | 0.010555706 | 0.003514126 | 6.28E−18 |
| G]T | | | | | | | | | | |
| G[C > | 0.002824908 | 0.009909093 | 0.067594373 | 0.002039135 | 0.01204999 | 0.006881926 | 0.014207844 | 0.003092734 | 0.007751371 | 0.009291143 |
| A]A | | | | | | | | | | |
| G[C > | 0.000464017 | 0.04970614 | 0.018906567 | 0.000620092 | 0.006219488 | 0.006856397 | 0.010065366 | 0.000713694 | 0.026573483 | 0.001665185 |
| A]C | | | | | | | | | | |
| G[C > | 0.000230014 | 0.000513044 | 0.004860701 | 0.000509796 | 0.001386615 | 0.004567197 | 0.002571639 | 0.000221941 | 0.001074138 | 0.000178234 |
| A]G | | | | | | | | | | |
| G[C > | 1.35E−05 | 0.022874732 | 0.068544836 | 0.001282949 | 0.009081824 | 0.00586193 | 0.010751458 | 0.00194188 | 0.018105331 | 0.001381671 |
| A]T | | | | | | | | | | |
| G[C > | 3.36E−05 | 0.003393388 | 0.000884618 | 0.00131944 | 0.001204999 | 0.024006719 | 0.005062795 | 0.002041805 | 0.002412878 | 0.013587048 |
| G]A | | | | | | | | | | |
| G[C > | 0.000191357 | 0.02118947 | 0.001063494 | 0.000924716 | 0.001069594 | 0.021013129 | 0.005299119 | 0.002655375 | 0.001407998 | 0.014976812 |
| G]C | | | | | | | | | | |
| G[C > | 2.25E−16 | 0.000598045 | 0.000173093 | 0.000581328 | 0.00103237 | 0.009940962 | 0.001200098 | 0.000157376 | 0.000225974 | 0.012889205 |
| G]G | | | | | | | | | | |
| G[C > | 2.69E−05 | 0.008730356 | 0.001043074 | 0.001263058 | 0.000916175 | 0.032245591 | 0.006948628 | 0.003545176 | 0.002770816 | 0.017892148 |
| G]T | | | | | | | | | | |
| G[C > | 0.000234236 | 0.007318874 | 0.003714196 | 0.002548918 | 0.002379873 | 0.008032248 | 0.0143079 | 0.006015317 | 0.033780292 | 0.000765271 |
| T]A | | | | | | | | | | |
| G[C > | 2.20E−16 | 0.017624886 | 0.003348038 | 0.002277285 | 0.002931479 | 0.004439394 | 0.011446887 | 0.008528789 | 0.150715278 | 4.04E−18 |
| T]C | | | | | | | | | | |
| G[C > | 0.016921718 | 0.002560161 | 0.005736232 | 0.004352406 | 0.004534333 | 0.005524997 | 3.96E−18 | 0.017250834 | 0.004610685 | 9.73E−06 |
| T]G | | | | | | | | | | |
| G[C > | 0.000517121 | 0.006023346 | 0.003606054 | 0.002307318 | 0.003197122 | 0.008568967 | 0.012642918 | 0.008374981 | 0.086525476 | 0.001848855 |
| T]T | | | | | | | | | | |
| G[T > | 0.018950116 | 0.000714161 | 0.000871695 | 0.001140444 | 0.000889646 | 0.004922248 | 0.002630546 | 0.002313663 | 0.011969213 | 0.006125406 |
| A]A | | | | | | | | | | |
| G[T > | 0.002419121 | 0.005550546 | 0.000626463 | 0.000769309 | 0.001118324 | 0.003501131 | 0.003412543 | 0.002279101 | 0.001839442 | 0.001321241 |
| A]C | | | | | | | | | | |
| G[T > | 0.006581643 | 0.001127348 | 0.001171199 | 0.001082337 | 0.001218187 | 0.006689143 | 0.004423862 | 0.001434971 | 0.00783192 | 0.004507356 |
| A]G | | | | | | | | | | |
| G[T > | 0.007612672 | 0.002906834 | 0.001132081 | 0.001700053 | 0.001870513 | 0.004141735 | 0.005844578 | 0.004579942 | 0.003307844 | 0.001324936 |
| A]T | | | | | | | | | | |
| G[T > | 0.000108714 | 0.000562763 | 0.002317917 | 0.030444886 | 0.001693615 | 0.007413143 | 0.00596588 | 0.006017511 | 0.005805068 | 7.43E−18 |
| C]A | | | | | | | | | | |
| G[T > | 0.000645765 | 0.002026548 | 0.001389934 | 0.018089696 | 0.002156767 | 0.006853066 | 0.00455669 | 0.003722201 | 0.003308996 | 0.021755769 |
| C]C | | | | | | | | | | |
| G[T > | 0.00044348 | 0.000192253 | 0.001841886 | 0.009851274 | 0.001882652 | 0.005756474 | 0.005667759 | 0.003522202 | 0.003235581 | 0.001201961 |
| C]G | | | | | | | | | | |
| G[T > | 2.26E−16 | 0.000427894 | 0.002678584 | 0.040477457 | 0.003100579 | 0.007604996 | 0.007404482 | 0.003941587 | 0.002432537 | 0.000523906 |
| C]T | | | | | | | | | | |
| G[T > | 0.000364042 | 0.001753031 | 0.000183254 | 0.011701943 | 0.000502107 | 0.003304654 | 0.002471721 | 0.001787379 | 0.001137075 | 0.060560247 |
| G]A | | | | | | | | | | |
| G[T > | 2.22E−16 | 0.005061379 | 0.000310749 | 0.006818424 | 0.000937595 | 0.005768909 | 0.002228599 | 0.000945479 | 7.05E−05 | 0.075102107 |
| G]C | | | | | | | | | | |
| G[T > | 5.69E−05 | 0.004066504 | 0.001531568 | 0.009851274 | 0.002174615 | 0.007742157 | 0.004463987 | 0.001204172 | 0.000413267 | 0.241393934 |
| G]G | | | | | | | | | | |
| G[T > | 0.000424396 | 0.003404858 | 0.000494275 | 0.017304113 | 0.003395388 | 0.006015136 | 0.005550829 | 0.003516017 | 0.000936374 | 0.075450567 |
| G]T | | | | | | | | | | |
| T[C > | 0.004309427 | 0.005311547 | 0.141330085 | 0.002006963 | 0.017843183 | 0.006005047 | 0.02611601 | 0.003325873 | 0.0051474 | 0.006318593 |
| A]A | | | | | | | | | | |
| T[C > | 0.001229022 | 0.023239692 | 0.077239741 | 0.001592629 | 0.011571906 | 0.00787854 | 0.021055268 | 0.001725099 | 0.054402582 | 5.38E−06 |
| A]C | | | | | | | | | | |
| T[C > | 2.28E−16 | 9.69E−05 | 0.014961997 | 0.001141259 | 0.002436311 | 0.003069293 | 0.003233326 | 0.000211205 | 0.000926495 | 3.13E−18 |
| A]G | | | | | | | | | | |
| T[C > | 3.24E−05 | 0.015486628 | 0.209379378 | 0.002458976 | 0.028144849 | 0.006605589 | 0.024311154 | 0.00595367 | 0.027671676 | 6.57E−18 |
| A]T | | | | | | | | | | |
| T[C > | 6.24E−07 | 0.006682268 | 0.003989317 | 0.001796232 | 0.002681518 | 0.034845337 | 0.012555774 | 0.01999543 | 0.000564195 | 4.43E−06 |
| G]A | | | | | | | | | | |
| T[C > | 0.000141035 | 0.005664046 | 0.002411241 | 0.001111835 | 0.002193631 | 0.036786566 | 0.014137108 | 0.014242099 | 0.001883941 | 0.001031158 |
| G]C | | | | | | | | | | |
| T[C > | 2.28E−16 | 0.000158635 | 0.000307383 | 0.000876324 | 0.001126026 | 0.009809502 | 0.001774759 | 0.001969208 | 0.000212151 | 0.001497111 |
| G]G | | | | | | | | | | |
| T[C > | 0.00217914 | 0.021458596 | 0.007640334 | 0.002569831 | 0.003998998 | 0.051130285 | 0.02390765 | 0.045005707 | 0.000386187 | 0.000252818 |
| G]T | | | | | | | | | | |
| T[C > | 0.001877174 | 0.002983336 | 0.010725049 | 0.004033996 | 0.006502177 | 0.003896251 | 0.030133857 | 0.011756107 | 0.004864798 | 3.73E−05 |
| T]A | | | | | | | | | | |
| T[C > | 0.000291137 | 0.012676196 | 0.006863531 | 0.002504133 | 0.007275207 | 0.005292454 | 0.023160794 | 0.006489182 | 0.032943785 | 5.07E−18 |
| T]C | | | | | | | | | | |
| T[C > | 0.000307447 | 0.00078294 | 0.003959331 | 0.003495105 | 0.003552101 | 0.004955736 | 0.00216235 | 0.005019951 | 0.000373058 | 3.13E−18 |
| T]G | | | | | | | | | | |
| T[C > | 0.000818951 | 0.008138071 | 0.008385241 | 0.005885418 | 0.006661623 | 0.0042659 | 0.022596259 | 0.011604611 | 0.015305579 | 0.000842056 |
| T]T | | | | | | | | | | |
| T[T > | 0.269571816 | 0.004255913 | 0.002421389 | 0.00241429 | 0.002415476 | 0.006597133 | 0.00703294 | 0.079529201 | 0.008424625 | 7.51E−05 |
| A]A | | | | | | | | | | |
| T[T > | 2.27E−16 | 0.00541324 | 0.001036064 | 0.001149103 | 0.000948021 | 0.003755023 | 0.010484355 | 0.014560783 | 0.002311518 | 0.000953353 |
| A]C | | | | | | | | | | |
| T[T > | 0.007857065 | 0.001393597 | 0.000875749 | 0.001286966 | 0.000812087 | 0.005493581 | 0.00505225 | 0.009352787 | 0.005390048 | 5.17E−05 |
| A]G | | | | | | | | | | |
| T[T > | 0.04634418 | 0.005443188 | 0.002701624 | 0.001953393 | 0.00251585 | 0.01072617 | 0.021057096 | 0.053663272 | 0.00354655 | 8.78E−18 |
| A]T | | | | | | | | | | |
| T[T > | 0.001387502 | 0.000364227 | 0.002923384 | 0.047004742 | 0.002088793 | 0.005857544 | 0.010061148 | 0.062755052 | 0.006759523 | 0.000134932 |
| C]A | | | | | | | | | | |
| T[T > | 0.00638186 | 0.000839563 | 0.002315908 | 0.045862418 | 0.002237248 | 0.006553481 | 0.007685134 | 0.02728874 | 0.00309907 | 0.036487601 |
| C]C | | | | | | | | | | |
| T[T > | 0.001112326 | 0.000442316 | 0.001385933 | 0.010054421 | 0.001939313 | 0.005010628 | 0.00604861 | 0.020638551 | 0.003448417 | 2.79E−05 |
| C]G | | | | | | | | | | |
| T[T > | 0.007132974 | 0.001066503 | 0.002801685 | 0.061506846 | 0.003079401 | 0.009503186 | 0.010227732 | 0.046240689 | 0.002377801 | 0.001842228 |
| C]T | | | | | | | | | | |
| T[T > | 2.20E−16 | 0.004246016 | 0.000775632 | 0.018033266 | 0.001158241 | 0.008273534 | 0.008344835 | 0.041343344 | 0.001139802 | 0.00020092 |
| G]A | | | | | | | | | | |
| T[T > | 2.27E−16 | 0.00198145 | 0.000755717 | 0.019829649 | 0.000922482 | 0.005169516 | 0.011604043 | 0.015782667 | 0.000280246 | 0.008385034 |
| G]C | | | | | | | | | | |
| T[T > | 0.001537032 | 0.00486749 | 0.000905878 | 0.03036435 | 0.001919112 | 0.007324774 | 0.008715635 | 0.019531116 | 0.002963009 | 0.022911966 |
| G]G | | | | | | | | | | |
| T[T > | 0.000233736 | 0.003642207 | 0.001811089 | 0.029150639 | 0.005323539 | 0.010926659 | 0.025067971 | 0.088168255 | 0.001753124 | 0.001321598 |
| G]T | | | | | | | | | | |
| Type | SBS44 | SBS45 | SBS46 | SBS47 | SBS48 | SBS49 | SBS50 | SBS51 | SBS52 | SBS53 |
| A[C > | 7.73E−18 | 0.009177748 | 0.00441212 | 0.068159835 | 0.000846457 | 0.025219647 | 0.11934628 | 0.141438096 | 0.015188294 | 0.005414513 |
| A]A | | | | | | | | | | |
| A[C > | 0.000148593 | 0.002827629 | 0.004641435 | 0.029404587 | 0.000819964 | 0.006956334 | 0.125436939 | 0.001669528 | 0.006435806 | 0.001942643 |
| A]C | | | | | | | | | | |
| A[C > | 9.46E−07 | 0.001717452 | 0.00030894 | 0.001424744 | 0.000262312 | 0.007336581 | 0.005942875 | 0.004944784 | 0.004248392 | 0.039378658 |
| A]G | | | | | | | | | | |
| A[C > | 0.005811547 | 0.009707596 | 0.003407928 | 0.011958115 | 3.85E−05 | 0.008004616 | 0.083406934 | 0.021256906 | 0.009228992 | 1.72E−05 |
| A]T | | | | | | | | | | |
| A[C > | 0.003198705 | 0.003133128 | 0.001203305 | 0.001035467 | 0.000196022 | 0.000289373 | 0.003861203 | 0.003972304 | 0.001728668 | 0.007075097 |
| G]A | | | | | | | | | | |
| A[C > | 0.000230814 | 0.00191485 | 0.001481309 | 0.000610863 | 0.000111148 | 0.000133585 | 0.001086462 | 0.004485005 | 0.001495784 | 0.005411648 |
| G]C | | | | | | | | | | |
| A[C > | 3.24E−05 | 0.000279345 | 0 | 0.000338635 | 8.16E−05 | 0.000210501 | 0.000851778 | 0.002029422 | 0.000839417 | 0.002594237 |
| G]G | | | | | | | | | | |
| A[C > | 0.003307957 | 0.001440027 | 0.00220513 | 0.0017284 | 0.000678859 | 0.000176764 | 0.0019749 | 0.000902416 | 0.001707963 | 0.003058212 |
| G]T | | | | | | | | | | |
| A[C > | 0.090428787 | 0.001339891 | 0.015241868 | 0.004292662 | 0.000284132 | 0.000577741 | 0.003530243 | 0.035911233 | 0.002687928 | 0.007266316 |
| T]A | | | | | | | | | | |
| A[C > | 0.016741467 | 0.000596282 | 0.0087891 | 0.00093065 | 0.000427044 | 0.000299825 | 0.001777846 | 0.005581559 | 0.002066543 | 0.001942643 |
| T]C | | | | | | | | | | |
| A[C > | 0.014772088 | 0.005597239 | 0.004737086 | 0.00314889 | 0.001230221 | 0.002074055 | 0.014316457 | 0.005820423 | 0.004186821 | 0.007183246 |
| T]G | | | | | | | | | | |
| A[C > | 0.030465377 | 0.001228555 | 0.006815856 | 0.00250216 | 0.000232554 | 0.000416802 | 0.006937211 | 0.00320859 | 0.001757903 | 0.001951622 |
| T]T | | | | | | | | | | |
| A[T > | 0.004764473 | 0.000524103 | 0.002682862 | 0.137473164 | 0.000647475 | 0.000128438 | 0.005326804 | 0.112322235 | 0.001752583 | 0.005873979 |
| A]A | | | | | | | | | | |
| A[T > | 0.002611054 | 0.000499884 | 0.001802032 | 0.007186332 | 0.000262915 | 7.80E−05 | 0.00394506 | 0.001351999 | 0.00117718 | 0.00260239 |
| A]C | | | | | | | | | | |
| A[T | 1.08E−06 | 0.002127027 | 0.000496982 | 0.007423877 | 0.000456318 | 0.000240026 | 0.000569629 | 0.036590705 | 0.001178659 | 0.002863088 |
| A]G | | | | | | | | | | |
| A[T > | 0.030727126 | 0.000604501 | 0.001989064 | 0.067401528 | 0.000662776 | 0.000352771 | 0.002397194 | 0.029349014 | 0.001179333 | 0.004621493 |
| A]T | | | | | | | | | | |
| A[T > | 0.019237309 | 0.00136766 | 0.018382574 | 0.002819196 | 0.001079124 | 0.000610331 | 0.002613712 | 0.002574465 | 0.0037131 | 0.011269264 |
| C]A | | | | | | | | | | |
| A[T > | 0.003253775 | 0.000420426 | 0.047853967 | 0.005690853 | 0.000186808 | 8.17E−05 | 0.009291918 | 0.019328584 | 0.001875507 | 0.003717699 |
| C]C | | | | | | | | | | |
| A[T > | 0.027120119 | 0.002067111 | 0.032701387 | 0.002082672 | 0.000279679 | 0.000199192 | 0.001123352 | 0.003927535 | 0.00259503 | 0.00823013 |
| C]G | | | | | | | | | | |
| A[T > | 0.000266368 | 0.001458797 | 0.030432683 | 0.001296183 | 0.001089898 | 0.000480326 | 0.004893857 | 0.001860429 | 0.004875899 | 0.007166808 |
| C]T | | | | | | | | | | |
| A[T > | 2.39E−08 | 0.0001168 | 9.94E−05 | 0.000170347 | 0.000507188 | 0.000195147 | 0.000738398 | 0.000718664 | 0.001712977 | 0.004577515 |
| G]A | | | | | | | | | | |
| A[T > | 3.27E−05 | 0.000587389 | 0.000800903 | 0.001726326 | 4.05E−05 | 8.31E−05 | 0.00060778 | 0.004096057 | 0.000527736 | 0.002913872 |
| G]C | | | | | | | | | | |
| A[T > | 7.45E−06 | 0.000982627 | 0.001590341 | 0.003667096 | 0.000344446 | 0.000125491 | 0.003916822 | 0.027641892 | 0.001178659 | 0.005047813 |
| G]G | | | | | | | | | | |
| A[T > | 0.00141664 | 0.000165863 | 0.000596719 | 0.008056277 | 0.000195396 | 0.000125563 | 0.000102453 | 0.002119098 | 0.001843328 | 0.002056215 |
| G]T | | | | | | | | | | |
| C[C > | 2.82E−05 | 0.237080364 | 0.015467499 | 0.024453152 | 0.001155106 | 0.227510616 | 0.022312351 | 0.006923148 | 0.056712421 | 0.332372026 |
| A]A | | | | | | | | | | |
| C[C > | 0.029934314 | 0.14940817 | 0.007090988 | 0.005203423 | 3.47E−05 | 0.028223508 | 0.002846685 | 0.003123102 | 0.018646557 | 0.003509014 |
| A]C | | | | | | | | | | |
| C[C > | 0.003753997 | 0.068259449 | 0.004502797 | 0.000271859 | 0.135342636 | 0.516354286 | 5.18E−18 | 0.004304186 | 0.013560621 | 0.067989374 |
| A]G | | | | | | | | | | |
| C[C > | 0.0862343 | 0.193256181 | 0.00822975 | 0.000677949 | 0.000827198 | 0.048682772 | 0.001140443 | 0.005028856 | 0.018575357 | 0.004896166 |
| A]T | | | | | | | | | | |
| C[C > | 5.33E−05 | 0.001424474 | 0.000495753 | 0.000950293 | 9.72E−05 | 0.000214595 | 6.51E−05 | 0.002826787 | 0.001165865 | 0.002706742 |
| G]A | | | | | | | | | | |
| C[C > | 2.46E−05 | 0.000791566 | 0.000295458 | 0.000234006 | 0.000118626 | 0.00025263 | 0.000284669 | 0.000118225 | 0.000819467 | 0.005960382 |
| G]C | | | | | | | | | | |
| C[C > | 7.71E−18 | 0.000646404 | 0.000100062 | 0.000672123 | 3.59E−05 | 0.000238626 | 0.001421103 | 0.002031976 | 0.000566355 | 0.001626924 |
| G]G | | | | | | | | | | |
| C[C > | 8.59E−05 | 0.001902677 | 0.001487304 | 0.000136186 | 0.000239838 | 0.000189763 | 0.000507745 | 0.002588819 | 0.001185661 | 0.000505539 |
| G]T | | | | | | | | | | |
| C[C > | 0.003550718 | 0.002131731 | 0.02815878 | 0.000858842 | 1.78E−05 | 0.000466943 | 0.003807975 | 0.003203692 | 0.00323083 | 0.014528837 |
| T]A | | | | | | | | | | |
| C[C > | 0.002825483 | 5.02E−05 | 0.013098631 | 0.004551761 | 0.000292675 | 0.00048651 | 0.004186302 | 0.004630467 | 0.001786144 | 0.004546892 |
| T]C | | | | | | | | | | |
| C[C > | 0.015558008 | 0.002211646 | 0.008305159 | 0.001524817 | 1.85E−05 | 0.001383629 | 0.001080839 | 0.020720151 | 0.002373109 | 0.013758559 |
| T]G | | | | | | | | | | |
| C[C > | 0.005012928 | 0.002719533 | 0.006345831 | 0.002982181 | 0.000468909 | 0.00051564 | 0.011801109 | 0.004027052 | 0.002539291 | 0.015922491 |
| T]T | | | | | | | | | | |
| C[T > | 1.24E−05 | 0.000698037 | 0.004186699 | 0.013991193 | 8.48E−05 | 7.40E−05 | 0.000192419 | 0.006092792 | 0.001211862 | 0.003691742 |
| A]A | | | | | | | | | | |
| C[T > | 0.003259646 | 0.001044699 | 0.003299504 | 0.000572368 | 0.000100688 | 5.15E−05 | 0.000535002 | 0.0014903 | 0.000230153 | 0.004515748 |
| A]C | | | | | | | | | | |
| C[T > | 0.000581977 | 0.003415133 | 0.004286458 | 5.82E−05 | 0.000305096 | 0.000231733 | 0.000514455 | 0.006501752 | 0.001221817 | 0.003301616 |
| A]G | | | | | | | | | | |
| C[T > | 1.39E−05 | 0.001350773 | 0.003612078 | 7.64E−05 | 0.000358598 | 0.000106569 | 0.000209734 | 0.003633422 | 0.000855853 | 0.008670422 |
| A]T | | | | | | | | | | |
| C[T > | 0.014599156 | 0.001111651 | 0.02222938 | 0.001908799 | 0.000142704 | 0.000120859 | 0.000836473 | 0.004168228 | 0.002374057 | 0.010104768 |
| C]A | | | | | | | | | | |
| C[T > | 0.003209498 | 0.002882967 | 0.083387454 | 0.006295041 | 0.000444213 | 0.00017833 | 0.017700081 | 0.071114302 | 0.001305199 | 0.004084243 |
| C]C | | | | | | | | | | |
| C[T > | 0.03379864 | 0.001562348 | 0.050241275 | 0.003098104 | 3.16E−05 | 0.000107876 | 0.002412753 | 0.0029218 | 0.001221817 | 0.008494157 |
| C]G | | | | | | | | | | |
| C[T > | 0.007759994 | 0.00060684 | 0.096121403 | 0.005482206 | 6.71E−05 | 9.79E−05 | 0.001384844 | 0.006664619 | 0.002339598 | 0.005458036 |
| C]T | | | | | | | | | | |
| C[T > | 0.000233986 | 0.000102152 | 0 | 0.000136914 | 0.000131878 | 0.000105876 | 0.000891307 | 0.002772171 | 0.000797643 | 0.001490703 |
| G]A | | | | | | | | | | |
| C[T > | 0.002537509 | 0.00028227 | 0.001799729 | 5.16E−05 | 9.16E−05 | 2.67E−05 | 6.18E−05 | 0.002560515 | 0.00077216 | 0.004254838 |
| G]C | | | | | | | | | | |
| C[T > | 0.00346986 | 0.000649977 | 0.001594961 | 0.003667756 | 0.000102355 | 0.000111871 | 0.003649039 | 0.005913403 | 0.000790704 | 0.002981459 |
| G]G | | | | | | | | | | |
| C[T > | 0.00038649 | 0.000733853 | 0.00200671 | 0.000107632 | 6.51E−05 | 9.34E−05 | 0.000472653 | 0.002308528 | 0.000862852 | 0.002739088 |
| G]T | | | | | | | | | | |
| G[C > | 7.70E−18 | 0.007577234 | 0.007192388 | 0.011116494 | 0.000741656 | 0.007887467 | 0.014187187 | 0.003937219 | 0.008202356 | 0.000433119 |
| A]A | | | | | | | | | | |
| G[C > | 0.004615295 | 0.005671631 | 0.002955639 | 0.00343727 | 0.000336551 | 0.002448232 | 0.007469073 | 0.003232635 | 0.007186411 | 0.003401505 |
| A]C | | | | | | | | | | |
| G[C > | 0.000611052 | 0.00211417 | 0.000402745 | 0.000790381 | 0.000734614 | 0.006890678 | 0.000245713 | 0.004310891 | 0.00405343 | 0.05133538 |
| A]G | | | | | | | | | | |
| G[C > | 0.011266143 | 0.006979828 | 0.002385369 | 0.001404972 | 1.95E−05 | 0.005816047 | 0.002027878 | 0.003430174 | 0.004169628 | 0.001137187 |
| A]T | | | | | | | | | | |
| G[C > | 0.000219451 | 0.000334201 | 9.99E−05 | 0.001682496 | 7.73E−05 | 0.000152144 | 0.026675908 | 0.003407593 | 0.001373696 | 0.001383573 |
| G]A | | | | | | | | | | |
| G[C > | 0.00117606 | 0.000999712 | 0.000788171 | 0.002252005 | 0.000211074 | 0.000108591 | 0.032517074 | 0.001123538 | 0.001423538 | 0.083752177 |
| G]C | | | | | | | | | | |
| G[C > | 7.76E−18 | 0.000703038 | 0 | 0.000469383 | 4.28E−05 | 3.70E−05 | 0.002819655 | 0.000268927 | 0.001013358 | 0.003031617 |
| G]G | | | | | | | | | | |
| G[C > | 0.001844457 | 0.000930644 | 0.000596342 | 0.002212084 | 6.41E−05 | 0.000207147 | 0.014215029 | 0.002833622 | 0.001010219 | 0.000571586 |
| G]T | | | | | | | | | | |
| G[C > | 0.095696579 | 0.001003607 | 0.01258668 | 0.001061575 | 0.000340255 | 0.000184174 | 0.002417816 | 0.004466845 | 0.001771868 | 0.009203771 |
| T]A | | | | | | | | | | |
| G]C > | 0.080446474 | 0.000923496 | 0.004433459 | 0.001689003 | 7.19E−06 | 0.000116488 | 0.001556878 | 0.003045379 | 0.001197735 | 0.011766835 |
| T]C | | | | | | | | | | |
| G[C > | 0.050096185 | 0.001527463 | 0.001510295 | 0.001736214 | 0.000301202 | 0.001049239 | 0.005538607 | 0.004310891 | 0.002839408 | 0.006053128 |
| T]G | | | | | | | | | | |
| G[C > | 0.066400453 | 0.001987104 | 0.003975616 | 0.004025594 | 0.00010794 | 0.000306737 | 0.016203145 | 0.005915808 | 0.001010219 | 0.00309235 |
| T]T | | | | | | | | | | |
| G[T > | 0.001153218 | 0.000298707 | 0.006045479 | 0.015897362 | 0.0001272 | 5.00E−05 | 0.00072557 | 0.020224775 | 0.000517491 | 0.004426326 |
| A]A | | | | | | | | | | |
| G[T > | 0.002351523 | 0.000636691 | 0.00099331 | 0.000227051 | 0.000121605 | 9.06E−05 | 0.001043137 | 0.003825588 | 0.000394938 | 0.002691727 |
| A]C | | | | | | | | | | |
| G[T > | 0.002220283 | 0.001036392 | 0.002403671 | 1.32E−05 | 0.000141398 | 0.000110389 | 0.000199335 | 0.004298072 | 0.001287432 | 0.003980535 |
| A]G | | | | | | | | | | |
| G[T > | 0.00322595 | 0.000518169 | 0.001213555 | 3.33E−06 | 0.00023064 | 0.000132746 | 0.000795002 | 0.000147701 | 0.000817278 | 0.00315661 |
| A]T | | | | | | | | | | |
| G[T > | 0.026842133 | 0.000651181 | 0.021902472 | 0.000414325 | 0.000153619 | 6.54E−05 | 0.001367878 | 0.001169864 | 0.00238994 | 0.005580154 |
| C]A | | | | | | | | | | |
| G[T > | 0.001155833 | 0.00056883 | 0.057214649 | 0.001085464 | 0.000126508 | 0.000150291 | 0.005344836 | 0.002255606 | 0.002029128 | 0.006659532 |
| C]C | | | | | | | | | | |
| G[T > | 0.017380499 | 0.000912629 | 0.03535399 | 0.003112646 | 4.62E−05 | 6.55E−05 | 0.00371625 | 0.00456858 | 0.00091218 | 0.002573275 |
| C]G | | | | | | | | | | |
| G]T > | 0.011260392 | 0.001412264 | 0.061385641 | 0.003234246 | 0.000109828 | 0.000177332 | 0.001183399 | 0.004056721 | 0.003134074 | 0.007094761 |
| C]T | | | | | | | | | | |
| G]T > | 7.64E−18 | 0.000438103 | 0 | 0.021858873 | 7.48E−06 | 6.56E−05 | 0.00859384 | 0.002171189 | 0.001115964 | 0.004356698 |
| G]A | | | | | | | | | | |
| G[T > | 1.82E−05 | 0.000518933 | 9.93E−05 | 0.010356722 | 0.000132392 | 4.35E−05 | 0.009527318 | 0.005266394 | 0.000555288 | 0.001734669 |
| G]C | | | | | | | | | | |
| G]T > | 0.000103479 | 0.000180111 | 0.001201835 | 0.027310954 | 6.56E−05 | 0.00014752 | 0.035159134 | 0.076343381 | 0.001367273 | 0.004754528 |
| G]G | | | | | | | | | | |
| G]T > | 0.002840459 | 0.000503945 | 0.000606777 | 0.089626122 | 1.15E−05 | 0.000103359 | 0.036918011 | 0.002114351 | 0.000870688 | 0.002060424 |
| G]T | | | | | | | | | | |
| T[C > | 0.000858096 | 0.064481831 | 0.012335006 | 0.016991245 | 0.122772228 | 0.033059847 | 0.019057029 | 0.035814193 | 0.463684986 | 0.002687373 |
| A]A | | | | | | | | | | |
| T[C > | 0.002229193 | 0.064565484 | 0.007107234 | 8.14E−05 | 0.003745637 | 0.005125472 | 0.00088704 | 0.002863921 | 0.046683016 | 0.00365701 |
| A]C | | | | | | | | | | |
| T[C > | 3.22E−07 | 0.018722558 | 0.002545839 | 2.44E−05 | 0.712822491 | 0.041325363 | 4.27E−05 | 0.00462487 | 0.135977346 | 0.000456858 |
| A]G | | | | | | | | | | |
| T[C > | 0.016770647 | 0.062734168 | 0.003021416 | 0.000193863 | 0.000185941 | 0.00883015 | 0.001863496 | 0.000372772 | 0.061219524 | 0.00088042 |
| A]T | | | | | | | | | | |
| T[C > | 0.00067702 | 0.003324844 | 0.002807969 | 0.000138745 | 0.000135644 | 4.82E−05 | 0.000934797 | 0.002056557 | 0.001978656 | 0.003764336 |
| G]A | | | | | | | | | | |
| T[C > | 9.44E−06 | 0.002021443 | 0.002402445 | 0.001194245 | 0.000345903 | 0.000153463 | 0.000493579 | 0.003614949 | 0.0014364 | 0.005013319 |
| G]C | | | | | | | | | | |
| T[C > | 7.85E−18 | 0.00018211 | 0.000101834 | 0 | 2.84E−05 | 6.46E−05 | 0.000104905 | 0.002200379 | 0.000589573 | 0.001911239 |
| G]G | | | | | | | | | | |
| T[C > | 0.002616625 | 0.003328958 | 0.00493498 | 0.000932965 | 0.000147162 | 0.000548983 | 0.000424071 | 0.003354951 | 0.002428709 | 0.003780449 |
| G]T | | | | | | | | | | |
| T[C > | 0.001408363 | 0.002418069 | 0.018552652 | 0.00727909 | 0.000211881 | 0.000187909 | 0.019458229 | 0.001705438 | 0.001469002 | 0.005767285 |
| T]A | | | | | | | | | | |
| T[C > | 0.002259317 | 0.0020315 | 0.012612838 | 0.037232346 | 0.000313289 | 0.000583762 | 0.1041221 | 0.001772426 | 0.001526176 | 0.005796964 |
| T]C | | | | | | | | | | |
| T[C > | 7.85E−18 | 0.001790409 | 0.003767842 | 0.001980601 | 0.000404167 | 0.000953034 | 0.004043419 | 0.006611323 | 0.002222316 | 0.010424942 |
| T]G | | | | | | | | | | |
| T[C > | 0.003051045 | 0.002984932 | 0.004431411 | 0.081684956 | 5.82E−06 | 0.000314858 | 0.082396725 | 0.042717701 | 0.001655938 | 0.007793386 |
| T]T | | | | | | | | | | |
| T[T > | 0.006875588 | 0.000402685 | 0.004431601 | 0.154019959 | 2.28E−05 | 0.000414446 | 0.001664571 | 0.019801446 | 0.001030406 | 0.003064025 |
| A]A | | | | | | | | | | |
| T[T > | 2.11E−05 | 0.000267504 | 0.004369923 | 1.25E−05 | 7.67E−06 | 5.80E−05 | 0.000674902 | 0.002622875 | 0.000474951 | 0.003631096 |
| A]C | | | | | | | | | | |
| T[T > | 5.06E−06 | 0.000660211 | 0.000502403 | 0.000455328 | 8.25E−06 | 7.58E−05 | 7.58E−05 | 0.004050789 | 0.000778991 | 0.001613557 |
| A]G | | | | | | | | | | |
| T[T > | 0.013958771 | 0.001398034 | 0.00340376 | 0.004365897 | 0.00042105 | 0.000332031 | 0.00295372 | 0.006389301 | 0.001336766 | 0.005104182 |
| A]T | | | | | | | | | | |
| T[T > | 0.019658648 | 0.00126643 | 0.009158643 | 0.001352611 | 0.000588231 | 0.000170712 | 0.001073599 | 0.004393754 | 0.002718308 | 0.00702749 |
| C]A | | | | | | | | | | |
| T[T > | 0.010704022 | 0.001123107 | 0.055182975 | 0.017829845 | 0.000405351 | 8.63E−05 | 0.029476139 | 0.00399531 | 0.002207662 | 0.005130454 |
| C]C | | | | | | | | | | |
| T[T > | 0.019251639 | 0.001251776 | 0.023813886 | 0.001591634 | 0.000294634 | 6.35E−05 | 0.00102506 | 0.002482742 | 0.001071363 | 0.004759994 |
| C]G | | | | | | | | | | |
| T[T > | 0.037256863 | 0.00054815 | 0.03654036 | 0.011542026 | 0.000525818 | 0.000208648 | 0.005116444 | 0.021331051 | 0.001995174 | 0.002200425 |
| C]T | | | | | | | | | | |
| T[T > | 7.59E−18 | 0.000281979 | 0.00068936 | 0.002379411 | 0.000243071 | 0.000148016 | 0.00032208 | 0.002512124 | 0.001403314 | 0.001284914 |
| G]A | | | | | | | | | | |
| T[T > | 7.83E−18 | 0.000137836 | 0.001016261 | 2.29E−05 | 0.000222742 | 0.000140525 | 0.000665754 | 0.003527666 | 0.001134212 | 0.004447073 |
| G]C | | | | | | | | | | |
| T[T > | 0.00092428 | 0.000714724 | 0.000904325 | 0.00155134 | 0.000347212 | 0.000112764 | 0.008763261 | 0.002774238 | 0.001481885 | 0.006202111 |
| G]G | | | | | | | | | | |
| T[T > | 0.009038053 | 0.000728185 | 0.001001106 | 0.003633229 | 3.16E−07 | 0.000365134 | 0.001602018 | 0.002914242 | 0.002523895 | 0.00708356 |
| G]T | | | | | | | | | | |
| Type | SBS54 | SBS55 | SBS56 | SBS57 | SBS58 | SBS59 | SBS60 | SBS84 | SBS85 | SBS86 |
| A[C > | 0.002170006 | 0.005899033 | 0.012605991 | 0.012329126 | 0.058964899 | 0.003616718 | 0.006163153 | 0.00350024 | 0.006107867 | 0.002968167 |
| A]A | | | | | | | | | | |
| A[C > | 0.000787553 | 0.00202543 | 0.01546913 | 0.001411637 | 0.006654905 | 0.00235141 | 0.000768821 | 0.004971448 | 0.000870521 | 0.003734676 |
| A]C | | | | | | | | | | |
| A[C > | 0.001692037 | 4.63E−05 | 0.000211657 | 0.003427917 | 0.000848186 | 0.000146915 | 2.30E−16 | 0.000468632 | 0.000315692 | 0.000397746 |
| A]G | | | | | | | | | | |
| A[C > | 0.000415742 | 0.001173292 | 0.023001237 | 0.008656809 | 0.005583794 | 0.014601757 | 0.000444762 | 0.00936762 | 0.002728468 | 0.003639476 |
| A]T | | | | | | | | | | |
| A[C > | 0.00155718 | 0.002859225 | 0.000418199 | 0.015436466 | 0.020622698 | 0.002961881 | 0.000377806 | 0.006813054 | 0.007267781 | 0.052762504 |
| G]A | | | | | | | | | | |
| A[C > | 0.000490737 | 0.000165986 | 0.000163559 | 0.003089807 | 0.001715487 | 0.002420861 | 0.000580317 | 0.010734524 | 0.002524052 | 0.077494661 |
| G]C | | | | | | | | | | |
| A[C > | 0.001753941 | 0.000525451 | 0.000439753 | 0.00285145 | 0.001737496 | 0.000858731 | 0.000284051 | 4.76E−05 | 0.000435554 | 0.005123162 |
| G]G | | | | | | | | | | |
| A[C > | 0.005201802 | 0.001273574 | 0.000123007 | 0.005079863 | 0.00203138 | 0.003856878 | 0.000250429 | 0.029822389 | 0.013073371 | 0.092475228 |
| G]T | | | | | | | | | | |
| A[C > | 0.001486856 | 0.005126541 | 0.007403518 | 0.032677195 | 0.080588699 | 0.003949174 | 0.002765903 | 0.054324129 | 0.001381024 | 0.008765251 |
| T]A | | | | | | | | | | |
| A[C > | 0.000523387 | 0.000230207 | 0.00096756 | 0.012734347 | 0.006664764 | 0.002837566 | 0.00132249 | 0.041805272 | 0.001462621 | 0.002027876 |
| T]C | | | | | | | | | | |
| A[C > | 0.000353883 | 0.005481179 | 0.00156174 | 0.018117517 | 0.008872539 | 0.002648616 | 0.004332801 | 0.002630427 | 0.000457815 | 0.024856926 |
| T]G | | | | | | | | | | |
| A[C > | 0.005011002 | 0.000778184 | 0.002840153 | 0.01542996 | 0.00731497 | 0.00341379 | 0.000298511 | 0.054764884 | 0.004582374 | 0.004594638 |
| T]T | | | | | | | | | | |
| A[T > | 0.000951711 | 0.004234997 | 5.01E−06 | 0.003257924 | 0.024304369 | 0.001996596 | 0.003942378 | 0.001560752 | 0.111346739 | 0.001839523 |
| A]A | | | | | | | | | | |
| A[T > | 0.003199582 | 0.000618995 | 0.004285082 | 0.006434778 | 0.001459239 | 0.002514523 | 0.000223115 | 0.000400045 | 0.013089267 | 0.003811548 |
| A]C | | | | | | | | | | |
| A[T > | 0.004212337 | 0.008054975 | 0.000508744 | 0.005762764 | 0.004654004 | 0.002186956 | 0.00034966 | 7.08E−05 | 0.021017213 | 0.001985895 |
| A]G | | | | | | | | | | |
| A[T > | 0.000299915 | 0.003900442 | 0.028775882 | 0.006273079 | 0.008707672 | 0.002797708 | 0.000723575 | 0.010651427 | 0.070028623 | 0.007232674 |
| A]T | | | | | | | | | | |
| A[T > | 0.029068992 | 0.004324469 | 0.012392429 | 0.026420973 | 0.079857213 | 0.009933065 | 0.001281025 | 0 | 0.029431693 | 0.00359482 |
| C]A | | | | | | | | | | |
| A[T > | 0.007773279 | 3.37E−06 | 0.006152939 | 0.009667179 | 0.004927432 | 0.002625162 | 0.000277142 | 0.000325234 | 0.020469935 | 0.002070728 |
| C]C | | | | | | | | | | |
| A[T > | 0.201156541 | 0.003997654 | 0.009470774 | 0.010233874 | 0.006698194 | 0.003515107 | 3.50E−06 | 1.15E−05 | 0.005049307 | 0.00350731 |
| C]G | | | | | | | | | | |
| A[T > | 0.007801769 | 0.013631645 | 0.01230417 | 0.017795263 | 0.008102007 | 0.010791161 | 3.61E−05 | 0.009380926 | 0.059015151 | 0.004452861 |
| C]T | | | | | | | | | | |
| A[T > | 3.85E−05 | 0.000260462 | 0.000119959 | 0.007012484 | 0.013392203 | 0.001786953 | 0.024826061 | 0.000127009 | 0.023729114 | 0.004759941 |
| G]A | | | | | | | | | | |
| A[T > | 0.001584746 | 0.000921482 | 7.98E−18 | 0.000789586 | 0.00078759 | 0.001569063 | 0.010505399 | 0.001279218 | 0.003444925 | 7.33E−05 |
| G]C | | | | | | | | | | |
| A[T > | 0.001145198 | 0.144193994 | 1.26E−05 | 0.001222103 | 0.002689201 | 0.001697637 | 0.025926506 | 0.000952029 | 0.004466366 | 0.001714915 |
| G]G | | | | | | | | | | |
| A[T > | 0.000338774 | 0.002268624 | 0.005705563 | 0.005935068 | 0.002065218 | 0.003437185 | 0.01630031 | 0.001764744 | 0.003166848 | 0.0011245 |
| G]T | | | | | | | | | | |
| C[C > | 6.28E−05 | 0.006199896 | 0.002453347 | 0.00862278 | 0.006117411 | 0.002221396 | 0.001169259 | 0 | 0.004235549 | 0.006075881 |
| A]A | | | | | | | | | | |
| C[C > | 0.002535829 | 0.003695 | 0.006563909 | 0.003878851 | 0.001799323 | 0.00586752 | 0.000249016 | 0.005167138 | 0.000302136 | 0.003109985 |
| A]C | | | | | | | | | | |
| C[C > | 0.005944798 | 0.000641707 | 6.71E−18 | 0.001070252 | 0.0014585 | 0.000819319 | 0.000379003 | 6.96E−05 | 0.000157635 | 0.000555537 |
| A]G | | | | | | | | | | |
| C[C > | 0.005493463 | 2.82E−05 | 0.07053585 | 0.008137374 | 0.001940208 | 0.002012267 | 5.36E−05 | 0.00562265 | 0.000855086 | 0.003292902 |
| A]T | | | | | | | | | | |
| C[C > | 0.0011225 | 0.002916431 | 0.000201808 | 0.000880118 | 0.000785959 | 0.002071975 | 4.67E−05 | 7.10E−06 | 0.001215711 | 0.021078471 |
| G]A | | | | | | | | | | |
| C[C > | 0.000871259 | 0.002818053 | 0.00022109 | 0.002825457 | 0.001258543 | 0.001711772 | 0.000139764 | 0.003406874 | 0.000207366 | 0.058334545 |
| G]C | | | | | | | | | | |
| C[C > | 0.000435083 | 0.000264291 | 6.65E−05 | 0.00076318 | 5.10E−05 | 0.000693657 | 0.000337002 | 2.21E−06 | 4.03E−05 | 0.00316407 |
| G]G | | | | | | | | | | |
| C[C > | 0.001321213 | 0.003283564 | 0.000760758 | 0.002844612 | 0.001237377 | 0.003446754 | 0.000144675 | 0.013266414 | 0.000480842 | 0.039969628 |
| G]T | | | | | | | | | | |
| C[C > | 0.003347634 | 0.007191879 | 0.000294797 | 0.01476775 | 0.008008067 | 0.002858927 | 0.001506164 | 0.017970918 | 0.001625987 | 0.003519304 |
| T]A | | | | | | | | | | |
| C[C | 0.000222995 | 0.003941333 | 0.000521772 | 0.011912206 | 0.009753705 | 0.000404691 | 0.001850399 | 0.040220939 | 0.000364429 | 0.004776433 |
| T]C | | | | | | | | | | |
| C[C > | 0.02837062 | 6.60E−05 | 0.007936862 | 0.00640151 | 0.00798179 | 0.001105829 | 0.004060029 | 0 | 2.42E−05 | 0.005929924 |
| T]G | | | | | | | | | | |
| C[C > | 0.003685488 | 0.000713257 | 0.000502556 | 0.031716925 | 0.027420285 | 0.002410735 | 0.002487227 | 0.04210452 | 0.002334565 | 0.002342962 |
| T]T | | | | | | | | | | |
| C[T > | 7.44E−18 | 7.37E−05 | 0.001183535 | 0.006367309 | 0.002468072 | 7.04E−05 | 6.61E−05 | 0.000136848 | 0.009882556 | 0.002115376 |
| A]A | | | | | | | | | | |
| C[T > | 0.00073326 | 1.10E−06 | 0.00097164 | 0.006586469 | 0.022259899 | 0.001647419 | 0.000513607 | 0 | 0.00113787 | 0.000508896 |
| A]C | | | | | | | | | | |
| C[T > | 0.001917537 | 0.015957285 | 0.00158139 | 0.001285442 | 0.000853888 | 0.00013921 | 3.80E−07 | 0.000125451 | 0.000178338 | 0.002619996 |
| A]G | | | | | | | | | | |
| C[T > | 6.69E−05 | 0.005250071 | 6.10E−06 | 0.01073176 | 0.001302212 | 0.003739849 | 0.000132362 | 0 | 0.010605415 | 0.003351756 |
| A]T | | | | | | | | | | |
| C[T > | 0.011385179 | 0.004168767 | 0.003351693 | 0.011957387 | 0.003901146 | 0.002203285 | 0.000875678 | 0.002274618 | 0.007174042 | 0.001282409 |
| C]A | | | | | | | | | | |
| C[T > | 0.017029273 | 0.004981646 | 0.002633604 | 0.004557557 | 0.009482917 | 0.005755922 | 0.002787867 | 3.27E−06 | 0.006219193 | 0.002957164 |
| C]C | | | | | | | | | | |
| C[T > | 0.192752464 | 0.000946466 | 0.000478395 | 0.008270672 | 0.005901582 | 0.001642478 | 0.000577818 | 0.003636296 | 0.000838799 | 0.000557129 |
| C]G | | | | | | | | | | |
| C[T > | 0.027040791 | 0.000686625 | 0.000206221 | 0.024372127 | 0.028247978 | 0.012298157 | 0.002105751 | 0.000926609 | 0.017000117 | 0.001802093 |
| C]T | | | | | | | | | | |
| C[T > | 0.004354331 | 0.001645566 | 0.005788384 | 0.000139503 | 0.000500581 | 0.001011508 | 0.003217794 | 0.000899151 | 0.003731278 | 0.004066656 |
| G]A | | | | | | | | | | |
| C[T > | 0.018932544 | 0.000466154 | 3.92E−07 | 0.000846546 | 0.00103813 | 0.002631853 | 0.00415682 | 0.002160078 | 0.002482212 | 0.000164383 |
| G]C | | | | | | | | | | |
| C[T > | 0.022071655 | 0.112698323 | 0.004425904 | 0.002182262 | 0.00069565 | 0.002023052 | 0.007083256 | 0.000790406 | 0.001118761 | 0.00176674 |
| G]G | | | | | | | | | | |
| C[T > | 0.000890637 | 0.001184528 | 6.79E−18 | 0.005175316 | 0.000473805 | 0.021370568 | 1.80E−05 | 0 | 0.001765905 | 0.002434122 |
| G]T | | | | | | | | | | |
| G[C > | 0.005194222 | 0.000854507 | 0.000216278 | 0.001887274 | 0.002732592 | 0.10337213 | 0.000516136 | 0.002715105 | 0.001440743 | 0.004846737 |
| A]A | | | | | | | | | | |
| G[C > | 0.00901184 | 0.000189252 | 0.007215029 | 0.000740595 | 0.001062277 | 0.127686657 | 0.000852671 | 0.01013168 | 0.000497697 | 0.003582352 |
| A]C | | | | | | | | | | |
| G[C > | 0.005689344 | 7.78E−05 | 9.83E−06 | 6.61E−18 | 0.002744207 | 0.012240004 | 0.000392436 | 0.000772967 | 0.000365419 | 0.000225005 |
| A]G | | | | | | | | | | |
| G[C > | 0.006701499 | 0.000733854 | 0.015568827 | 0.001937366 | 0.000347293 | 0.498276673 | 0.000132108 | 0.010171399 | 0.000759647 | 0.008335663 |
| A]T | | | | | | | | | | |
| G[C > | 0.002191782 | 0.000850509 | 0.000186378 | 0.003784534 | 0.002114269 | 0.001445203 | 0.000162728 | 0.011199042 | 0.001597077 | 0.005563663 |
| G]A | | | | | | | | | | |
| G[C > | 0.000217153 | 0.001389819 | 9.66E−05 | 0.002531022 | 0.001465548 | 0.001702489 | 0.000132922 | 0.015608631 | 0.004994418 | 0.107931552 |
| G]C | | | | | | | | | | |
| G[C > | 0.001603911 | 0.00576203 | 5.65E−06 | 0.001872044 | 0.001718899 | 0.000363154 | 0.000203262 | 0.001718794 | 2.70E−05 | 0.00077053 |
| G]G | | | | | | | | | | |
| G[C > | 0.00491908 | 6.78E−05 | 1.74E−06 | 0.000433175 | 0.001071647 | 0.002256724 | 6.39E−05 | 0.075999526 | 0.003172323 | 0.01240202 |
| G]T | | | | | | | | | | |
| G[C > | 0.000222181 | 0.006496252 | 9.46E−06 | 0.005132587 | 0.00676167 | 0.002448815 | 0.001237928 | 0.089561283 | 0.000887674 | 0.004537566 |
| T]A | | | | | | | | | | |
| G[C > | 0.012930461 | 0.001488388 | 0.000497385 | 0.006824895 | 0.003098307 | 0.002326075 | 0.001417836 | 0.110569021 | 0.000478054 | 0.003330834 |
| T]C | | | | | | | | | | |
| G[C > | 0.019267104 | 0.011987439 | 0.002069425 | 0.008494652 | 0.007398302 | 3.28E−18 | 0.003380984 | 0.009337066 | 0.000117432 | 0.00804224 |
| T]G | | | | | | | | | | |
| G[C > | 0.00024695 | 0.005260281 | 0.000823065 | 0.006139959 | 0.004991097 | 0.002955709 | 0.000888004 | 0.180302772 | 0.004149081 | 0.007106216 |
| T]T | | | | | | | | | | |
| G[T > | 0.002313499 | 0.002409439 | 0.0004153 | 0.003298965 | 0.002048117 | 0.000253902 | 0.000107959 | 0.001094662 | 0.017115589 | 0.001250359 |
| A]A | | | | | | | | | | |
| G[T > | 0.008946578 | 0.000183851 | 0.002348794 | 0.000592777 | 0.000620787 | 0.001975948 | 0.000109197 | 6.55E−05 | 0.001000163 | 0.0003548 |
| A]C | | | | | | | | | | |
| G[T > | 0.002648989 | 0.012525141 | 0.000435674 | 0.002462813 | 0.020397529 | 0.000508137 | 0.000360329 | 0.00033971 | 0.003531831 | 0.003072514 |
| A]G | | | | | | | | | | |
| G[T > | 0.0018744 | 0.005180324 | 0.005418315 | 0.008016483 | 0.002796672 | 0.001534195 | 0.0006438 | 0.000138027 | 0.011811196 | 0.002675288 |
| A]T | | | | | | | | | | |
| G[T > | 0.011319265 | 0.002587916 | 0.000721831 | 0.003824025 | 0.002156955 | 0.000706944 | 0.000753735 | 0 | 0.021435853 | 0.004417068 |
| C]A | | | | | | | | | | |
| G[T > | 0.004408603 | 0.003150307 | 2.71E−05 | 0.002273803 | 0.000773505 | 0.003173493 | 0.000630364 | 0.000864767 | 0.011549028 | 0.006827683 |
| C]C | | | | | | | | | | |
| G[T > | 0.121412001 | 0.006022088 | 0.001648768 | 0.004725397 | 0.011098656 | 0.000992125 | 0.000500457 | 0.000199273 | 0.006484104 | 0.002689993 |
| C]G | | | | | | | | | | |
| G[T > | 0.005481354 | 0.012748455 | 0.002603213 | 0.005418455 | 0.00203945 | 0.001209068 | 0.000703429 | 0.003723717 | 0.054797422 | 0.008663199 |
| C]T | | | | | | | | | | |
| G[T > | 0.000168796 | 0.005622025 | 6.64E−18 | 0.001386952 | 0.00798469 | 0.001224705 | 0.027930786 | 1.88E−05 | 0.007644401 | 0.001385603 |
| G]A | | | | | | | | | | |
| G[T > | 0.00152261 | 0.003180121 | 9.11E−07 | 0.000169791 | 0.0026676 | 0.001646623 | 0.028589756 | 0.001227764 | 0.000796131 | 0.000683963 |
| G]C | | | | | | | | | | |
| G[T > | 7.48E−18 | 0.299601384 | 6.71E−18 | 0.000861984 | 0.023397166 | 0.002304225 | 0.662605332 | 0.00021422 | 0.001952964 | 0.000456055 |
| G]G | | | | | | | | | | |
| G[T > | 1.23E−05 | 0.001456966 | 0.000115026 | 0.002860863 | 0.017062728 | 0.007569374 | 0.047905971 | 0.001362488 | 0.002966254 | 0.002237158 |
| G]T | | | | | | | | | | |
| T[C > | 0.00067216 | 0.001745792 | 0.219124135 | 0.014335171 | 0.007378794 | 2.39E−05 | 0.002024505 | 0.002100706 | 0.001953395 | 0.008602673 |
| A]A | | | | | | | | | | |
| T[C > | 4.06E−05 | 8.29E−18 | 0.013183418 | 0.000709449 | 0.000428729 | 0.020315113 | 0.000163066 | 9.12E−06 | 0.000169903 | 0.003960268 |
| A]C | | | | | | | | | | |
| T[C > | 0.000844761 | 0.000100966 | 0.005547476 | 0.000227001 | 0.002775475 | 0.000648643 | 0.000208631 | 0 | 4.07E−09 | 0.001099379 |
| A]G | | | | | | | | | | |
| T[C > | 0.007264978 | 0.004090951 | 0.40394963 | 0.006231786 | 0.014780541 | 0.003875375 | 0.000408647 | 0.001218878 | 0.002075872 | 0.009165232 |
| A]T | | | | | | | | | | |
| T[C > | 0.004712156 | 0.007083499 | 0.001130641 | 0.002285608 | 0.002132542 | 2.37E−06 | 0.00053018 | 0 | 0.000952387 | 0.042427073 |
| G]A | | | | | | | | | | |
| T[C > | 0.000518496 | 0.003284922 | 0.000293631 | 0.002801772 | 0.001798864 | 0.00210191 | 0.000348141 | 0.000114671 | 0.001874644 | 0.047527025 |
| G]C | | | | | | | | | | |
| T[C > | 0.001254898 | 0.000233311 | 8.52E−05 | 0.001353864 | 0.000640494 | 0.000414354 | 6.71E−05 | 7.38E−07 | 0.000173422 | 0.004730033 |
| G]G | | | | | | | | | | |
| T[C > | 0.000313807 | 0.002126085 | 0.000412993 | 0.012987082 | 0.018199169 | 0.001538008 | 0.000765962 | 0.005015513 | 0.006299923 | 0.111847169 |
| G]T | | | | | | | | | | |
| T[C > | 7.49E−18 | 5.84E−11 | 0.000200113 | 0.00816002 | 0.004295119 | 0.000162213 | 4.08E−05 | 7.14E−05 | 0.003841325 | 0.008630807 |
| T]A | | | | | | | | | | |
| T[C > | 0.000662913 | 0.004116167 | 0.000183769 | 0.017310951 | 0.015290347 | 0.000281596 | 0.000958387 | 0.027081287 | 0.003006523 | 0.0081459 |
| T]C | | | | | | | | | | |
| T[C > | 1.21E−13 | 0.004900551 | 0.000156467 | 0.004682539 | 0.003395636 | 4.64E−18 | 0.001760639 | 7.08E−06 | 0.000161142 | 0.013968999 |
| T]G | | | | | | | | | | |
| T[C > | 0.00023813 | 0.031639373 | 8.24E−05 | 0.037451122 | 0.179980734 | 0.002114761 | 0.003935494 | 0.017050886 | 0.038996164 | 0.013102991 |
| T]T | | | | | | | | | | |
| T[T > | 0.001529299 | 0.003556839 | 0.009255735 | 0.00297295 | 0.01169981 | 0.002067855 | 0.001112141 | 0.006504763 | 0.059370774 | 0.003870153 |
| A]A | | | | | | | | | | |
| T[T > | 0.000182251 | 7.07E−05 | 0.000816231 | 0.009376474 | 0.000911098 | 0.001899082 | 2.26E−16 | 0.000883279 | 0.007935762 | 0.001743509 |
| A]C | | | | | | | | | | |
| T[T > | 0.0003312 | 0.009530137 | 0.006376038 | 0.004419439 | 0.000831611 | 0.000457305 | 4.26E−05 | 0.000792027 | 0.00677781 | 0.001401898 |
| A]G | | | | | | | | | | |
| T[T > | 7.47E−18 | 0.002103334 | 0.00074313 | 0.028920804 | 0.014592056 | 0.0030073 | 0.00053026 | 0.007105686 | 0.062979727 | 0.002372495 |
| A]T | | | | | | | | | | |
| T[T > | 0.004666828 | 0.004808138 | 0.000558095 | 0.004823661 | 0.003254317 | 0.002028279 | 0.000236207 | 0.000428646 | 0.012789252 | 0.001513507 |
| C]A | | | | | | | | | | |
| T[T > | 0.008338764 | 0.000996415 | 0.001338416 | 0.027123711 | 0.019885889 | 0.002287067 | 0.001107044 | 0.000550157 | 0.023745959 | 0.002031362 |
| C]C | | | | | | | | | | |
| T[T > | 0.088991126 | 0.004182001 | 0.001854665 | 0.002973077 | 0.002166803 | 0.001362829 | 0.000109457 | 2.26E−05 | 0.003848753 | 0.001858357 |
| C]G | | | | | | | | | | |
| T[T > | 0.012537256 | 0.000888408 | 0.000284667 | 0.195140376 | 0.031982588 | 0.002625101 | 0.001320648 | 0.009884377 | 0.094326134 | 0.003449762 |
| C]T | | | | | | | | | | |
| T[T > | 3.20E−05 | 0.005970761 | 0.009295037 | 0.003169835 | 0.000784575 | 0.001583047 | 0.001584555 | 0.001857099 | 0.007010864 | 0.007040633 |
| G]A | | | | | | | | | | |
| T[T > | 0.000718824 | 0.005744637 | 1.01E−06 | 0.004022843 | 0.001613192 | 0.002378958 | 0.010765751 | 0.00075394 | 0.006520193 | 0.00241167 |
| G]C | | | | | | | | | | |
| T[T > | 0.007741423 | 0.079920455 | 0.000101255 | 0.005423857 | 0.006761227 | 0.001978626 | 0.024000099 | 0 | 0.003432491 | 0.00202427 |
| G]G | | | | | | | | | | |
| T[T > | 0.000500487 | 0.041265401 | 0.028966085 | 0.119085665 | 0.021688192 | 1.31E−17 | 0.031815612 | 0.006645905 | 0.012745482 | 0.004902067 |
| G]T | | | | | | | | | | |

| Type | SBS87 | SBS88 | SBS89 | SBS90 | SBS91 | SBS92 | SBS93 | SBS94 |
|---|---|---|---|---|---|---|---|---|
| A[C > | 0.008946076 | 0 | 0.032296782 | 0.002222002 | 0.002934397 | 0.011395596 | 0.011627704 | 0.015676822 |
| A]A | | | | | | | | |
| A[C > | 0.004489907 | 0 | 0.017494551 | 0.000703924 | 0.052012979 | 0.009652994 | 0.008011028 | 0.024522706 |
| A]C | | | | | | | | |
| A[C > | 0.006357214 | 0 | 0.009971177 | 0.000143619 | 0.000208985 | 0.004851456 | 0.00181662 | 0.001626815 |
| A]G | | | | | | | | |
| A[C > | 0.004940762 | 0.001737757 | 0.020817534 | 0.001770639 | 0.000130304 | 0.007800061 | 0.008456891 | 0.011140516 |
| A]T | | | | | | | | |
| A[C > | 0.007843246 | 0 | 0.014876093 | 0.00051277 | 0.00024248 | 0.003073731 | 0.008898488 | 0.007047548 |
| G]A | | | | | | | | |
| A[C > | 0.003862855 | 0.003994926 | 0.005816986 | 0.000275448 | 0.00012226 | 0.001673952 | 0.005361007 | 0.003954052 |
| G]C | | | | | | | | |
| A[C > | 0.031395949 | 0 | 0.006238471 | 4.79E−05 | 4.84E−05 | 0.000937221 | 0.00168346 | 0.00056871 |
| G]G | | | | | | | | |
| A[C > | 0.009687581 | 0.004054767 | 0.010939422 | 0.000931916 | 8.06E−05 | 0.004138235 | 0.013961245 | 0.007165036 |
| G]T | | | | | | | | |
| A[C > | 0.009397239 | 0.01043094 | 0.050096541 | 0.000714563 | 0.015709056 | 0.023905359 | 0.008983093 | 0.00757732 |
| T]A | | | | | | | | |
| A[C > | 0.003491299 | 0 | 0.013369602 | 0.000290751 | 0.001192374 | 0.013964827 | 0.00501777 | 0.014161035 |
| T]C | | | | | | | | |
| A[C > | 0.124633581 | 0.013092798 | 0.006295248 | 9.58E−05 | 0.002279735 | 0.0101085 | 0.018198733 | 0.023870348 |
| T]G | | | | | | | | |
| A[C > | 0.010665867 | 0 | 0.029741843 | 0.00065234 | 0.002317195 | 0.016541309 | 0.006536906 | 0.016605952 |
| T]T | | | | | | | | |
| A[T > | 0.009778275 | 0.047661646 | 0.011457108 | 0.061358775 | 0.000212575 | 0.010038976 | 0.010367584 | 0.010494335 |
| A]A | | | | | | | | |
| A[T > | 0.000691354 | 0.02372085 | 0.003074882 | 0.000356805 | 0.000168379 | 0.003603284 | 0.005806687 | 0.005634446 |
| A]C | | | | | | | | |
| A[T > | 0.003798442 | 0.000574416 | 0.010619079 | 0.000354251 | 0.000158248 | 0.006990903 | 0.005295638 | 0.004018512 |
| A]G | | | | | | | | |
| A[T > | 0.004159378 | 0.042531125 | 0.004429556 | 0.023486458 | 0.000369511 | 0.009159209 | 0.013229474 | 0.015878199 |
| A]T | | | | | | | | |
| A[T > | 0.009104463 | 0.110827683 | 0.015701245 | 0.005712448 | 0.000296095 | 0.088895359 | 0.034292415 | 0.013594447 |
| C]A | | | | | | | | |
| A[T > | 0.003104739 | 0.045706028 | 0.003461508 | 0.000186159 | 0.000148072 | 0.004656919 | 0.003301877 | 0.005177576 |
| C]C | | | | | | | | |
| A[T > | 0.000961836 | 0.006318577 | 0.014619506 | 0.000107815 | 0.000603164 | 0.039992845 | 0.006266997 | 0.002821467 |
| C]G | | | | | | | | |
| A[T > | 0.005513535 | 0.16897501 | 0.01176182 | 0.002866457 | 0.000232479 | 0.029495992 | 0.010638957 | 0.022891461 |
| C]T | | | | | | | | |
| A[T > | 0.007859591 | 0.008039314 | 0.005210918 | 0.005435294 | 0.002358351 | 0.002062868 | 0.025371281 | 0.003708851 |
| G]A | | | | | | | | |
| A[T > | 0.000670341 | 0.005207015 | 0.001303296 | 4.66E−05 | 0.000130921 | 0.000360484 | 0.00240039 | 0.000921605 |
| G]C | | | | | | | | |
| A[T > | 0.00265636 | 0.002297664 | 0.005085754 | 0.000231033 | 0.001205627 | 0.001234692 | 0.005117646 | 0.002071737 |
| G]G | | | | | | | | |
| A[T > | 0.0021498 | 0.007471685 | 0.003977219 | 0.002049671 | 0.000211459 | 0.003532703 | 0.00793488 | 0.002285032 |
| G]T | | | | | | | | |
| C[C > | 0.008436496 | 0 | 0.024491736 | 0.001183042 | 0.008071325 | 0.018449535 | 0.006455838 | 0.079521783 |
| A]A | | | | | | | | |
| C[C > | 0.005915067 | 0 | 0.014312131 | 0.000442574 | 0.00448227 | 0.01378663 | 0.00473232 | 0.075103384 |
| A]C | | | | | | | | |
| C[C > | 0.019905429 | 0 | 0.003295933 | 0.000217539 | 0.002313399 | 0.005301619 | 0.001512139 | 0.006250465 |
| A]G | | | | | | | | |
| C[C > | 0.004816662 | 0 | 0.016612291 | 0.001014066 | 0.003192337 | 0.012475307 | 0.009996422 | 0.039703675 |
| A]T | | | | | | | | |
| C[C > | 0.006670032 | 0.00458397 | 0.015313054 | 0.000338011 | 6.54E−05 | 0.002396495 | 0.00335589 | 0.009149311 |
| G]A | | | | | | | | |
| C[C > | 0.003269914 | 0.00113831 | 0.010470474 | 7.63E−05 | 0.000197767 | 0.003914269 | 0.003165982 | 0.014898071 |
| G]C | | | | | | | | |
| C[C > | 0.085548473 | 0 | 0.00417752 | 1.55E−05 | 5.31E−05 | 0.001606948 | 0.000777629 | 0.001850228 |
| G]G | | | | | | | | |
| C[C > | 0.009475088 | 0.002865067 | 0.015718742 | 0.000291928 | 9.92E−05 | 0.005336998 | 0.006012309 | 0.008508496 |
| G]T | | | | | | | | |
| C[C > | 0.00826466 | 0 | 0.009375413 | 0.000614567 | 0.036078546 | 0.036408401 | 0.008644312 | 0.013588477 |
| T]A | | | | | | | | |
| C[C > | 0.007878367 | 0 | 0.011397843 | 0.000305224 | 0.000265033 | 0.034340627 | 0.0039387 | 0.017735488 |
| T]C | | | | | | | | |
| C[C > | 0.185951149 | 0 | 0.002338117 | 0.000201571 | 0.00126946 | 0.010864103 | 0.00726387 | 0.01890233 |
| T]G | | | | | | | | |
| C[C > | 0.006863861 | 0.004011094 | 0.012792777 | 0.000368751 | 0.001204139 | 0.045078515 | 0.006860733 | 0.024922408 |
| T]T | | | | | | | | |
| C[T > | 0.008278336 | 0 | 0.031491276 | 0.102396297 | 0.000314306 | 0.007045276 | 0.010620025 | 0.011936135 |
| A]A | | | | | | | | |
| C[T > | 0.004882199 | 0.003466907 | 0.017728675 | 0.000356349 | 0.000141008 | 0.003943427 | 0.008532673 | 0.008817228 |
| A]C | | | | | | | | |
| C[T > | 0.002742875 | 0 | 0.036803272 | 0.000370728 | 0.000243944 | 0.010244004 | 0.007495176 | 0.007737049 |
| A]G | | | | | | | | |
| C[T > | 0.010167957 | 0 | 0.020038998 | 0.101511446 | 0.000459009 | 0.006490277 | 0.024718944 | 0.014539332 |
| A]T | | | | | | | | |
| C[T > | 0.005076703 | 0 | 0.004880079 | 0.004788483 | 0.000153184 | 0.030761778 | 0.029522601 | 0.006296677 |
| C]A | | | | | | | | |
| C[T > | 0.001823281 | 0.001733454 | 0.004220423 | 0.000216908 | 0.000145156 | 0.004022549 | 0.00837112 | 0.014976041 |
| C]C | | | | | | | | |
| C[T > | 0.00082872 | 0 | 0.008081885 | 0.000200811 | 0.000149061 | 0.014121767 | 0.002375243 | 0.007046347 |
| C]G | | | | | | | | |
| C[T > | 0.008836306 | 0 | 0.004333476 | 0.006421233 | 0.000162974 | 0.01375853 | 0.014937199 | 0.016216498 |
| C]T | | | | | | | | |
| C[T > | 0.008440093 | 0 | 0.006889544 | 0.008974543 | 0.00075114 | 0.000928315 | 0.009309268 | 0.002228736 |
| G]A | | | | | | | | |
| C[T > | 0.001077565 | 0.002311271 | 0.003223259 | 4.65E−05 | 5.84E−05 | 0.000420009 | 0.006378134 | 0.002059381 |
| G]C | | | | | | | | |
| C[T > | 0.001941709 | 0.002304339 | 0.009012645 | 0.000123576 | 0.000424865 | 0.002090366 | 0.002549378 | 0.002514323 |
| G]G | | | | | | | | |
| C[T > | 0.003074929 | 0 | 0.004198977 | 0.009795786 | 0.000121925 | 0.000548707 | 0.038423196 | 0.002816053 |
| G]T | | | | | | | | |
| G[C > | 0.007054884 | 0.008659409 | 0.006783083 | 0.000975203 | 0.00349916 | 0.00895741 | 0.002244197 | 0.013926149 |
| A]A | | | | | | | | |
| G[C > | 0.004570825 | 0 | 0.005943841 | 0.0004122 | 0.005747712 | 0.007732998 | 0.001678369 | 0.030103854 |
| A]C | | | | | | | | |
| G[C > | 0.008297261 | 0.000581871 | 0.001693528 | 0.000124649 | 5.91E−05 | 0.004500163 | 0.000298292 | 0.001782784 |
| A]G | | | | | | | | |
| G[C > | 0.00105495 | 0.003446291 | 0.007201857 | 0.000400434 | 0.001152728 | 0.006536227 | 0.003087681 | 0.011314222 |
| A]T | | | | | | | | |
| G[C > | 0.002599942 | 0 | 0.006523034 | 0.000433424 | 3.73E−05 | 0.002353566 | 0.003535258 | 0.003121515 |
| G]A | | | | | | | | |
| G[C > | 0.001173858 | 0.001708078 | 0.004528049 | 0.000290066 | 0.000185421 | 0.00213593 | 0.004604714 | 0.006808761 |
| G]C | | | | | | | | |
| G[C > | 0.005461662 | 0.000581871 | 0.002096749 | 4.68E−05 | 6.39E−05 | 0.000902277 | 0.000919151 | 0.000711417 |
| G]G | | | | | | | | |
| G[C > | 0.004446306 | 0.006892583 | 0.005688644 | 0.000261823 | 0.000155966 | 0.001522852 | 0.005650132 | 0.003682055 |
| G]T | | | | | | | | |
| G[C > | 0.001869006 | 0.000577294 | 0.006854793 | 0.000448903 | 0.011225434 | 0.012851799 | 0.006119676 | 0.005604839 |
| T]A | | | | | | | | |
| G[C > | 0.002564697 | 0 | 0.008413382 | 0.0003206 | 0.00012871 | 0.013352193 | 0.005066104 | 0.016582289 |
| T]C | | | | | | | | |
| G[C > | 0.017729002 | 0.006400583 | 0.001895139 | 0.000171266 | 0.001192618 | 0.005998089 | 0.009093597 | 0.017108422 |
| T]G | | | | | | | | |
| G[C > | 0.001429396 | 0.003446291 | 0.009180787 | 0.000508244 | 0.000148598 | 0.016795091 | 0.004493161 | 0.008415772 |
| T]T | | | | | | | | |
| G[T > | 0.009264605 | 0 | 0.00576554 | 0.038869302 | 0.000269607 | 0.005590889 | 0.005738048 | 0.005638393 |
| A]A | | | | | | | | |
| G[T > | 0.003357033 | 0.005166347 | 0.002507388 | 0.000169313 | 0.000314163 | 0.001917981 | 0.004352242 | 0.001486309 |
| A]C | | | | | | | | |
| G[T > | 0.006782172 | 0.002893943 | 0.009157538 | 0.000155194 | 0.000118472 | 0.006023899 | 0.004453867 | 0.002237109 |
| A]G | | | | | | | | |
| G[T > | 0.006994008 | 0.015195248 | 0.003923442 | 0.016924491 | 0.000731651 | 0.004177211 | 0.005265565 | 0.007363717 |
| A]T | | | | | | | | |
| G[T > | 0.002965714 | 0 | 0.005010774 | 0.003271807 | 0.000168824 | 0.029037579 | 0.011320941 | 0.004121938 |
| C]A | | | | | | | | |
| G[T > | 0.003846711 | 0.003444231 | 0.001635862 | 9.24E−05 | 0.000133713 | 0.002676023 | 0.002797591 | 0.001134976 |
| C]C | | | | | | | | |
| G[T > | 0.003945263 | 0.000578789 | 0.007279691 | 7.76E−05 | 0.000144153 | 0.009302585 | 0.000207996 | 0.000952321 |
| C]G | | | | | | | | |
| G[T > | 0.006577214 | 0.01344195 | 0.004110126 | 0.001347691 | 0.000153303 | 0.012855638 | 0.005956889 | 0.010904778 |
| C]T | | | | | | | | |
| G[T > | 0.003476555 | 0 | 0.01266258 | 0.006342561 | 9.92E−05 | 0.000547253 | 0.00371358 | 0.001271904 |
| G]A | | | | | | | | |
| G[T > | 0.001223406 | 0.001722116 | 0.001263204 | 4.62E−05 | 0.001373572 | 0.000293163 | 0.002691363 | 0.000630883 |
| G]C | | | | | | | | |
| G[T > | 0.003222991 | 0.002893943 | 0.04861622 | 0.000232792 | 7.30E−05 | 0.001551484 | 0.000869003 | 0.001984302 |
| G]G | | | | | | | | |
| G[T > | 0.002474223 | 0.01344195 | 0.003817087 | 0.002931457 | 5.42E−05 | 0.000202477 | 0.006229481 | 0.002152909 |
| G]T | | | | | | | | |
| T[C > | 0.003994024 | 0 | 0.015804054 | 0.002035726 | 0.332762881 | 0.014936781 | 0.009938121 | 0.02539913 |
| A]A | | | | | | | | |
| T[C > | 0.004159435 | 0.002313974 | 0.016822356 | 0.00079109 | 0.045004556 | 0.012884482 | 0.004049406 | 0.020394945 |
| A]C | | | | | | | | |
| T[C > | 0.003212069 | 0.000588501 | 0.0019881 | 7.89E−05 | 0.322250265 | 0.002444569 | 0.000178249 | 0.0026647 |
| A]G | | | | | | | | |
| T[C > | 0.004139601 | 0.010476547 | 0.018870497 | 0.001467002 | 0.07655879 | 0.013321366 | 0.008828422 | 0.008239534 |
| A]T | | | | | | | | |
| T[C > | 0.000637708 | 0 | 0.022563078 | 0.000652675 | 4.86E−05 | 0.0195667 | 0.012872177 | 0.006131227 |
| G]A | | | | | | | | |
| T[C > | 0.002827898 | 0 | 0.016628201 | 0.000325743 | 0.000152582 | 0.004104288 | 0.007083268 | 0.005372867 |
| G]C | | | | | | | | |
| T[C > | 0.026444414 | 0.000588501 | 0.00371112 | 6.31E−05 | 2.61E−05 | 0.000304809 | 0.000714394 | 0.001065493 |
| G]G | | | | | | | | |
| T[C > | 0.012648725 | 0.004074212 | 0.026298275 | 0.001451397 | 0.000267473 | 0.023224828 | 0.025961234 | 0.012744829 |
| G]T | | | | | | | | |
| T[C > | 8.70E−06 | 0 | 0.009508168 | 0.002129755 | 0.036332017 | 0.017206191 | 0.00860529 | 0.009542037 |
| T]A | | | | | | | | |
| T[C > | 0.006146389 | 0 | 0.010567698 | 0.000992741 | 0.000133195 | 0.017440739 | 0.007849932 | 0.019835102 |
| T]C | | | | | | | | |
| T[C > | 0.06030262 | 0.009416019 | 0.002488485 | 0.000299818 | 0.002072144 | 0.003676961 | 0.009876758 | 0.005196765 |
| T]G | | | | | | | | |
| T[C > | 0.010963363 | 0 | 0.009561569 | 0.002809155 | 0.000139907 | 0.024834625 | 0.010547239 | 0.013064588 |
| T]T | | | | | | | | |
| T[T > | 0.008107416 | 0.021057472 | 0.00699048 | 0.35385827 | 0.002244746 | 0.008078692 | 0.020389347 | 0.010189004 |
| A]A | | | | | | | | |
| T[T > | 0.003761589 | 0 | 0.003220092 | 0.000897623 | 0.000226416 | 0.002902344 | 0.010559976 | 0.004266688 |
| A]C | | | | | | | | |
| T[T > | 0.004861838 | 0.004064773 | 0.010123294 | 0.00060724 | 0.000740724 | 0.006804833 | 0.010386399 | 0.004318307 |
| A]G | | | | | | | | |
| T[T > | 0.005236625 | 0.07463216 | 0.006365322 | 0.163909572 | 0.000505798 | 0.006069531 | 0.031081454 | 0.017402136 |
| A]T | | | | | | | | |
| T[T > | 0.009565727 | 0.003983846 | 0.007411779 | 0.01002599 | 0.000311951 | 0.034116132 | 0.093089366 | 0.008778842 |
| C]A | | | | | | | | |
| T[T > | 0.007233879 | 0.002349209 | 0.00260158 | 0.000456686 | 0.00017969 | 0.004434578 | 0.013531951 | 0.004953162 |
| C]C | | | | | | | | |
| T[T > | 0.001709731 | 0 | 0.008742964 | 0.000140133 | 0.000150067 | 0.013037538 | 0.011029689 | 0.002117696 |
| C]G | | | | | | | | |
| T[T > | 0.006557527 | 0.096616828 | 0.006805536 | 0.004030444 | 0.001267283 | 0.014346834 | 0.014123111 | 0.01684277 |
| C]T | | | | | | | | |
| T[T > | 0.008033781 | 0.030163406 | 0.006769399 | 0.018419133 | 0.005827902 | 0.002181572 | 0.052960657 | 0.004023837 |
| G]A | | | | | | | | |
| T[T > | 0.002523456 | 0.021142879 | 0.002980271 | 0.000188973 | 0.000144874 | 0.00030641 | 0.013518171 | 0.001259883 |
| G]C | | | | | | | | |
| T[T > | 0.00199417 | 0 | 0.005067641 | 0.000295835 | 0.00062736 | 0.001756618 | 0.012791136 | 0.003073752 |
| G]G | | | | | | | | |
| T[T > | 0.006222075 | 0.102402266 | 0.005703019 | 0.0083934 | 0.001003551 | 0.001801876 | 0.055121485 | 0.003348646 |
| G]T | | | | | | | | |

### REFERENCES

1. J. Vijg, X. Dong, Pathogenic Mechanisms of Somatic Mutation and Genome Mosaicism in Aging. Cell 182, 12-23 (2020).
2. B. N. Ames, F. D. Lee, W. E. Durston, An improved bacterial test system for the detection and classification of mutagens and carcinogens. Proc Natl Acad Sci USA 70, 782-786 (1973).
3. J. McCann, E. Choi, E. Yamasaki, B. N. Ames, Detection of carcinogens as mutagens in the Salmonella/microsome test: assay of 300 chemicals. Proc Natl Acad Sci USA 72, 5135-5139 (1975).
4. R. J. Albertini, K. L. Castle, W. R. Borcherding, T-cell cloning to detect the mutant 6-thioguanine-resistant lymphocytes present in human peripheral blood. Proc Natl Acad Sci USA 79, 6617-6621 (1982).
5. J. Vijg, H. van Steeg, Transgenic assays for mutations and cancer: current status and future perspectives. Mutat Res 400, 337-354 (1998).
6. M. Gundry, W. Li, S. B. Maqbool, J. Vijg, Direct, genome-wide assessment of DNA mutations in single cells. Nucleic Acids Res 40, 2032-2040 (2012).
7. X. Dong et al., Accurate identification of single-nucleotide variants in whole-genome-amplified single cells. Nat Methods 14, 491-493 (2017).
8. M. W. Schmitt et al., Detection of ultra-rare mutations by next-generation sequencing. Proc Natl Acad Sci USA 109, 14508-14513 (2012).
9. A. Y. Maslov, W. Quispe-Tintaya, T. Gorbacheva, R. R. White, J. Vijg, High-throughput sequencing in mutation detection: A new generation of genotoxicity tests? Mutat Res 776, 136-143 (2015).
10. C. C. Valentine, 3rd et al., Direct quantification of in vivo mutagenesis and carcinogenesis using duplex sequencing. Proc Natl Acad Sci USA 117, 33414-33425 (2020).
11. K. B. Ignatov et al., A strong strand displacement activity of thermostable DNA polymerase markedly improves the results of DNA amplification. Biotechniques 57, 81-87 (2014).
12. F. Abascal et al., Somatic mutation landscapes at single-molecule resolution. Nature, (2021).
13. C. W. Op het Veld, S. van Hees-Stuivenberg, A. A. van Zeeland, J. G. Jansen, Effect of nucleotide excision repair on hprt gene mutations in rodent cells exposed to DNA ethylating agents. Mutagenesis 12, 417-424 (1997).
14. K. Brazhnik et al., Single-cell analysis reveals different age-related somatic mutation profiles between stem and differentiated cells in human liver. Sci Adv 6, eaax2659-eaax2659 (2020).
15. F. Blokzijl, R. Janssen, R. van Boxtel, E. Cuppen, MutationalPatterns: comprehensive genome-wide analysis of mutational processes. Genome Med 10, 33 (2018).
16. L. B. Alexandrov et al., Signatures of mutational processes in human cancer. Nature 500, 415-421 (2013).
17. M. Petljak et al., Characterizing Mutational Signatures in Human Cancer Cell Lines Reveals Episodic APOBEC Mutagenesis. Cell 176, 1282-1294 e1220 (2019).
18. M. L. Hoang et al., Genome-wide quantification of rare somatic mutations in normal human tissues using massively parallel sequencing. Proc Natl Acad Sci USA 113, 9846-9851 (2016).
19. W. Quispe-Tintaya et al., Quantitative detection of low-abundance somatic structural variants in normal cells by high-throughput sequencing. Nat Methods, (2016).
20. W. Quispe-Tintaya et al., Bleomycin-induced genome structural variations in normal, non-tumor cells. Sci Rep 8, 16523 (2018).
21. Spielmann, M., D. G. Lupianez and S. Mundlos (2018). “Structural variation in the 3D genome.” Nat Rev Genet 19(7): 453-467.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A single-stranded nucleic acid molecule comprising:
(a) a hairpin structure, wherein the hairpin comprises:
(i) a blunt end or an overhang;
(ii) a unique molecular identifier (UMI) in a hairpin stem; and
(iii) at least one priming site for polymerase chain reaction (PCR) and/or rolling circle-based linear amplification (RCA), preferably in a hairpin loop;
(b) two PCR priming sites and an RCA priming site;
(c) the sequence of TCTTC TACAGT NNNNNN AGATCG GAAGAG CACACG TCTGAA CTCCAG TC/at least one deoxyuridine (deoxyU)/ACACTC TTTCCC TACACG ACGCTC TTCCGA TCT, wherein N is any nucleotide, and/or wherein the at least one deoxyU comprises at least one Int deoxyuridine (ideoxyU) (SEQ ID NO: 3); and/or
(d) at least one genomic DNA fragment.

2. (canceled)

3. The single-stranded nucleic acid molecule of claim 1, wherein:
(a) the hairpin loop comprises:
(i) at least 1, 2, or 3 uracils; or
(ii) at least 1, 2, or 3 uracils that are not present in one or more PCR priming sites; and/or
(b) the hairpin loop is:
(i) at least 3-nucleotide-long; and/or
(ii) no more than 3000-nucleotide-long.

4. The single-stranded nucleic acid molecule of claim 1, wherein the two PCR priming sites do not overlap.

5. The single-stranded nucleic acid molecule claim 1, wherein (a) the RCA priming site overlaps with at least one PCR priming site; or (b) the RCA priming site overlaps with two PCR priming sites.

6. The single-stranded nucleic acid molecule of claim 1, wherein:
(a) the overhang is a 3′ overhang; or
(b) the overhang comprises at least one thymidine or at least one uracil; or
(c) the overhang comprises at least 1, 2, 3, 4, 5, 6, 7, 8, 9, 10, 11, 12, 13, 14, 15, 16, 17, 18, 19, or 20 nucleotides; or
(d) the overhang consists of one thymidine; or
(e) the overhang comprises at least 1, 2, or 3 uracils.

7-8. (canceled)

9. The single-stranded nucleic acid molecule of claim 1, wherein the UMI comprises at least 4, 5, 6, 7, 8, 9, 10, 11, 12, 13, 14, 15, 16, 17, 18, 19, or 20 nucleotides, preferably at least 6 nucleotides.

10. (canceled)

11. The single-stranded nucleic acid molecule of claim 1, wherein the hairpin stem is:
(a) at least 3-nucleotide-long; and/or
(b) no more than 3000-nucleotide-long.

12-13. (canceled)

14. A method of:
a) preparing a genomic DNA library, the method comprising:
(i) preparing genomic DNA fragments that are ligated at both ends to the hairpin structure formed by the single-stranded nucleic acid molecule of claim 1;
(ii) preparing single-stranded DNA (ssDNA) concatemers by performing a pulse-RCA on the genomic DNA fragments generated in step (a), wherein the pulse-RCA comprises at least one cycle of denaturation-annealing-extension by a DNA polymerase; and
(iii) preparing double-stranded DNA comprising the genomic DNA fragments by performing a PCR reaction on the ssDNA concatemers;
or
b) preparing a genomic DNA library for detecting a genome Structural Variant (SV), the method comprising:
(iv) tagmenting genomic DNA using Tn5-mediated transposition reaction with transposon comprising a uracil residue 5′ to the Tn5 Mosaic End (ME);
(v) extending to fill a 9-nucleotide gap created by Tn5 and reconstituting the same strand;
(vi) digesting using Uracil-DNA Glycosylase (UDG) to release the uracil residue and expose a 3′ overhang;
(vii) ligating the genomic DNA fragments generated by steps (d)-(f) to the single-stranded nucleic acid molecule of claim 1;
(viii) preparing single-stranded DNA (ssDNA) concatemers by performing a pulse-RCA on the genomic DNA fragments generated in step (d), wherein the pulse-RCA comprises at least one cycle of denaturation-annealing-extension by a DNA polymerase; and
(ix) preparing double-stranded DNA comprising the genomic DNA fragments by performing a PCR reaction on the ssDNA concatemers.

15. The method of claim 14, wherein the preparation of genomic DNA fragments of step (i) comprises:
(a) creating the genomic DNA fragments by digestion with at least one endonuclease or by sonication;
(b) repairing the ends of the genomic DNA fragments, phosphorylating 5′ end, and/or dA tailing; and/or
(c) ligating the genomic DNA fragments to the single-stranded nucleic acid molecule of claim 1.

16. The method of claim 14, wherein the at least one endonuclease comprises an endonuclease that creates a blunt end and/or an endonuclease that creates an overhang.

17. (canceled)

18. The method of claim 14, wherein the DNA polymerase for the pulse-RCA and/or PCR reaction is strong strand displacement or a high-fidelity DNA polymerase.

19. The method of claim 14, wherein:
(a) the pulse-RCA comprises at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 cycles of the denaturation-annealing-extension by a DNA polymerase;
(b) the PCR reaction comprises at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 cycles of the PCR reaction.

20. (canceled)

21. The method of claim 14, wherein
(a) the pulse-RCA comprises at least 1 cycle of denaturation-annealing-extension by a DNA polymerase; and/or
(b) the PCR reaction comprises at least or about 6 cycles.

22. A genomic DNA library prepared by the method according to claim 14.

23. A method of detecting at least one mutation or at least one structural variant (SV) in a cell or a plurality of cells, or of detecting the DNA damage profile of a subject afflicted with a cancer, the method comprising:
(a) obtaining the cell or the plurality of cells;
(b) preparing a library comprising the genomic DNA fragments of the cell or the plurality of cells according to the method of claim 14; and
(c) sequencing the library;
or
(d) aligning one or more UMIs corresponding to sequencing reads of the DNA fragments; and/or
(e) analyzing the sequences according to the computational algorithm shown in FIG. 1B and/or using the computing node shown in FIG. 4.

24-25. (canceled)

26. The method of claim 23, wherein:
a) the cell is a primary cell of a subject or a cell from an immortalized cell line;
b) the method detects a Catalogue of Somatic Mutations in Cancer (COSMIC) signature;
c) the method is performed in vivo, in vitro, or ex vivo; and/or
d) the library is sequenced by Next-Generation Sequencing (NGS) or Deep Sequencing.

27. (canceled)

28. The method of claim 23, wherein the at least one mutation comprises a single nucleotide variant (SNV), a deletion of one or more nucleotides, a insertion of one or more nucleotides, a duplication of one or more nucleotides, a substitution of one or more nucleotides, a point mutation, a translocation, a copy number variation, a loss of heterozygosity, a retrotransposon, or any combination thereof.

29. The method of claim 23, wherein the at least one SV comprises:
(a) a deletion, inversion, insertion, duplication, translocation, or any combination thereof; and/or
(b) at least or about 40, 41, 42, 43, 44, 45, 46, 47, 48, 49, 50, 51, 52, 53, 54, 55, 56, 57, 58, 59, or 60 basepairs of a genome.

30. (canceled)

31. The method of claim 23, wherein:
(a) the at least one mutation or at least one SV comprises at least, about, or no more than 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 mutations or SVs;
(b) the at least one mutation is a somatic mutation or a germline mutation;
(c) the at least one mutation or at least one SV is induced by a chemical agent and/or a radioactive agent; and/or
(d) the at least one mutation or at least one SV is related to aging.

32-36. (canceled)

37. The method of claim 23, wherein the subject:
(a) is healthy or diseased;
(b) has been exposed to a chemical agent and/or a radioactive agent;
(c) is old or young; and/or
(d) received a chemotherapy or a radiation therapy.

38-40. (canceled)

41. A method of diagnosing a disease risk and/or a disease in a subject, the method comprising:
(a) detecting at least one mutation or at least one SV in the subject according to the method of claim 1; and
(b) diagnosing the subject as having a disease risk or a disease, if the at least one mutation or at least one SV identified in (a) is associated with said disease.

42. The method of claim 41, wherein the disease is:
a) a cancer; or
b) a disease other than a cancer.

43. The method of claim 42, wherein:
a) the cancer is selected from: sarcomas, carcinomas, leukemias, chronic leukemia polycythemia vera, lymphoma, multiple myeloma, Waldenstrom's macroglobulinemia, heavy chain disease, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, lung cancer, skin cancer, nonpapillary renal cell carcinoma, cervical carcinoma, and ovarian carcinoma; or
(b) the disease other than cancer is
i) selected from a neurological disease, a hematological disease, an autoimmune disease, or
ii) selected from Alzheimer's disease, a neurodegenerative disease, a psychiatric disorder, schizophrenia, myelodysplastic syndrome, Neurofibromatosis 1, Cockayne syndrome, xeroderma pigmentosum, Alport syndrome, epilepsy, an autism spectrum disorder, Rett syndrome, intellectual disability, hemimegalencephaly, Lissencephaly, mental retardation, spasticity, and autoimmune lymphoproliferative syndrome.

44-45. (canceled)

46. The method of claim 41, wherein:
a) the at least one mutation comprises a COSMIC signature; and/or
b) wherein the disease and/or the at least one mutation is selected from those listed in Table 1.

47. (canceled)

48. A method of:
(a) testing mutagenicity of an agent comprising:
(i) exposing a cell to the agent;
(ii) detecting at least one mutation or at least one SV in the cell exposed to the agent using the method according to claim 23; and
(iii) comparing the number and/or types of mutations identified in (ii) cells to a control,
wherein the number and/or types of mutations in (ii) cells relative to the control indicates the mutagenicity of the chemical or radioactive compound;
(b) testing in vivo mutagenicity of an agent comprising:
(i) exposing an animal to the agent;
(ii) obtaining a cell from the animal exposed to the agent;
(iii) detecting at least one mutation or at least one SV in the cell from (b), using the method according to claim 23; and
(iv) comparing the number and/or types of the at least one mutation or the at least one SV identified in (iii) and a control,
wherein the number and/or types of the at least one mutation or the at least one SV in (iii) relative to the control indicate the mutagenicity of the agent;
or
(c) determining a subject's exposure to a biohazard material comprising:
(i) obtaining a cell from the subject exposed to the biohazard material;
(ii) detecting at least one mutation or at least one SV in the cell from (a), using the method according claim 23;
(iii) comparing the number and/or type of the at least one mutation or the at least one SV identified in (ii) and a control
wherein the number and/or type of the at least one mutation or the at least one SV in (ii) relative to the control indicates the subject's exposure to the biohazard material.

49. The method of claim 48, wherein:
(a) the cell is:
(i) a primary cell of a subject or a cell from an immortalized cell line; or
(ii) a mammalian cell or a human cell; or
(b) the animal is a mouse, rat, guinea pig, dog, chicken, monkey, or cat; or
(c) the subject is a mammal, a mouse, rat, guinea pig, dog, cat, monkey, or human.

50. The method of claim 48, wherein:
(a) the control is the number and/or type of mutations identified in a cell that is not exposed to the agent, preferably wherein the control cell is of the same cell type as the cell that is exposed to the agent;
(b) the control is the number and/or type of a mutation identified in the cell of an animal that is not exposed to the agent;
(c) the control is:
(i) from an animal of the same species as the animal that is exposed to the agent; and/or
(ii) the same cell type as the cell of the animal exposed to the agent;
(d) the control is the number and/or type of at least one mutation or at least one SV identified in a cell of a subject who is not exposed to the biohazard material; or
(e) the control is:
(i) from a subject of the same species as the subject that is exposed to the biohazard material; and/or
(ii) the control is the same cell type as the cell of the subject exposed to the agent.

51-60. (canceled)

61. A kit comprising the single-stranded nucleic acid molecule of claim 1 and/or the genomic library of claim 22.

62. A method for identifying one or more single nucleotide mutations, the method comprising:
receiving a plurality of sequencing reads of a DNA fragment, wherein the plurality of sequencing reads of the DNA fragment comprise first and second strand families, each strand family including reads uniquely associated with the respective strand;
receiving a unique molecular identifier (UMI), the UMI corresponding to the sequencing reads of the DNA fragment, wherein the plurality of sequencing reads of the DNA fragment correspond to a UMI family;
identifying the one or more single nucleotide mutations in the plurality of sequencing reads when:
each sequencing read corresponds to a paired read with a mapping quality score greater than or equal to a predetermined score;
a length of each strand family is greater than or equal to a predetermined length;
one or more variants are determined from the plurality of sequencing reads relative to a reference genome, wherein a predetermined amount of the plurality of sequencing reads correspond to the one or more variants;
the one or more variants are not known variants;
the one or more variants are located within a predetermined number of nucleotides from an end of the plurality of sequencing reads; and
the one or more variants are not found in other UMI families.

63. The method of claim 62, wherein:
a) the predetermined score is 60;
b) the predetermined length is 7;
c) the predetermined amount is 100%;
d) the predetermined number of nucleotides is 5;
e) known variants comprise germline variants and variants from a known variant database; or
f) the known variant database comprises dbSNP.

64-68. (canceled)

69. A computer program product for distributed order processing, the computer program product comprising a computer readable storage medium having program instructions embodied therewith, the program instructions executable by a processor to cause the processor to perform a method comprising:
receiving a plurality of sequencing reads of a DNA fragment, wherein the plurality of sequencing reads of the DNA fragment comprise first and second strand families, each strand family including reads uniquely associated with the respective strand;
receiving a unique molecular identifier (UMI), the UMI corresponding to the sequencing reads of the DNA fragment, wherein the plurality of sequencing reads of the DNA fragment correspond to a UMI family;
identifying the one or more single nucleotide mutations in the plurality of sequencing reads when:
each sequencing read corresponds to a paired read with a mapping quality score greater than or equal to a predetermined score;
a length of each strand family is greater than or equal to a predetermined length;
one or more variants are determined from the plurality of sequencing reads relative to a reference genome, wherein a predetermined amount of the plurality of sequencing reads correspond to the one or more variants;
the one or more variants are not known variants;
the one or more variants are located within a predetermined number of nucleotides from an end of the plurality of sequencing reads; and
the one or more variants are not found in other UMI families.
